(19) United States
(12) Patent Application Publication
(10) Pub. No.: US 2020/0016136 A1
(43) Pub. Date: Jan. 16, 2020

(54) PYRIDIN-3-YL ACETIC ACID DERIVATIVES AS INHIBITORS OF HUMAN IMMUNODEFICIENCY VIRUS REPLICATION

(71) Applicant: VIIV HEALTHCARE UK (NO. 5) LIMITED, Brentford, Middlesex (GB)

(72) Inventors: Michael S. BOWSHER, Wallingford, CT (US); Jeffrey DESKUS, Wallingford, CT (US); Kyle J. EASTMAN, Wallingford, CT (US); Eric P. GILLIS, Branford, CT (US); David B. FRENNESSON, Wallingford, CT (US); Christiana IWUAGWU, Wallingford, CT (US); B. Narasimhulu NAIDU, Branford, CT (US); Kyle E. PARCELLA, Branford, CT (US); Kevin M. PEESE, Branford, CT (US); Mark G. SAULNIER, Wallingford, CT (US); Prasanna SIVAPRAKASAM, Wallingford, CT (US)

(21) Appl. No.: 16/465,622

(22) PCT Filed: Jan. 2, 2018
(86) PCT No.: PCT/IB2018/050022

§ 371 Date: May 31, 2019

Related U.S. Application Data
(60) Provisional application No. 62/441,614, filed on Jan. 3, 2017.

(51) Int. Cl. A61K 31/444; C07D 401/14; C07D 413/14; C07D 491/048; C07D 491/08; A61K 31/5377; A61K 31/506; A61K 31/519; A61K 31/497; A61K 31/5386; A61K 31/4995; C07D 471/08; C07D 417/14; A61K 31/541; C07D 491/147; A61P 31/18

(52) U.S. Cl. CPC A61K 31/444; A61P 31/18; C07D 413/14; C07D 491/048; C07D 491/08; A61K 31/5377; A61K 31/506; A61K 31/519; A61K 31/497; A61K 31/5386; A61K 31/4995; C07D 471/08; C07D 417/14; A61K 31/541; C07D 491/147; C07D 401/14

(57) ABSTRACT

Disclosed are compounds of Formula I, including pharmaceutically acceptable salts, pharmaceutical compositions comprising the compounds, methods for making the compounds and their use in inhibiting HIV integrase and treating those infected with HIV or AIDS.

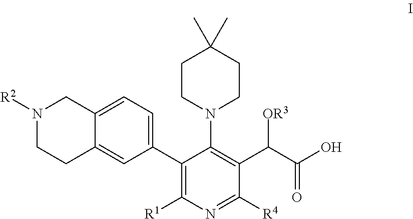

### FIELD OF THE INVENTION

The invention relates to compounds, compositions, and methods for the treatment of human immunodeficiency virus (HIV) infection. More particularly, the invention provides novel inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Human immunodeficiency virus (HIV) has been identified as the etiological agent responsible for acquired immune deficiency syndrome (AIDS), a fatal disease characterized by destruction of the immune system and the inability to fight off life threatening opportunistic infections. Recent statistics indicate that an estimated 35.3 million people worldwide are infected with the virus (UNAIDS: Report on the Global HIV/AIDS Epidemic, 2013). In addition to the large number of individuals already infected, the virus continues to spread. Estimates from 2013 point to close to 3.4 million new infections in that year alone. In the same year there were approximately 1.6 million deaths associated with HIV and AIDS.

Current therapy for HIV-infected individuals consists of a combination of approved anti-retroviral agents. Over two dozen drugs are currently approved for HIV infection, either as single agents or as fixed dose combinations or single tablet regimens, the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase inhibitors (INIs), or entry inhibitors (one, maraviroc, targets the host CCR5 protein, while the other, enfuvirtide, is a peptide that targets the gp41 region of the viral gp160 protein). In addition, a pharmacokinetic enhancer with no antiviral activity, i.e., cobicistat, available from Gilead Sciences, Inc. under the tradename TYBOST™ (cobicistat) tablets, has recently been approved for use in combinations with certain antiretroviral agents (ARVs) that may benefit from boosting.

In the US, where combination therapy is widely available, the number of HIV-related deaths has dramatically declined (Palella, F. J.; Delany, K. M.; Moorman, A. C.; Loveless, M. O.; Furher, J.; Satten, G. A.; Aschman, D. J.; Holmberg, S. D. N. Engl. J. Med. 1998, 338, 853-860).

Unfortunately, not all patients are responsive and a large number fail this therapy. In fact, initial studies suggest that approximately 30-50% of patients ultimately fail at least one drug in the suppressive combination. Treatment failure in most cases is caused by the emergence of viral resistance. Viral resistance in turn is caused by the replication rate of HIV-1 during the course of infection combined with the relatively high viral mutation rate associated with the viral polymerase and the lack of adherence of HIV-infected individuals in taking their prescribed medications. Clearly, there is a need for new antiviral agents, preferably with activity against viruses already resistant to currently approved drugs. Other important factors include improved safety and a more convenient dosing regimen than many of the currently approved drugs.

Compounds which inhibit HIV replication have been disclosed. See, for example, the following patent applications: WO2007131350, WO2009062285, WO2009062288, WO2009062289, WO2009062308, WO2010130034, WO2010130842, WO2011015641, WO2011076765, WO2012033735, WO2013123148, WO2013134113, WO2014164467, WO2014159959, and WO2015126726.

What is now needed in the art are additional compounds which are novel and useful in the treatment of HIV. Additionally, these compounds may desireably provide advantages for pharmaceutical uses, for example, with regard to one or more of their mechanisms of action, binding, inhibition efficacy, target selectivity, solubility, safety profiles, or bioavailability. Also needed are new formulations and methods of treatment which utilize these compounds.

### SUMMARY OF THE INVENTION

Briefly, in one aspect, the present invention discloses compounds of Formula I

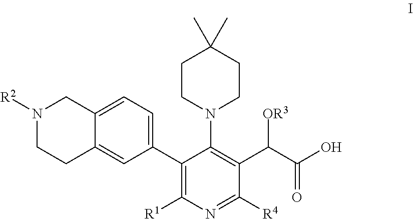

or a pharmaceutically acceptable salt thereof wherein:
R¹ is hydrogen, C₁₋₆alkyl, Ar¹, carboxy, cyano, hydroxy, C₁₋₆haloalkyl, —C₁₋₆alkyl-OH, —N(R⁵)(R⁶), —C(O)N(R⁷)(R⁸), or (R⁹)(R¹⁰)NC₁₋₆alkyl-;
R² is Ar³—C₁₋₆alkyl-, or Ar⁴;
R³ is C₁₋₆alkyl;
R⁴ is hydrogen, C₁₋₆alkyl, cyano, halo, C₁₋₆haloalkyl, or —C₁₋₆alkyl-OH;
R⁵ is hydrogen or C₁₋₆alkyl;
R⁶ is hydrogen, C₁₋₆alkyl, C₁₋₆alkyl-O—C₁₋₆alkyl, C₃₋₆cycloalkyl, C₃₋₆cycloalkyl-C₁₋₆alkyl-, 1-(C₁₋₆alkyl)piperidinyl-, (C₁₋₆alkyl)₂N—C₁₋₆alkyl-, (tetrahydropyranyl)C₁₋₆alkyl-, morpholinoC₁₋₆alkyl-, piperidinylC₁₋₆alkyl-, 1-(C₁₋₆alkyl)piperazinylC₁₋₆alkyl-, Ar²—C₁₋₆alkyl-, or 1-(C₁₋₆alkylsulfonyl)piperidinyl-;
R⁷ is hydrogen or C₁₋₆alkyl;
R⁸ is hydrogen, C₁₋₆alkyl, C₃₋₆cycloalkyl, or C₁₋₆alkyl-C₃₋₆cycloalkyl-;
R⁹ is hydrogen or C₁₋₆alkyl;
R¹⁰ is hydrogen, C₁₋₆alkyl, Ar³—C₁₋₆alkyl-, or (tetrahydropyranyl) C₁₋₆alkyl-;
R¹¹ is azaspirononanyl, azetidinyl, 1,4-diazabicyclo[3.2.2]nonanyl, 3,8-diazabicyclo[3.2.1]octanyl, 3,7-dioxa-9-azabicyclo[3.3.1]nonanyl, 1,1-dioxidothiomorpholinyl, imidazolyl, morpholinyl, 3-oxa-9-azabicyclo[3.3.1]nonanyl, 8-oxa-3-azabicyclo[3.2.1]octanyl, oxadiazolyl, phenyl, piperazinyl, piperidinyl, pyrazinyl, pyridinyl, pyrrolidinyl, ((R⁵)(R⁹)NC₁₋₆alkyl)(R⁵)N—, (R⁵)(R⁹)N—, and is substituted with 0-3 substituents selected from C₁₋₆alkyl, —O—C₁₋₆alkyl, halo, C₁₋₆haloalky, —C₁₋₆alkyl-OH, morpholinyl, piperazinyl, or piperidinyl;
(R⁷)(R⁸)N taken together form an azetidinyl, pyrrolidinyl, piperidinyl, 1,1-dioxidothiomorpholinyl, or morpholinyl and is substituted with 0-3 C₁₋₆alkyl substituents;
(R⁹)(R¹⁰)N taken together form an azetidinyl, pyrrolidinyl, piperidinyl, or azaspirononanyl, and is substituted with 0-3 C₁₋₆alkyl substitutents;
Ar¹ is selected from imidazolyl, pyrazolyl, pyridinyl, pyrimidinyl, pyrrolyl and is substituted with 0-3 substitutents selected from amino, C₁₋₆alkyl, and C₃₋₆cycloalkyl;
Ar² is selected from imidazolyl, pyrazolyl, pyridinyl, pyrimidinyl, and pyrrolyl, substituted with 0-3 C₁₋₆alkyl and halo substitutents;
Ar³ is phenyl, and is substituted with 0-3 substituents selected from C₁₋₆alkyl, —O—C₁₋₆alkyl, cyano, halo, or C₁₋₆haloalkyl; and
Ar⁴ is selected from benzofuropyrimidinyl, pyrazinyl, pyridazinyl, pyridinyl, pyridofuropyrimidinyl, pyrimidinyl, pyrrolotriazinyl, triazinyl and is substituted with 0-3 substituents selected from R¹¹, C₁₋₆alkyl, —O—C₁₋₆alkyl, —CO₂H, cyano, halo, C₁₋₆haloalkyl,
or hydroxy;
and wherein each reference to “haloalkyl includes all halogenated isomers from monohalo to perhalo.

The invention also provides a compound of Formula (I) or a pharmaceutically acceptable salt thereof for use in therapy.

The invention also provides a compound of Formula (I) or a pharmaceutically acceptable salt thereof for use in the treatment of HIV infection

The invention also provides the use of a compound of Formula (I) or a pharmaceutically acceptable salt thereof in the manufacture of a medicament for the treatment of HIV infection.

The invention also provides pharmaceutical compositions comprising a compound or salt of the invention.

In addition, the invention provides methods of treating HIV infection comprising administering a compound or salt of the invention to a patient.

In addition, the invention provides methods for inhibiting HIV integrase.

Also provided in accordance with the invention are methods for making the compounds and salts of the invention.

### DESCRIPTION OF THE INVENTION

Preferably, R¹ is hydrogen, C₁₋₆alkyl, C₁₋₆haloalkyl, —C₁₋₆alkyl-OH, —N(R⁵)(R⁶), or (R⁹)(R¹⁰)NC₁₋₆alkyl-, wherein R⁵, R⁶, R⁹, and R¹⁰ are as defined above. More preferably, R¹ is hydrogen or (R⁹)(R¹⁰)NC₁₋₆alkyl-, wherein R⁵, R⁶, R⁹, and R¹⁰ are as defined above.

Preferably, R² is Ar³—C₁₋₆alkyl- wherein Ar³ is as defined above; or Ar⁴ wherein Ar⁴ is selected from benzofuropyrimidinyl, pyrazinyl, pyridinyl, pyridofuropyrimidinyl, or pyrimidinyl, and is substituted with 0-3 substituents selected from R¹¹, C₁₋₆alkyl, —O—C₁₋₆alkyl, —CO₂H, cyano, halo, C₁₋₆haloalkyl, or hydroxy wherein R¹¹ is as defined above. More preferably, R² is Ar³—C₁₋₆alkyl- wherein Ar³ is as defined above; or Ar⁴ wherein Ar⁴ is selected from benzofuropyrimidinyl, pyridinyl, or pyridofuropyrimidinyl, and is substituted with 0-3 substituents selected from R¹¹, C₁₋₆alkyl, —O—C₁₋₆alkyl, —CO₂H, cyano, halo, C₁₋₆haloalkyl, or hydroxy wherein R¹¹ is as defined above.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms. The invention includes all stereoisomeric forms of the compounds including enantiomers and diastereromers. Methods of making and separating stereoisomers are known in the art. The invention includes all tautomeric forms of the compounds. The invention includes atropisomers and rotational isomers.

The invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

In one embodiment, a method for treating or preventing an HIV infection in a human having or at risk of having the infection is provided, comprising administering to the human a therapeutically effective amount of a compound disclosed herein, or a pharmaceutically acceptable salt thereof, in combination with a therapeutically effective amount of one or more additional therapeutic agents.

In one embodiment, pharmaceutical compositions comprising a compound disclosed herein, or a pharmaceutically acceptable salt thereof, in combination with one or more additional therapeutic agents, and a pharmaceutically acceptable carrier, diluent or excipient are provided.

In one embodiment, combination pharmaceutical agents comprising a compound disclosed herein, or a pharmaceutically acceptable salt thereof, in combination with one or more additional therapeutic agents are provided.

In the above embodiments, the additional therapeutic agent may be an anti-HIV agent. For example, in some embodiments, the additional therapeutic agent is selected from the group consisting of HIV protease inhibitors, HIV non-nucleoside inhibitors of reverse transcriptase, HIV nucleoside inhibitors of reverse transcriptase, HIV maturation inhibitors, HIV integrase inhibitors, HIV non-catalytic site (or allosteric) integrase inhibitors, entry inhibitors (e.g., CCR5 inhibitors, gp41 inhibitors (i.e., fusion inhibitors) and CD4 attachment inhibitors), CXCR4 inhibitors, gp120 inhibitors, G6PD and NADH-oxidase inhibitors, compounds that target the HIV capsid (“capsid inhibitors”; e.g., capsid polymerization inhibitors or capsid disrupting compounds such as those disclosed in WO 2013/006738 (Gilead Sciences), US 2013/0165489 (University of Pennsylvania), and WO 2013/006792 (Pharma Resources), pharmacokinetic enhancers, and other drugs for treating HIV, and combinations thereof.

In further embodiments, the additional therapeutic agent is selected from one or more of:

(1) HIV protease inhibitors selected from the group consisting of amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, GW640385X, DG17, PPL-100, DG35, and AG 1859;
(2) HIV non-nucleoside or non-nucleotide inhibitors of reverse transcriptase selected from the group consisting of capravirine, emivirine, delaviridine, efavirenz, nevirapine, (+) calanolide A, etravirine, GW5634, DPC-083, DPC-961, DPC-963, MIV-150, TMC-120, rilpivirene, BILR 355 BS, VRX 840773, lersivirine (UK-453061), RDEA806, KM023 and MK-1439;
(3) HIV nucleoside inhibitors of reverse transcriptase selected from the group consisting of zidovudine, emtricitabine, didanosine, stavudine, zalcitabine, lamivudine, abacavir, amdoxovir, elvucitabine, alovudine, MIV-210, .+−.−FTC, D-d4FC, emtricitabine, phosphazide, fozivudine tidoxil, apricitibine (AVX754), KP-1461, GS-9131 (Gilead Sciences) and fosalvudine tidoxil (formerly HDP 99.0003);
(4) HIV nucleotide inhibitors of reverse transcriptase selected from the group consisting oftenofovir, tenofovir disoproxil fumarate, tenofovir alafenamide fumarate (Gilead Sciences), GS-7340 (Gilead Sciences), GS-9148 (Gilead Sciences), adefovir, adefovir dipivoxil, CMX-001 (Chimerix) or CMX-157 (Chimerix);
(5) HIV integrase inhibitors selected from the group consisting of curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, AR-177, L-870812, and L-870810, raltegravir, BMS-538158, GSK364735C, BMS-707035, NMK-2048, BA 011, elvitegravir, dolutegravir, bictegravir and GSK-744; (6) HIV non-catalytic site, or allosteric, integrase inhibitors (NCINI) including, but not limited to, BI-224436, CX0516, CX05045, CX14442, compounds disclosed in WO 2009/062285 (Boehringer Ingelheim), WO 2010/130034 (Boehringer Ingelheim), WO 2013/159064 (Gilead Sciences), WO 2012/145728 (Gilead Sciences), WO 2012/003497 (Gilead Sciences), WO 2012/003498 (Gilead Sciences) each of which is incorporated by references in its entirety herein;
(7) gp41 inhibitors selected from the group consisting of enfuvirtide, sifuvirtide, albuvirtide, FB006M, and TRI-1144;
(8) the CXCR4 inhibitor AMD-070;
(9) the entry inhibitor SP01A;
(10) the gp120 inhibitor BMS-488043;
(11) the G6PD and NADH-oxidase inhibitor immunitin;
(12) CCR5 inhibitors selected from the group consisting of aplaviroc, vicriviroc, maraviroc, cenicriviroc, PRO-140, INCB 115050, PF-232798 (Pfizer), and CCR5 mAb004;
(13) CD4 attachment inhibitors selected from the group consisting of ibalizumab (TMB-355) and BMS-068 (BMS-663068);
(14) pharmacokinetic enhancers selected from the group consisting of cobicistat and SPI-452; and
(15) other drugs for treating HIV selected from the group consisting of BAS-100, SPI-452, REP 9, SP-01A, TNX-355, DES6, ODN-93, ODN-112, VGV-1, PA-457 (bevirimat), HRG214, VGX-410, KD-247, AMZ 0026, CYT 99007A-221 HIV, DEBIO-025, BAY 50-4798, MDX010 (ipilimumab), PBS 119, ALG 889, and PA-1050040 (PA-040),
and combinations thereof.

Unless specified otherwise, these terms have the following meanings.

“Combination,” “coadministration,” “concurrent” and similar terms referring to the administration of a compound of Formula I with at least one anti-HIV agent mean that the components are part of a combination antiretroviral therapy or highly active antiretroviral therapy (“HAART”) as understood by practitioners in the field of AIDS and HIV infection.

“Therapeutically effective” means the amount of agent required to provide a benefit to a patient as understood by practitioners in the field of AIDS and HIV infection. In general, the goals of treatment are suppression of viral load, restoration and preservation of immunologic function, improved quality of life, and reduction of HIV-related morbidity and mortality.

“Patient” means a person infected with the HIV virus.

“Treatment,” “therapy,” “regimen,” “HIV infection,” “ARC,” “AIDS” and related terms are used as understood by practitioners in the field of AIDS and HIV infection.

Those terms not specifically set forth herein shall have the meaning which is commonly understood and accepted in the art.

Solid compositions which are normally formulated in dosage units and compositions providing from about 1 to 1000 milligram (“mg”) of the active ingredient per dose are typical. Some examples of dosages are 1 mg, 10 mg, 100 mg, 250 mg, 500 mg, and 1000 mg. Generally, other antiretroviral agents will be present in a unit range similar to agents of that class used clinically. Typically, this is about 0.25-1000 mg/unit.

Liquid compositions are usually in dosage unit ranges. Generally, the liquid composition will be in a unit dosage range of about 1-100 milligram per milliliter (“mg/mL”). Some examples of dosages are 1 mg/mL, 10 mg/mL, 25 mg/mL, 50 mg/mL, and 100 mg/mL. Generally, other antiretroviral agents will be present in a unit range similar to agents of that class used clinically. Typically, this is about 1-100 mg/mL.

The invention encompasses all conventional modes of administration; oral and parenteral methods are preferred. Generally, the dosing regimen will be similar to other antiretroviral agents used clinically. Typically, the daily dose will be about 1-100 milligram per kilogram (“mg/kg”) body weight daily. Generally, more compound is required orally and less parenterally. The specific dosing regimen, however, will be determined by a physician using sound medical judgment.

### Methods of Synthesis

The compounds of this invention can be made by various methods known in the art including those of the following schemes and in the specific embodiments section. The structure numbering and variable numbering shown in the synthetic schemes are distinct from, and should not be confused with, the structure or variable numbering in the claims or the rest of the specification. The variables in the schemes are meant only to illustrate how to make some of the compounds of this invention. The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

Abbreviations used in the schemes and examples generally follow conventions used in the art. Chemical abbreviations used in the specification and examples are defined as follows: “KHMDS” for potassium bis(trimethylsilyl)amide; “DMF” for N,N-dimethylformamide; “HATU” for O-(t-Azabenzotriazol-1-yl)-N,N,N′,N′-tetramethyluronium hexafluorophosphate, “MeOH” for methanol; “Ar” for aryl; “TFA” for trifluoroacetic acid, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HOBt” for 1-hydroxybenzotriazole hydrate; and “DIEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “atm” for atmosphere, “psi” for pounds per square inch, “conc.” for concentrate, “sat” or “sat'd” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

Some compounds can be synthesized form an appropriately substituted heterocycle I-1 according to Scheme I, Compounds I-1 and I-6 are commercially available or synthesized by reactions well known in the art. Treatment of compound I-1 with bromine provided the dibromo intermediates I-2 which was converted to the chloropyridine I-3 by reacting with POCl₃. Intermediate I-3 conveniently transformed to ketoester I-5 using conditions well-known to those skilled in the art, including reacting I-3 with Grignard reagent in the presence of catalytic copper(I) bromide dimethylsulfide complex followed by alkyl 2-chloro-2-oxoacetate. Coupling of amines 1-5 with intermediate 1-6 in the presence of an organic base such as Hunig's base provided intermediate 1-7. Chiral Lewis acid such as I-8 mediated reduction of ketoester I-7 with catecholborane furnished chiral alcohol I-9. Tertiary butylation of alcohol I-9 by well-known conditions, including but not limited to tertiary-butyl acetate and perchloric acid, gave intermediate 1-10. Intermediates 1-10 are conveniently transformed to intermediates I-11 using conditions well-known in the art, including but not limited to the Suzuki coupling between intermediates I-10 and R⁶B(OR)₂. The boronate or boronic acid coupling reagents, well-known in the art, are commercially available or are prepared by reactions well-known to those skilled in the art. Hydrolysis of intermediate I-11 by using conditions well-known to those skilled in the art furnished carboxylic acid 1-12.

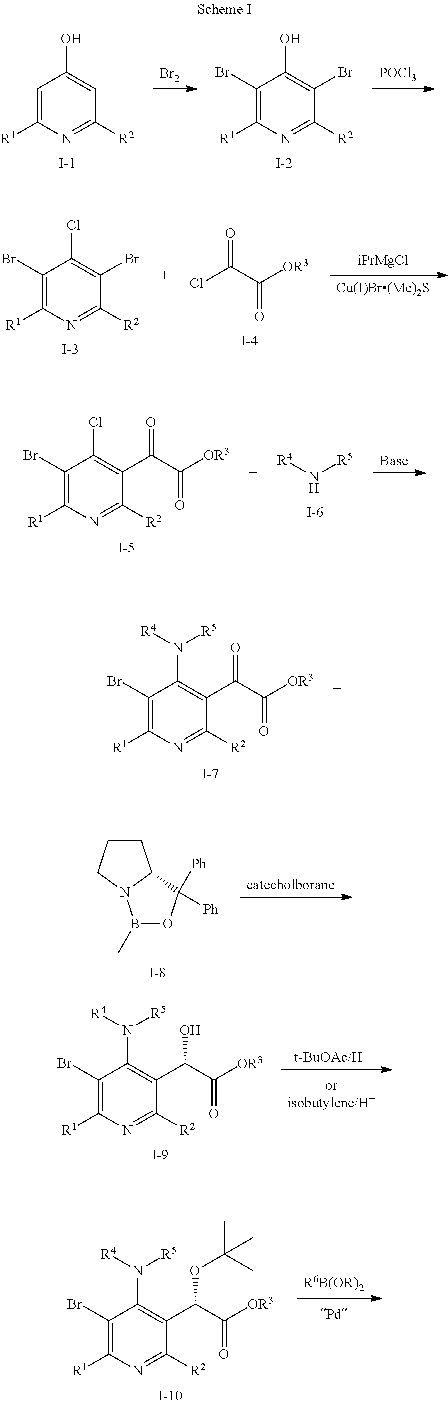

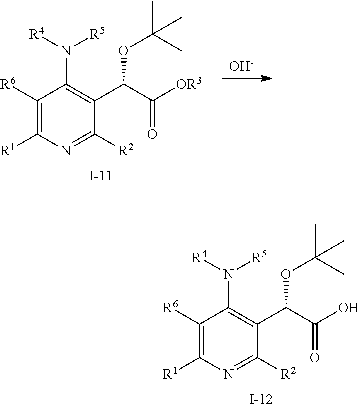

In yet another method, some compounds of this invention can be synthesized according to Scheme II.

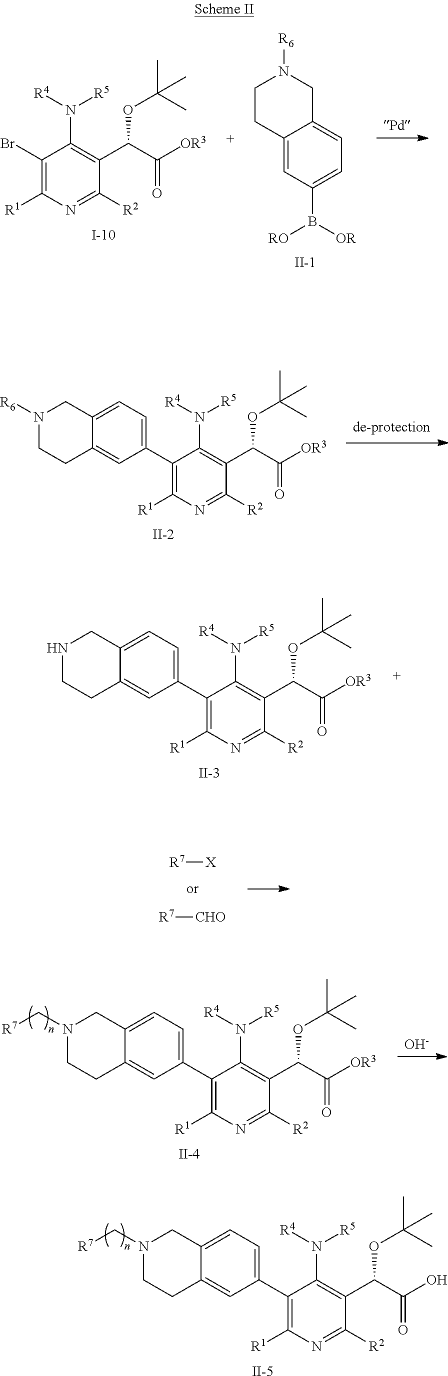

In yet another method, some compounds of this invention can be synthesized according to Scheme III. Intermediate can be transformed to the final products by several paths. In one, the C2 and C6 alkyl groups can be oxidized to furnish intermediates III-2 and/or III-3 which can be further transformed to final compounds III-8 or III-9 by several paths.

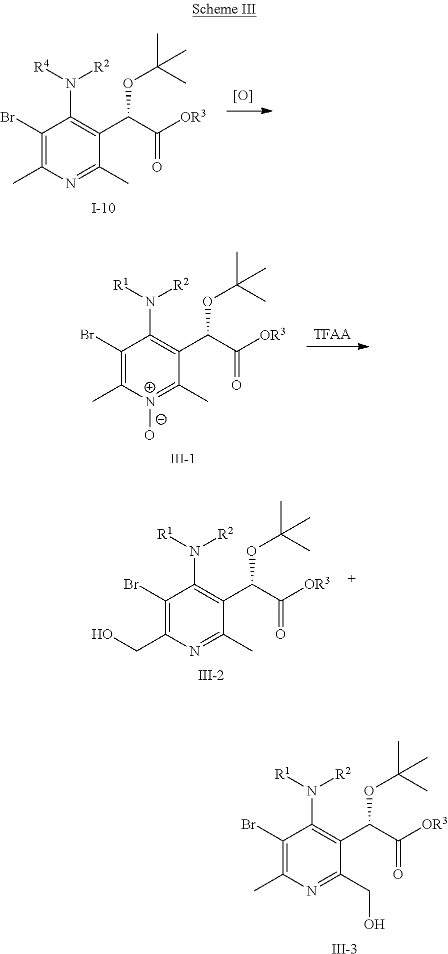

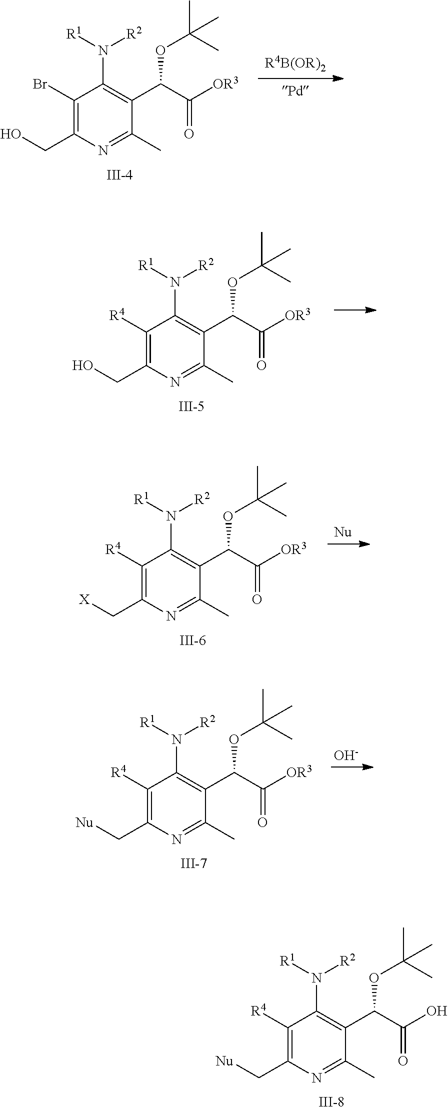

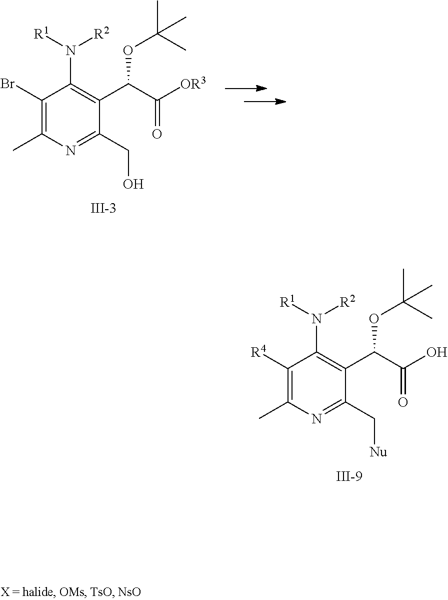

The compounds described herein were purified by the methods well known to those skilled in art by normal phase column chromatography on silica gel column using appropriate solvent system described. Preparative HPLC purifications mentioned in this experimentation section were carried out gradient elution either on Sunfire Prep C18 ODB column (5 μm; 19 or 30×100 mm) or Waters Xbridge C18 column (5 μM; 19×200 or 30×100 mm) or Water Atlantis (5 μm; 19 or 30×100 mm) using the following mobile phases. Mobile phase A: 9:1 H₂O/acetonitrile with 10 mM NH₄OAc and mobile phase B:A:9:1 acetonitrile/H₂O with 10 mM NH₄OAc; or mobile phase A: 9:1 H₂O/acetonitrile with 0.1% TFA and mobile phase B:A: 9:1 acetonitrile/H₂O with 0.1% TFA; or mobile phase A: water/MeOH (9:1) with 20 mM NH₄OAc and mobile phase B: 95:5 MeOH/H₂O with 20 mM NH₄OAc or mobile phase A: water/MeOH (9:1) with 0.1% TFA and mobile phase B: 95:5 MeOH/H₂O with 0.1% TFA or mobile Phase A: 5:95 acetonitrile: water with 10-mM ammonium acetate; Mobile Phase B: 95:5 acetonitrile: water with 10-mM ammonium acetate.

All Liquid Chromatography (LC) data were recorded on a Shimadzu LC-10AS or LC-20AS liquid chromotograph using a SPD-10AV or SPD-20A UV-Vis detector and Mass Spectrometry (MS) data were determined with a Micromass Platform for LC in electrospray mode.

Compounds purified by preparative HPLC were diluted in methanol (1.2 mL) or DMF and purified using a Shimadzu LC-8A or LC-10A automated preparative HPLC system.

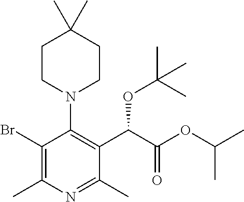 [SMILES: Cc1nc(C)c([C@H](OC(C)(C)C)C(=O)OC(C)C)c(N2CCC(C)(C)CC2)c1Br]

### (S)-Isopropyl 2-(5-bromo-4-(4,4-dimethylpiperidin-1-yl)-2,6-dimethylpyridin-3-yl)-2-(tert-butoxy)acetate

Prepared according to the procedure described in WO2015126726.

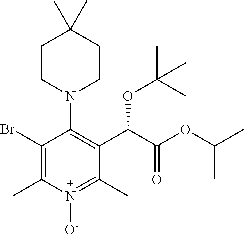 [SMILES: Cc1c(Br)c(N2CCC(C)(C)CC2)c([C@H](OC(C)(C)C)C(=O)OC(C)C)c(C)[n+]1[O-]]

### (S)-3-Bromo-5-(1-(tert-butoxy)-2-isopropoxy-2-oxoethyl)-4-(4,4-dimethylpiperidin-1-yl)-2,6-dimethylpyridine 1-oxide

To a stirred solution of (S)-isopropyl 2-(5-bromo-4-(4,4-dimethylpiperidin-1-yl)-2,6-dimethylpyridin-3-yl)-2-(tert-butoxy)acetate (15 g, 32.0 mmol) in DCM (150 mL) was added 77% mCPBA (10.74 g, 47.9 mmol) at rt over 5 min. After 4 h, the reaction mixture was washed with 1M NaOH (2×100 mL), dried (MgSO₄), filtered and concentrated to give (S)-3-bromo-5-(1-(tert-butoxy)-2-isopropoxy-2-oxoethyl)-4-(4,4-dimethylpiperidin-1-yl)-2,6-dimethylpyridine 1-oxide (15.3 g, 31.5 mmol, 99% yield) which was used in the next step without purification. LCMS (M+1)=485.1 and 487.1.

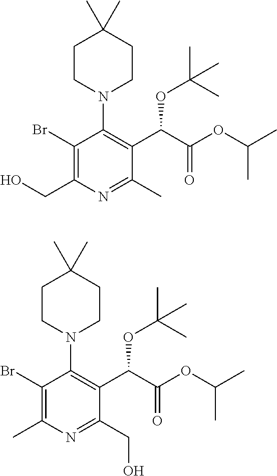

### (S)-Isopropyl 2-(5-bromo-4-(4,4-dimethylpiperidin-1-yl)-6-(hydroxymethyl)-2-methylpyridin-3-yl)-2-(tert-butoxy)acetate and (S)-isopropyl 2-(5-bromo-4-(4,4-dimethylpiperidin-1-yl)-2-(hydroxymethyl)-6-methylpyridin-3-yl)-2-(tert-butoxy)acetate

To a stirred solution of (S)-3-bromo-5-(1-(tert-butoxy)-2-isopropoxy-2-oxoethyl)-4-(4,4-dimethylpiperidin-1-yl)-2,6-dimethylpyridine 1-oxide (5.24 g, 10.79 mmol) anhydrous DCM (50 ml) was added trifluoroacetic anhydride (3.05 ml, 21.59 mmol) at RT. After 3 h, sat NaHCO₃ (50 mL) was added and stirred vigorously for 10 minutes. The solution phases were separated and organic phase collected and volatiles evaporated. The residue was taken up in EtOAc and washed with 50 mL of 1 M HCl followed by a wash with sat. sodium bicarbonate. The organic layer was then washed with brine, dried over MgSO₄, filtered and volatiles evaporated to afford the crude product. The crude product was purified via silica gel (120 g column, 5-20% EtOAc:Hex) to give two products:

### (S)-Isopropyl 2-(5-bromo-4-(4,4-dimethylpiperidin-1-yl)-6-(hydroxymethyl)-2-methylpyridin-3-yl)-2-(tert-butoxy)acetate

Viscous colorless oil which turns into white solid over the time, 4.0 g (76%). ¹H NMR (500 MHz, CDCl₃) δ 6.25 (br. s., 1H), 5.06 (spt, J=6.3 Hz, 1H), 4.75-4.79 (m, 1H), 4.74-4.62 (m, 2H), 4.02-4.12 (br. s., 1H), 3.54-3.46 (m, 1H), 2.93 (d, J=11.5 Hz, 1H), 2.70-2.63 (m, 1H), 2.61 (s, 3H), 1.65-1.56 (m, 2H), 1.50-1.43 (m, 1H), 1.35-1.40 (m, 1H), 1.23 (d, J=6.2 Hz, 3H), (1.22 (s, 9H), 1.16 (d, J=6.3 Hz, 3H), 1.09 (s, 3H), 1.05 (s, 3H). LCMS (M+H)=485.35 and 487.2.

### (S)-Isopropyl 2-(5-bromo-4-(4,4-dimethylpiperidin-1-yl)-2-(hydroxymethyl)-6-methylpyridin-3-yl)-2-(tert-butoxy)acetate

Clear viscous oil, 0.430 g (8.2%). ¹H NMR (500 MHz, CDCl₃) δ 6.21 (br. s., 1H), 5.03 (spt, J=6.3 Hz, 1H), 4.95 (d, J=15.1 Hz, 1H), 4.64 (dd, J=15.3, 5.0 Hz, 1H), 4.50 (br. s., 1H), 4.05-3.97 (m, 1H), 3.57 (td, J=12.1, 2.5 Hz, 1H), 2.84 (d, J=11.8 Hz, 1H), 2.69 (s, 3H), 2.62 (d, J=11.8 Hz, 1H), 1.66-1.55 (m, 2H), 1.47 (dd, J=13.2, 2.0 Hz, 1H), 1.40-1.34 (m, 1H), 1.23 (d, J=6.3 Hz, 3H), 1.22 (s, 9H), 1.16 (d, J=6.1 Hz, 3H), 1.09 (s, 3H), 1.05 (s, 3H). LCMS (M+H)=485.2 and 487.05.

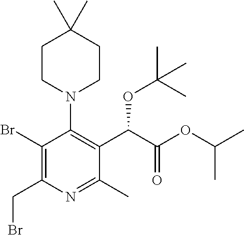 [SMILES: Cc1nc(CBr)c(Br)c(N2CCC(C)(C)CC2)c1[C@H](OC(C)(C)C)C(=O)OC(C)C]

### (S)-Isopropyl 2-(5-bromo-6-(bromomethyl)-4-(4,4-dimethylpiperidin-1-yl)-2-methylpyridin-3-yl)-2-(tert-butoxy)acetate

To a solution of (S)-isopropyl 2-(5-bromo-4-(4,4-dimethylpiperidin-1-yl)-6-(hydroxymethyl)-2-methylpyridin-3-yl)-2-(tert-butoxy)acetate (500 mg, 1.030 mmol) in CH₂Cl₂ (10 mL) was added CBr₄ (376 mg, 1.133 mmol) followed by Ph₃P (297 mg, 1.133 mmol) and the resulting mixture was stirred at room temp for 16 h. Water (2 mL) was then added and the mixture was extracted with dichloromethane (10 mL), dried (Na₂SO₄), filtered and concentrated. The residue was then purified by Biotage (5-30% EtOAc/hexane) to afford (S)-isopropyl 2-(5-bromo-6-(bromomethyl)-4-(4,4-dimethylpiperidin-1-yl)-2-methylpyridin-3-yl)-2-(tert-butoxy)acetate (350 mg, 0.638 mmol, 62.0% yield) as white solid. ¹H NMR (500 MHz, CDCl₃) δ 6.25 (br. s., 1H), 5.14-4.94 (m, 1H), 4.76 (d, J=9.6 Hz, 1H), 4.69 (d, J=9.6 Hz, 1H), 4.04 (br. s., 1H), 3.51 (t, J=11.9 Hz, 1H), 2.91 (d, J=11.5 Hz, 1H), 2.66 (d, J=12.1 Hz, 1H), 2.58 (s, 3H), 1.68-1.55 (m, 2H), 1.47 (d, J=12.5 Hz, 1H), 1.37 (d, J=12.8 Hz, 1H), 1.26-1.23 (m, 3H), 1.22 (s, 9H), 1.16 (d, J=6.1 Hz, 3H), 1.09 (s, 3H), 1.04 (s, 3H). LCMS (M+2H)=549.2.

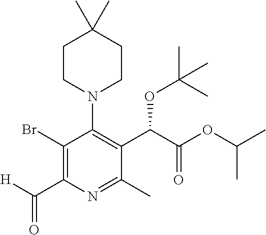 [SMILES: Cc1nc(C=O)c(Br)c(N2CCC(C)(C)CC2)c1[C@H](OC(C)(C)C)C(=O)OC(C)C]

### (S)-Isopropyl 2-(5-bromo-4-(4,4-dimethylpiperidin-1-yl)-6-formyl-2-methylpyridin-3-yl)-2-(tert-butoxy)acetate

To a stirred solution of (S)-isopropyl 2-(5-bromo-4-(4,4-dimethylpiperidin-1-yl)-6-(hydroxymethyl)-2-methylpyridin-3-yl)-2-(tert-butoxy)acetate (15.4 g, 31.7 mmol) in DCM (288 ml) and acetonitrile (28.8 ml) was added Dess-Martin Periodinane (16.15 g, 38.1 mmol) at once at rt. After 5 h, the reaction mixture was diluted with ether (250 mL), washed with 1M NaOH (2×100 ml), brine (100 mL), dried (MgSO₄), filtered and concentrated to afford (S)-isopropyl 2-(5-bromo-4-(4,4-dimethylpiperidin-1-yl)-6-formyl-2-methylpyridin-3-yl)-2-(tert-butoxy)acetate (15.3 g, 31.6 mmol, 99% yield) as yellow solid. ¹H NMR (500 MHz, CDCl₃) δ 10.30 (s, 1H), 6.27 (br s, 1H), 5.13-5.03 (m, 1H), 4.12 (br s, 1H), 3.61-3.52 (m, 1H), 2.96 (br d, J=9.8 Hz, 1H), 2.76-2.70 (m, 1H), 2.66 (s, 3H), 1.66-1.54 (m, 4H), 1.23-1.21 (m, 12H), 1.17 (d, J=6.1 Hz, 3H), 1.11 (s, 3H), 1.06 (s, 3H). LCMS (M+1)=483.1 and 485.1.

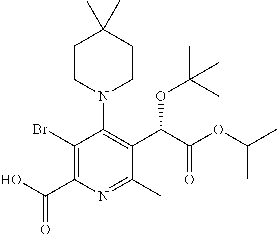 [SMILES: Cc1nc(C(=O)O)c(Br)c(N2CCC(C)(C)CC2)c1[C@H](OC(C)(C)C)C(=O)OC(C)C]

### (S)-3-Bromo-5-(1-(tert-butoxy)-2-isopropoxy-2-oxoethyl)-4-(4,4-dimethylpiperidin-1-yl)-6-methylpicolinic Acid

To a solution of (S)-isopropyl 2-(5-bromo-4-(4,4-dimethylpiperidin-1-yl)-6-formyl-2-methylpyridin-3-yl)-2-(tert-butoxy)acetate (15.3 g, 31.6 mmol) in acetonitrile (127 ml) and water (31.6 ml) was added oxone (14.79 g, 24.05 mmol) and the mixture was stirred at RT for 1 hr. The reaction was diluted with water and EtOAc. The organic layer was washed with water (2×) and brine, dried (MgSO₄), filtered and concentrated to afford the product (S)-3-bromo-5-(1-(tert-butoxy)-2-isopropoxy-2-oxoethyl)-4-(4,4-dimethylpiperidin-1-yl)-6-methylpicolinic acid (15.6 g, 31.2 mmol, 99% yield) as a yellow crispy foam. ¹H NMR (500 MHz, CDCl₃) δ 6.33-6.17 (m, 1H), 5.09 (dt, J=12.5, 6.3 Hz, 1H), 4.26-4.14 (m, 1H), 3.67-3.49 (m, 1H), 3.03-2.83 (m, 1H), 2.77-2.65 (m, 1H), 2.62 (s, 3H), 1.64-1.35 (m, 4H), 1.24 (d, J=6.3 Hz, 3H), 1.22 (s, 9H), 1.18 (d, J=6.1 Hz, 3H), 1.10-1.06 (m, 6H). LCMS (M+1)=499.1 and 501.1.

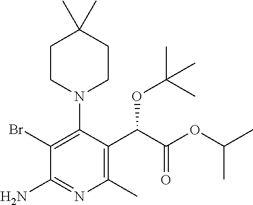 [SMILES: Cc1nc(N)c(Br)c(N2CCC(C)(C)CC2)c1[C@H](OC(C)(C)C)C(=O)OC(C)C]

### A0FB6-379

### Isopropyl (S)-2-(6-amino-5-bromo-4-(4,4-dimethylpiperidin-1-yl)-2-methylpyridin-3-yl)-2-(tert-butoxy)acetate

To a solution of ((S)-3-bromo-5-(1-(tert-butoxy)-2-isopropoxy-2-oxoethyl)-4-(4,4-dimethylpiperidin-1-yl)-6-methylpicolinic acid (6.5 g, 13.01 mmol) and triethylamine (3.63 ml, 26.0 mmol) in toluene (130 mL) was added water (1.172 ml, 65.1 mmol) followed by diphenyl phosphorazidate (5.78 ml, 26.0 mmol). The resulting mixture was heated at 90° C. for 2 h. The reaction mixture was then cooled to ambient temperature, diluted with EtOAc (200 mL), and washed with saturated aqueous NaHCO₃, water, and brine. The organic layer was dried (Na₂SO₄), filtered, and concentrated in vacuo. The residue was then purified on silica gel (220 g column) using 5-80% EtOAc/hexane. The desired fractions were concentrated in vacuo to afford (S)-isopropyl 2-(6-amino-5-bromo-4-(4,4-dimethylpiperidin-1-yl)-2-methylpyridin-3-yl)-2-(tert-butoxy)acetate (5.6 g, 91%) as an off white solid. ¹H NMR (500 MHz, CDCl₃) δ 5.57 (s, 1H), 4.90 (spt, J=6.2 Hz, 1H), 3.04 (dd, J=12.9, 3.0 Hz, 1H), 3.04 (dd, J=12.9, 3.0 Hz, 1H), 3.04 (ddd, J=12.9, 11.7, 3.0 Hz, 1H), 3.04 (ddd, J=12.9, 11.7, 3.0 Hz, 1H), 2.57 (s, 3H), 1.45 (ddd, J=14.2, 3.0, 2.7 Hz, 1H), 1.45 (ddd, J=14.2, 3.0, 2.7 Hz, 1H), 1.45 (ddd, J=14.2, 11.7, 3.0 Hz, 1H), 1.45 (ddd, J=14.2, 11.7, 3.0 Hz, 1H), 1.10 (s, 9H), 1.09 (d, J=6.2 Hz, 6H), 0.98 (s, 3H), 0.91 (s, 3H). LCMS (M+1)=470.10.

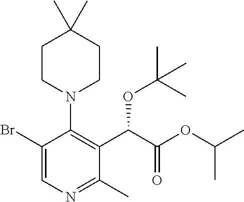 [SMILES: Cc1ncc(Br)c(N2CCC(C)(C)CC2)c1[C@H](OC(C)(C)C)C(=O)OC(C)C]

### (S)-Isopropyl 2-(5-bromo-4-(4,4-dimethylpiperidin-1-yl)-2-methylpyridin-3-yl)-2-(tert-butoxy)acetate

Water (2.81 ml, 156 mmol) followed by acetic acid (4.65 ml, 81 mmol) was added to a stirring solution of (S)-3-bromo-5-(1-(tert-butoxy)-2-isopropoxy-2-oxoethyl)-4-(4,4-dimethylpiperidin-1-yl)-6-methylpicolinic acid (15.6 g, 31.2 mmol) in toluene (156 ml) at rt. The reaction was stirred at 90° C. for 7 hrs. The reaction volatiles were evaporated and the crude material purified via silica gel (330 g column, 5-20% EtOAc:Hex) to afford the product (S)-isopropyl 2-(5-bromo-4-(4,4-dimethylpiperidin-1-yl)-2-methylpyridin-3-yl)-2-(tert-butoxy)acetate (12.8 g, 28.1 mmol, 90% yield) as a clear oil that later crystallized. ¹H NMR (500 MHz, CDCl₃) δ 8.46 (s, 1H), 6.35-6.17 (m, 1H), 5.06 (dt, J=12.5, 6.2 Hz, 1H), 4.10-3.96 (m, 1H), 3.45 (br s, 1H), 2.92 (br s, 1H), 2.72-2.62 (m, 1H), 2.57 (s, 3H), 1.58-1.28 (m, 4H), 1.22 (s, 12H), 1.15 (d, J=6.3 Hz, 3H), 1.07 (br s, 6H). LCMS (M+1)=455.3 and 457.3.

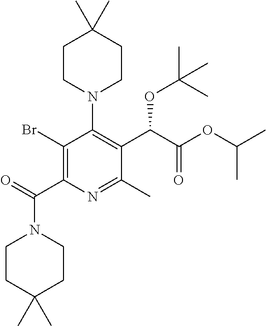 [SMILES: Cc1nc(C(=O)N2CCC(C)(C)CC2)c(Br)c(N2CCC(C)(C)CC2)c1[C@H](OC(C)(C)C)C(=O)OC(C)C]

### (S)-Isopropyl 2-(5-bromo-4-(4,4-dimethylpiperidin-1-yl)-6-(4,4-dimethylpiperidine-1-carbonyl)-2-methylpyridin-3-yl)-2-(tert-butoxy)acetate

To a solution of (S)-3-bromo-5-(1-(tert-butoxy)-2-isopropoxy-2-oxoethyl)-4-(4,4-dimethylpiperidin-1-yl)-6-methylpicolinic acid (40 mg, 0.080 mmol), Hunig's Base (0.031 mL, 0.176 mmol), and 4,4-dimethylpiperidine (9.97 mg, 0.088 mmol) in DMF (1 mL) was added HATU (33.5 mg, 0.088 mmol) and stirred at RT for 1 hr. The reaction was diluted with water and extracted with EtOAc. The organic layer was washed with brine, collected, dried over MgSO₄, filtered and volatiles evaporated to afford the crude product. The crude product was purified via silica gel (4 g column, 10-60% EtOAc:Hex) to afford the product (S)-isopropyl 2-(5-bromo-4-(4,4-dimethylpiperidin-1-yl)-6-(4,4-dimethylpiperidine-1-carbonyl)-2-methylpyridin-3-yl)-2-(tert-butoxy)acetate (38 mg, 0.064 mmol, 80% yield) as a clear oil. LCMS (M+1)=594.2 and 596.2.

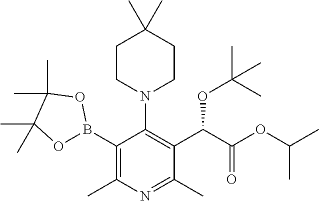 [SMILES: Cc1nc(C)c([C@H](OC(C)(C)C)C(=O)OC(C)C)c(N2CCC(C)(C)CC2)c1B1OC(C)(C)C(C)(C)O1]

### Isopropyl (S)-2-(tert-butoxy)-2-(4-(4,4-dimethylpiperidin-1-yl)-2,6-dimethyl-5-(4,4,5,5-tetramethyl-1,3,2-dioxaborolan-2-yl)pyridin-3-yl)acetate

To a microwave vial under nitrogen was added isopropyl-(S)-2-(5-bromo-4-(4,4-dimethylpiperidin-1-yl)-2,6-dimethylpyridin-3-yl)-2-(tert-butoxy)acetate (739 mg, 1.574 mmol), 4,4,5,5-tetramethyl-1,3,2-dioxaborolane (640 μl, 4.41 mmol), degassed dioxane (12 mL), and degassed triethylamine (1.05 mL, 7.53 mmol). The reaction was flushed with argon, treated with [1,1′-bis(diphenylphosphino)ferrocene]dichloropalladium(II) (63 mg, 0.086 mmol), sealed, stirred at room temp for 10 min then placed in microwave reactor at 100° C. for 36 h. Additional 4,4,5,5-tetramethyl-1,3,2-dioxaborolane (300 ulit, 3.53 mmol)), triethylamine (500 ulit, 3.59 mmol), and [1,1′-bis(diphenylphosphino)ferrocene]dichloropalladium(II) (29 mg, 0.04 mmol) were added and the reaction was placed in a 115° C. oil bath for 48 h. The crude reaction was diluted with ethyl acetate, filtered through a small pad of celite, concentrated and purified via silica gel chromatography (80 g SiO₂ column, dichloromethane:EtOAc 100:0->25:75) to afford isopropyl (S)-2-(tert-butoxy)-2-(4-(4,4-dimethylpiperidin-1-yl)-2,6-dimethyl-5-(4,4,5,5-tetramethyl-1,3,2-dioxaborolan-2-yl)pyridin-3-yl)acetate, 218 mg (25%). LCMS (M+1)=517.4. H NMR (500 MHz, CDCl₃) δ 5.98 (br s, 1H), 5.08 (dt, J=12.5, 6.3 Hz, 1H), 3.45-3.12 (m, 2H), 3.02-2.71 (m, 2H), 2.58 (s, 3H), 2.52 (s, 3H), 1.62 (br s, 4H), 1.48 (s, 6H), 1.42 (s, 6H), 1.21-1.13 (m, 15H), 1.05 (br s, 6H).

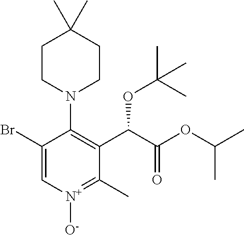 [SMILES: Cc1c([C@H](OC(C)(C)C)C(=O)OC(C)C)c(N2CCC(C)(C)CC2)c(Br)c[n+]1[O-]]

### (S)-5-Bromo-3-(1-(tert-butoxy)-2-isopropoxy-2-oxoethyl)-4-(4,4-dimethylpiperidin-1-yl)-2-methylpyridine 1-oxide

To a stirred solution of (S)-isopropyl 2-(5-bromo-4-(4,4-dimethylpiperidin-1-yl)-2-methylpyridin-3-yl)-2-(tert-butoxy)acetate (0.9 g, 1.976 mmol) in DCM (19.76 ml) was added 77% mCPBA (0.664 g, 2.96 mmol) at rt over 5 min. After 1 h, the reaction mixture was diluted with DCM and washed with sat. Na₂CO₃ (3×25 mL), dried (MgSO₄), filtered and concentrated to afford (S)-5-bromo-3-(1-(tert-butoxy)-2-isopropoxy-2-oxoethyl)-4-(4,4-dimethylpiperidin-1-yl)-2-methylpyridine 1-oxide (873 mg, 1.852 mmol, 94% yield) which was used in the next step without purification. LCMS (M+1)=471.1 and 473.1.

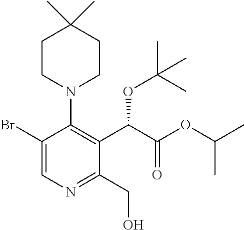 [SMILES: CC(C)OC(=O)[C@@H](OC(C)(C)C)c1c(CO)ncc(Br)c1N1CCC(C)(C)CC1]

### (S)-Isopropyl 2-(5-bromo-4-(4,4-dimethylpiperidin-1-yl)-2-(hydroxymethyl)pyridin-3-yl)-2-(tert-butoxy)acetate

To a stirred solution of (S)-5-bromo-3-(1-(tert-butoxy)-2-isopropoxy-2-oxoethyl)-4-(4,4-dimethylpiperidin-1-yl)-2-methylpyridine 1-oxide (0.873 g, 1.852 mmol) anhydrous DCM (18.52 ml) was added trifluoroacetic anhydride (0.785 ml, 5.56 mmol) at RT. After 3 h, sat. sodium carbonate (50 mL) was added and stirred vigorously for 10 mintues. The solution phases were separated and organic phase collected and volatiles evaporated. The residue was taken up in EtOAc and washed with 50 mL of 1 M HCl followed by a wash with sat. sodium carbonate. The organic layer was then washed with 2 M aq. sodium carbonate (50 mL) for 18 hrs. The crude product was purified via silica gel (40 g column, 5-30% EtOAc:Hex) to afford the product (S)-isopropyl 2-(5-bromo-4-(4,4-dimethylpiperidin-1-yl)-2-(hydroxymethyl)pyridin-3-yl)-2-(tert-butoxy)acetate (720 mg, 1.527 mmol, 82% yield). ¹H NMR (500 MHz, CDCl₃) δ 8.56 (s, 1H), 6.30-6.17 (m, 1H), 5.04 (dt, J=12.5, 6.3 Hz, 1H), 4.97 (d, J=15.3 Hz, 1H), 4.66 (d, J=15.3 Hz, 1H), 4.04-3.94 (m, 1H), 3.58-3.45 (m, 1H), 2.88 (br d, J=3.6 Hz, 1H), 2.71-2.59 (m, 1H), 1.57-1.36 (m, 4H), 1.25-1.22 (m, 12H), 1.16 (d, J=6.3 Hz, 3H), 1.07 (br d, J=12.0 Hz, 6H). LCMS (M+1)=471.1 and 473.1.

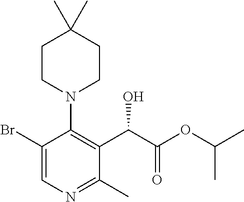 [SMILES: Cc1ncc(Br)c(N2CCC(C)(C)CC2)c1[C@H](O)C(=O)OC(C)C]

### (S)-Isopropyl 2-(5-bromo-4-(4,4-dimethylpiperidin-1-yl)-2-methylpyridin-3-yl)-2-hydroxyacetate

(S)-Isopropyl 2-(5-bromo-4-(4,4-dimethylpiperidin-1-yl)-2-methylpyridin-3-yl)-2-(tert-butoxy)acetate (500 mg, 0.621 mmol) was taken up in a 2:1 solution of TFA/DCE (0.15 M). The reaction was stirred for 2 days and then the mixture was diluted with EtOAc and washed with sat Na₂CO₃. The organic phase was dried over Na₂SO₄, filtered and concentrated, adsorbed onto celite and was purified on silica gel (Biotage, EtOAc/hexanes gradient, 0-100% over 10 CVs) to give the expected product (S)-isopropyl 2-(5-bromo-4-(4,4-dimethylpiperidin-1-yl)-2-methylpyridin-3-yl)-2-hydroxyacetate (450 mg, quant yield). LCMS (M+H)=399 and 401.

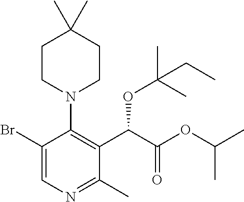 [SMILES: CCC(C)(C)O[C@H](C(=O)OC(C)C)c1c(C)ncc(Br)c1N1CCC(C)(C)CC1]

### (S)-Isopropyl 2-(5-bromo-4-(4,4-dimethylpiperidin-1-yl)-2-methylpyridin-3-yl)-2-(tert-pentyloxy)acetate

In a 20 mL microwave vial, (S)-isopropyl 2-(5-bromo-4-(4,4-dimethylpiperidin-1-yl)-2-methylpyridin-3-yl)-2-hydroxyacetate (440 mg, 1.10 mmol), CH₂Cl₂ (9.1 ml), and 2-methylbut-2-ene (3.7 ml, 44 mmol) were combined. Then, the vial was capped and perchloric acid (0.284 ml, 3.31 mmol) was added at once to the reaction (see ppt form and then go back into a clear solution). The reaction was stirred for 4 hrs. The mixture was diluted with EtOAc and washed with sat Na₂CO₃. The organic phase was dried over Na₂SO₄, filtered and concentrated, adsorbed onto celite and was purified on silica gel (Biotage, EtOAc/hexanes gradient, 0-100% over 10 CVs) to give 112 mg of (S)-isopropyl 2-(5-bromo-4-(4,4-dimethylpiperidin-1-yl)-2-methylpyridin-3-yl)-2-(tert-pentyloxy)acetate (21% yield). LCMS (M+H)=469, and 471. Also 297 mg of starting material was recovered.

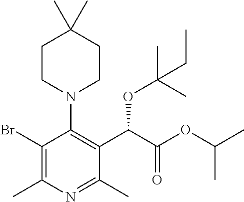 [SMILES: CCC(C)(C)O[C@H](C(=O)OC(C)C)c1c(C)nc(C)c(Br)c1N1CCC(C)(C)CC1]

### Isopropyl (S)-2-(5-bromo-4-(4,4-dimethylpiperidin-1-yl)-2,6-dimethylpyridin-3-yl)-2-(tert-pentyloxy)acetate

To a dried pressure bottle (15 mL) under nitrogen was added isopropyl-(S)-2-(5-bromo-4-(4,4-dimethylpiperidin-1-yl)-2,6-dimethylpyridin-3-yl)-2-hydroxyacetate (490 mg, 1.185 mmol; for preparation see WO2015126726), 4 A activated molecular sieves, CH₂Cl₂ (10 mL), and nitromethane (5 mL). The resulting solution was then treated with 2-methylbut-2-ene (3 mL, 25.4 mmol), flushed with nitrogen, capped and allowed to stir at room temp for 25 min. The reaction was then slowly treated (over 1 min) with a solution of triflic acid (110 μL, 1.239 mmol) dissolved in dry CH₂Cl₂ (32 mL). The reaction was securely capped and allowed to stir at room temp for 3.5h. Additional 2-methylbut-2-ene (0.5 mL, 4.23 mmol) was added and the reaction was stirred for 18 h at room temp. The reaction was treated with additional 2-methylbut-2-ene (1.5 mL, 12.7 mmol) stirred at room temp for 18 h. Additional 2-methylbut-2-ene (0.7 mL, 5.93 mmol) was added and the reaction was stirred for 18 h at room temp. The reaction was diluted with ethyl acetate, extracted with aq sodium bicarbonate and brine, dried over Na₂SO₄ and evaporated to dryness. The crude material was purified via silica gel chromatography (80 g SiO₂ column, dichloromethane:EtOAc 100:0->50:50) to afford isopropyl-(S)-2-(5-bromo-4-(4,4-dimethylpiperidin-1-yl)-2,6-dimethylpyridin-3-yl)-2-(tert-pentyloxy)acetate, 203.8 mg (36%). LCMS (M+1)=483.2 and 485.2.

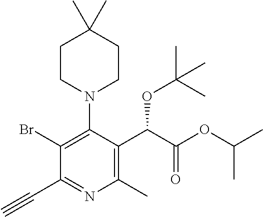 [SMILES: C#Cc1nc(C)c([C@H](OC(C)(C)C)C(=O)OC(C)C)c(N2CCC(C)(C)CC2)c1Br]

### (S)-Isopropyl 2-(5-bromo-4-(4,4-dimethylpiperidin-1-yl)-6-ethynyl-2-methylpyridin-3-yl)-2-(tert-butoxy)acetate

Dimethyl (2-diazo-3-oxobutanoyl)phosphonite (507 mg, 2.48 mmol) was added to a stirring solution of (S)-isopropyl 2-(5-bromo-4-(4,4-dimethylpiperidin-1-yl)-6-formyl-2-methylpyridin-3-yl)-2-(tert-butoxy)acetate (1.0 g, 2.1 mmol) and K₂CO₃ (1.3 g, 9.3 mmol) in MeOH (21 ml) at rt. The reaction was stirred for 1 hr. The reaction was then concentrated, adsorbed onto celite and was purified on silica gel (Biotage, EtOAc/hexanes gradient) to afford (S)-isopropyl 2-(5-bromo-4-(4,4-dimethylpiperidin-1-yl)-6-ethynyl-2-methylpyridin-3-yl)-2-(tert-butoxy)acetate (923 mg, 1.925 mmol, 93% yield) was isolated. LCMS (M+H)=479.10 and 481.05.

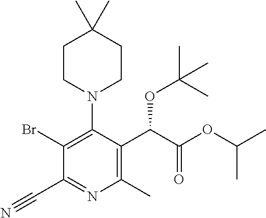 [SMILES: Cc1nc(C#N)c(Br)c(N2CCC(C)(C)CC2)c1[C@H](OC(C)(C)C)C(=O)OC(C)C]

### (S)-Isopropyl 2-(5-bromo-6-cyano-4-(4,4-dimethylpiperidin-1-yl)-2-methylpyridin-3-yl)-2-(tert-butoxy)acetate

Under N₂, tert-butyl nitrite (25 μl, 0.21 mmol) was added to a stirring solution of (S)-isopropyl 2-(5-bromo-4-(4,4-dimethylpiperidin-1-yl)-6-ethynyl-2-methylpyridin-3-yl)-2-(tert-butoxy)acetate (50 mg, 0.10 mmol) and 2-methylpyridine 1-oxide (23 mg, 0.209 mmol) in THF (1 ml) at 70° C. under N₂. The reaction was stirred overnight at 70° C. LCMS showed ˜1:1 mixture of sm and product. The reaction was concentrated, adsorbed onto celite and was purified on silica gel (Biotage, EtOAc/hexanes gradient) to afford (S)-isopropyl 2-(5-bromo-6-cyano-4-(4,4-dimethylpiperidin-1-yl)-2-methylpyridin-3-yl)-2-(tert-butoxy)acetate (18 mg, 0.037 mmol, 35.9% yield). LCMS (M+H)=480.05 and 482.10.

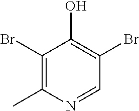

### 3,5-Dibromo-2-methylpyridin-4-ol

To a stirred solution of 2-methylpyridin-4-ol (5 g, 45.8 mmol) in DCM (56.4 ml) and MeOH (6.80 ml) was added tert-butylamine (9.81 ml, 93 mmol) and cooled to 0° C. Bromine (4.72 ml, 92 mmol) was added dropwise over 60 minutes. The reaction mixture was stirred at RT for 3 hours. The reaction mixture was filtered through a fine frit filter and the solid white material dried under vacuum for 18 hrs. ¹H NMR (500 MHz, DMSO-d₆) δ 12.32 (br. s., 1H), 8.21 (s, 1H), 2.40 (s, 3H). LCMS (M+1)=267.7.

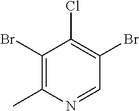

### 3,5-Dibromo-4-chloro-2-methylpyridine

To a solution of 3,5-dibromo-2-methylpyridin-4-ol (13.12 g, 49.2 mmol) in POCl₃ (13.74 ml, 147 mmol) was added triethylamine (6.85 ml, 49.2 mmol) at 0° C. slowly over 80 min. After addition ice bath was removed, and the reaction was heated to 80° C. and stirred for 3h. The reaction mixture was then cooled to rt and slowly quenched by adding it to crushed ice. The resulting suspension was extracted with DCM (250 ml). The organic layer was washed with saturated NaHCO₃ solution (250 mL) followed by water (250 mL) and brine (250 mL). The organic layer was dried (MgSO₄), filtered and concentrated to get 3,5-dibromo-4-chloro-2-methylpyridine (14.7 g, 51.5 mmol, 105% yield) as a off white solid. ¹H NMR (500 MHz, CDCl₃) δ 8.55 (s, 1H), 2.72 (s, 3H). LCMS (M+1)=285.7.

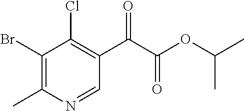 [SMILES: Cc1ncc(C(=O)C(=O)OC(C)C)c(Cl)c1Br]

### Isopropyl 2-(5-bromo-4-chloro-6-methylpyridin-3-yl)-2-oxoacetate

To a −78° C. solution of 3,5-dibromo-4-chloro-2-methylpyridine (9.42 g, 33.0 mmol) and copper(I) bromide-dimethyl sulfide complex (0.339 g, 1.651 mmol) in THF (75 mL) was added dropwise isopropylmagnesium chloride (17.33 mL, 34.7 mmol) over 20 min. The reaction was allowed to warm to −10° C. for 60 min. The reaction mixture was then transferred via cannula to another flask containing a solution of isopropyl 2-chloro-2-oxoacetate (4.97 g, 33.0 mmol) in THF (75 ml) at −60° C. and allowed to warm to −10° C. for 2.5 hr. The reaction was then quenched with 10% solution of ammonium chloride and diethyl ether. The organic layer was washed with brine, collected, dried (MgSO₄), filtered and volatiles evaporated to give the crude material. The crude material was purified via silica gel (330g column, 10-40% EtOAc:Hex) to give the product isopropyl 2-(5-bromo-4-chloro-6-methylpyridin-3-yl)-2-oxoacetate (3.45 g, 9.15 mmol, 27.7% yield) as a yellow oil that later solidified. ¹H NMR (500 MHz, methanol-d4) δ 8.79 (s, 1H), 5.09 (dt, J=12.6, 6.2 Hz, 1H), 2.76 (s, 3H), 1.24-1.22 (m, 3H), 1.20 (d, J=6.3 Hz, 3H). LCMS (M+1)=321.8.

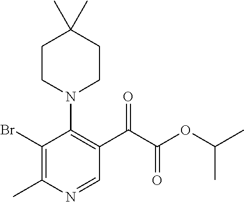 [SMILES: Cc1ncc(C(=O)C(=O)OC(C)C)c(N2CCC(C)(C)CC2)c1Br]

### Isopropyl 2-(5-bromo-4-(4,4-dimethylpiperidin-1-yl)-6-methylpyridin-3-yl)-2-oxoacetate

To a 40 mL vial equipped with a stir bar was added isopropyl 2-(5-bromo-4-chloro-6-methylpyridin-3-yl)-2-oxoacetate (5 g, 15.60 mmol), DIPEA (3.00 ml, 17.16 mmol) and acetonitrile (10.40 ml), then 4,4-dimethylpiperidine (1.942 g, 17.16 mmol). The vial was capped and then placed in a heating block at 85° C. with stirring. After 18 hrs the reaction mixture was dissolved in Et₂O (100 mL) and water (100 mL) and transferred to a 500 mL separatory funnel. The mixture was agitated; the phases were separated. The aq. phase was back extracted with Et₂O (100 mL). The combined organics were washed with brine (50 mL). The solution was dried over MgSO₄; filtered; then concentrated in vacuo. The crude product was purified via silica gel purification (120 g column, 0-30% EtOAc:Hex) to give the product isopropyl 2-(5-bromo-4-(4,4-dimethylpiperidin-1-yl)-6-methylpyridin-3-yl)-2-oxoacetate (4.56 g, 11.48 mmol, 73.6% yield) as a yellow oil that partially solidified. ¹H NMR (500 MHz, CDCl₃) δ 8.45 (s, 1H), 5.26 (dt, J=12.5, 6.3 Hz, 1H), 3.20-3.14 (m, 4H), 2.76 (s, 3H), 1.52-1.48 (m, 4H), 1.42 (d, J=6.3 Hz, 6H), 1.04 (s, 6H). LCMS (M+1)=399.0.

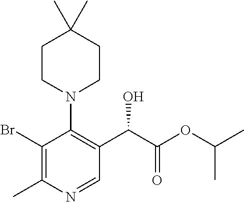 [SMILES: Cc1ncc([C@H](O)C(=O)OC(C)C)c(N2CCC(C)(C)CC2)c1Br]

### (S)-Isopropyl 2-(5-bromo-4-(4,4-dimethylpiperidin-1-yl)-6-methylpyridin-3-yl)-2-hydroxyacetate

To a 100 mL R B-flask equipped with a stir bar was added isopropyl 2-(5-bromo-4-(4,4-dimethylpiperidin-1-yl)-6-methylpyridin-3-yl)-2-oxoacetate (2.5 g, 6.29 mmol). The flask was fitted with a rubber septum and then placed under N₂ atm (vac/fill×3). To the flask was added toluene (17.98 ml). The flask was placed in a −35° C. bath (dichloroethane/dry ice). A thermometer was used to monitor the internal temperature. When the internal temp was −30° C., to the flask was added (R)-1-methyl-3,3-diphenylhexahydropyrrolo[1,2-c][1,3,2]oxazaborole (0.944 ml, 0.944 mmol). To the stirred solution was added 50% catecholborane/toluene (1.886 ml, 8.81 mmol) over 2 minutes. Within 5 minutes following the addition the temperature rose to −25° C. before falling to −30° C. The solution was stirred at −30° C. for 3 h. The flask was transferred to a −15 to −12° C. cold bath (chiller/circulator). The yellow solution was stirred for 1 day at −15 to −12° C. The reaction was quenched with 5 mL of 2M aq. sodium carbonate. The reaction was then diluted with 100 mL EtOAc and 100 mL 2M aq sodium carbonate and stirred vigorously for 2 hrs. The layers were separated and the organic layer collected and stirred vigorously for an additional 1 hr. The organic layer was washed with brine, dried over MgSO₄, filtered and evaporated to give the crude product. The crude product was purified on silica gel chromatography (80 g column, 10-40% EtOAc:Hex) to afford the product (S)-isopropyl 2-(5-bromo-4-(4,4-dimethylpiperidin-1-yl)-6-methylpyridin-3-yl)-2-hydroxyacetate (2 g, 5.01 mmol, 80% yield) as a yellow oil that solidified at RT. ¹H NMR (500 MHz, CDCl₃) δ 8.33 (s, 1H), 5.31 (d, J=6.9 Hz, 1H), 5.13-5.03 (m, 2H), 3.80 (br. s., 2H), 2.87-2.75 (m, 1H), 2.71 (s, 3H), 2.69-2.60 (m, 1H), 1.71-1.59 (m, 2H), 1.43 (d, J=14.8 Hz, 2H), 1.28 (d, J=6.1 Hz, 3H), 1.16 (d, J=6.3 Hz, 3H), 1.04 (s, 3H), 1.08 (s, 3H). LCMS (M+1)=399.0.

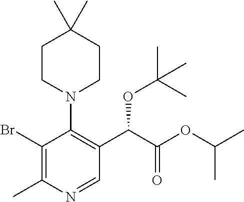 [SMILES: Cc1ncc([C@H](OC(C)(C)C)C(=O)OC(C)C)c(N2CCC(C)(C)CC2)c1Br]

### (S)-Isopropyl 2-(5-bromo-4-(4,4-dimethylpiperidin-1-yl)-6-methylpyridin-3-yl)-2-(tert-butoxy)acetate

In a 250 ml round bottom flask fitted with a shlenk adaptor with rubber septum (with empty balloon attached), isobutylene gas was vigorously bubbled for 30 minutes into a 0° C. solution of (S)-isopropyl 2-(5-bromo-4-(4,4-dimethylpiperidin-1-yl)-6-methylpyridin-3-yl)-2-hydroxyacetate (2 g, 5.01 mmol) and perchloric acid (0.861 mL, 10.02 mmol) in DCM (100 mL) until the volume doubled and the balloon filled to firmness. After 2 hrs, the isobutylene line was disconnected and needle pulled to just above the solution line then connected to a bubbler to monitor isobutylene gas exit. The ice bath was removed and warmed up to RT while monitoring for conversion. After 2 hrs the reaction appeared to go to full conversion according to LCMS. The reaction mixture was poured into a 1L Erlenmeyer flask and made basic with 2M sodium carbonate while vigorously stirring. The organic layer was separated and washed with water, followed by brine, collected, dried (MgSO₄), filtered and volatiles evaporated to afford the crude product. The crude product was purified on silica gel (40 g column, 5-40% EtOAc:Hex) to afford the product (S)-isopropyl 2-(5-bromo-4-(4,4-dimethylpiperidin-1-yl)-6-methylpyridin-3-yl)-2-(tert-butoxy)acetate (1.95 g, 4.28 mmol, 85% yield) as a clear oil that later crystallized to a white solid. ¹H NMR (500 MHz, CDCl₃) δ 8.61 (s, 1H), 5.61 (s, 1H), 5.01 (dt, J=12.5, 6.3 Hz, 1H), 3.81 (t, J=10.9 Hz, 1H), 3.60 (t, J=11.0 Hz, 1H), 2.76 (d, J=11.5 Hz, 1H), 2.69 (s, 3H), 2.64 (d, J=12.1 Hz, 1H), 1.63-1.51 (m, 2H), 1.46 (d, J=11.2 Hz, 1H), 1.38 (d, J=14.2 Hz, 1H), 1.26-1.22 (m, 12H), 1.20 (d, J=6.1 Hz, 3H), 1.09-1.03 (m, 6H). LCMS (M+1)=457.1.

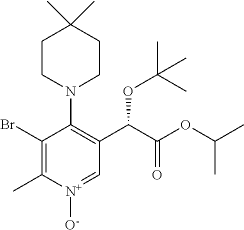 [SMILES: Cc1c(Br)c(N2CCC(C)(C)CC2)c([C@H](OC(C)(C)C)C(=O)OC(C)C)c[n+]1[O-]]

### (S)-3-Bromo-5-(1-(tert-butoxy)-2-isopropoxy-2-oxoethyl)-4-(4,4-dimethylpiperidin-1-yl)-2-methylpyridine 1-oxide

To a stirred solution of (S)-isopropyl 2-(5-bromo-4-(4,4-dimethylpiperidin-1-yl)-6-methylpyridin-3-yl)-2-(tert-butoxy)acetate (89 mg, 0.195 mmol) in DCM (2 mL) was added 77% mCPBA (65.7 mg, 0.293 mmol) at rt over 5 min. After 4 h, the reaction mixture was washed with sat. Na₂CO₃ (3×25 mL), dried (MgSO₄), filtered and concentrated to give (S)-3-bromo-5-(1-(tert-butoxy)-2-isopropoxy-2-oxoethyl)-4-(4,4-dimethylpiperidin-1-yl)-2-methylpyridine 1-oxide (93 mg, 0.197 mmol, 100% yield) which was used in the next step without purification. ¹H NMR (500 MHz, CDCl₃) δ 8.47 (s, 1H), 5.47 (s, 1H), 5.01 (dt, J=12.5, 6.3 Hz, 1H), 3.72 (td, J=11.8, 2.5 Hz, 1H), 3.62 (td, J=11.8, 2.5 Hz, 1H), 2.73 (s, 3H), 2.69-2.62 (m, 2H), 1.46 (br dd, J=12.6, 2.0 Hz, 1H), 1.39-1.34 (m, 1H), 1.26-1.23 (m, 15H), 1.05 (d, J=11.8 Hz, 6H). 2 protons on the piperidine ring were not observed due to water in the ¹H NMR experiment. LCMS (M+1)=471.1 and 473.1.

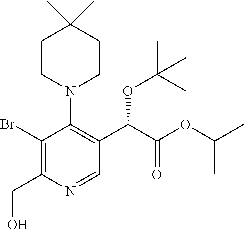 [SMILES: CC(C)OC(=O)[C@@H](OC(C)(C)C)c1cnc(CO)c(Br)c1N1CCC(C)(C)CC1]

### (S)-Isopropyl 2-(5-bromo-4-(4,4-dimethylpiperidin-1-yl)-6-(hydroxymethyl)pyridin-3-yl)-2-(tert-butoxy)acetate

To a stirred solution of (S)-3-bromo-5-(1-(tert-butoxy)-2-isopropoxy-2-oxoethyl)-4-(4,4-dimethylpiperidin-1-yl)-2-methylpyridine 1-oxide (276 mg, 0.585 mmol) anhydrous DMF (3 ml) was added trifluoroacetic anhydride (0.331 ml, 2.342 mmol) at RT. After 3 h, sat NaHCO₃ (10 mL) was added and stirred for 10 minutes before extracting with EtOAc. The organic layer was washed with water (2×), followed by brine, collected, dried over MgSO₄, filtered and volatiles evaporated to afford the crude product. The crude product was purified on silica gel (40 g column, 5-40% EtOAc:Hex) to afford the product (S)-isopropyl 2-(5-bromo-4-(4,4-dimethylpiperidin-1-yl)-6-(hydroxymethyl)pyridin-3-yl)-2-(tert-butoxy)acetate (180 mg, 0.382 mmol, 65.2% yield) as a clear oil. ¹H NMR (500 MHz, CDCl₃) δ 8.72 (s, 1H), 5.60 (s, 1H), 5.02 (dt, J=12.5, 6.2 Hz, 1H), 4.73 (s, 2H), 1.63-1.44 (m, 4H), 1.27-1.25 (m, 12H), 1.21 (d, J=6.1 Hz, 3H), 1.07 (s, 6H) LCMS (M+1)=471.1 and 473.1. 4 protons on the piperidine ring were not resolved in the ¹H NMR experiment.

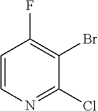

### 3-Bromo-2-chloro-4-fluoropyridine

To a dry 1000 mL round bottom flask under nitrogen was added 2-chloro-4-fluoropyridine (13 g, 99 mmol) and THF (250 mL). The reaction was flushed with argon, securely capped, cooled to −78° C. and slowly (over 20-25 min) treated with LDA, 1 M in THF:hexanes (100 ml, 100 mmol). After the addition was complete, the reaction was stirred at −78° C. for 85 min. The reaction was then treated (over 10 min) with a solution of 1,2-dibromo-1,1,2,2-tetrachloroethane (35 g, 107 mmol) in THF (150 mL). After the addition was complete, the reaction was stirred at −78° C. for 30 min, then the bath was removed and the reaction was allowed to warm to room temp. The reaction was diluted with dichloromethane (500 mL), extracted with water (1×150 mL), brine (1×100 mL), dried over Na₂SO₄, and concentrated. The crude material was purified via silica gel chromatography (330g SiO₂ column, hexane:dichloromethane 100:0->65:35) to afford 3-bromo-2-chloro-4-fluoropyridine, 12 g (58%). ¹H NMR (500 MHz, CDCl₃) δ 8.33 (dd, J=7.4, 5.4 Hz, 1H), 7.07 (dd, J=7.1, 5.4 Hz, 1H).

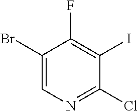

### 5-Bromo-2-chloro-4-fluoro-3-iodopyridine

To a flame dried 500 mL conical flask under nitrogen was added 2,2,6,6-tetramethylpiperidine (12.7 mL, 75 mmol) and THF (225 mL). The reaction was flushed with argon, cooled to −78° C. and treated with n-butyllithium, 1.6M in hexanes (39 mL, 62.4 mmol). The reaction was stirred at −78 C, then the bath was removed and the reaction was allowed to warm to 0° C. over 20 min. The reaction was recooled to −78° C. and transferred to a solution of 3-bromo-2-chloro-4-fluoropyridine (13.55 g, 64.4 mmol) in THF (225 mL) at −78° C. over 10 min. After the addition was complete, the reaction was stirred at −78° C. for 50 min, then treated with a solution of iodine (18.8 g, 74.1 mmol) in THF (225 mL) at −50 C. The reaction was packed in dry ice and allowed to stir while slowly warming to room temp over 18 h. The reaction was diluted with ethyl acetate (600 mL), extracted with aq Na₂S₂O₃ (5 g dissolved in water (250 mL)), water (1×60 mL), brine (1×60 mL), dried over Na₂SO₄, and concentrated. The crude material was purified via silica gel chromatography (330 g SiO₂ column, hexane:dichloromethane: 100:0->65:35) to afford 5-bromo-2-chloro-4-fluoro-3-iodopyridine, 15.7 g (72%). ¹H NMR (500 MHz, CDCl₃) δ 8.42 (d, J=8.4 Hz, 1H).

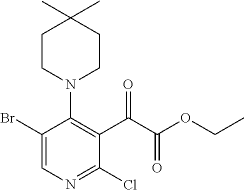 [SMILES: CCOC(=O)C(=O)c1c(Cl)ncc(Br)c1N1CCC(C)(C)CC1]

### Ethyl 2-(5-bromo-2-chloro-4-(4,4-dimethylpiperidin-1-yl)pyridin-3-yl)-2-oxoacetate

To a dry 500 mL conical flask under nitrogen was added 5-bromo-2-chloro-4-fluoro-3-iodopyridine (11.34 g, 33.7 mmol), copper(I) bromide-dimethyl sulfide (1.36 g, 6.62 mmol) and THF (400 mL). The resulting suspension was flushed very well with argon, cooled to −78 C. and treated with isopropylmagnesium chloride, 1.92 M in THF (17.56 mL, 33.7 mmol). After the addition was complete, the reaction was stirred at −78° C. for 33 min, then allowed to warm to −10° C. over 2 h. The reaction was recooled to −60° C. and transferred via cannulae to a solution of ethyl oxalyl chloride (6.34 mL, 56.8 mmol) in THF (100 mL) at −78 C. The reaction was stirred at −78° C. for 5 min, slowly allowed to warm to −12 C over 85 min then held at −12° C. for 2.5 h. The reaction was cooled to −35° C. and treated (via cannulae) with a solution of 4,4-dimethylpiperidine (16 g, 141 mmol) in N,N-diisopropylethylamine (19.2 mL, 110 mmol), followed by acetonitrile (100 mL) and allowed to stir while slowly warming to room temp over 18 h. The reaction was treated with diethanol amine (805 mg, 7.66 mmol), diluted with ethyl acetate (1100 mL), extracted with water (1×150 mL), dried over Na₂SO₄ and concentrated. The crude material was purified via silica gel chromatography (330g SiO₂ column, hexane:dichloromethane: 100:0->0:100) to afford ethyl 2-(5-bromo-2-chloro-4-(4,4-dimethylpiperidin-1-yl)pyridin-3-yl)-2-oxoacetate, 10.81 g (79%). LCMS (M+1)=403.1 and 405.0.

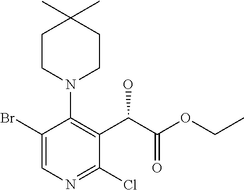 [SMILES: CCOC(=O)[C@@H](O)c1c(Cl)ncc(Br)c1N1CCC(C)(C)CC1]

### Ethyl (S)-2-(5-bromo-2-chloro-4-(4,4-dimethylpiperidin-1-yl)pyridin-3-yl)-2-hydroxyacetate

To a solution of ethyl 2-(5-bromo-2-chloro-4-(4,4-dimethylpiperidin-1-yl)pyridin-3-yl)-2-oxoacetate (11.6 g, 28.7 mmol) in anhydrous Toluene (500 mL) was added (R)-2-methyl-CBS-oxazaborolidine, 1.0M in toluene (35.5 mL, 35.5 mmol). The reaction was cooled to −78° C. and treated (over 33 min) with catecholborane, 50 wt % in toluene (29 mL, 135 mmol). After the addition was complete, the dry ice was removed from the bath and the reaction was allowed to warm to −5° C. over 5 h, then held at −5° C. for 40 min. The reaction was cooled to −55° C. and transferred (via cannulae) to an ice cold solution of aqueous saturated K₂CO₃ (200 mL) and EtOAc (400 mL) and the resulting two phase mixture was stirred at room temp for 18 h. The reaction was further diluted with ethyl acetate, extracted with water (1×30 mL), brine (1×30 mL), dried over Na₂SO₄ and concentrated. The crude material was purified via silica gel chromatography (330g SiO₂ column, dichloromethane:EtOAc 100:0->50:50) to afford ethyl (S)-2-(5-bromo-2-chloro-4-(4,4-dimethylpiperidin-1-yl)pyridin-3-yl)-2-hydroxyacetate, 11.5 g (99%). LCMS (M+1)=405.0 and 407.0.

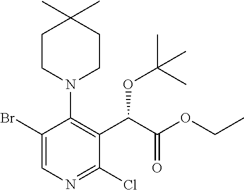 [SMILES: CCOC(=O)[C@@H](OC(C)(C)C)c1c(Cl)ncc(Br)c1N1CCC(C)(C)CC1]

### Ethyl-(S)-2-(5-bromo-2-chloro-4-(4,4-dimethylpiperidin-1-yl)pyridin-3-yl)-2-(tert-butoxy)acetate

To a dry 1000 mL pressure bottle under nitrogen was added (S)-ethyl 2-(5-bromo-2-chloro-4-(4,4-dimethylpiperidin-1-yl)pyridin-3-yl)-2-hydroxyacetate (5.23 g, 12.89 mmol), CH₂Cl₂ (300 mL) and 4A mol sieves. The reaction was allowed to stir at room temp for 2h, then cooled to −78° C. The reaction was then treated with perchloric acid (2.33 mL, 38.7 mmol), and isobutylene was bubbled into the reaction until the reaction volume roughly doubled. The flask was securely capped, removed from the bath and allowed to stir for 18 h while slowly warming to room temp. The reaction was cooled to −60° C. and quenched into an erlenmeyer flask containing a mixture of CH₂Cl₂ (200 mL) and NaHCO₃ (12.8 g, 152 mmol) dissolved in water (250 mL). The reaction was further diluted with dichloromethane (300 mL), extracted with water (1×75 mL), brine (1×75 mL), dried over Na₂SO₄ and concentrated. The crude material was purified via silica gel chromatography (330g SiO₂ column, dichloromethane:EtOAc 100:0->55:45) to afford ethyl-(S)-2-(5-bromo-2-chloro-4-(4,4-dimethylpiperidin-1-yl)pyridin-3-yl)-2-(tert-butoxy)acetate, 4.62 g (78%). LCMS (M+1)=461.1, 463.1. ¹H NMR (500 MHz, CDCl₃) δ 8.37 (s, 1H), 5.60 (br s, 1H), 4.28-4.11 (m, 2H), 3.91-3.63 (m, 1H), 3.33 (br s, 1H), 3.07 (br d, J=2.3 Hz, 1H), 2.58 (br d, J=1.5 Hz, 1H), 1.81-1.63 (m, 1H), 1.49-1.35 (m, 2H), 1.27 (br d, J=5.0 Hz, 1H), 1.25 (s, 9H), 1.02 (s, 6H).

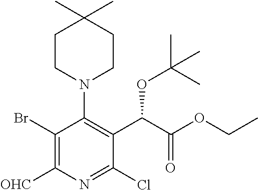 [SMILES: CCOC(=O)[C@@H](OC(C)(C)C)c1c(Cl)nc(C=O)c(Br)c1N1CCC(C)(C)CC1]

### Ethyl-(S)-2-(5-bromo-2-chloro-4-(4,4-dimethylpiperidin-1-yl)-6-formylpyridin-3-yl)-2-(tert-butoxy)acetate

To a dry 100 mL round bottom flask under argon was added ethyl-(S)-2-(5-bromo-2-chloro-4-(4,4-dimethylpiperidin-1-yl)pyridin-3-yl)-2-(tert-butoxy)acetate (1.05 g, 2.274 mmol) and THF (48 mL). The resulting solution was cooled to −25 C and then treated slowly (over 2 min) 2,2,6,6-tetramethylpiperidinyl magnesium chloride lithium chloride complex 1.0 M in THF/toluene (4.65 mL, 4.65 mmol). The reaction was stirred at −21° C. (+/−4 C) for 33 min, then recooled to −20° C. and treated with anhydrous DMF (1.00 mL, 12.91 mmol). The reaction was allowed to warm to −8° C. over 70 min, then recooled to −20° C. and quenched with aqueous saturated NH₄Cl. The crude reaction was diluted with ethyl acetate (150 mL), extracted with water (1×15 mL, brine (1×15 mL), dried over Na₂SO₄, and concentrated. The crude material was purified via silica gel chromatography (80 g SiO₂ column, dichloromethane:EtOAc 100:0->80:20) to afford ethyl (S)-2-(5-bromo-2-chloro-4-(4,4-dimethylpiperidin-1-yl)-6-formylpyridin-3-yl)-2-(tert-butoxy)acetate, 1.04 g (93%). LCMS (M+1)=489.1, 491.1.

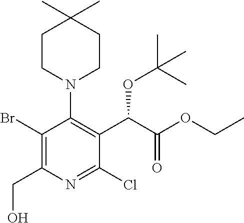 [SMILES: CCOC(=O)[C@@H](OC(C)(C)C)c1c(Cl)nc(CO)c(Br)c1N1CCC(C)(C)CC1]

### Ethyl (S)-2-(5-bromo-2-chloro-4-(4,4-dimethylpiperidin-1-yl)-6-(hydroxymethyl)pyridin-3-yl)-2-(tert-butoxy)acetate

To a dry reaction vial under nitrogen was added (S)-ethyl 2-(5-bromo-2-chloro-4-(4,4-dimethylpiperidin-1-yl)-6-formylpyridin-3-yl)-2-(tert-butoxy)acetate (20 mg, 0.041 mmol) and EtOH (1.5 mL). The resulting solution was treated with sodium borohydride (8.5 mg, 0.225 mmol), capped and stirred at room temp for 2 h. The reaction was diluted with ethyl acetate (75 mL), extracted with aq sat'd NH₄Cl (1 10 mL), water (1×10 mL), brine (1×10 mL), dried over Na₂SO₄ and concentrated to afford ethyl (S)-2-(5-bromo-2-chloro-4-(4,4-dimethylpiperidin-1-yl)-6-(hydroxymethyl)pyridin-3-yl)-2-(tert-butoxy)acetate (12 mg, 60%) was used “as is” without further purification. LCMS (M+1)=491.2, 493.1.

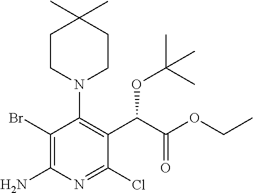 [SMILES: CCOC(=O)[C@@H](OC(C)(C)C)c1c(Cl)nc(N)c(Br)c1N1CCC(C)(C)CC1]

### Ethyl (S)-2-(6-amino-5-bromo-2-chloro-4-(4,4-dimethylpiperidin-1-yl)pyridin-3-yl)-2-(tert-butoxy)acetate

To a reaction vial under nitrogen was added (S)-ethyl 2-(5-bromo-2-chloro-4-(4,4-dimethylpiperidin-1-yl)-6-formylpyridin-3-yl)-2-(tert-butoxy)acetate (149 mg, 0.304 mmol) and acetonitrile (4 mL). The reaction was stirred at room temp, treated with a solution of oxone (380 mg, 0.618 mmol) in water (1.75 mL) and stirred at room temp for 20h. The reaction was diluted with ethyl acetate (100 mL), extracted with water (1×8 mL) and evaporated to dryness. The crude residue was dissolved in toluene (4 mL) and treated with triethylamine (100.4 μL, 0.720 mmol), water (32.45 μL, 1.801 mmol), and diphenylphosphoryl azide (198 mg, 0.719 mmol). The reaction was flushed very briefly with nitrogen, capped and heated at 90° C. oil bath for 90 min. The reaction was diluted with ethyl acetate (100 mL), extracted with aq sat'd NaHCO₃ (1×10 mL), water (1×10 mL), brine (1×10 mL) dried over Na₂SO₄ and concentrated. The crude residue was purified via silica gel chromotography (40g SiO₂ column, hexane:EtOAc 100:0->70:30) to afford ethyl (S)-2-(6-amino-5-bromo-2-chloro-4-(4,4-dimethylpiperidin-1-yl)pyridin-3-yl)-2-(tert-butoxy)acetate, 100 mg (58%). LCMS (M+1)=476.1, 478.1.

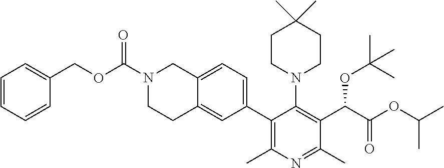 [SMILES: Cc1nc(C)c([C@H](OC(C)(C)C)C(=O)OC(C)C)c(N2CCC(C)(C)CC2)c1-c1ccc2c(c1)CCN(C(=O)OCc1ccccc1)C2]

### (S)-Benzyl 6-(5-(1-(tert-butoxy)-2-isopropoxy-2-oxoethyl)-4-(4,4-dimethylpiperidin-1-yl)-2,6-dimethylpyridin-3-yl)-3,4-dihydroisoquinoline-2(1H)-carboxylate

(S)-isopropyl 2-(5-bromo-4-(4,4-dimethylpiperidin-1-yl)-2,6-dimethylpyridin-3-yl)-2-(tert-butoxy)acetate (1 g, 2.1 mmol), benzyl 6-(6-methyl-4,8-dioxo-1,3,6,2-dioxazaborocan-2-yl)-3,4-dihydroisoquinoline-2(1H)-carboxylate (0.989 g, 2.34 mmol), 2-dicyclohexylphosphino-2′,6′-dimethoxybiphenyl (0.175 g, 0.426 mmol), potassium phosphate tribasic (3.39 g, 16.0 mmol), Pd(OAc)₂ (0.048 g, 0.213 mmol) were combined under N₂ (g). 1,4-Dioxane (35.5 ml) and Water (7.10 ml) were added under N₂ (g). The mixture was heated at 80° C. for 2 h. The reaction was concentrated, adsorbed onto celite and was purified on silica gel (Biotage, EtOAc/hexanes gradient, 0-100% over 10 CVs) to give the expected product (S)-benzyl 6-(5-(1-(tert-butoxy)-2-isopropoxy-2-oxoethyl)-4-(4,4-dimethylpiperidin-1-yl)-2,6-dimethylpyridin-3-yl)-3,4-dihydroisoquinoline-2(1H)-carboxylate (1.1 g, 1.7 mmol, 79% yield). LCMS (M+H)=656.75.

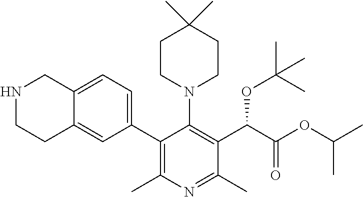 [SMILES: Cc1nc(C)c([C@H](OC(C)(C)C)C(=O)OC(C)C)c(N2CCC(C)(C)CC2)c1-c1ccc2c(c1)CCNC2]

### (S)-Isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate

A mixture of (S)-benzyl 6-(5-(1-(tert-butoxy)-2-isopropoxy-2-oxoethyl)-4-(4,4-dimethylpiperidin-1-yl)-2,6-dimethylpyridin-3-yl)-3,4-dihydroisoquinoline-2(1H)-carboxylate (1.1 g, 1.7 mmol), 1M HCl (3.35 ml, 3.35 mmol) and 10% Pd—C(178 mg, 0.168 mmol) in EtOH (21 ml) was evacuated and vented to H₂ (g) three times and then left under balloon of H₂ (g) atmosphere for overnight, 15 h. The reaction was then filtered through celite, concentrated and the residue was partitioned between sat. Na₂CO₃ (20 mL) and CH₂Cl₂ (50 mL). Org layer separated and aq layer extracted with CH₂Cl₂ (25 mL) and combined org layers dried (Na₂SO₄), filtered and concentrated to give (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (798 mg, 1.53 mmol, 91% yield). LCMS (M+H)=522.35.

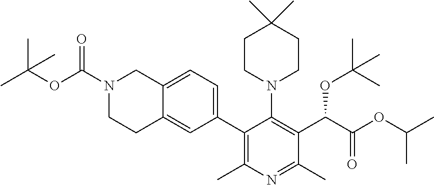 [SMILES: Cc1nc(C)c([C@H](OC(C)(C)C)C(=O)OC(C)C)c(N2CCC(C)(C)CC2)c1-c1ccc2c(c1)CCN(C(=O)OC(C)(C)C)C2]

### tert-Butyl (S)-6-(5-(1-(tert-butoxy)-2-isopropoxy-2-oxoethyl)-4-(4,4-dimethylpiperidin-1-yl)-2,6-dimethylpyridin-3-yl)-3,4-dihydroisoquinoline-2(1H)-carboxylate

To a mixture of isopropyl (S)-2-(5-bromo-4-(4,4-dimethylpiperidin-1-yl)-2,6-dimethylpyridin-3-yl)-2-(tert-butoxy)acetate and (2-(tert-butoxycarbonyl)-1,2,3,4-tetrahydroisoquinolin-6-yl)boronic acid (1.6 g, 5.77 mmol) in THF (100 mL) was added 0.5 M potassium phosphate tribasic (22 ml, 11.00 mmol). The reaction was flushed with argon, treated with 2^{nd} generation X-Phos precatalyst (138 mg, 0.175 mmol), sealed and heated at 80° C. for 18 h. The crude material was dissolved in EtOAc, extracted and purified via silica gel chromatography (80g SiO₂ column, hexane:EtOAc 100:0->0:100) to afford tert-butyl (S)-6-(5-(1-(tert-butoxy)-2-isopropoxy-2-oxoethyl)-4-(4,4-dimethylpiperidin-1-yl)-2,6-dimethylpyridin-3-yl)-3,4-dihydroisoquinoline-2(1H)-carboxylate, 1.75g (76%). LCMS (M+1)=622.5.

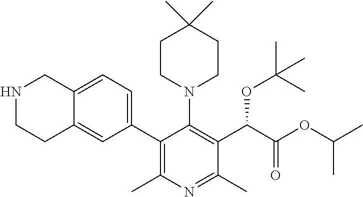 [SMILES: Cc1nc(C)c([C@H](OC(C)(C)C)C(=O)OC(C)C)c(N2CCC(C)(C)CC2)c1-c1ccc2c(c1)CCNC2]

### Isopropyl (S)-2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate

A solution of (S)-tert-butyl 6-(5-(1-(tert-butoxy)-2-isopropoxy-2-oxoethyl)-4-(4,4-dimethylpiperidin-1-yl)-2,6-dimethylpyridin-3-yl)-3,4-dihydroisoquinoline-2(1H)-carboxylate (1.75 g, 2.81 mmol) in DCM (30 mL) treated with TFA (8 ml, 104 mmol) over 2 min. After the addition was complete, the reaction was allowed to stand at room temperature for 10 min. The crude reaction was purified via reverse phase Prep-HPLC to afford isopropyl (S)-2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate, 1.02 g (66%). LCMS (M+1)=522.4.

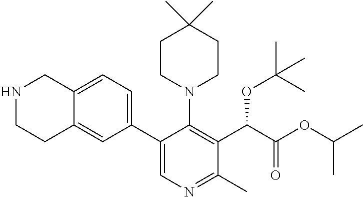 [SMILES: Cc1ncc(-c2ccc3c(c2)CCNC3)c(N2CCC(C)(C)CC2)c1[C@H](OC(C)(C)C)C(=O)OC(C)C]

### (S)-Isopropyl 2-(tert-butoxy)-2-(4-(4,4-dimethylpiperidin-1-yl)-2-methyl-5-(1,2,3,4-tetrahydroisoquinolin-6-yl)pyridin-3-yl)acetate

A mixture of (S)-benzyl 6-(5-(1-(tert-butoxy)-2-isopropoxy-2-oxoethyl)-4-(4,4-dimethylpiperidin-1-yl)-6-methylpyridin-3-yl)-3,4-dihydroisoquinoline-2(1H)-carboxylate (324 mg, 0.505 mmol), 1M HCl (1 ml, 1 mmol) and 10% Pd—C(54 mg, 0.050 mmol) in EtOH (6.3 ml) was evacuated and vented to H₂ (g) three times and then left under balloon of H₂ (g) atmosphere overnight, 15 h.

Then the reaction was filtered through celite, concentrated and the residue was partitioned between sat. Na₂CO₃ and CH₂Cl₂. The organic layer was separated and the aqueous layer was extracted with CH₂Cl₂ and combined organic layers were dried (Na₂SO₄), filtered and concentrated to give (S)-isopropyl 2-(tert-butoxy)-2-(4-(4,4-dimethylpiperidin-1-yl)-2-methyl-5-(1,2,3,4-tetrahydroisoquinolin-6-yl)pyridin-3-yl)acetate (quant yield). LCMS (M+H)=508.30.

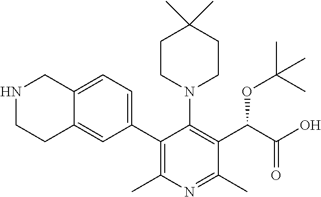 [SMILES: Cc1nc(C)c([C@H](OC(C)(C)C)C(=O)O)c(N2CCC(C)(C)CC2)c1-c1ccc2c(c1)CCNC2]

### (S)-2-(tert-Butoxy)-2-(4-(4,4-dimethylpiperidin-1-yl)-2,6-dimethyl-5-(1,2,3,4-tetrahydroisoquinolin-6-yl)pyridin-3-yl)acetic Acid

(S)-benzyl 6-(5-(1-(tert-butoxy)-2-isopropoxy-2-oxoethyl)-4-(4,4-dimethylpiperidin-1-yl)-2,6-dimethylpyridin-3-yl)-3,4-dihydroisoquinoline-2(1H)-carboxylate (150 mg, 0.229 mmol) was suspended in ethanol (10 mL) and water (1 mL) within a pressure vial equipped with a magnetic stirring bar. Ground potassium hydroxide (128 mg, 2.287 mmol) was added and the vial was capped and heated to 90° C. with stirring. After several days of heating and after the addition of more potassium hydroxide (128 mg, 2.287 mmol), LC/MS showed that the reaction was mostly the desired product. Dried down the reaction solvent under nitrogen and then took up the residue in methanol. Filtered and purified by prep HPLC. Like fractions were dried down under a stream of nitrogen overnight giving 90 mg (82%) of (S)-2-(tert-butoxy)-2-(4-(4,4-dimethylpiperidin-1-yl)-2,6-dimethyl-5-(1,2,3,4-tetrahydroisoquinolin-6-yl)pyridin-3-yl)acetic acid as a residue. LCMS (M+1)=480.5.

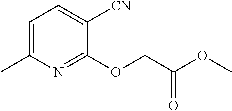

### Methyl 2-((3-cyano-6-methylpyridin-2-yl)oxy)acetate

To a stirred mixture of 2-hydroxy-6-methylnicotinonitrile (2.0 g, 15 mmol) and K₂CO₃ (2.3 g, 16 mmol) in acetone (75 ml) was added methyl bromoacetate (1.37 ml, 14.9 mmol) at rt. The reaction was warmed to 56° C. and allowed to stir for 3 hrs and then overnight while cooling to rt. The reaction mixture was diluted with hexanes, filtered and the ppt was discarded. The filtrate was allowed to sit for 5 min and ppt formed was collected (1 g) consistent with the expected product by NMR. The remaining filtrate was concentrated to give an additional amount of 1.7 g of product consistent by NMR and in total that makes methyl 2-((3-cyano-6-methylpyridin-2-yl)oxy)acetate (2.7 g, 13 mmol, 88% yield) as white solid which was used in the next step without purification. ¹H NMR (500 MHz, methanol-d4) δ 7.99 (d, J=7.6 Hz, 1H), 6.45 (d, J=7.6 Hz, 1H), 5.00-4.90 (m, 2H), 3.86-3.71 (m, 3H), 2.53-2.41 (m, 3H).

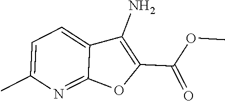

### Methyl 3-amino-6-methylfuro[2,3-b]pyridine-2-carboxylate

To a stirred solution of KOtBu (1.02 g, 9.07 mmol) in THF (20 mL) was added a solution of methyl 2-((3-cyano-6-methylpyridin-2-yl)oxy)acetate (1.7 g, 8.2 mmol) in THF (20 mL) over 5 min at rt. The resulting think slurry was stirred for 4 h, then the reaction mixture was quenched with 1M HCl (9 mL), diluted with EtOAc, washed with water, brine, dried (Na₂SO₄), filtered and concentrated to give methyl 3-amino-6-methylfuro[2,3-b]pyridine-2-carboxylate (1.1 g, 5.33 mmol, 64.7% yield) consistent by LCMS as dark brown solid. LCMS (M+H)=207.00.

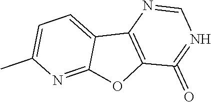

### 7-Methylpyrido[3′,2′:4,5]furo[3,2-d]pyrimidin-4(3H)-one

A mixture of methyl 3-amino-6-methylfuro[2,3-b]pyridine-2-carboxylate (100 mg, 0.485 mmol) and ethyl formimidate, HCl (106 mg, 0.970 mmol) in formamide (2.5 mL) was heated at 180° C. for 2 h. LCMS showed a major peak with the expected product. The reaction was cooled to rt and purified by prep HPLC (TFA buffered) and lyophylized to give the expected product 7-methylpyrido[3′,2′:4,5]furo[3,2-d]pyrimidin-4(3H)-one (20 mg, 0.099 mmol, 20.50% yield). LCMS (M+H)=202.00.

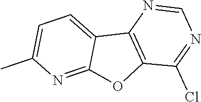

### 4-Chloro-7-methylpyrido[3′,2′:4,5]furo[3,2-d]pyrimidine

To a stirred solution of oxalyl chloride (20 μl, 0.22 mmol) in DCE (1.2 ml) was added dropwise DMF (17 μl, 0.22 mmol) at rt. After the vigorous gas evolution had ceased, 7-methylpyrido[3′,2′:4,5]furo[3,2-d]pyrimidin-4(3H)-one (25 mg, 0.12 mmol) was added and the mixture was heated at 75° C. for 2 h. LCMS of the reaction showed partial conversion. The reaction was concentrated, adsorbed onto celite and was purified on silica gel (Biotage, EtOAc/hexanes gradient, 0-100% over 10 CVs) to afford 4-chloro-7-methylpyrido[3′,2′:4,5]furo[3,2-d]pyrimidine (5 mg, 0.023 mmol, 18.32% yield). ¹H NMR (500 MHz, methanol-d4) δ 9.00 (s, 1H), 8.60 (d, J=7.9 Hz, 1H), 7.59 (d, J=8.0 Hz, 1H), 2.84-2.61 (m, 3H).

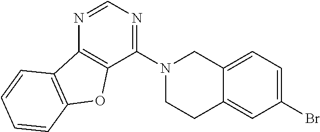 [SMILES: Brc1ccc2c(c1)CCN(c1ncnc3c1oc1ccccc13)C2]

### 4-(6-Bromo-3,4-dihydroisoquinolin-2(1H)-yl)benzofuro[3,2-d]pyrimidine

A mixture of 4-chloro-benzo[4,5]furo[3,2-d]pyrimidine (1.06 g, 5.19 mmol), 6-bromo-1,2,3,4-tetrahydroisoquinoline (1.0 g, 4.7 mmol), potassium carbonate (1.95 g, 14.1 mmol) and sodium iodide (0.71 g, 4.7 mmol) in dioxane (50 mL) was heated at 90° C. for 6 hrs. The mixture was diluted with EtOAc and washed with water, brine, dried over Na₂SO₄ and concentrated. The residue was purified by recrystallization with EtOAc to give 4-(6-bromo-3,4-dihydroisoquinolin-2(1H)-yl)benzofuro[3,2-d]pyrimidine (1.2 g, 3.16 mmol, 66.9% yield). LCMS (M+H): 379.90, 381.90.

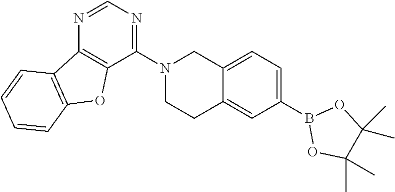 [SMILES: CC1(C)OB(c2ccc3c(c2)CCN(c2ncnc4c2oc2ccccc24)C3)OC1(C)C]

### 4-(6-(4,4,5,5-Betramethyl-1,3,2-dioxaborolan-2-yl)-3,4-dihydroisoquinolin-2(1H)-yl)benzofuro[3,2-d]pyrimidine

4-(6-Bromo-3,4-dihydroisoquinolin-2(1H)-yl)benzofuro[3,2-d]pyrimidine (1.4 g, 3.7 mmol), 4,4,4′,4′,5,5,5′,5′-octamethyl-2,2′-bi(1,3,2-dioxaborolane) (1.4 g, 5.5 mmol), Pd(dppf)Cl₂ (0.269 g, 0.368 mmol) and potassium acetate (1.08 g, 11.1 mmol) were combined in dioxane (15 mL) in a sealed microwave vial. The mixture was degassed and heated at 85° C. for 8 hrs. The mixture was diluted with EtOAc, washed with water, brine, dried over Na₂SO₄ and concentrated. The residue was purified by silica gel column (EtOAc/hexanes gradient 0-100% over 10CVs) to give 4-(6-(4,4,5,5-tetramethyl-1,3,2-dioxaborolan-2-yl)-3,4-dihydroisoquinolin-2(1H)-yl)benzofuro[3,2-d]pyrimidine (1.2 g, 2.8 mmol, 76% yield). ¹H NMR (500 MHz, CDCl₃) δ 8.65 (s, 1H), 8.18 (d, J=7.7 Hz, 1H), 7.73-7.59 (m, 5H), 7.45 (ddd, J=7.8, 6.8, 1.3 Hz, 1H), 7.30 (d, J=7.6 Hz, 1H), 5.25 (s, 2H), 4.39 (t, J=5.9 Hz, 2H), 3.10 (t, J=5.8 Hz, 2H), 1.36 (s, 12H). LCMS (M+H): 428.10.

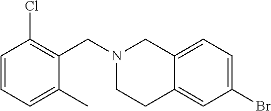 [SMILES: Cc1cccc(Cl)c1CN1CCc2cc(Br)ccc2C1]

### 6-Bromo-2-(2-chloro-6-methylbenzyl)-1,2,3,4-tetrahydroisoquinoline

To a solution of 6-bromo-1,2,3,4-tetrahydroisoquinoline (1.25 g, 5.88 mmol) in DCM (25 mL) was added 2-chloro-6-methylbenzaldehyde (1.0 g, 6.5 mmol) and acetic acid (0.337 mL, 5.88 mmol) in DCM (25 mL). Then sodium triacetoxyborohydride (1.62 g, 7.64 mmol) was added. The mixture was stirred at r.t for 16 hrs. The mixture was quenched with water and extracted with EtOAc. The organic layer was washed with brine, dried over Na₂SO₄ and concentrated. The residue was purified by recrystallization with EtOAc to give 6-bromo-2-(2-chloro-6-methylbenzyl)-1,2,3,4-tetrahydroisoquinoline (1.44 g, 4.11 mmol, 69.8% yield). LCMS (M+H): 350.00, 352.00. ¹H NMR (400 MHz, DMSO-d₆) δ 7.32-7.14 (m, 5H), 6.99 (d, J=8.1 Hz, 1H), 3.77 (s, 2H), 3.56 (s, 2H), 2.78-2.72 (m, 2H), 2.71-2.66 (m, 2H), 2.41 (s, 3H).

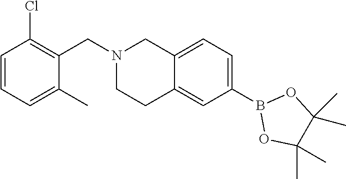 [SMILES: Cc1cccc(Cl)c1CN1CCc2cc(B3OC(C)(C)C(C)(C)O3)ccc2C1]

### 2-(2-Chloro-6-methylbenzyl)-6-(4,4,5,5-tetramethyl-1,3,2-dioxaborolan-2-yl)-1,2,3,4-tetrahydroisoquinoline

6-Bromo-2-(2-chloro-6-methylbenzyl)-1,2,3,4-tetrahydroisoquinoline (1.00 g, 2.85 mmol), 4,4,4′,4′,5,5,5′,5′-octamethyl-2,2′-bi(1,3,2-dioxaborolane) (1.09 g, 4.28 mmol), Pd(dppf)Cl₂ (0.209 g, 0.285 mmol) and potassium acetate (0.840 g, 8.55 mmol) were combined in dioxane (10 mL) in a sealed bottle. The mixture was degassed and heated at 85° C. for 8 hrs. The mixture was diluted with EtOAc, washed with water, brine, dried over MgSO₄ and concentrated. The residue was purified by silica gel column (EtOAc/hexanes gradient) to give 2-(2-chloro-6-methylbenzyl)-6-(4,4,5,5-tetramethyl-1,3,2-dioxaborolan-2-yl)-1,2,3,4-tetrahydroisoquinoline (1.05 g, 2.64 mmol, 93% yield). 1H NMR (400 MHz, CDCl₃) δ 7.57-7.51 (m, 2H), 7.23 (d, J=7.6 Hz, 1H), 7.14-7.06 (m, 2H), 7.02 (d, J=7.6 Hz, 1H), 3.83 (s, 2H), 3.71 (s, 2H), 2.88-2.76 (m, 4H), 2.46 (s, 3H), 1.34 (s, 12H). LCMS (M+H): 398.05.

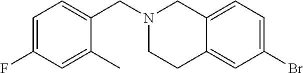 [SMILES: Cc1cc(F)ccc1CN1CCc2cc(Br)ccc2C1]

### 6-Bromo-2-(4-fluoro-2-methylbenzyl)-1,2,3,4-tetrahydroisoquinoline

To a solution of 6-bromo-1,2,3,4-tetrahydroisoquinoline (4.19 g, 19.7 mmol) in DCM (75 mL) was added 4-fluoro-2-methylbenzaldehyde (3.0 g, 22 mmol) and acetic acid (1.13 mL, 19.7 mmol). Then sodium triacetoxyborohydride (5.4 g, 26 mmol) was added. The mixture was stirred at RT for 16 hrs. The mixture was quenched with water and extracted with DCM. The organic layer was washed with brine, dried over Na₂SO₄ and concentrated. The residue was purified by recrystallization with EtOAc to give 6-bromo-2-(4-fluoro-2-methylbenzyl)-1,2,3,4-tetrahydroisoquinoline (3.4 g, 10.17 mmol, 51.5% yield). LCMS (M+H)=333.95 and 335.90.

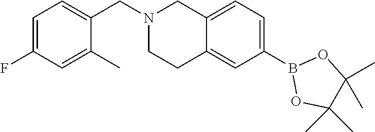 [SMILES: Cc1cc(F)ccc1CN1CCc2cc(B3OC(C)(C)C(C)(C)O3)ccc2C1]

### 2-(4-Fluoro-2-methylbenzyl)-6-(4,4,5,5-tetramethyl-1,3,2-dioxaborolan-2-yl)-1,2,3,4-tetrahydroisoquinoline

6-Bromo-2-(4-fluoro-2-methylbenzyl)-1,2,3,4-tetrahydroisoquinoline (3.4 g, 10.2 mmol), PdCl₂(dppf) (0.74 g, 1.0 mmol), 4,4,4′,4′,5,5,5′,5′-octamethyl-2,2′-bi(1,3,2-dioxaborolane) (3.1 g, 12 mmol) and potassium acetate (3.0 g, 30 mmol) were combined in Dioxane (35.7 ml) under N₂. The mixture was degassed and heated at 85° C. overnight. The mixture was diluted with EtOAc, washed with water, brine, dried over Na₂SO₄ and concentrated. The residue was purified by silica gel column (EtOAc/hexanes gradient (0-100%)) to give 2-(4-fluoro-2-methylbenzyl)-6-(4,4,5,5-tetramethyl-1,3,2-dioxaborolan-2-yl)-1,2,3,4-tetrahydroisoquinoline (1.98 g, 5.19 mmol, 51.0% yield). LCMS (M+H)=382.30.

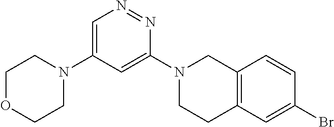 [SMILES: Brc1ccc2c(c1)CCN(c1cc(N3CCOCC3)cnn1)C2]

### 4-(6-(6-Bromo-3,4-dihydroisoquinolin-2(1H)-yl)pyridazin-4-yl)morpholine

To a microwave vial containing 3,5-dichloropyridazine (130.9 mg, 0.879 mmol) was added a solution of morpholine (81 μL, 0.920 mmol) and triethylamine (245 μL, 1.758 mmol) in NMP (2.5 mL). The reaction vial was flushed briefly with nitrogen and capped and allowed to stir at room temp for 30 min. The reaction was then heated to 100° C. for 3 h 20 min. The reaction was then treated with 6-bromo-1,2,3,4-tetrahydroisoquinoline (240 mg, 1.132 mmol) and additional triethylamine (300 μlit, 2.15 mmol), flushed briefly with nitrogen, securely capped and heated at 100° C. for 18 h. The reaction was further heated in a microwave reactor at 150-160° C. for 15.5h. The crude reaction was purified via reverse phase prep-HPLC to afford 4-(6-(6-bromo-3,4-dihydroisoquinolin-2(1H)-yl)pyridazin-4-yl)morpholine, 50.4 mg (15%). LCMS (M+1)=375.1, 377.1. ¹H NMR (500 MHz, CDCl₃) δ 8.42 (br s, 1H), 7.37-7.33 (m, 2H), 7.09 (d, J=8.2 Hz, 1H), 6.03 (d, J=2.3 Hz, 1H), 4.75 (s, 2H), 3.88 (t, J=5.0 Hz, 6H), 3.36-3.32 (m, 4H), 2.98 (t, J=5.9 Hz, 2H).

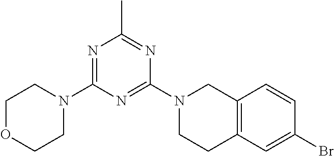 [SMILES: Cc1nc(N2CCOCC2)nc(N2CCc3cc(Br)ccc3C2)n1]

### 4-(4-(6-Bromo-3,4-dihydroisoquinolin-2(1H)-yl)-6-methyl-1,3,5-triazin-2-yl)morpholine

To a dry microwave vial under nitrogen was added 2,4-dichloro-6-methyl-1,3,5-triazine (150 mg, 0.915 mmol), 6-bromo-1,2,3,4-tetrahydroisoquinoline (195 mg, 0.919 mmol) and anhydrous NMP (2.5 mL). The reaction was flushed with nitrogen, then treated with triethylamine (400 μL, 2.87 mmol), capped and allowed to stir at room temp for 30 min. The reaction was then treated with morpholine (800 mg, 9.18 mmol) and stirred at room temp for 2 h. The resulting white solid was collected by vacuum filtration to afford 4-(4-(6-bromo-3,4-dihydroisoquinolin-2(1H)-yl)-6-methyl-1,3,5-triazin-2-yl)morpholine, 200 mg (54%). LCMS (M+1)=390.1, 392.1. ¹H NMR (500 MHz, DMSO-d₆) δ 7.41 (d, J=1.7 Hz, 1H), 7.38 (dd, J=8.2, 2.1 Hz, 1H), 7.23 (d, J=8.2 Hz, 1H), 4.86-4.77 (m, 2H), 3.93 (br s, 2H), 3.78-3.69 (m, 4H), 3.62 (br d, J=4.6 Hz, 4H), 2.84 (br t, J=5.6 Hz, 2H), 2.20 (s, 3H).

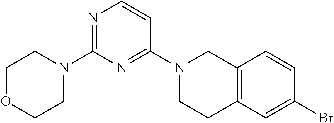 [SMILES: Brc1ccc2c(c1)CCN(c1ccnc(N3CCOCC3)n1)C2]

### 4-(4-(6-bromo-3,4-dihydroisoquinolin-2(1H)-yl)pyrimidin-2-yl)morpholine

To solid 2,4-dichloropyrimidine (184.8 mg, 1.240 mmol) was slowly added a solution of 6-bromo-1,2,3,4-tetrahydroisoquinoline (262 mg, 1.235 mmol) and triethylamine (390 μL, 2.80 mmol) in NMP (3 mL). The reaction was stirred at rt for 20 min then heated to 80° C. for 1 h 45 min. The reaction was then treated with morpholine (500 μL, 5.74 mmol) and heated to 100° C. for 18 h. The crude reaction was purified via reverse phase Prep HPLC to afford 4-(4-(6-bromo-3,4-dihydroisoquinolin-2(1H)-yl)pyrimidin-2-yl)morpholine, 386 mg (83%). LCMS (M+1)=375.1, 377.1. ¹H NMR (500 MHz, CDCl₃) δ 8.02-7.98 (m, 1H), 7.38-7.34 (m, 2H), 7.08 (d, J=8.7 Hz, 1H), 5.96 (d, J=6.0 Hz, 1H), 4.67 (s, 2H), 3.86-3.76 (m, 10H), 2.92 (t, J=5.8 Hz, 2H).

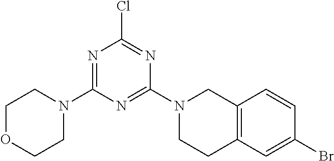 [SMILES: Clc1nc(N2CCOCC2)nc(N2CCc3cc(Br)ccc3C2)n1]

### 4-(4-(6-Bromo-3,4-dihydroisoquinolin-2(1H)-yl)-6-chloro-1,3,5-triazin-2-yl)morpholine

A solution of 6-bromo-1,2,3,4-tetrahydroisoquinoline (100 mg, 0.472 mmol) and triethylamine (180 μL, 1.291 mmol) in anhydrous NMP (2.5 mL) was added to 2,4-dichloro-6-morpholino-1,3,5-triazine (100 mg, 0.425 mmol). The reaction was flushed briefly with nitrogen, capped, stirred at room temp for 30 min at room temp, followed by heating at 80° C. for 1 h. The crude reaction was purified via reverse phase Prep HPLC to afford 4-(4-(6-bromo-3,4-dihydroisoquinolin-2(1H)-yl)-6-chloro-1,3,5-triazin-2-yl)morpholine, 150 mg (86%). LCMS (M+1)=410.1, 412.1. ¹H NMR (500 MHz, DMSO-d₆) δ 7.48-7.35 (m, 2H), 7.24 (br dd, J=11.8, 8.3 Hz, 1H), 4.79 (br d, J=17.4 Hz, 2H), 3.90 (dt, J=14.6, 5.9 Hz, 2H), 3.76 (br s, 2H), 3.71-3.58 (m, 6H), 2.86 (br d, J=6.1 Hz, 2H).

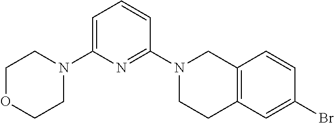 [SMILES: Brc1ccc2c(c1)CCN(c1cccc(N3CCOCC3)n1)C2]

### 4-(6-(6-Bromo-3,4-dihydroisoquinolin-2(1H)-yl)pyridin-2-yl)morpholine

To a dry microwave vial under nitrogen was added 4-(6-chloropyridin-2-yl)morpholine (100 mg, 0.503 mmol), 6-bromo-1,2,3,4-tetrahydroisoquinoline (160 mg, 0.754 mmol) and NMP (2.5 mL). The reaction was flushed briefly with nitrogen, treated with triethylamine (175 μL, 1.256 mmol) and placed in a microwave heating unit at 150-160° C. for 75 h. The crude reaction was purified via reverse phase Prep HPLC to afford 4-(6-(6-bromo-3,4-dihydroisoquinolin-2(1H)-yl)pyridin-2-yl)morpholine, 64.5 mg (34%). LCMS (M+1)=374.0, 376.0. ¹H NMR (500 MHz, CDCl₃) δ 7.40 (t, J=8.0 Hz, 1H), 7.35-7.31 (m, 2H), 7.07 (d, J=8.7 Hz, 1H), 6.10 (d, J=8.1 Hz, 1H), 6.00 (d, J=8.1 Hz, 1H), 4.62 (s, 2H), 3.90-3.78 (m, 6H), 3.56-3.47 (m, 4H), 2.93 (t, J=5.7 Hz, 2H).

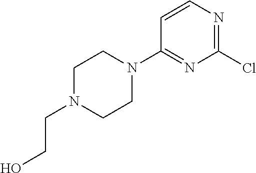 [SMILES: OCCN1CCN(c2ccnc(Cl)n2)CC1]

### 2-(4-(2-Chloropyrimidin-4-yl)piperazin-1-yl)ethan-1-ol

To a dry microwave vial under nitrogen was added 2,4-dichloropyrimidine (350 mg, 2.349 mmol) and acetonitrile (14 mL). The reaction was flushed briefly with nitrogen, then treated with N-(2-hydroxyethyl)piperazine (289 μL, 2.353 mmol), followed by N,N-diisopropylethylamine (1.2 mL, 6.87 mmol). The reaction was capped and placed in a microwave reactor at 100° C. for 8 h. The crude material was purified via silica gel chromatography (40 g SiO₂ column, dichloromethane:acetone 100:0->0:100) to afford 2-(4-(2-chloropyrimidin-4-yl)piperazin-1-yl)ethan-1-ol, 242.8 mg (43%). LCMS (M+1)=243.0, 245.1. ¹H NMR (500 MHz, CDCl₃) δ 8.07 (d, J=6.1 Hz, 1H), 6.41 (d, J=6.1 Hz, 1H), 3.77-3.65 (m, 6H), 2.62 (dt, J=7.1, 5.3 Hz, 6H).

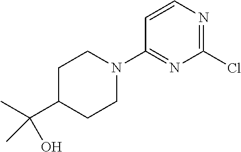 [SMILES: CC(C)(O)C1CCN(c2ccnc(Cl)n2)CC1]

### 2-(1-(2-Chloropyrimidin-4-yl)piperidin-4-yl)propan-2-ol

To a dry microwave vial under nitrogen was added 2,4-dichloropyrimidine (400 mg, 2.68 mmol), 2-(4-piperidyl)-2-propanol (385 mg, 2.68 mmol) and acetonitrile (14 mL). The reaction was flushed briefly with nitrogen, treated with N,N-diisopropylethylamine (1.2 mL, 6.87 mmol), capped and placed in a microwave reactor at 100° C. for 8 h. The crude material was purified via silica gel chromatography (40 g SiO₂ column, dichloromethane:ethyl acetate 100:0->0:100) to afford 2-(1-(2-chloropyrimidin-4-yl)piperidin-4-yl)propan-2-ol, 606 mg (88%). LCMS (M+1)=256.0, 258.1. ¹H NMR (500 MHz, CDCl₃) δ 8.02 (d, J=6.3 Hz, 1H), 6.41 (d, J=6.3 Hz, 1H), 2.88 (br t, J=12.7 Hz, 2H), 1.91 (br d, J=13.3 Hz, 2H), 1.67-1.55 (m, 2H), 1.40-1.26 (m, 3H), 1.22 (s, 6H).

Also isolated from this reaction was

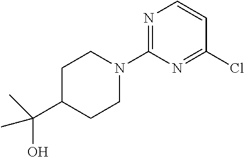 [SMILES: CC(C)(O)C1CCN(c2nccc(Cl)n2)CC1]

### 2-(1-(4-Chloropyrimidin-2-yl)piperidin-4-yl)propan-2-ol (75.3 mg, 11%)

LCMS (M+1)=256.1, 258.1. ¹H NMR (500 MHz, CDCl₃) δ 8.15 (d, J=5.2 Hz, 1H), 6.47 (d, J=5.0 Hz, 1H), 4.87 (dt, J=13.3, 2.2 Hz, 2H), 2.83 (td, J=13.0, 2.4 Hz, 2H), 1.93-1.84 (m, 2H), 1.57 (m, 1H), 1.31 (dd, J=12.5, 4.3 Hz, 2H), 1.22 (s, 7H).

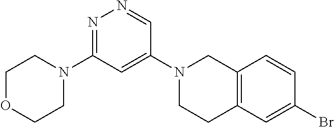 [SMILES: Brc1ccc2c(c1)CCN(c1cnnc(N3CCOCC3)c1)C2]

### 4-(5-(6-Bromo-3,4-dihydroisoquinolin-2(1H)-yl)pyridazin-3-yl)morpholine

A solution of 6-bromo-1,2,3,4-tetrahydroisoquinoline (185 mg, 0.872 mmol) and triethylamine (260 μL, 1.865 mmol) in NMP (3 mL) was slowly added to solid 3,5-dichloropyridazine (130 mg, 0.873 mmol) with stirring at room temperature in a microwave vial. The reaction was capped and stirred at room temp for 30 min, followed by heating at 100° C. for 100 min. The reaction was then treated with morpholine (400 μL, 4.59 mmol) and placed in a microwave reactor at 160° C. for 1 h. The crude reaction was purified via reverse phase Prep-HPLC followed by silica gel chromatography (12 g SiO₂ column, dichloromethane:ethyl acetate 100:0->0:100) to afford 4-(5-(6-bromo-3,4-dihydroisoquinolin-2(1H)-yl)pyridazin-3-yl)morpholine, 63 mg (19%). LCMS (M+1)=375.1, 377.1. ¹H NMR (500 MHz, CDCl₃) δ 8.50 (br s, 1H), 7.42-7.35 (m, 2H), 7.08 (d, J=8.9 Hz, 1H), 6.00 (d, J=2.1 Hz, 1H), 4.45 (s, 2H), 3.91-3.80 (m, 4H), 3.69-3.55 (m, 6H), 2.98 (t, J=5.9 Hz, 2H).

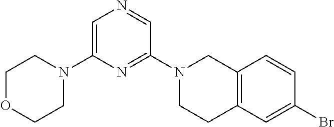 [SMILES: Brc1ccc2c(c1)CCN(c1cncc(N3CCOCC3)n1)C2]

### 4-(6-(6-Bromo-3,4-dihydroisoquinolin-2(1H)-yl)pyrazin-2-yl)morpholine

A solution of 6-bromo-1,2,3,4-tetrahydroisoquinoline (220 mg, 1.037 mmol) and triethylamine (300 μL, 2.152 mmol) in NMP (3 mL) was slowly added to solid 2,6-dichloropyrazine (153.2 mg, 1.028 mmol) with stirring in a microwave vial. The reaction was stirred at room temp for 2 min, then heated to 100° C. for 1.75 h. The reaction was then treated with morpholine (400 μL, 4.59 mmol) and heated at 100° C. sand bath for 65 h. The crude reaction was purified via reverse phase Prep-HPLC to afford 4-(6-(6-bromo-3,4-dihydroisoquinolin-2(1H)-yl)pyrazin-2-yl)morpholine, 270 mg (70%). LCMS (M+1)=375.1, 377.1. ¹H NMR (400 MHz, CDCl₃) δ 7.55 (s, 1H), 7.45 (s, 1H), 7.34 (dd, J=4.3, 2.3 Hz, 2H), 7.15-7.03 (m, 1H), 4.63 (s, 2H), 3.89-3.79 (m, 6H), 3.59-3.48 (m, 4H), 2.94 (t, J=5.8 Hz, 2H).

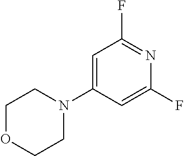 [SMILES: Fc1cc(N2CCOCC2)cc(F)n1]

### 4-(2,6-Difluoropyridin-4-yl)morpholine

To a magnetically stirred solution of 2,4,6-trifluoropyridine (878 mg, 6.60 mmol) in acetonitrile (10 mL) was added a solution of morpholine (586 mg, 6.73 mmol) and Hunig's base (1.35 mL, 7.73 mmol) in acetonitrile (10 mL) and the reaction was allowed to stir at room temp for 1.5 h. The crude material was purified via silica gel chromatography (80 g SiO₂ column, hexane:dichloromethane 100:0->0:100) to afford 4-(2,6-difluoropyridin-4-yl)morpholine, 635.8 mg (48%). LCMS (M+1)=201.0. ¹H NMR (500 MHz, CDCl₃) δ 6.12 (s, 2H), 3.95-3.76 (m, 4H), 3.37-3.30 (m, 4H).

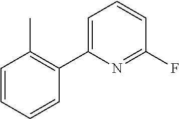

### 2-Fluoro-6-(o-tolyl)pyridine

A solution of 2-bromo-6-fluoropyridine (2.4 g, 13.64 mmol), o-tolylboronic acid (2.039 g, 15.00 mmol) and Tetrakis (0.158 g, 0.136 mmol) in Dioxane was degassed by nitrogen bubble for 10 min. A solution of Phosphoric acid, potassium salt (8.68 g, 40.9 mmol) in H₂O (2 ml) was then added and the solution heated to reflux for 18 h. The crude material was purified via silica gel chromatography (90 g SiO₂ column, hexane:EtOAc 100:0->90:10) to afford 2-fluoro-6-(o-tolyl)pyridine, 2.41 g (94%). ¹H NMR (400 MHz, CDCl₃) δ 7.87 (q, J=8.2 Hz, 1H), 7.46-7.41 (m, 1H), 7.37-7.26 (m, 4H), 6.92 (dd, J=8.2, 2.9 Hz, 1H), 2.43 (s, 3H).

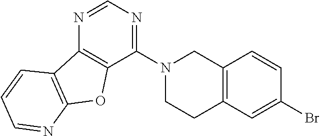 [SMILES: Brc1ccc2c(c1)CCN(c1ncnc3c1oc1ncccc13)C2]

### 4-(6-Bromo-3,4-dihydroisoquinolin-2(1H)-yl)pyrido[3′,2′:4,5]furo[3,2-d]pyrimidine

To a dry microwave vial under nitrogen was added 4-chloropyrido[3′,2′:4,5]furo[3,2-d]pyrimidine (200 mg, 0.973 mmol), 6-bromo-1,2,3,4-tetrahydroisoquinoline*HCl (300 mg, 1.207 mmol) and acetonitrile (12 mL). The reaction was then treated with Hunig's base (510 μL, 2.92 mmol), capped, heated 85° C. (sand bath) then placed in a microwave reactor at 130° C. for 4 h. The resulting solid was colledted by filtration to afford 4-(6-bromo-3,4-dihydroisoquinolin-2(1H)-yl)pyrido[3′,2′:4,5]furo[3,2-d]pyrimidine, 32 mg (87%). LCMS (M+1)=381.0 and 383.0. ¹H NMR (500 MHz, CDCl₃) δ 8.67 (s, 1H), 8.64 (dd, J=4.9, 1.7 Hz, 1H), 8.55 (dd, J=7.6, 1.7 Hz, 1H), 7.49 (dd, J=7.6, 4.9 Hz, 1H), 7.40-7.36 (m, 2H), 7.17 (d, J=8.7 Hz, 1H), 5.20 (s, 2H), 4.40 (t, J=5.7 Hz, 2H), 3.08 (t, J=5.9 Hz, 2H).

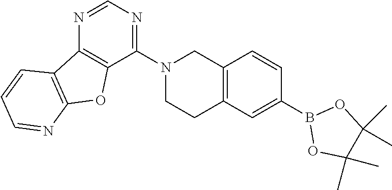 [SMILES: CC1(C)OB(c2ccc3c(c2)CCN(c2ncnc4c2oc2ncccc24)C3)OC1(C)C]

### 4-(6-(4,4,5,5-Tetramethyl-1,3,2-dioxaborolan-2-yl)-3,4-dihydroisoquinolin-2(1H)-yl)pyrido[3′,2′:4,5]furo[3,2-d]pyrimidine

To a dry reaction vial under nitrogen was added 4-(6-bromo-3,4-dihydroisoquinolin-2(1H)-yl)pyrido[3′,2′:4,5]furo[3,2-d]pyrimidine (302 mg, 0.792 mmol), 4,4,4′,4′,5,5,5′,5′-octamethyl-2,2′-bi(1,3,2-dioxaborolane) (350 mg, 1.378 mmol), potassium acetate (324 mg, 3.30 mmol) and dioxane (6 mL). The reaction was then treated with [1,1′-bis(diphenylphosphino)ferrocene]dichloropalladium(II) (45 mg, 0.062 mmol), flushed with argon, capped and heated at 90° C. for 13 h. The reaction was diluted with ethyl acetate (200 mL), extracted with water (1×20 mL), brine (1×50 mL), dried over Na₂SO₄ and concentrated. The crude material was purified via silica gel chromatography (12 g SiO₂ column, dichloromethane:ethyl acetate 100:0->0:100) to afford 4-(6-(4,4,5,5-tetramethyl-1,3,2-dioxaborolan-2-yl)-3,4-dihydroisoquinolin-2(1H)-yl)pyrido[3′,2′:4,5]furo[3,2-d]pyrimidine, 282 mg (83%). LCMS (M+1)=429.2. ¹H NMR (500 MHz, CDCl₃) δ 8.66-8.65 (m, 1H), 8.67 (s, 1H), 8.64 (dd, J=4.9, 1.7 Hz, 1H), 8.54 (dd, J=7.6, 1.7 Hz, 1H), 7.73-7.67 (m, 2H), 7.49 (dd, J=7.7, 4.8 Hz, 1H), 7.31 (d, J=7.5 Hz, 1H), 5.28 (br s, 2H), 4.42 (br s, 2H), 3.12 (br t, J=5.8 Hz, 2H), 1.37 (s, 12H).

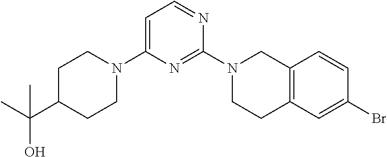 [SMILES: CC(C)(O)C1CCN(c2ccnc(N3CCc4cc(Br)ccc4C3)n2)CC1]

### 2-(1-(2-(6-Bromo-3,4-dihydroisoquinolin-2(1H)-yl)pyrimidin-4-yl)piperidin-4-yl)propan-2-ol, 2.0 methyl-2-pyrrolidinone

To a dry microwave vial under nitrogen was added 2-(1-(2-chloropyrimidin-4-yl)piperidin-4-yl)propan-2-ol (540 mg, 2.111 mmol), 6-bromo-1,2,3,4-tetrahydroisoquinoline (570 mg, 2.69 mmol) and anhydrous NMP (12 mL). The reaction was flushed with argon, treated with N,N-diisopropylethylamine (1.11 mL, 6.36 mmol), capped and heated in a microwave reactor at 160° C. for 4 h. The solvent was removed under a gentle stream of nitrogen and the crude material was purified via silica gel chromatography (40 g SiO₂ column, dichloromethane:ethyl acetate 100:0->0:100) to afford 2-(1-(2-(6-bromo-3,4-dihydroisoquinolin-2(1H)-yl)pyrimidin-4-yl)piperidin-4-yl)propan-2-ol, 2.0 methyl-2-pyrrolidinone, 642.2 mg (48%). LCMS (M+1)=431.2 and 433.1.

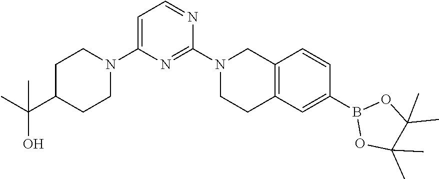 [SMILES: CC(C)(O)C1CCN(c2ccnc(N3CCc4cc(B5OC(C)(C)C(C)(C)O5)ccc4C3)n2)CC1]

### 2-(1-(2-(6-(4,4,5,5-Tetramethyl-1,3,2-dioxaborolan-2-yl)-3,4-dihydroisoquinolin-2(1H)-yl)pyrimidin-4-yl)piperidin-4-yl)propan-2-ol

To a dry pressure bottle under nitrogen was added 2-(1-(2-(6-bromo-3,4-dihydroisoquinolin-2(1H)-yl)pyrimidin-4-yl)piperidin-4-yl)propan-2-ol, 2.01-methyl-2-pyrrolidinone (642.2 mg, 1.020 mmol), bis(pinacolato)diboron (450 mg, 1.772 mmol), potassium acetate (417 mg, 4.25 mmol) and dioxane (12 mL). The reaction was flushed with argon, treated with [1,1′-bis(diphenylphosphino)ferrocene]dichloropalladium(II) (77 mg, 0.105 mmol), capped and heated at 90° C. for 13 h. The reaction was dissolved in EtOAc (200 mL), extracted with water (1×25 mL), brine 91×25 mL), dried over Na₂SO₄ and concentrated. The crude material was purified via silica gel chromatography (80 g SiO₂ column, dichloromethane:ethyl acetate 100:0->0:100) to afford 2-(1-(2-(6-(4,4,5,5-tetramethyl-1,3,2-dioxaborolan-2-yl)-3,4-dihydroisoquinolin-2(1H)-yl)pyrimidin-4-yl)piperidin-4-yl)propan-2-ol, 473 mg (97%). LCMS (M+1)=479.3. ¹H NMR (500 MHz, CDCl₃) δ 7.98 (d, J=6.1 Hz, 1H), 7.66-7.61 (m, 2H), 7.23 (d, J=7.9 Hz, 1H), 5.91 (d, J=6.1 Hz, 1H), 4.91 (s, 2H), 4.52 (br d, J=10.8 Hz, 2H), 4.03 (t, J=5.8 Hz, 2H), 2.94 (t, J=5.7 Hz, 2H), 2.80 (td, J=12.8, 2.1 Hz, 2H), 1.87 (br d, J=13.0 Hz, 2H), 1.36 (s, 12H), 1.22 (s, 6H).

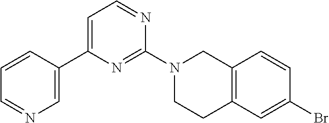 [SMILES: Brc1ccc2c(c1)CCN(c1nccc(-c3cccnc3)n1)C2]

### 6-Bromo-2-(4-(pyridin-3-yl)pyrimidin-2-yl)-1,2,3,4-tetrahydroisoquinoline

To a dry 100 mL pressure bottle under nitrogen was added 2-chloro-4-(pyridin-3-yl)pyrimidine (961 mg, 5.02 mmol), 6-bromo-1,2,3,4-tetrahydroisoquinoline, HCl (1.40 g, 5.63 mmol) and acetonitrile (60 mL). The reaction was flushed briefly with argon, treated with Hunig's base (2.6 mL, 14.89 mmol), capped and heated at 130° C. for 18 h. The resulting tan solid was collected by vacuum filtration to afford 6-bromo-2-(4-(pyridin-3-yl)pyrimidin-2-yl)-1,2,3,4-tetrahydroisoquinoline, 1.62 g (88%). LCMS (M+1)=367.0 and 369.0.

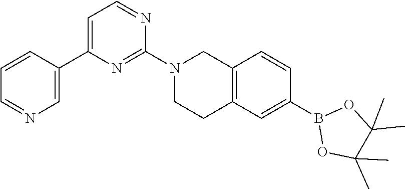 [SMILES: CC1(C)OB(c2ccc3c(c2)CCN(c2nccc(-c4cccnc4)n2)C3)OC1(C)C]

### 2-(4-(Pyridin-3-yl)pyrimidin-2-yl)-6-(4,4,5,5-tetramethyl-1,3,2-dioxaborolan-2-yl)-1,2,3,4-tetrahydroisoquinoline

To a dry 150 mL pressure bottle under nitrogen was added 6-bromo-2-(4-(pyridin-3-yl)pyrimidin-2-yl)-1,2,3,4-tetrahydroisoquinoline (1.62 g, 4.41 mmol), 4,4,4′,4′,5,5,5′,5′-octamethyl-2,2′-bi(1,3,2-dioxaborolane) (1.8 g, 7.09 mmol), potassium acetate (1.8 g, 18.34 mmol) and dioxane (40 mL). The reaction was flushed with argon, treated with [1,1′-bis(diphenylphosphino)ferrocene]dichloropalladium(II) (171 mg, 0.234 mmol), capped and heated at 100° C. for 18 h. The reaction was diluted with ethyl acetate (350 mL), filtered thru a pad of Celite, extracted with water (1×150 mL), brine (1×150 mL), dried over Na₂SO₄ and concentrated. The crude product was purified via silica gel chromatography (80 g SiO₂ column, dichloromethane:EtOAc 100:0->25:75) to afford 2-(4-(pyridin-3-yl)pyrimidin-2-yl)-6-(4,4,5,5-tetramethyl-1,3,2-dioxaborolan-2-yl)-1,2,3,4-tetrahydroisoquinoline, 1.72 g (94%). LCMS (M+1)=415.2.

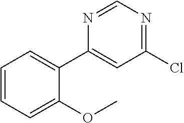

### 4-Chloro-6-(2-methoxyphenyl)pyrimidine

In a vial equipped with a magnetic stirring bar was added 4,6-dichloropyrimidine (200 mg, 1.342 mmol) and (2-methoxyphenyl)boronic acid (204 mg, 1.342 mmol). The solids were suspended in THF (5 mL). The mixture was treated with 0.5M K₃PO₄ (5.91 mL, 2.95 mmol) and X-Phos precatalyst G2 (57.0 mg, 0.072 mmol). Argon was streamed over the mixture for 5 minutes. The vial was capped and stirred at RT for 16 hours. LC/MS showed about a 1:1 mixture of mono- and bis-Suzuki coupling products. Separated layers and concentrated down under vacuum to give crude 4-chloro-6-(2-methoxyphenyl)pyrimidine that was used as is to prepare the following example. LCMS (M+1)=221.2.

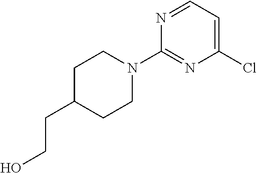 [SMILES: OCCC1CCN(c2nccc(Cl)n2)CC1]

### 2-(1-(4-Chloropyrimidin-2-yl)piperidin-4-yl)ethanol

To a dry 10-20 mL microwave vial under nitrogen is added 2,4-dichloropyrimidine (422 mg, 2.83 mmol) and 2-(piperidin-4-yl)ethanol (382 mg, 2.96 mmol), and acetonitrile (14 mL). The reaction is flushed briefly with nitrogen, then treated with Hunig's base (1.2 mL, 6.87 mmol). The reaction is capped and stirred at RT for 1 h then placed in a microwave reactor at 105° C. for 10 hours. LCMS shows about 3:1 mix of major and minor product isomer peaks. Removed solvent under a nitrogen stream. The residue was purified by flash column chromatography to give 2-(1-(4-chloropyrimidin-2-yl)piperidin-4-yl)ethanol (50 mg; 7%). LCMS (M+1)=242.1.

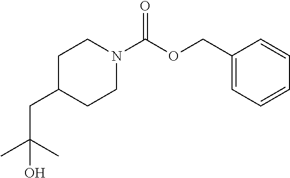 [SMILES: CC(C)(O)CC1CCN(C(=O)OCc2ccccc2)CC1]

### Benzyl 4-(2-hydroxy-2-methylpropyl)piperidine-1-carboxylate

A round flask containing a magnetic stirrer bar was added benzyl 4-(2-ethoxy-2-oxoethyl)piperidine-1-carboxylate (0.5 g, 1.637 mmol) in THF (10 mL). The solution was cooled to −78° C. (dry ice/acetone bath) under nitrogen and 3M methyl magnesium bromide/Et₂O (1.364 mL, 4.09 mmol) was added dropwise over 10 minutes. The resulting mixture was allowed to slowly warm to RT over several hours while stirring. LC/MS showed that reaction still contained some starting material as well as desired M+1 product. The solution was cooled to −78° C. (dry ice/acetone bath) under nitrogen and 3M methyl magnesium bromide/Et₂O (1.25 equiv.; 0.7 mL was added dropwise over a few minutes. The resulting mixture was allowed to slowly warm to RT over several hours while stirring. LC/MS now showed no remaining starting material and desired M+1 peak as major. The reaction was quenched with 5 mL of sat. NH₄Cl solution while stirring. The organic solvents were removed under vacuum. The remaining water layer and solids were extracted with ethyl acetate, dried over MgSO₄, filtered, and concentrated down to give an oil. The oil was purified by flash column chromatography to give 320 mg (67%) of benzyl 4-(2-hydroxy-2-methylpropyl)piperidine-1-carboxylate (320 mg, 1.098 mmol) as a colorless oil. LCMS (M+1)=291.85.

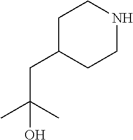

### 2-Methyl-1-(piperidin-4-yl)propan-2-ol

A round bottom flask equipped with a magnetic stirring bar was added benzyl 4-(2-hydroxy-2-methylpropyl)piperidine-1-carboxylate (320 mg, 1.098 mmol) in MeOH (50 mL). The solution was treated with palladium on carbon (146 mg, 0.137 mmol) and the flask was capped with a rubber septum, purged with nitrogen, and put under 1 atm of hydrogen via a balloon. The reaction was allowed to stir for 16 hours. LC/MS showed that the starting material was consumed. Filter reaction mixture thru Celite, washed Celite with excess methanol and dried down under high vacuum to give 146 mg (85%) of 2-methyl-1-(piperidin-4-yl)propan-2-ol as a pale yellow oil that solidified into yellow needles. ¹H NMR (400 MHz, CDCl₃) δ 2.97 (br d, J=11.3 Hz, 2H), 2.13-1.92 (m, 1H), 1.90-1.60 (m, 4H), 1.55-1.36 (m, 4H), 1.36-1.16 (m, 6H).

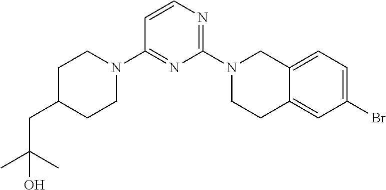 [SMILES: CC(C)(O)CC1CCN(c2ccnc(N3CCc4cc(Br)ccc4C3)n2)CC1]

### 1-(1-(2-(6-Bromo-3,4-dihydroisoquinolin-2(1H)-yl)pyrimidin-4-yl)piperidin-4-yl)-2-methylpropan-2-ol

In a 20 mL septa top pressure vial equipped with a magnetic stirring bar was added 6-bromo-2-(4-chloropyrimidin-2-yl)-1,2,3,4-tetrahydroisoquinoline (77 mg, 0.237 mmol), and 2-methyl-1-(piperidin-4-yl)propan-2-ol (56.0 mg, 0.356 mmol) in acetonitrile (2 mL). Hunig's base (0.104 mL, 0.593 mmol) was added and the mixture was heated to 100° C. in a preheated aluminum block and allowed to stir for 16 hours overnight. LC/MS showed some desired product but also remaining bromide starting material. Added 28 mg (1 equiv.) of 2-methyl-1-(piperidin-4-yl)propan-2-ol, capped vial and heated again at 100° C. The next day, the reaction mixture was concentrated down on a stream of nitrogen. The residue was purified by column chromatography to give 41 mg (35%) of 1-(1-(2-(6-bromo-3,4-dihydroisoquinolin-2(1H)-yl)pyrimidin-4-yl)piperidin-4-yl)-2-methylpropan-2-ol. LCMS (M+1)=444.7/446.7.

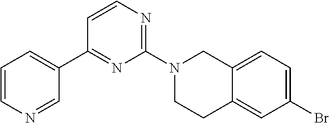 [SMILES: Brc1ccc2c(c1)CCN(c1nccc(-c3cccnc3)n1)C2]

### 6-Bromo-2-(4-(pyridin-3-yl)pyrimidin-2-yl)-1,2,3,4-tetrahydroisoquinoline

In a pressure vessel equipped with a magnetic stirring bar was added 6-bromo-1,2,3,4-tetrahydroisoquinoline (1.328 g, 6.26 mmol), and 2-chloro-4-(pyridin-3-yl)pyrimidine (1 g, 5.22 mmol) in acetonitrile (25 mL). Hunig's base (2.73 mL, 15.66 mmol) was added and the mixture was heated to 80° C. in a preheated oil bath and allowed to stir for 16 hours overnight. Reaction appears complete by LC/MS, cooled to RT and filtered solids, washed with ethyl acetate, concentrated in vacuo to a solid. Took up solid in EtOAc: heated to dissolve most material in minimum amount of solvent, filtered while hot, and allowed to cool to RT. After filtration and drying under vacuum, 1.5 g (87%) of 6-bromo-2-(4-(pyridin-3-yl)pyrimidin-2-yl)-1,2,3,4-tetrahydroisoquinoline was obtained as a light brown solid. LCMS (M+1)=366.7 and 368.6.

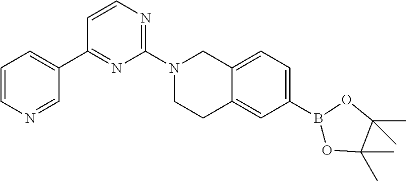 [SMILES: CC1(C)OB(c2ccc3c(c2)CCN(c2nccc(-c4cccnc4)n2)C3)OC1(C)C]

### 2-(4-(Pyridin-3-yl)pyrimidin-2-yl)-6-(4,4,5,5-tetramethyl-1,3,2-dioxaborolan-2-yl)-1,2,3,4-tetrahydroisoquinoline

In a pressure vessel equipped with a magnetic stirring bar was added recrystallized 6-bromo-2-(4-(pyridin-3-yl)pyrimidin-2-yl)-1,2,3,4-tetrahydroisoquinoline (1.5 g, 4.08 mmol), PdCl₂(dppf).CH₂Cl₂ adduct (0.334 g, 0.408 mmol), potassium acetate (1.203 g, 12.25 mmol) and 4,4,4′,4′,5,5,5′,5′-octamethyl-2,2′-bi(1,3,2-dioxaborolane) (1.556 g, 6.13 mmol). The solids were suspended in dioxane (20 mL). Argon was bubbled through the mixture for 5 minutes while sonicating. The flask was capped and heated to 80° C. within a preheated oil bath and allowed to continue for 16 hours. After 16 h at 80° C., LC/MS showed the desired product as major. The reaction mixture was filtered, then concentrated down. The residue was purified by flash column chromatography to give 270 mg (16%) of 2-(4-(pyridin-3-yl)pyrimidin-2-yl)-6-(4,4,5,5-tetramethyl-1,3,2-dioxaborolan-2-yl)-1,2,3,4-tetrahydroisoquinoline as an oil. LCMS (M+1)=414.8.

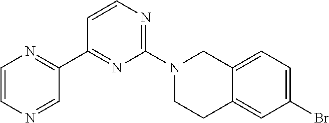 [SMILES: Brc1ccc2c(c1)CCN(c1nccc(-c3cnccn3)n1)C2]

### 6-Bromo-2-(4-(pyrazin-2-yl)pyrimidin-2-yl)-1,2,3,4-tetrahydroisoquinoline

In a pressure vessel equipped with a magnetic stirring bar was added 6-bromo-1,2,3,4-tetrahydroisoquinoline (1.321 g, 6.23 mmol), and 2-chloro-4-(pyrazin-2-yl)pyrimidine (1 g, 5.19 mmol) in acetonitrile (25 mL). Hunig's base (2.72 mL, 15.58 mmol) was added and the mixture was heated to 80° C. in a preheated oil bath and allowed to stir for 16 hours overnight. Reaction appears complete by LC/MS. Cool to RT and removed solvents under a stream of nitrogen, triturated solids with hexanes and filtered under vacuum to give a pale yellow solid. Store under vacuum to dry material. The solid was triturated with 15 mL of methanol and stirred. After 15 minutes of stirring the solid was allowed to settle and most of the methanol was decanted away from the solids. The remaining material was dried under vacuum to give 1.9 g (89%) of 6-bromo-2-(4-(pyrazin-2-yl)pyrimidin-2-yl)-1,2,3,4-tetrahydroisoquinoline as a pale yellow solid. LCMS (M+1)=367.7 and 369.7.

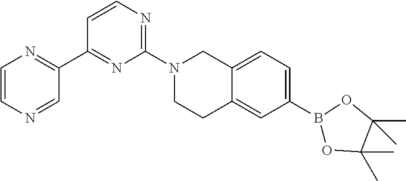 [SMILES: CC1(C)OB(c2ccc3c(c2)CCN(c2nccc(-c4cnccn4)n2)C3)OC1(C)C]

### 2-(4-(Pyrazin-2-yl)pyrimidin-2-yl)-6-(4,4,5,5-tetramethyl-1,3,2-dioxaborolan-2-yl)-1,2,3,4-tetrahydroisoquinoline

In a pressure vessel equipped with a magnetic stirring bar was added 6-bromo-2-(4-(pyrazin-2-yl)pyrimidin-2-yl)-1,2,3,4-tetrahydroisoquinoline (0.9 g, 2.444 mmol), PdCl₂ (dppf).CH₂Cl₂ adduct (0.200 g, 0.244 mmol), potassium acetate (0.720 g, 7.33 mmol) and 4,4,4′,4′,5,5,5′,5′-octamethyl-2,2′-bi(1,3,2-dioxaborolane) (0.931 g, 3.67 mmol). The solids were suspended in dioxane (15 mL). Argon was bubbled through the mixture for 5 minutes while sonicating. The flask was capped and heated to 80° C. within a preheated oil bath and allowed to continue for 16 hours. After 16 h at 80° C., LC/MS showed the desired product (as the boronic ester) as major. The reaction mixture was filtered, then concentrated down. The residue was purified by flash column chromatography to give 0.71 g (42%) of 2-(4-(pyrazin-2-yl)pyrimidin-2-yl)-6-(4,4,5,5-tetramethyl-1,3,2-dioxaborolan-2-yl)-1,2,3,4-tetrahydroisoquinoline as a yellow sticky solid that appeared to be a mixture of boronate and des-bromide starting material. LCMS (M+1)=415.9.

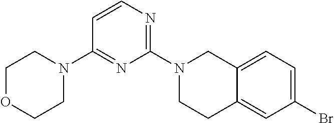 [SMILES: Brc1ccc2c(c1)CCN(c1nccc(N3CCOCC3)n1)C2]

### 4-(2-(6-Bromo-3,4-dihydroisoquinolin-2(1H)-yl)pyrimidin-4-yl)morpholine

To a mixture of 4-(2-chloro-4-pyrimidinyl)morpholine (910 mg, 4.56 mmol) and 6-bromo-1,2,3,4-tetrahydroisoquinoline (967 mg, 4.56 mmol) is added anhydrous NMP (12 mL) and the reaction is flushed well with nitrogen. The reaction is then treated with N,N-diisopropylethylamine (2.0 mL, 11.45 mmol), capped and placed in a 120° C. oil bath for 16 hours. Crude LC/MS seems to indicate complete conversion to the desired product.

Removed NMP and Hunig's base under a stream of nitrogen while heating at 80° C. overnight. A yellow solid remained. Took up the solid in EtOAc, heated to dissolve most material in minimum amount of solvent, filtered while hot, allow to cool to RT slowly to give 1.65 g (91%) of 4-(2-(6-bromo-3,4-dihydroisoquinolin-2(1H)-yl)pyrimidin-4-yl)morpholine as a pale yellow solid as recrystallized material. LCMS (M+1)=374.7 and 376.7.

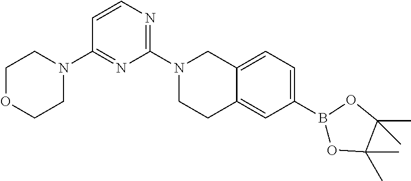 [SMILES: CC1(C)OB(c2ccc3c(c2)CCN(c2nccc(N4CCOCC4)n2)C3)OC1(C)C]

### 4-(2-(6-(4,4,5,5-Tetramethyl-1,3,2-dioxaborolan-2-yl)-3,4-dihydroisoquinolin-2(1H)-yl)pyrimidin-4-yl)morpholine

In a pressure vessel equipped with a magnetic stirring bar was added recrystallized 4-(2-(6-bromo-3,4-dihydroisoquinolin-2(1H)-yl)pyrimidin-4-yl)morpholine (1.2 g, 3.20 mmol), PdCl₂(dppf).CH₂Cl₂ adduct (0.261 g, 0.320 mmol), potassium acetate (0.942 g, 9.59 mmol) and 4,4,4′,4′,5,5,5′,5′-octamethyl-2,2′-bi(1,3,2-dioxaborolane) (1.218 g, 4.80 mmol). The solids were suspended in dioxane (15 mL). Argon was bubbled through the mixture for 5 minutes while sonicating. The flask was capped and heated to 80° C. within a preheated oil bath and allowed to continue for 16 hours. After 16 h at 80° C., LC/MS showed the desired product as major. The reaction mixture was filtered, then concentrated down. The residue was purified by flash column chromatography to give 1.13 g (79%) of 4-(2-(6-(4,4,5,5-tetramethyl-1,3,2-dioxaborolan-2-yl)-3,4-dihydroisoquinolin-2(1H)-yl)pyrimidin-4-yl)morpholine. LCMS (M+1)=422.8.

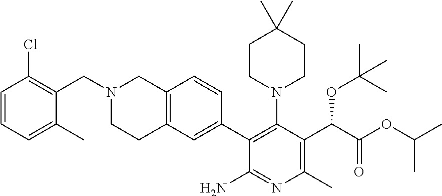 [SMILES: Cc1cccc(Cl)c1CN1CCc2cc(-c3c(N)nc(C)c([C@H](OC(C)(C)C)C(=O)OC(C)C)c3N3CCC(C)(C)CC3)ccc2C1]

### Isopropyl (S)-2-(6-amino-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetate

A mixture of (S)-isopropyl 2-(6-amino-5-bromo-4-(4,4-dimethylpiperidin-1-yl)-2-methylpyridin-3-yl)-2-(tert-butoxy)acetate (0.373 g, 0.793 mmol), 2-(2-chloro-6-methylbenzyl)-6-(4,4,5,5-tetramethyl-1,3,2-dioxaborolan-2-yl)-1,2,3,4-tetrahydroisoquinoline (0.473 g, 1.189 mmol), 2-dicyclohexylphosphino-2′,6′-dimethoxybiphenyl (0.065 g, 0.159 mmol), palladium(II) acetate (0.018 g, 0.079 mmol) and 2 M K₃PO₄ (1.189 mL, 2.379 mmol) was added dioxane (13 mL) and water (3 mL).

The reaction was heated at 80° C. for 1 h. After cooling to ambient temperature, the reaction was partitioned between EtOAc and water. The organic layer was dried (Na₂SO₄) and concentrated in vacuo. The crude product was purified on silica gel (120 g column) using 0-85% ethyl acetate in hexanes. The desired fractions were concentrated in vacuo to give title compound as light brown foamy solid (0.320 g, 62%). LCMS (M+1)=661.40.

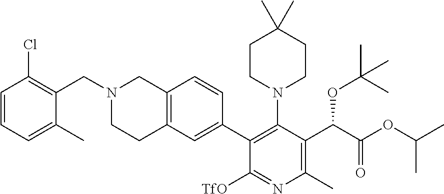 [SMILES: Cc1cccc(Cl)c1CN1CCc2cc(-c3c(OS(=O)(=O)C(F)(F)F)nc(C)c([C@H](OC(C)(C)C)C(=O)OC(C)C)c3N3CCC(C)(C)CC3)ccc2C1]

### Isopropyl (S)-2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate

(S)-Isopropyl 2-(6-amino-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetate (0.588 g, 0.889 mmol) and sodium nitrite (0.153 g, 2.223 mmol) were sequentially added to a mixture of hexanes (2.2 mL), DMSO (0.22 mL) and trifluoromethanesulfonic acid (0.237 ml, 2.67 mmol) at 5° C. The mixture was stirred for 10 min at 5° C. and then for 18 h at ambient temperature. Water was added to the reaction mixture and it was extracted with DCM, dried (Na₂SO₄), and concentrated in vacuo. The residue was purified on silica gel (80 g column) using 0-40% ethyl acetate in hexanes, then 100% ethyl acetate. The desired fractions were concentrated in vacuo to give title compound as yellow solid (0.222 g, 31%). LCMS (M+1)=794.25.

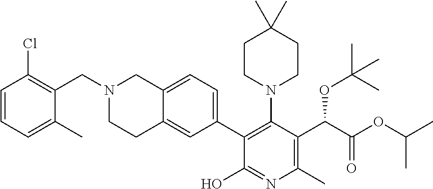 [SMILES: Cc1cccc(Cl)c1CN1CCc2cc(-c3c(O)nc(C)c([C@H](OC(C)(C)C)C(=O)OC(C)C)c3N3CCC(C)(C)CC3)ccc2C1]

### Isopropyl (S)-2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-oxo-1,6-dihydropyridin-3-yl)acetate

(S)-isopropyl 2-(6-amino-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetate (0.588 g, 0.889 mmol) and sodium nitrite (0.153 g, 2.223 mmol) were sequentially added to a mixture of hexanes (2.2 mL), DMSO (0.2 mL) and trifluoromethanesulfonic acid (0.237 ml, 2.67 mmol) at 5° C. The mixture was stirred for 10 min at 5° C. and then at ambient temperature overnight. Water was added to the reaction mixture and it was extracted with DCM, dried (Na₂SO₄), and concentrated in vacuo. The residue was purified on silica gel (80 g column) using 0-40% ethyl acetate in hexanes, then 100% ethyl acetate. The desired fractions (second eluent) were concentrated in vacuo to give title compound as yellow solid (0.035 g, 6%). LCMS (M+1)=662.35.

### Example 1

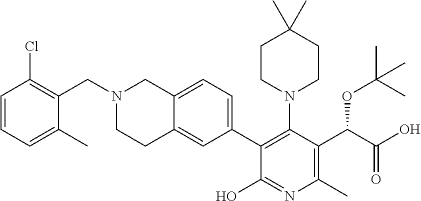 [SMILES: Cc1cccc(Cl)c1CN1CCc2cc(-c3c(O)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-oxo-1,6-dihydropyridin-3-yl)acetic Acid

5 M NaOH (0.211 mL, 1.057 mmol) was added to a solution of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-hydroxy-2-methylpyridin-3-yl)acetate (0.035 g, 0.053 mmol) in ethanol (1.5 mL). The mixture was heated at 80° C. for 6 h and then cooled to ambient temperature and filtered. The crude mixture was purified via preparative LC/MS to afford desired product (13.0 mg, 39%). LCMS (M+1)=620.35.

### Example 2

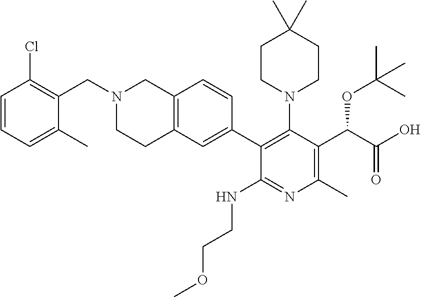 [SMILES: COCCNc1nc(C)c([C@H](OC(C)(C)C)C(=O)O)c(N2CCC(C)(C)CC2)c1-c1ccc2c(c1)CCN(Cc1c(C)cccc1Cl)C2]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-6-((2-methoxyethyl)amino)-2-methylpyridin-3-yl)acetic Acid

A mixture of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate (0.019 g, 0.024 mmol) and 2-methoxyethanamine (7.19 mg, 0.096 mmol) in NMP (1 mL) was heated at 180° C. for 3 h. Ethanol (0.5 mL) and 5 M NaOH (0.048 mL, 0.239 mmol) were added and the mixture was heated at 80° C. for 4.5 h. The reaction was cooled to ambient temperature and filtered. The crude mixture was purified via preparative LC/MS to afford desired product (1.8 mg, 11%). LCMS (M+1)=677.2.

### Example 3

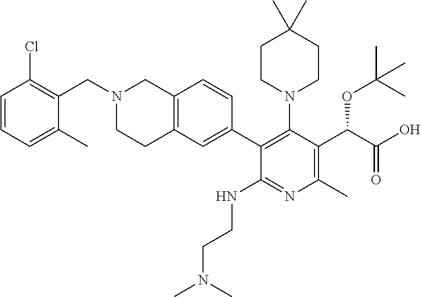 [SMILES: Cc1cccc(Cl)c1CN1CCc2cc(-c3c(NCCN(C)C)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-6-((2-(dimethylamino)ethyl)amino)-4-(4,4-dimethylpiperidin-1-yl)-2-methylpyridin-3-yl)acetic Acid

A mixture of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate (0.019 g, 0.024 mmol) and N1,N1-dimethylethane-1,2-diamine (8.43 mg, 0.096 mmol) in NMP (1 mL) was heated at 180° C. for 3 h. Ethanol (0.5 mL) and 5 M NaOH (0.048 mL, 0.239 mmol) were added and the mixture was heated at 80° C. for 4.5 h. The reaction was cooled to ambient temperature and filtered. The crude mixture was purified via preparative LC/MS to afford desired product (1.8 mg, 11%). LCMS (M+1)=690.2.

### Example 4

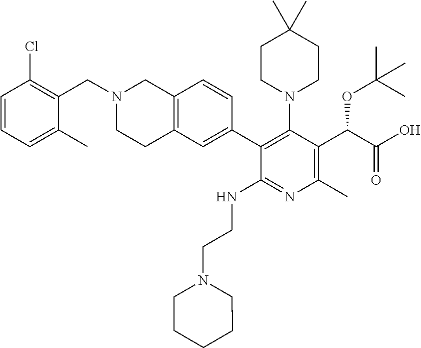 [SMILES: Cc1cccc(Cl)c1CN1CCc2cc(-c3c(NCCN4CCCCC4)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-((2-(piperidin-1-yl)ethyl)amino)pyridin-3-yl)acetic Acid

A mixture of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate (0.019 g, 0.024 mmol) and 2-(piperidin-1-yl)ethanamine (0.012 g, 0.096 mmol) in NMP (1 mL) was heated at 180° C. for 3 h. Ethanol (0.5 mL) and 5 M NaOH (0.048 mL, 0.239 mmol) were added and the mixture was heated at 80° C. for 4.5 h. The reaction was cooled to ambient temperature and filtered. The crude mixture was purified via preparative LC/MS to afford desired product (3.6 mg, 20%). LCMS (M+1)=730.2.

### Example 5

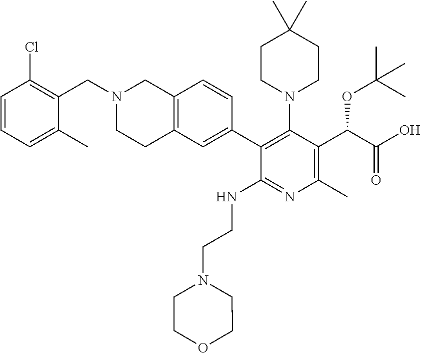 [SMILES: Cc1cccc(Cl)c1CN1CCc2cc(-c3c(NCCN4CCOCC4)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-((2-morpholinoethyl)amino)pyridin-3-yl)acetic Acid

A mixture of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate (0.019 g, 0.024 mmol) and 2-morpholinoethanamine (0.012 g, 0.096 mmol) in NMP (1 mL) was heated at 180° C. for 3 h. Ethanol (0.5 mL) and 5 M NaOH (0.048 mL, 0.239 mmol) were added and the mixture was heated at 80° C. for 4.5 h, cooled to ambient temperature, and filtered. The crude mixture was purified via preparative LC/MS to afford desired product (2.0 mg, 11%). LCMS (M+1)=732.2.

### Example 6

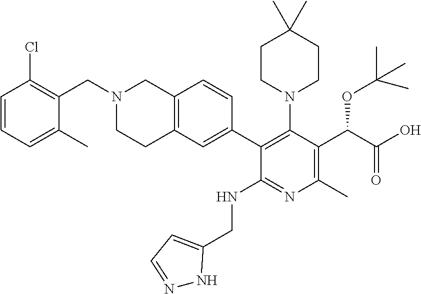 [SMILES: Cc1cccc(Cl)c1CN1CCc2cc(-c3c(NCc4ccn[nH]4)nc(C)c([C@H](OC(C)(C)C)C(=O)O)c3N3CCC(C)(C)CC3)ccc2C1]

### (S)-2-(6-(((1H-Pyrazol-5-yl)methyl)amino)-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetic Acid

A mixture of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate (0.019 g, 0.024 mmol) and (1H-pyrazol-5-yl)methanamine (9.29 mg, 0.096 mmol) in NMP (1 mL) was heated at 180° C. for 3 h. Ethanol (0.5 mL) and 5 M NaOH (0.048 mL, 0.239 mmol) were added and the mixture was heated at 80° C. for 4.5 h, cooled to ambient temperature, and filtered. The crude mixture was purified via preparative LC/MS to afford desired product (5.6 mg, 31%). LCMS (M+1)=699.2.

### Example 7

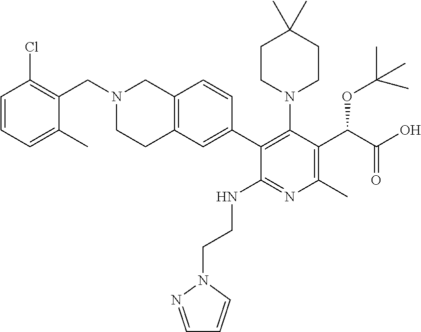 [SMILES: Cc1cccc(Cl)c1CN1CCc2cc(-c3c(NCCn4cccn4)nc(C)c([C@H](OC(C)(C)C)C(=O)O)c3N3CCC(C)(C)CC3)ccc2C1]

### (S)-2-(6-((2-(1H-Pyrazol-1-yl)ethyl)amino)-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetic Acid

A mixture of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate (0.019 g, 0.024 mmol) and 2-(1H-pyrazol-1-yl)ethanamine (10.63 mg, 0.096 mmol) in NMP (1 mL) was heated at 180° C. for 3 h. Ethanol (0.5 mL) and 5 M NaOH (0.048 mL, 0.239 mmol) were added and the mixture was heated at 80° C. for 4.5 h, cooled to ambient temperature, and filtered. The crude mixture was purified via preparative LC/MS to afford desired product (1.4 mg, 8%). LCMS (M+1)=713.2.

### Example 8

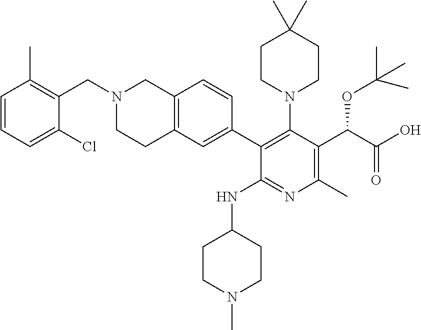 [SMILES: Cc1cccc(Cl)c1CN1CCc2cc(-c3c(NC4CCN(C)CC4)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-((1-methylpiperidin-4-yl)amino)pyridin-3-yl)acetic Acid

A mixture of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate (0.068 g, 0.086 mmol) and 1-methylpiperidin-4-amine (0.039 g, 0.342 mmol) in NMP (1 mL) was heated at 180° C. for 3 h. Ethanol (0.5 mL) and 5 M NaOH (0.171 mL, 0.856 mmol) were added and the mixture was heated at 80° C. for 4.5 h, cooled to ambient temperature, and filtered. The crude mixture was purified via preparative LC/MS to afford desired product (5.3 mg, 8%). LCMS (M+1)=716.4.

### Example 9

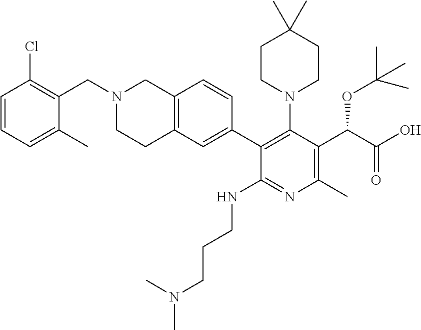 [SMILES: Cc1cccc(Cl)c1CN1CCc2cc(-c3c(NCCCN(C)C)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-6-((3-(dimethylamino)propyl)amino)-4-(4,4-dimethylpiperidin-1-yl)-2-methylpyridin-3-yl)acetic Acid

A mixture of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate (0.02 g, 0.025 mmol) and N1,N1-dimethylpropane-1,3-diamine (10.29 mg, 0.101 mmol) in NMP (1 mL) was heated at 180° C. for 2 h. Ethanol (0.5 mL) and 5 M NaOH (0.050 mL, 0.252 mmol) were added and the mixture was heated at 80° C. for 4.5 h, cooled to ambient temperature, and. The crude mixture was purified via preparative LC/MS to afford desired product (8.5 mg, 47%). LCMS (M+1)=704.1.

### Example 10

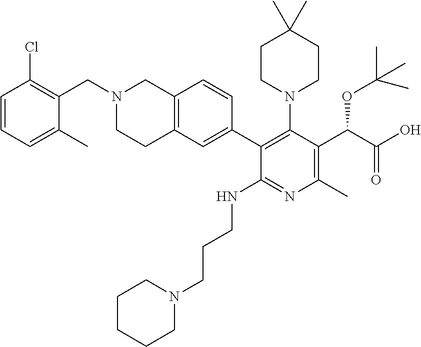 [SMILES: Cc1cccc(Cl)c1CN1CCc2cc(-c3c(NCCCN4CCCCC4)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-((3-(piperidin-1-yl)propyl)amino)pyridin-3-yl)acetic Acid

A mixture of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate (0.02 g, 0.025 mmol) and 3-(piperidin-1-yl)propan-1-amine (0.014 g, 0.101 mmol) in NMP (1 mL) was heated at 180° C. for 2 h. Ethanol (0.5 mL) and 5 M NaOH (0.050 mL, 0.252 mmol) were added and the mixture was heated at 80° C. for 4.5 h, cooled to ambient temperature, and filtered. The crude mixture was purified via preparative LC/MS to afford desired product (9.1 mg, 48%). LCMS (M+1)=744.2.

### Example 11

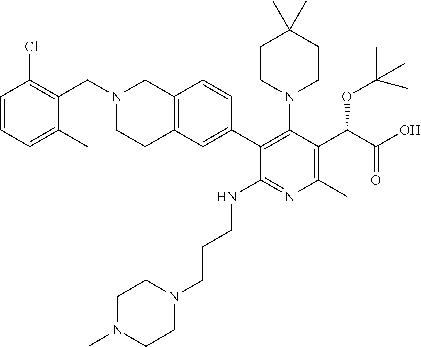 [SMILES: Cc1cccc(Cl)c1CN1CCc2cc(-c3c(NCCCN4CCN(C)CC4)nc(C)c([C@H](OC(C)(C)C)C(=O)O)c3N3CCC(C)(C)CC3)ccc2C1]

### (S)-2-(5-(2-(Benzofuro[3,2-d]pyrimidin-4-yl)-1,2,3,4-tetrahydroisoquinolin-6-yl)-4-(4,4-dimethylpiperidin-1-yl)-2-methyl-6-((3-(4-methylpiperazin-1-yl)propyl)amino)pyridin-3-yl)-2-(tert-butoxy)acetic Acid

A mixture of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)acetate (0.02 g, 0.025 mmol) and 3-(4-methylpiperazin-1-yl)propan-1-amine (0.016 g, 0.101 mmol) in NMP (1 mL) was heated at 180° C. for 2 h (LCMS showed the desired product peak as the major peak).

Ethanol (0.5 mL) and 5 M NaOH (0.050 mL, 0.252 mmol) were added and the mixture was heated at 80° C. for 4.5 h, cooled to ambient temperature, and filtered. The crude mixture was purified via preparative LC/MS to afford desired product (11.0 mg, 58%). LCMS (M+1)=759.2.

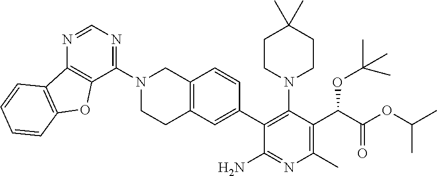 [SMILES: Cc1nc(N)c(-c2ccc3c(c2)CCN(c2ncnc4c2oc2ccccc24)C3)c(N2CCC(C)(C)CC2)c1[C@H](OC(C)(C)C)C(=O)OC(C)C]

### Isopropyl (S)-2-(6-amino-5-(2-(benzofuro[3,2-d]pyrimidin-4-yl)-1,2,3,4-tetrahydroisoquinolin-6-yl)-4-(4,4-dimethylpiperidin-1-yl)-2-methylpyridin-3-yl)-2-(tert-butoxy)acetate

A mixture of (S)-isopropyl 2-(6-amino-5-bromo-4-(4,4-dimethylpiperidin-1-yl)-2-methylpyridin-3-yl)-2-(tert-butoxy)acetate (0.35 g, 0.744 mmol), 4-(6-(4,4,5,5-tetramethyl-1,3,2-dioxaborolan-2-yl)-3,4-dihydroisoquinolin-2(1H)-yl)benzofuro[3,2-d]pyrimidine (0.477 g, 1.116 mmol), 2-dicyclohexylphosphino-2′,6′-dimethoxybiphenyl (0.061 g, 0.149 mmol), palladium(II) acetate (0.017 g, 0.074 mmol) and 2 M K₃PO₄ (1.116 ml, 2.232 mmol) in dioxane (9 mL) was purged with nitrogen. The reaction was heated at 80° C. for 1 h. After cooling to ambient temperature, the reaction was partitioned between EtOAc and water. The organic layer was dried (Na₂SO₄) and concentrated in vacuo. The crude product was purified on silica gel (120 g column) using 0-100% ethyl acetate in hexanes, then 0-10% methanol in ethyl acetate. The desired fractions were concentrated in vacuo to give title compound as light orange solid (0.26 g, 51%). LCMS (M+1)=691.30.

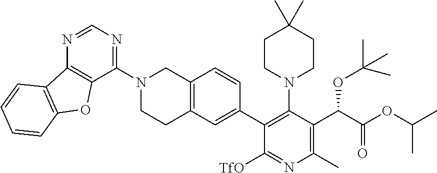 [SMILES: Cc1nc(OS(=O)(=O)C(F)(F)F)c(-c2ccc3c(c2)CCN(c2ncnc4c2oc2ccccc24)C3)c(N2CCC(C)(C)CC2)c1[C@H](OC(C)(C)C)C(=O)OC(C)C]

### Isopropyl (S)-2-(5-(2-(benzofuro[3,2-d]pyrimidin-4-yl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)-2-(tert-butoxy)acetate

(S)-Isopropyl 2-(6-amino-5-(2-(benzofuro[3,2-d]pyrimidin-4-yl)-1,2,3,4-tetrahydroisoquinolin-6-yl)-4-(4,4-dimethylpiperidin-1-yl)-2-methylpyridin-3-yl)-2-(tert-butoxy)acetate (0.25 g, 0.362 mmol) and sodium nitrite (0.062 g, 0.905 mmol) were sequentially added to a mixture of hexanes (2 mL), DMSO (0.2 mL) and trifluoromethanesulfonic acid (0.096 ml, 1.086 mmol) at 5° C. and the mixture was stirred for 10 min at 5° C., then for 18 h at ambient temperature. Water was added to the reaction mixture and it was extracted with DCM, dried over Na₂SO₄, and concentrated in vacuo. The residue was purified on silica gel (80 g column) using 0-50% ethyl acetate in hexanes, then 50-100% ethyl acetate. The desired fractions were concentrated in vacuo to give title compound as pale yellow solid (0.1 g, 34%). LCMS (M+1)=824.20.

### Example 12

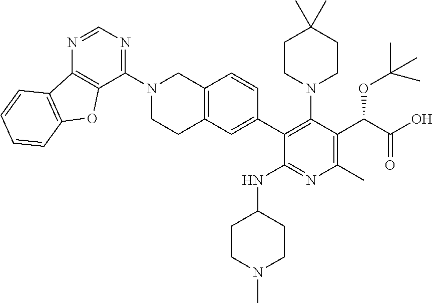 [SMILES: Cc1nc(NC2CCN(C)CC2)c(-c2ccc3c(c2)CCN(c2ncnc4c2oc2ccccc24)C3)c(N2CCC(C)(C)CC2)c1[C@H](OC(C)(C)C)C(=O)O]

### (S)-2-(5-(2-(Benzofuro[3,2-d]pyrimidin-4-yl)-1,2,3,4-tetrahydroisoquinolin-6-yl)-4-(4,4-dimethylpiperidin-1-yl)-2-methyl-6-((1-methylpiperidin-4-yl)amino)pyridin-3-yl)-2-(tert-butoxy)acetic Acid

A mixture of (S)-isopropyl 2-(5-(2-(benzofuro[3,2-d]pyrimidin-4-yl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)-2-(tert-butoxy)acetate (0.055 g, 0.067 mmol) and 1-methylpiperidin-4-amine (0.034 mL, 0.267 mmol) in NMP (2 mL) was heated at 180° C. for 3 h. The reaction was cooled to ambient temperature and ethanol (0.5 mL) and 5 M NaOH (0.134 mL, 0.668 mmol) were added. The mixture was heated at 80° C. for 3 h and then cooled to ambient temperature. The reaction mixture was purified via preparative LC/MS to afford desired product (6.0 mg, 12%). LCMS (M+1)=746.2.

### Example 13

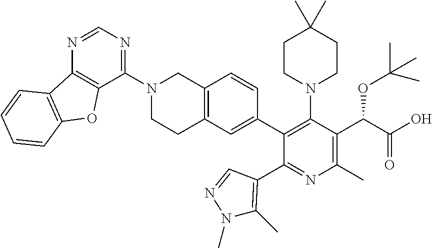 [SMILES: Cc1nc(-c2cnn(C)c2C)c(-c2ccc3c(c2)CCN(c2ncnc4c2oc2ccccc24)C3)c(N2CCC(C)(C)CC2)c1[C@H](OC(C)(C)C)C(=O)O]

### (S)-2-(5-(2-(Benzofuro[3,2-d]pyrimidin-4-yl)-1,2,3,4-tetrahydroisoquinolin-6-yl)-6-(1,5-dimethyl-1H-pyrazol-4-yl)-4-(4,4-dimethylpiperidin-1-yl)-2-methylpyridin-3-yl)-2-(tert-butoxy)acetic Acid

A mixture of (S)-isopropyl 2-(5-(2-(benzofuro[3,2-d]pyrimidin-4-yl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)-2-(tert-butoxy)acetate (0.02 g, 0.024 mmol), 1,5-dimethyl-4-(4,4,5,5-tetramethyl-1,3,2-dioxaborolan-2-yl)-1H-pyrazole (10.78 mg, 0.049 mmol), Pd(Ph₃P)₄ (5.61 mg, 4.85 μmol) and 2 M Na₂CO₃ (0.030 ml, 0.061 mmol) in toluene (0.162 mL) and ethanol (0.162 mL) was heated at 90° C. for 2 h. The reaction mixture was filtered through a pad of celite and concentrated in vacuo. The solid was taken up in ethanol (1.5 mL), 5 M NaOH (0.049 ml, 0.243 mmol) was added and the mixture was heated at 80° C. for 3 h, cooled to ambient temperature, and filtered. The crude mixture was purified via preparative LC/MS to afford desired product (8.4 mg, 48%). LCMS (M+1)=728.2.

### Example 14

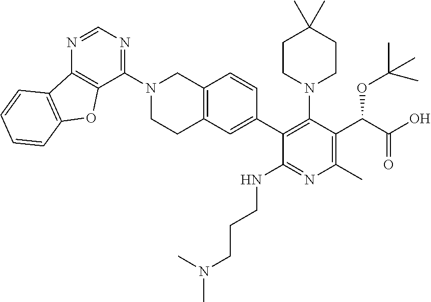 [SMILES: Cc1nc(NCCCN(C)C)c(-c2ccc3c(c2)CCN(c2ncnc4c2oc2ccccc24)C3)c(N2CCC(C)(C)CC2)c1[C@H](OC(C)(C)C)C(=O)O]

### (S)-2-(5-(2-(Benzofuro[3,2-d]pyrimidin-4-yl)-1,2,3,4-tetrahydroisoquinolin-6-yl)-6-((3-(dimethylamino)propyl)amino)-4-(4,4-dimethylpiperidin-1-yl)-2-methylpyridin-3-yl)-2-(tert-butoxy)acetic Acid

A mixture of (S)-isopropyl 2-(5-(2-(benzofuro[3,2-d]pyrimidin-4-yl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)-2-(tert-butoxy)acetate (0.02 g, 0.024 mmol) and N1,N1-dimethylpropane-1,3-diamine (9.92 mg, 0.097 mmol) in NMP (1 mL) was heated at 180° C. for 2 h. Ethanol (0.5 mL) and 5 M NaOH (0.049 mL, 0.243 mmol) were added and the mixture was heated at 80° C. for 2 h, cooled to ambient temperature, and filtered. The crude mixture was purified via preparative LC/MS to afford desired product (6.6 mg, 36%). LCMS (M+1)=734.1.

### Example 15

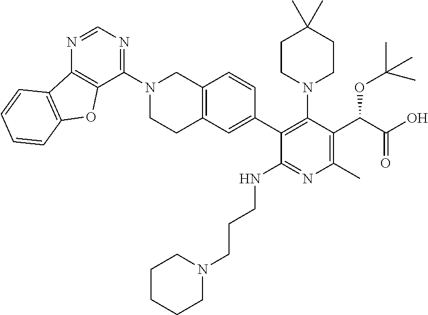 [SMILES: Cc1nc(NCCCN2CCCCC2)c(-c2ccc3c(c2)CCN(c2ncnc4c2oc2ccccc24)C3)c(N2CCC(C)(C)CC2)c1[C@H](OC(C)(C)C)C(=O)O]

### (S)-2-(5-(2-(Benzofuro[3,2-d]pyrimidin-4-yl)-1,2,3,4-tetrahydroisoquinolin-6-yl)-4-(4,4-dimethylpiperidin-1-yl)-2-methyl-6-((3-(piperidin-1-yl)propyl)amino)pyridin-3-yl)-2-(tert-butoxy)acetic Acid

A mixture of (S)-isopropyl 2-(5-(2-(benzofuro[3,2-d]pyrimidin-4-yl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)-2-(tert-butoxy)acetate (0.02 g, 0.024 mmol) and 3-(piperidin-1-yl)propan-1-amine (0.014 g, 0.097 mmol) in NMP (1 mL) was heated at 180° C. for 2 h. Ethanol (0.5 mL) and 5 M NaOH (0.049 mL, 0.243 mmol) were added and the mixture was heated at 80° C. for 4.5 h, cooled to ambient temperature, and filtered. The crude mixture was purified via preparative LC/MS to afford desired product (8.3 mg, 42%). LCMS (M+1)=774.2.

### Example 16

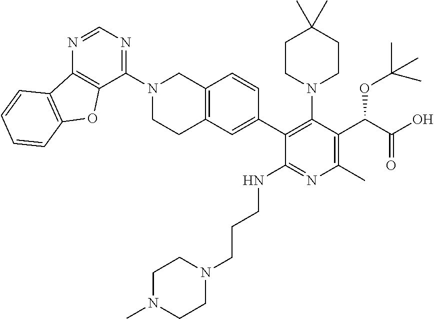 [SMILES: Cc1nc(NCCCN2CCN(C)CC2)c(-c2ccc3c(c2)CCN(c2ncnc4c2oc2ccccc24)C3)c(N2CCC(C)(C)CC2)c1[C@H](OC(C)(C)C)C(=O)O]

### (S)-2-(5-(2-(Benzofuro[3,2-d]pyrimidin-4-yl)-1,2,3,4-tetrahydroisoquinolin-6-yl)-4-(4,4-dimethylpiperidin-1-yl)-2-methyl-6-((3-(4-methylpiperazin-1-yl)propyl)amino)pyridin-3-yl)-2-(tert-butoxy)acetic Acid

A mixture of (S)-isopropyl 2-(5-(2-(benzofuro[3,2-d]pyrimidin-4-yl)-1,2,3,4-tetrahydroisoquinolin-6-yl)-4-(4,4-dimethylpiperidin-1-yl)-2-methyl-6-(((trifluoromethyl)sulfonyl)oxy)pyridin-3-yl)-2-(tert-butoxy)acetate (0.02 g, 0.024 mmol) and 3-(4-methylpiperazin-1-yl)propan-1-amine (0.015 g, 0.097 mmol) in NMP (1 mL) was heated at 180° C. for 2 h. Ethanol (0.5 mL) and 5 M NaOH (0.049 mL, 0.243 mmol) were added and the mixture was heated at 80° C. for 4.5 h, cooled to ambient temperature, and filtered. The crude mixture was purified via preparative LC/MS to afford desired product (4.9 mg, 25%). LCMS (M+1)=789.2.

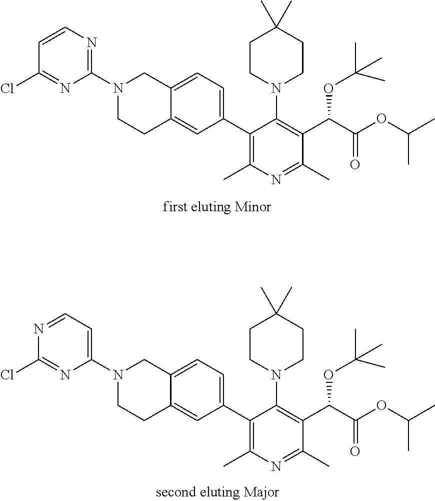 [SMILES: Cc1nc(C)c([C@H](OC(C)(C)C)C(=O)OC(C)C)c(N2CCC(C)(C)CC2)c1-c1ccc2c(c1)CCN(c1nccc(Cl)n1)C2 ; Cc1nc(C)c([C@H](OC(C)(C)C)C(=O)OC(C)C)c(N2CCC(C)(C)CC2)c1-c1ccc2c(c1)CCN(c1ccnc(Cl)n1)C2]

### (S)-Isopropyl 2-(tert-butoxy)-2-(5-(2-(4-chloropyrimidin-2-yl)-1,2,3,4-tetrahydroisoquinolin-6-yl)-4-(4,4-dimethylpiperidin-1-yl)-2,6-dimethylpyridin-3-yl)acetate and (S)-isopropyl 2-(tert-butoxy)-2-(5-(2-(2-chloropyrimidin-4-yl)-1,2,3,4-tetrahydroisoquinolin-6-yl)-4-(4,4-dimethylpiperidin-1-yl)-2,6-dimethylpyridin-3-yl)acetate

2,4-Dichloropyrimidine (56 mg, 0.38 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (178 mg, 0.341 mmol) and K₂CO₃ (94 mg, 0.68 mmol) in acetonitrile (3.4 ml) at 80° C. The reaction was stirred overnight. The reaction was concentrated and was purified on silica gel (Biotage, EtOAc/hexanes gradient, 0-100% over 10 CVs) to afford: First eluting minor isomer: (S)-isopropyl 2-(tert-butoxy)-2-(5-(2-(4-chloropyrimidin-2-yl)-1,2,3,4-tetrahydroisoquinolin-6-yl)-4-(4,4-dimethylpiperidin-1-yl)-2,6-dimethylpyridin-3-yl)acetate (36 mg, 0.057 mmol, 16.6% yield) LCMS (M+H)=634.25 and second eluting major isomer: (S)-isopropyl 2-(tert-butoxy)-2-(5-(2-(2-chloropyrimidin-4-yl)-1,2,3,4-tetrahydroisoquinolin-6-yl)-4-(4,4-dimethylpiperidin-1-yl)-2,6-dimethylpyridin-3-yl)acetate (97 mg, 0.153 mmol, 44.8% yield). LCMS (M+H)=634.25.

### General Synthesis of Aminopyrimidines Intermediates

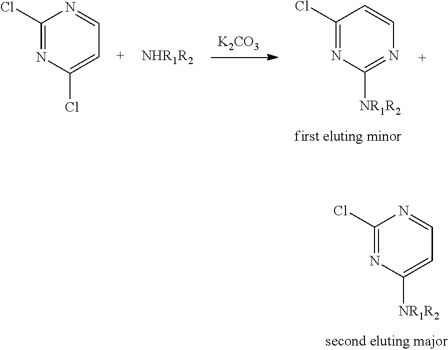

The appropriate amine (1.0 mmol) was added to a stirring solution of 2,4-dichloropyrimidine (1.0 mmol) and K₂CO₃ (2.2 mmol) in THF or ACN (0.4M) at rt or 80° C. respectively. The reaction was stirred at the appropriate temperature overnight. The reaction was concentrated, adsorbed onto celite and was purified on silica gel (Biotage, EtOAc/hexanes gradient, 0-100% over 10 CVs) to afford both separable isomers: first eluting minor and second eluting major.

### Example 17

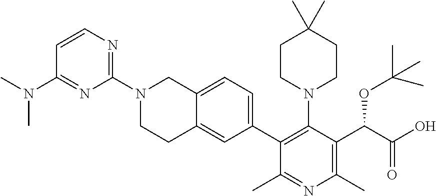 [SMILES: Cc1nc(C)c([C@H](OC(C)(C)C)C(=O)O)c(N2CCC(C)(C)CC2)c1-c1ccc2c(c1)CCN(c1nccc(N(C)C)n1)C2]

### (2S)-2-(tert-Butoxy)-2-(5-{2-[4-(dimethylamino)pyrimidin-2-yl]-1,2,3,4-tetrahydroisoquinolin-6-yl}-4-(4,4-dimethylpiperidin-1-yl)-2,6-dimethylpyridin-3-yl)acetic Acid

2-Chloro-N,N-dimethylpyrimidin-4-amine (15 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at rt. The reaction was allowed to stir at 80° C. for 2 days. Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(tert-butoxy)-2-(5-{2-[4-(dimethylamino)pyrimidin-2-yl]-1,2,3,4-tetrahydroisoquinolin-6-yl}-4-(4,4-dimethylpiperidin-1-yl)-2,6-dimethylpyridin-3-yl)acetic acid (12.1 mg). LCMS (M+H)=601.32. ¹H NMR (500 MHz, DMSO-d₆) δ 7.90 (d, J=5.9 Hz, 1H), 7.35-7.29 (m, 1H), 7.16-7.12 (m, 1H), 6.98-6.94 (m, 1H), 5.98 (d, J=5.9 Hz, 1H), 5.83 (br d, J=11.7 Hz, 1H), 4.98-4.92 (m, 1H), 4.85-4.78 (m, 1H), 3.98-3.85 (m, 2H), 3.04 (s, 3H), 2.89 (br d, J=6.2 Hz, 2H), 2.83 (br s, 1H), 2.56 (s, 3H), 2.50-2.39 (m, 3H), 2.18-2.05 (m, 3H), 1.92 (s, 5H), 1.26 (br s, 1H), 1.20 (br s, 1H), 1.13 (s, 9H), 0.87-0.79 (m, 3H), 0.59 (s, 1H), 0.51 (s, 2H).

### Example 18

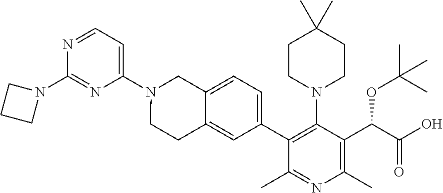 [SMILES: Cc1nc(C)c([C@H](OC(C)(C)C)C(=O)O)c(N2CCC(C)(C)CC2)c1-c1ccc2c(c1)CCN(c1ccnc(N3CCC3)n1)C2]

### (2S)-2-(5-{2-[2-(Azetidin-1-yl)pyrimidin-4-yl]-1,2,3,4-tetrahydroisoquinolin-6-yl}-4-(4,4-dimethylpiperidin-1-yl)-2,6-dimethylpyridin-3-yl)-2-(tert-butoxy)acetic Acid

Azetidine hydrochloride (13 mg, 0.14 mmol) was added to a stirring solution of (S)-isopropyl 2-(tert-butoxy)-2-(5-(2-(2-chloropyrimidin-4-yl)-1,2,3,4-tetrahydroisoquinolin-6-yl)-4-(4,4-dimethylpiperidin-1-yl)-2,6-dimethylpyridin-3-yl)acetate (18 mg, 0.028 mmol) and K₂CO₃ (28 mg, 0.20 mmol) in acetonitrile (1 ml) at rt. The reaction was stirred at 80° C. overnight. Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(5-{2-[2-(azetidin-1-yl)pyrimidin-4-yl]-1,2,3,4-tetrahydroisoquinolin-6-yl}-4-(4,4-dimethylpiperidin-1-yl)-2,6-dimethylpyridin-3-yl)-2-(tert-butoxy)acetic acid (13.0 mg). LCMS (M+H)=613.15. ¹H NMR (500 MHz, DMSO-d₆) δ 7.88 (d, J=5.9 Hz, 1H), 7.35-7.29 (m, 1H), 7.18-7.14 (m, 1H), 6.99 (br s, 1H), 6.18 (br d, J=5.9 Hz, 1H), 3.97 (br t, J=7.5 Hz, 4H), 2.91 (br s, 3H), 2.56-2.54 (m, 2H), 2.49-2.40 (m, 4H), 2.29-2.18 (m, 3H), 2.12 (s, 2H), 2.10-2.03 (m, 2H), 1.92 (s, 6H), 1.13 (s, 10H), 0.88-0.79 (m, 3H), 0.59 (br s, 1H), 0.50 (s, 2H).

### Example 19

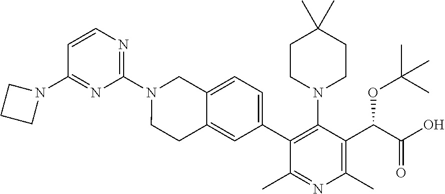 [SMILES: Cc1nc(C)c([C@H](OC(C)(C)C)C(=O)O)c(N2CCC(C)(C)CC2)c1-c1ccc2c(c1)CCN(c1nccc(N3CCC3)n1)C2]

### (2S)-2-(5-{2-[4-(Azetidin-1-yl)pyrimidin-2-yl]-1,2,3,4-tetrahydroisoquinolin-6-yl}-4-(4,4-dimethylpiperidin-1-yl)-2,6-dimethylpyridin-3-yl)-2-(tert-butoxy)acetic Acid

4-(Azetidin-1-yl)-2-chloropyrimidine (16 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at rt. The reaction was allowed to stir at 80° C. for 2 days. Then the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(5-{2-[4-(azetidin-1-yl)pyrimidin-2-yl]-1,2,3,4-tetrahydroisoquinolin-6-yl}-4-(4,4-dimethylpiperidin-1-yl)-2,6-dimethylpyridin-3-yl)-2-(tert-butoxy)acetic acid (8.8 mg). LCMS (M+H)=613.2. ¹H NMR (500 MHz, DMSO-d₆) δ 7.89 (d, J=5.5 Hz, 1H), 7.33-7.28 (m, 1H), 7.16-7.11 (m, 1H), 6.98-6.94 (m, 1H), 5.86 (br d, J=11.7 Hz, 1H), 5.67 (d, J=5.5 Hz, 1H), 4.93 (br d, J=17.2 Hz, 1H), 4.80 (dd, J=17.1, 5.7 Hz, 1H), 4.02-3.84 (m, 5H), 2.93-2.79 (m, 2H), 2.55 (s, 4H), 2.44 (s, 3H), 2.34 (quin, J=7.4 Hz, 2H), 2.16-2.06 (m, 3H), 1.92 (s, 2H), 1.47 (br s, 1H), 1.18 (br s, 1H), 1.13 (s, 9H), 0.97 (br d, J=12.5 Hz, 1H), 0.89-0.79 (m, 3H), 0.59 (s, 1H), 0.52 (s, 2H).

### Example 20

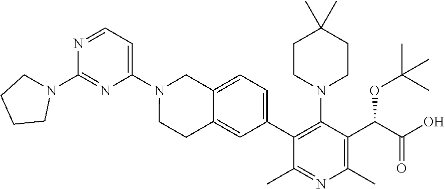 [SMILES: Cc1nc(C)c([C@H](OC(C)(C)C)C(=O)O)c(N2CCC(C)(C)CC2)c1-c1ccc2c(c1)CCN(c1ccnc(N3CCCC3)n1)C2]

### (2S)-2-(tert-Butoxy)-2-[4-(4,4-dimethylpiperidin-1-yl)-2,6-dimethyl-5-{2-[2-(pyrrolidin-1-yl)pyrimidin-4-yl]-1,2,3,4-tetrahydroisoquinolin-6-yl}pyridin-3-yl]acetic Acid

4-Chloro-2-(pyrrolidin-1-yl)pyrimidine (18 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at rt. The reaction was allowed to stir at 80° C. for 2 days. Then, the sample was concentrated and taken up in 1.5 mL of EtOH and treated with 0.1 mL of 5N NaOH. The mixture was then stirred at 100° C. for 5 hrs. The crude material was purified via preparative LC/MS to afford (2S)-2-(tert-butoxy)-2-[4-(4,4-dimethylpiperidin-1-yl)-2,6-dimethyl-5-{2-[2-(pyrrolidin-1-yl)pyrimidin-4-yl]-1,2,3,4-tetrahydroisoquinolin-6-yl}pyridin-3-yl]acetic acid (12.1 mg). LCMS (M+H)=627.17. ¹H NMR (500 MHz, DMSO-d₆) δ 7.89 (d, J=5.9 Hz, 1H), 7.36-7.31 (m, 1H), 7.17 (br d, J=5.9 Hz, 1H), 6.99 (br s, 1H), 6.13 (dd, J=6.1, 2.4 Hz, 1H), 5.86 (br d, J=11.7 Hz, 1H), 4.71 (br s, 2H), 3.92 (s, 1H), 2.91 (br s, 3H), 2.56-2.55 (m, 3H), 2.50-2.42 (m, 3H), 2.12 (s, 2H), 2.08 (s, 1H), 1.95-1.87 (m, 8H), 1.48 (br s, 1H), 1.31-1.17 (m, 2H), 1.17-1.09 (m, 10H), 0.98-0.78 (m, 4H), 0.58 (br s, 1H), 0.49 (s, 2H).

### Example 21

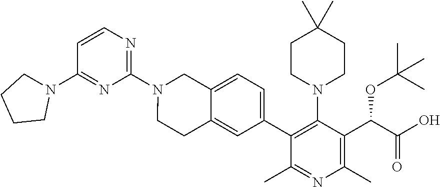 [SMILES: Cc1nc(C)c([C@H](OC(C)(C)C)C(=O)O)c(N2CCC(C)(C)CC2)c1-c1ccc2c(c1)CCN(c1nccc(N3CCCC3)n1)C2]

### (2S)-2-(tert-Butoxy)-2-[4-(4,4-dimethylpiperidin-1-yl)-2,6-dimethyl-5-{2-[4-(pyrrolidin-1-yl)pyrimidin-2-yl]-1,2,3,4-tetrahydroisoquinolin-6-yl}pyridin-3-yl]acetic Acid

2-Chloro-4-(pyrrolidin-1-yl)pyrimidine (18 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at rt. The reaction was allowed to stir at 80° C. for 2 days. Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(tert-butoxy)-2-[4-(4,4-dimethylpiperidin-1-yl)-2,6-dimethyl-5-{2-[4-(pyrrolidin-1-yl)pyrimidin-2-yl]-1,2,3,4-tetrahydroisoquinolin-6-yl}pyridin-3-yl]acetic acid (7.9 mg). LCMS (M+H)=627.2. ¹H NMR (500 MHz, DMSO-d₆) δ 7.87 (d, J=5.9 Hz, 1H), 7.33-7.29 (m, 1H), 7.15-7.11 (m, 1H), 6.97-6.93 (m, 1H), 5.87-5.78 (m, 2H), 4.94 (d, J=17.6 Hz, 1H), 4.82 (dd, J=17.4, 6.1 Hz, 1H), 3.99-3.85 (m, 2H), 2.92-2.78 (m, 2H), 2.55 (s, 4H), 2.49-2.39 (m, 3H), 2.14-2.06 (m, 4H), 1.92 (s, 7H), 1.47 (br s, 1H), 1.26 (br d, J=12.5 Hz, 1H), 1.18 (br d, J=12.5 Hz, 1H), 1.13 (s, 9H), 1.06-0.90 (m, 1H), 0.89-0.77 (m, 3H), 0.59 (s, 1H), 0.51 (s, 2H).

### Example 22

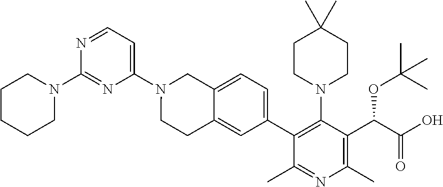 [SMILES: Cc1nc(C)c([C@H](OC(C)(C)C)C(=O)O)c(N2CCC(C)(C)CC2)c1-c1ccc2c(c1)CCN(c1ccnc(N3CCCCC3)n1)C2]

### (2S)-2-(tert-Butoxy)-2-[4-(4,4-dimethylpiperidin-1-yl)-2,6-dimethyl-5-{2-[2-(piperidin-1-yl)pyrimidin-4-yl]-1,2,3,4-tetrahydroisoquinolin-6-yl}pyridin-3-yl]acetic Acid

Piperidine (6 μl, 0.059 mmol) was added to a stirring solution of (S)-isopropyl 2-(tert-butoxy)-2-(5-(2-(2-chloropyrimidin-4-yl)-1,2,3,4-tetrahydroisoquinolin-6-yl)-4-(4,4-dimethylpiperidin-1-yl)-2,6-dimethylpyridin-3-yl)acetate (25 mg, 0.039 mmol) and K₂CO₃ (16 mg, 0.12 mmol) in acetonitrile (1 ml) at rt. The reaction was stirred at 80° C. overnight. Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(tert-butoxy)-2-[4-(4,4-dimethylpiperidin-1-yl)-2,6-dimethyl-5-{2-[2-(piperidin-1-yl)pyrimidin-4-yl]-1,2,3,4-tetrahydroisoquinolin-6-yl}pyridin-3-yl]acetic acid (21.7 mg). LCMS (M+H)=641.26. ¹H NMR (500 MHz, DMSO-d₆) δ 7.90 (d, J=5.9 Hz, 1H), 7.37-7.32 (m, 1H), 7.18-7.15 (m, 1H), 6.99 (s, 1H), 6.12 (d, J=5.9 Hz, 1H), 5.83 (br d, J=12.5 Hz, 1H), 4.67 (br d, J=16.9 Hz, 1H), 3.92-3.73 (m, 2H), 2.92 (br s, 2H), 2.82 (br s, 1H), 2.55 (s, 7H), 2.49-2.39 (m, 3H), 2.12 (s, 2H), 2.08 (s, 1H), 1.92 (s, 1H), 1.61 (br s, 2H), 1.50 (br d, J=4.4 Hz, 5H), 1.25 (br s, 1H), 1.19-1.09 (m, 10H), 0.96 (br s, 1H), 0.88-0.77 (m, 3H), 0.58 (s, 1H), 0.49 (s, 2H).

### Example 23

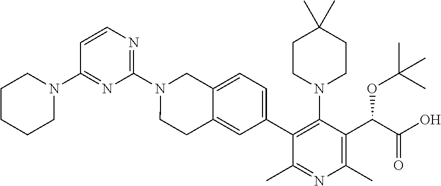 [SMILES: Cc1nc(C)c([C@H](OC(C)(C)C)C(=O)O)c(N2CCC(C)(C)CC2)c1-c1ccc2c(c1)CCN(c1nccc(N3CCCCC3)n1)C2]

### (2S)-2-(tert-Butoxy)-2-[4-(4,4-dimethylpiperidin-1-yl)-2,6-dimethyl-5-{2-[4-(piperidin-1-yl)pyrimidin-2-yl]-1,2,3,4-tetrahydroisoquinolin-6-yl}pyridin-3-yl]acetic Acid

2-Chloro-4-(piperidin-1-yl)pyrimidine (19 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at 90° C. The reaction was stirred at 90° C. overnight. LCMS showed partial conversion. Sodium iodide (7 mg, 0.05 mmol) was added and the reaction was stirred at 90° C. overnight. The reaction was concentrated and then taken up in 1.5 mL of EtOH and treated with 0.1 mL of 5N NaOH and stirred at 80° C. overnight. The crude material was purified via preparative LC/MS to afford (2S)-2-(tert-butoxy)-2-[4-(4,4-dimethylpiperidin-1-yl)-2,6-dimethyl-5-{2-[4-(piperidin-1-yl)pyrimidin-2-yl]-1,2,3,4-tetrahydroisoquinolin-6-yl}pyridin-3-yl]acetic acid (9.0 mg). LCMS (M+H)=641.26. ¹H NMR (500 MHz, DMSO-d₆) δ 7.89 (d, J=5.8 Hz, 1H), 7.39-7.23 (m, 1H), 7.18-7.08 (m, 1H), 7.01-6.88 (m, 1H), 6.10 (d, J=6.1 Hz, 1H), 5.80 (br d, J=13.1 Hz, 1H), 5.01-4.87 (m, 1H), 4.79 (t, J=16.9 Hz, 1H), 2.91-2.83 (m, 3H), 2.46-2.42 (m, 3H), 2.15-2.04 (m, 4H), 1.94-1.88 (m, 6H), 1.84-1.73 (m, 1H), 1.70-1.58 (m, 2H), 1.57-1.44 (m, 5H), 1.32-1.17 (m, 2H), 1.15-1.07 (m, 9H), 1.04-0.92 (m, 2H), 0.87-0.76 (m, 3H), 0.63-0.54 (m, 1H), 0.50 (s, 2H).

### Example 24

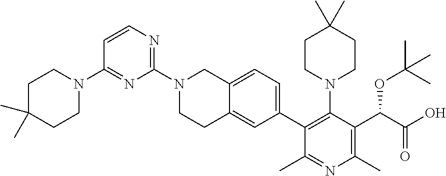 [SMILES: Cc1nc(C)c([C@H](OC(C)(C)C)C(=O)O)c(N2CCC(C)(C)CC2)c1-c1ccc2c(c1)CCN(c1nccc(N3CCC(C)(C)CC3)n1)C2]

### (2S)-2-(tert-Butoxy)-2-[4-(4,4-dimethylpiperidin-1-yl)-5-{2-[4-(4,4-dimethylpiperidin-1-yl)pyrimidin-2-yl}-1,2,3,4-tetrahydroisoquinolin-6-yl}-2,6-dimethylpyridin-3-yl]acetic Acid

2-Chloro-4-(4,4-dimethylpiperidin-1-yl)pyrimidine (22 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at rt. The reaction was allowed to stir at 80° C. for 2 days. Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(tert-butoxy)-2-[4-(4,4-dimethylpiperidin-1-yl)-5-{2-[4-(4,4-dimethylpiperidin-1-yl)pyrimidin-2-yl]-1,2,3,4-tetrahydroisoquinolin-6-yl}-2,6-dimethylpyridin-3-yl]acetic acid (5.9 mg). LCMS (M+H)=669.26. ¹H NMR (500 MHz, DMSO-d₆) δ 7.89 (d, J=6.2 Hz, 1H), 7.36-7.30 (m, 1H), 7.17-7.13 (m, 1H), 6.99-6.96 (m, 1H), 6.14 (br d, J=5.5 Hz, 1H), 5.85 (br d, J=11.4 Hz, 1H), 4.99-4.91 (m, 1H), 4.79 (t, J=18.2 Hz, 1H), 3.98-3.82 (m, 2H), 3.60 (br s, 2H), 2.88 (br s, 2H), 2.55 (s, 6H), 2.50-2.42 (m, 3H), 2.13 (s, 2H), 2.09 (s, 1H), 1.49-1.29 (m, 5H), 1.24 (br s, 2H), 1.17-1.10 (m, 9H), 0.98 (s, 7H), 0.82 (br s, 3H), 0.60 (br s, 1H), 0.50 (br s, 2H).

### Example 25

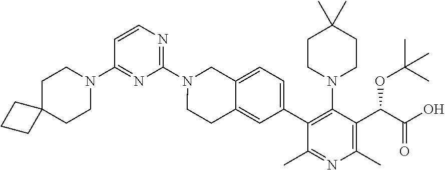 [SMILES: Cc1nc(C)c([C@H](OC(C)(C)C)C(=O)O)c(N2CCC(C)(C)CC2)c1-c1ccc2c(c1)CCN(c1nccc(N3CCC4(CCC4)CC3)n1)C2]

### (2S)-2-{5-[2-(4-{7-Azaspiro[3.5]nonan-7-yl}pyrimidin-2-yl)-1,2,3,4-tetrahydroisoquinolin-6-yl]-4-(4,4-dimethylpiperidin-1-yl)-2,6-dimethylpyridin-3-yl}-2-(tert-butoxy)acetic Acid

7-(2-Chloropyrimidin-4-yl)-7-azaspiro[3.5]nonane (23 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at rt. The reaction was allowed to stir at 80° C. for 2 days. Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-{5-[2-(4-{7-azaspiro[3.5]nonan-7-yl}pyrimidin-2-yl)-1,2,3,4-tetrahydroisoquinolin-6-yl]-4-(4,4-dimethylpiperidin-1-yl)-2,6-dimethylpyridin-3-yl}-2-(tert-butoxy)acetic acid (6.9 mg). LCMS (M+H)=681.24. ¹H NMR (500 MHz, DMSO-d₆) δ 7.89 (d, J=5.9 Hz, 1H), 7.35-7.30 (m, 1H), 7.17-7.13 (m, 1H), 6.99-6.94 (m, 1H), 6.13 (br d, J=5.9 Hz, 1H), 5.86 (br d, J=13.6 Hz, 1H), 4.98 (br d, J=17.2 Hz, 1H), 4.83-4.73 (m, 1H), 3.97 (br d, J=5.9 Hz, 1H), 3.93-3.82 (m, 1H), 3.52 (br s, 1H), 2.88 (br s, 2H), 2.57-2.54 (m, 7H), 2.49-2.40 (m, 3H), 2.13 (s, 2H), 2.08 (s, 1H), 1.95-1.84 (m, 2H), 1.78 (br d, J=7.0 Hz, 4H), 1.55 (br d, J=5.1 Hz, 4H), 1.46 (br s, 1H), 1.34-1.16 (m, 2H), 1.13 (d, J=3.3 Hz, 9H), 0.82 (br s, 4H), 0.58 (br s, 1H), 0.48 (br s, 2H).

### Example 26

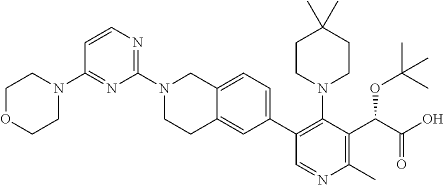 [SMILES: Cc1ncc(-c2ccc3c(c2)CCN(c2nccc(N4CCOCC4)n2)C3)c(N2CCC(C)(C)CC2)c1[C@H](OC(C)(C)C)C(=O)O]

### (2S)-2-(tert-Butoxy)-2-[4-(4,4-dimethylpiperidin-1-yl)-2-methyl-5-{2-[4-(morpholin-4-yl)pyrimidin-2-yl]-1,2,3,4-tetrahydroisoquinolin-6-yl}pyridin-3-yl]acetic Acid

4-(2-Chloropyrimidin-4-yl)morpholine (20 mg, 0.098 mmol) was added to a stirring solution of (S)-isopropyl 2-(tert-butoxy)-2-(4-(4,4-dimethylpiperidin-1-yl)-2-methyl-5-(1,2,3,4-tetrahydroisoquinolin-6-yl)pyridin-3-yl)acetate (25 mg, 0.049 mmol) and potassium carbonate (20 mg, 0.15 mmol) in ACN (1 ml) at rt. The reaction was allowed to stir at 80° C. over the weekend. Then, the sample was concentrated and taken up in 1.5 mL of EtOH and treated with 0.1 mL of 5 N NaOH. The mixture was then stirred at 100° C. for 5 hrs. Then, the crude material was purified via preparative LC/MS to afford (2S)-2-(tert-butoxy)-2-[4-(4,4-dimethylpiperidin-1-yl)-2-methyl-5-{2-[4-(morpholin-4-yl)pyrimidin-2-yl]-1,2,3,4-tetrahydroisoquinolin-6-yl}pyridin-3-yl]acetic acid (19.1 mg). LCMS (M+H)=629.31. ¹H NMR (500 MHz, DMSO-d₆) δ 7.99 (s, 1H), 7.96 (d, J=5.7 Hz, 1H), 7.32 (d, J=8.1 Hz, 1H), 7.16-7.10 (m, 2H), 6.13 (d, J=5.9 Hz, 1H), 5.65 (s, 1H), 4.88 (br d, J=17.6 Hz, 2H), 3.97 (br s, 1H), 3.67 (br d, J=5.1 Hz, 3H), 3.56 (br s, 1H), 2.89 (br d, J=5.1 Hz, 3H), 2.56 (s, 3H), 2.50-2.45 (m, 3H), 1.91 (s, 7H), 1.29 (br s, 2H), 1.10 (s, 9H), 0.88 (br s, 3H), 0.73 (br s, 3H).

### Example 27

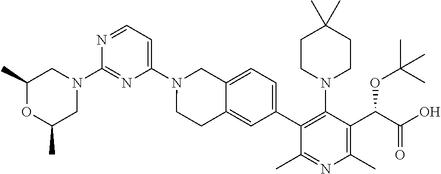 [SMILES: Cc1nc(C)c([C@H](OC(C)(C)C)C(=O)O)c(N2CCC(C)(C)CC2)c1-c1ccc2c(c1)CCN(c1ccnc(N3C[C@@H](C)O[C@@H](C)C3)n1)C2]

### (2S)-2-(tert-Butoxy)-2-[5-(2-{2-[(2R,6S)-2,6-dimethylmorpholin-4-yl]pyrimidin-4-yl}-1,2,3,4-tetrahydroisoquinolin-6-yl)-4-(4,4-dimethylpiperidin-1-yl)-2,6-dimethylpyridin-3-yl]acetic Acid

(2S,6R)-4-(4-Chloropyrimidin-2-yl)-2,6-dimethylmorpholine (12 mg, 0.053 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and K₂CO₃ (15 mg, 0.11 mmol) in acetonitrile (1 ml) at rt. The reaction was stirred at 80° C. overnight. Then, 4 mg of (2S,6R)-4-(4-chloropyrimidin-2-yl)-2,6-dimethylmorpholine was added and the reaction was stirred overnight at 80° C. The reaction was concentrated and taken up in 1.5 mL of EtOH and treated with 0.1 mL of 5 N NaOH and stirred at 80° C. overnight. The crude material was purified via preparative LC/MS to afford title compound (5.4 mg). LCMS (M+H)=671.23. ¹H NMR (500 MHz, DMSO-d₆) δ 7.92 (d, J=5.9 Hz, 1H), 7.38-7.32 (m, 1H), 7.17 (br d, J=4.8 Hz, 1H), 6.98 (br s, 1H), 6.19 (d, J=5.9 Hz, 1H), 5.79 (br d, J=12.5 Hz, 1H), 4.76 (br s, 1H), 4.50 (br d, J=12.8 Hz, 2H), 3.82 (br s, 1H), 2.91 (br d, J=5.5 Hz, 2H), 2.55 (s, 6H), 2.49-2.38 (m, 5H), 2.11 (s, 2H), 2.07 (s, 1H), 1.91 (s, 4H), 1.47 (br s, 1H), 1.26 (br d, J=19.1 Hz, 1H), 1.17-1.09 (m, 14H), 0.94 (br d, J=11.7 Hz, 1H), 0.88-0.77 (m, 3H), 0.58 (br s, 1H), 0.49 (s, 2H).

### Example 28

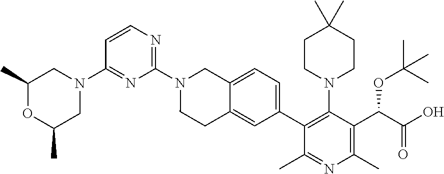 [SMILES: Cc1nc(C)c([C@H](OC(C)(C)C)C(=O)O)c(N2CCC(C)(C)CC2)c1-c1ccc2c(c1)CCN(c1nccc(N3C[C@@H](C)O[C@@H](C)C3)n1)C2]

### (2S)-2-(tert-Butoxy)-2-[5-(2-{4-[(2R,6S)-2,6-dimethylmorpholin-4-yl]pyrimidin-2-yl}-1,2,3,4-tetrahydroisoquinolin-6-yl)-4-(4,4-dimethylpiperidin-1-yl)-2,6-dimethylpyridin-3-yl]acetic Acid

(2S,6R)-4-(2-Chloropyrimidin-4-yl)-2,6-dimethylmorpholine (12 mg, 0.053 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and K₂CO₃ (15 mg, 0.11 mmol) in acetonitrile (1 ml) at rt. The reaction was stirred at 80° C. overnight. Then additional (2S,6R)-4-(2-chloropyrimidin-4-yl)-2,6-dimethylmorpholine (12 mg, 0.053 mmol) was added and the reaction was stirred overnight at 80° C. The reaction was concentrated and taken up in 1.5 mL of EtOH and treated with 0.1 mL of 5N NaOH and stirred at 80° C. overnight. The crude material was purified via preparative LC/MS to afford title compound (10.3 mg). LCMS (M+H)=671.25. ¹H NMR (500 MHz, DMSO-d₆) δ 7.94 (d, J=5.9 Hz, 1H), 7.35-7.30 (m, 1H), 7.15-7.12 (m, 1H), 6.97-6.94 (m, 1H), 6.14 (d, J=6.2 Hz, 1H), 5.79 (br d, J=12.8 Hz, 1H), 4.93 (t, J=16.3 Hz, 1H), 4.81-4.75 (m, 1H), 4.24 (br s, 1H), 3.98-3.84 (m, 1H), 3.55 (br s, 1H), 2.92-2.76 (m, 2H), 2.55 (s, 6H), 2.48-2.41 (m, 4H), 2.11 (s, 2H), 2.07 (s, 1H), 1.91 (s, 3H), 1.47 (br s, 1H), 1.26 (br d, J=13.9 Hz, 1H), 1.18-1.09 (m, 15H), 1.07-0.90 (m, 1H), 0.88-0.77 (m, 3H), 0.59 (s, 1H), 0.50 (s, 2H).

### Example 29

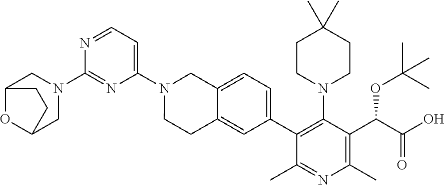 [SMILES: Cc1nc(C)c([C@H](OC(C)(C)C)C(=O)O)c(N2CCC(C)(C)CC2)c1-c1ccc2c(c1)CCN(c1ccnc(N3CC4CCC(C3)O4)n1)C2]

### (2S)-2-(tert-Butoxy)-2-[4-(4,4-dimethylpiperidin-1-yl)-2,6-dimethyl-5-[2-(2-{8-oxa-3-azabicyclo[3.2.1]octan-3-yl}pyrimidin-4-yl)-1,2,3,4-tetrahydroisoquinolin-6-yl]pyridin-3-yl]acetic Acid

8-Oxa-3-azabicyclo[3.2.1]octane hydrochloride (9 mg, 0.059 mmol) was added to a stirring solution of (S)-isopropyl 2-(tert-butoxy)-2-(5-(2-(2-chloropyrimidin-4-yl)-1,2,3,4-tetrahydroisoquinolin-6-yl)-4-(4,4-dimethylpiperidin-1-yl)-2,6-dimethylpyridin-3-yl)acetate (25 mg, 0.039 mmol) and K₂CO₃ (22 mg, 0.16 mmol) in acetonitrile (1 ml) at rt. The reaction was stirred at 80° C. for 2 days. Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(tert-butoxy)-2-[4-(4,4-dimethylpiperidin-1-yl)-2,6-dimethyl-5-[2-(2-{8-oxa-3-azabicyclo[3.2.1]octan-3-yl}pyrimidin-4-yl)-1,2,3,4-tetrahydroisoquinolin-6-yl]pyridin-3-yl]acetic acid (7.7 mg). LCMS (M+H)=669.21. ¹H NMR (500 MHz, DMSO-d₆) δ 7.91 (d, J=5.9 Hz, 1H), 7.38-7.31 (m, 1H), 7.20-7.15 (m, 1H), 7.01-6.97 (m, 1H), 6.20 (d, J=5.9 Hz, 1H), 5.79 (br d, J=12.5 Hz, 1H), 4.39 (br s, 2H), 4.20 (br d, J=12.8 Hz, 2H), 2.98 (br d, J=15.4 Hz, 2H), 2.92 (br s, 2H), 2.55 (s, 6H), 2.49-2.39 (m, 3H), 2.12 (s, 2H), 2.08 (s, 1H), 1.92 (s, 3H), 1.84-1.73 (m, 2H), 1.66 (br t, J=6.1 Hz, 2H), 1.48 (br s, 1H), 1.30 (br d, J=7.0 Hz, 1H), 1.24 (br s, 1H), 1.19-1.05 (m, 9H), 0.96-0.78 (m, 3H), 0.58 (s, 1H), 0.47 (s, 2H).

### Example 30

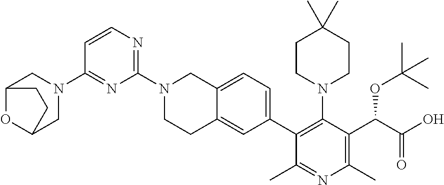 [SMILES: Cc1nc(C)c([C@H](OC(C)(C)C)C(=O)O)c(N2CCC(C)(C)CC2)c1-c1ccc2c(c1)CCN(c1nccc(N3CC4CCC(C3)O4)n1)C2]

### (2S)-2-(tert-Butoxy)-2-[4-(4,4-dimethylpiperidin-1-yl)-2,6-dimethyl-5-[2-(4-{8-oxa-3-azabicyclo[3.2.1]octan-3-yl}pyrimidin-2-yl)-1,2,3,4-tetrahydroisoquinolin-6-yl]pyridin-3-yl]acetic Acid

(1R,5S)-3-(2-Chloropyrimidin-4-yl)-8-oxa-3-azabicyclo[3.2.1]octane (22 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at 90° C. The reaction was stirred at 90° C. overnight. LCMS showed partial conversion. Sodium iodide (7 mg, 0.05 mmol) was added and the reaction was stirred at 90° C. overnight. The reaction was concentrated and then taken up in 1.5 mL of EtOH and treated with 0.1 mL of 5N NaOH and stirred at 80° C. overnight. The crude material was purified via preparative LC/MS to afford (2S)-2-(tert-butoxy)-2-[4-(4,4-dimethylpiperidin-1-yl)-2,6-dimethyl-5-[2-(4-{8-oxa-3-azabicyclo[3.2.1]octan-3-yl}pyrimidin-2-yl)-1,2,3,4-tetrahydroisoquinolin-6-yl]pyridin-3-yl]acetic acid (7.0 mg).

LCMS (M+H)=669.2. ¹H NMR (500 MHz, DMSO-d₆) δ 7.98-7.84 (m, 1H), 7.36-7.26 (m, 1H), 7.19-7.06 (m, 1H), 7.01-6.90 (m, 1H), 6.03 (d, J=6.1 Hz, 1H), 5.78 (br d, J=13.7 Hz, 1H), 4.95 (t, J=16.9 Hz, 1H), 4.84-4.67 (m, 1H), 4.42 (br s, 2H), 4.12-3.80 (m, 5H), 3.06-2.95 (m, 3H), 2.92-2.73 (m, 4H), 2.16-2.05 (m, 4H), 2.01-1.91 (m, 1H), 1.86-1.73 (m, 3H), 1.72-1.60 (m, 2H), 1.55-1.40 (m, 1H), 1.31-1.21 (m, 1H), 1.20-1.06 (m, 10H), 1.05-0.90 (m, 1H), 0.88-0.75 (m, 3H), 0.59 (s, 1H), 0.48 (s, 2H).

### Example 31

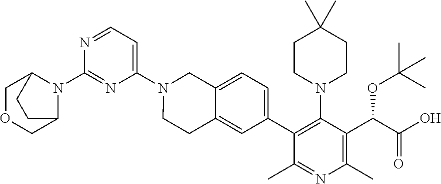 [SMILES: Cc1nc(C)c([C@H](OC(C)(C)C)C(=O)O)c(N2CCC(C)(C)CC2)c1-c1ccc2c(c1)CCN(c1ccnc(N3C4CCC3COC4)n1)C2]

### (2S)-2-(tert-Butoxy)-2-[4-(4,4-dimethylpiperidin-1-yl)-2,6-dimethyl-5-[2-(2-{3-oxa-8-azabicyclo[3.2.1]octan-8-yl}pyrimidin-4-yl)-1,2,3,4-tetrahydroisoquinolin-6-yl]pyridin-3-yl]acetic Acid

(1R,5S)-8-(4-Chloropyrimidin-2-yl)-3-oxa-8-azabicyclo[3.2.1]octane (22 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.144 mmol) and sodium iodide (14 mg, 0.096 mmol) in ACN (1 ml) at rt. The reaction was allowed to stir at 80° C. over the weekend (for 3 days). Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(tert-butoxy)-2-[4-(4,4-dimethylpiperidin-1-yl)-2,6-dimethyl-5-[2-(2-{3-oxa-8-azabicyclo[3.2.1]octan-8-yl}pyrimidin-4-yl)-1,2,3,4-tetrahydroisoquinolin-6-yl]pyridin-3-yl]acetic acid (8.2 mg). LCMS (M+H)=669.19. ¹H NMR (500 MHz, DMSO-d₆) δ 7.94 (d, J=5.9 Hz, 1H), 7.35 (dd, J=13.6, 8.1 Hz, 1H), 7.17 (br d, J=8.8 Hz, 1H), 7.01-6.97 (m, 1H), 6.22 (br d, J=5.9 Hz, 1H), 5.82 (br d, J=13.9 Hz, 1H), 4.64 (br d, J=16.1 Hz, 1H), 4.58 (br s, 2H), 3.83 (br s, 1H), 3.64-3.52 (m, 2H), 2.92 (br s, 2H), 2.56 (s, 7H), 2.49-2.39 (m, 3H), 2.12 (s, 2H), 2.08 (s, 1H), 1.92 (s, 5H), 1.84 (br s, 2H), 1.47 (br s, 1H), 1.25 (br s, 1H), 1.18-1.09 (m, 9H), 0.88-0.77 (m, 3H), 0.58 (s, 1H), 0.47 (s, 2H).

### Example 32

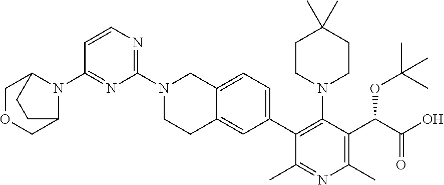 [SMILES: Cc1nc(C)c([C@H](OC(C)(C)C)C(=O)O)c(N2CCC(C)(C)CC2)c1-c1ccc2c(c1)CCN(c1nccc(N3C4CCC3COC4)n1)C2]

### (2S)-2-(tert-Butoxy)-2-[4-(4,4-dimethylpiperidin-1-yl)-2,6-dimethyl-5-[2-(4-{3-oxa-8-azabicyclo[3.2.1]octan-8-yl}pyrimidin-2-yl)-1,2,3,4-tetrahydroisoquinolin-6-yl]pyridin-3-yl]acetic Acid

(1R,5S)-8-(2-Chloropyrimidin-4-yl)-3-oxa-8-azabicyclo[3.2.1]octane (22 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) and sodium iodide (14 mg, 0.096 mmol) in ACN (1 ml) at rt. The reaction was allowed to stir at 80° C. over the weekend (for 3 days). Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(tert-butoxy)-2-[4-(4,4-dimethylpiperidin-1-yl)-2,6-dimethyl-5-[2-(4-{3-oxa-8-azabicyclo[3.2.1]octan-8-yl}pyrimidin-2-yl)-1,2,3,4-tetrahydroisoquinolin-6-yl]pyridin-3-yl]acetic acid (11.7 mg). LCMS (M+H)=669.23. ¹H NMR (500 MHz, DMSO-d₆) δ 7.94 (d, J=5.9 Hz, 1H), 7.35-7.30 (m, 1H), 7.14 (br s, 1H), 6.98-6.94 (m, 1H), 6.08 (d, J=6.2 Hz, 1H), 5.84 (br d, J=15.0 Hz, 1H), 4.99 (br d, J=16.9 Hz, 1H), 4.74 (br d, J=17.2 Hz, 1H), 3.92 (s, 1H), 3.62-3.53 (m, 2H), 2.88 (br s, 2H), 2.55 (s, 7H), 2.44 (s, 3H), 2.12 (s, 2H), 2.08 (s, 1H), 2.00-1.85 (m, 6H), 1.47 (br s, 1H), 1.25 (br s, 1H), 1.19-1.10 (m, 10H), 0.96 (br s, 1H), 0.88-0.77 (m, 3H), 0.59 (s, 1H), 0.49 (s, 2H).

### Example 33

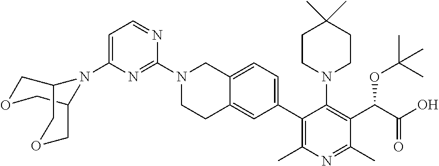 [SMILES: Cc1nc(C)c([C@H](OC(C)(C)C)C(=O)O)c(N2CCC(C)(C)CC2)c1-c1ccc2c(c1)CCN(c1nccc(N3C4COCC3COC4)n1)C2]

### (2S)-2-(tert-Butoxy)-2-[4-(4,4-dimethylpiperidin-1-yl)-5-[2-(4-{3,7-dioxa-9-azabicyclo[3.3.1]nonan-9-yl}pyrimidin-2-yl)-1,2,3,4-tetrahydroisoquinolin-6-yl]-2,6-dimethylpyridin-3-yl]acetic Acid

9-(2-Chloropyrimidin-4-yl)-3,7-dioxa-9-azabicyclo[3.3.1]nonane (23 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and K₂CO₃ (20 mg, 0.14 mmol) in dioxane (1 ml) at rt. The reaction was stirred at 100° C. overnight. The mixture was concentrated and then taken up in 1.5 mL of EtOH and treated with 0.1 mL of 5N NaOH. The mixture was then stirred at 100° C. for 5 hr. After cooling to rt, the crude material was purified via preparative LC/MS to afford title compound (7.7 mg). LCMS (M+H)=685.22. ¹H NMR (500 MHz, DMSO-d₆) δ 7.98 (d, J=6.2 Hz, 1H), 7.34-7.29 (m, 1H), 7.15-7.12 (m, 1H), 6.97-6.94 (m, 1H), 6.13 (d, J=5.9 Hz, 1H), 5.82 (br d, J=14.7 Hz, 1H), 4.98 (br d, J=17.2 Hz, 1H), 4.82-4.60 (m, 1H), 3.99 (br d, J=11.0 Hz, 5H), 3.95-3.80 (m, 2H), 3.73 (br d, J=7.7 Hz, 3H), 2.91-2.77 (m, 3H), 2.55 (s, 3H), 2.44 (s, 3H), 2.12 (s, 2H), 2.07 (s, 1H), 1.91 (s, 1H), 1.47 (br s, 1H), 1.31-1.21 (m, 1H), 1.18-1.09 (m, 10H), 1.01 (br d, J=13.2 Hz, 1H), 0.94 (br d, J=11.7 Hz, 1H), 0.87-0.78 (m, 3H), 0.58 (s, 1H), 0.49 (s, 2H).

### Example 34

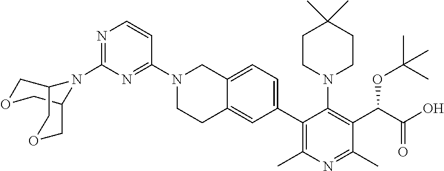 [SMILES: Cc1nc(C)c([C@H](OC(C)(C)C)C(=O)O)c(N2CCC(C)(C)CC2)c1-c1ccc2c(c1)CCN(c1ccnc(N3C4COCC3COC4)n1)C2]

### (2S)-2-(tert-Butoxy)-2-[4-(4,4-dimethylpiperidin-1-yl)-5-[2-(2-{3,7-dioxa-9-azabicyclo[3.3.1]nonan-9-yl}pyrimidin-4-yl)-1,2,3,4-tetrahydroisoquinolin-6-yl]-2,6-dimethylpyridin-3-yl]acetic Acid

9-(4-Chloropyrimidin-2-yl)-3,7-dioxa-9-azabicyclo[3.3.1]nonane (15 mg, 0.062 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and K₂CO₃ (20 mg, 0.14 mmol) in dioxane (1 ml) at rt. The reaction was stirred at 100° C. overnight. The mixture was concentrated and then taken up in 1.5 mL of EtOH and treated with 0.1 mL of 5N NaOH. The mixture was then stirred at 100° C. for 5 hr. After cooling to rt, the crude material was purified via preparative LC/MS to afford title compound (8.1 mg). LCMS (M+H)=685.19. ¹H NMR (500 MHz, DMSO-d₆) δ 7.96 (d, J=5.9 Hz, 1H), 7.36-7.31 (m, 1H), 7.18-7.14 (m, 1H), 6.98-6.95 (m, 1H), 6.24-6.20 (m, 1H), 5.71 (br d, J=14.7 Hz, 1H), 4.63 (br d, J=16.5 Hz, 1H), 4.45 (br s, 1H), 4.39 (br s, 1H), 3.96 (br d, J=11.4 Hz, 3H), 3.82 (br s, 1H), 3.70 (br d, J=11.0 Hz, 3H), 2.91 (br d, J=5.9 Hz, 2H), 2.55 (s, 6H), 2.43 (s, 3H), 2.11 (s, 2H), 2.07 (s, 1H), 1.91 (s, 2H), 1.47 (br s, 1H), 1.24 (br s, 1H), 1.16-1.07 (m, 10H), 0.92 (br d, J=12.5 Hz, 1H), 0.86-0.77 (m, 3H), 0.58 (s, 1H), 0.47 (s, 2H).

### Example 35

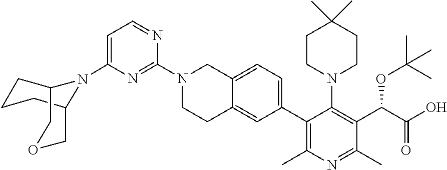 [SMILES: Cc1nc(C)c([C@H](OC(C)(C)C)C(=O)O)c(N2CCC(C)(C)CC2)c1-c1ccc2c(c1)CCN(c1nccc(N3C4CCCC3COC4)n1)C2]

### (2S)-2-(tert-Butoxy)-2-[4-(4,4-dimethylpiperidin-1-yl)-2,6-dimethyl-5-[2-(4-{3-oxa-9-azabicyclo[3.3.1]nonan-9-yl}pyrimidin-2-yl)-1,2,3,4-tetrahydroisoquinolin-6-yl]pyridin-3-yl]acetic Acid

9-(2-Chloropyrimidin-4-yl)-3-oxa-9-azabicyclo[3.3.1]nonane (23 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and K₂CO₃ (20 mg, 0.14 mmol) in dioxane (1 ml) at rt. The reaction was stirred at 100° C. overnight. The mixture was concentrated and then taken up in 1.5 mL of EtOH and treated with 0.1 mL of 5N NaOH. The mixture was then stirred at 100° C. for 5 hr. After cooling to rt, the crude material was purified via preparative LC/MS to afford title compound (8.4 mg). LCMS (M+H)=683.2. ¹H NMR (500 MHz, DMSO-d₆) δ 7.94 (d, J=5.9 Hz, 1H), 7.34-7.28 (m, 1H), 7.15-7.11 (m, 1H), 6.96-6.92 (m, 1H), 6.09 (d, J=5.9 Hz, 1H), 5.77 (br d, J=15.4 Hz, 1H), 5.02-4.91 (m, 1H), 4.82-4.66 (m, 1H), 4.05 (br s, 1H), 3.96 (br d, J=9.9 Hz, 2H), 3.93-3.80 (m, 1H), 3.70 (br d, J=11.4 Hz, 2H), 2.87 (br s, 2H), 2.84-2.75 (m, 1H), 2.55 (s, 5H), 2.49-2.40 (m, 4H), 2.12 (s, 2H), 2.07 (s, 1H), 1.91 (s, 1H), 1.78 (br s, 4H), 1.52 (br s, 1H), 1.46 (br s, 1H), 1.30-1.20 (m, 1H), 1.17-1.08 (m, 9H), 0.93 (br s, 1H), 0.87-0.76 (m, 3H), 0.58 (s, 1H), 0.47 (br s, 2H).

### Example 36

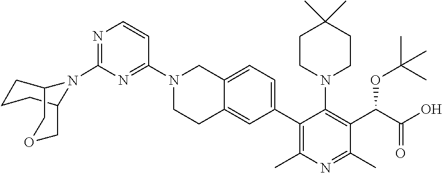 [SMILES: Cc1nc(C)c([C@H](OC(C)(C)C)C(=O)O)c(N2CCC(C)(C)CC2)c1-c1ccc2c(c1)CCN(c1ccnc(N3C4CCCC3COC4)n1)C2]

### (2S)-2-(tert-Butoxy)-2-[4-(4,4-dimethylpiperidin-1-yl)-2,6-dimethyl-5-[2-(2-{3-oxa-9-azabicyclo[3.3.1]nonan-9-yl}pyrimidin-4-yl)-1,2,3,4-tetrahydroisoquinolin-6-yl]pyridin-3-yl]acetic Acid

9-(4-Chloropyrimidin-2-yl)-3-oxa-9-azabicyclo[3.3.1]nonane (15 mg, 0.062 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and K₂CO₃ (20 mg, 0.14 mmol) in dioxane (1 ml) at 100° C. The reaction was stirred at 100° C. overnight. The mixture was concentrated and then taken up in 1.5 mL of EtOH and treated with 0.1 mL of 5N NaOH. The mixture was then stirred at 100° C. for 5 hr. After cooling to rt, the crude material was purified via preparative LC/MS to afford title compound (7.0 mg). LCMS (M+H)=683.2. ¹H NMR (500 MHz, DMSO-d₆) δ 7.94 (d, J=5.9 Hz, 1H), 7.33 (dd, J=13.6, 7.7 Hz, 1H), 7.18-7.13 (m, 1H), 6.99-6.94 (m, 1H), 6.19-6.15 (m, 1H), 5.75 (br d, J=15.8 Hz, 1H), 4.62 (br s, 1H), 4.52 (br s, 2H), 3.92 (br d, J=12.5 Hz, 2H), 3.80 (br s, 1H), 3.68 (br d, J=9.9 Hz, 2H), 2.91 (br s, 2H), 2.78 (br t, J=10.3 Hz, 1H), 2.55 (s, 5H), 2.49-2.41 (m, 3H), 2.12 (s, 2H), 2.07 (s, 1H), 1.91 (s, 1H), 1.82-1.66 (m, 4H), 1.48 (br d, J=14.3 Hz, 2H), 1.24 (br s, 1H), 1.13-1.09 (m, 9H), 0.87-0.76 (m, 3H), 0.57 (s, 1H), 0.45 (br s, 2H).

### Example 37

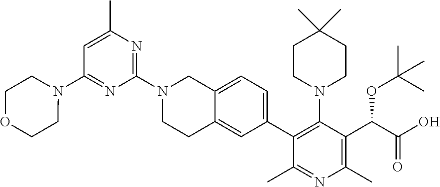 [SMILES: Cc1cc(N2CCOCC2)nc(N2CCc3cc(-c4c(C)nc(C)c([C@H](OC(C)(C)C)C(=O)O)c4N4CCC(C)(C)CC4)ccc3C2)n1]

### (2S)-2-(tert-Butoxy)-2-[4-(4,4-dimethylpiperidin-1-yl)-2,6-dimethyl-5-{2-[4-methyl-6-(morpholin-4-yl)pyrimidin-2-yl]-1,2,3,4-tetrahydroisoquinolin-6-yl}pyridin-3-yl]acetic Acid

4-(2-Chloro-6-methylpyrimidin-4-yl)morpholine (20 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at 90° C. The reaction was stirred at 90° C. overnight. LCMS showed partial conversion. Sodium iodide (7 mg, 0.05 mmol) was added and the reaction was stirred at 90° C. overnight. The reaction was concentrated and then taken up in 1.5 mL of EtOH and treated with 0.1 mL of 5N NaOH and stirred at 80° C. overnight. The crude material was purified via preparative LC/MS to afford (2S)-2-(tert-butoxy)-2-[4-(4,4-dimethylpiperidin-1-yl)-2,6-dimethyl-5-{2-[4-methyl-6-(morpholin-4-yl)pyrimidin-2-yl]-1,2,3,4-tetrahydroisoquinolin-6-yl}pyridin-3-yl]acetic acid (6.7 mg). LCMS (M+H)=657.24. ¹H NMR (500 MHz, DMSO-d₆) δ 7.36-7.29 (m, 1H), 7.18-7.07 (m, 1H), 7.00-6.87 (m, 1H), 6.02 (s, 1H), 5.85 (br d, J=11.3 Hz, 1H), 5.00-4.86 (m, 1H), 4.84-4.74 (m, 1H), 4.12-3.83 (m, 3H), 3.66 (br s, 5H), 2.96-2.74 (m, 3H), 2.55 (s, 2H), 2.44 (s, 3H), 2.26-2.16 (m, 3H), 2.14-2.06 (m, 4H), 2.02-1.90 (m, 1H), 1.88-1.75 (m, 1H), 1.54-1.42 (m, 1H), 1.33-1.24 (m, 1H), 1.22-1.16 (m, 1H), 1.15-1.09 (m, 9H), 1.06-0.93 (m, 1H), 0.89-0.77 (m, 3H), 0.59 (s, 1H), 0.52 (s, 2H).

### Example 38

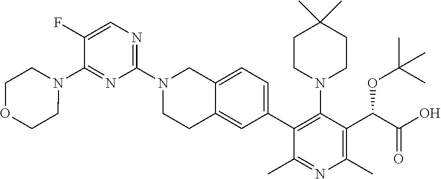 [SMILES: Cc1nc(C)c([C@H](OC(C)(C)C)C(=O)O)c(N2CCC(C)(C)CC2)c1-c1ccc2c(c1)CCN(c1ncc(F)c(N3CCOCC3)n1)C2]

### (2S)-2-(tert-Butoxy)-2-[4-(4,4-dimethylpiperidin-1-yl)-5-{2-[5-fluoro-4-(morpholin-4-yl)pyrimidin-2-yl]-1,2,3,4-tetrahydroisoquinolin-6-yl}-2,6-dimethylpyridin-3-yl]acetic Acid

4-(2-Chloro-5-fluoropyrimidin-4-yl)morpholine (21 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at rt. The reaction was stirred at 90° C. overnight. LCMS showed partial conversion. Sodium iodide (7 mg, 0.048 mmol) was added and the reaction was stirred at 90° C. overnight. The reaction was concentrated and then taken up in 1.5 mL of EtOH and treated with 0.1 mL of 5N NaOH and stirred at 80° C. overnight. The crude material was purified via preparative LC/MS to afford (2S)-2-(tert-butoxy)-2-[4-(4,4-dimethylpiperidin-1-yl)-5-{2-[5-fluoro-4-(morpholin-4-yl)pyrimidin-2-yl]-1,2,3,4-tetrahydroisoquinolin-6-yl}-2,6-dimethylpyridin-3-yl]acetic acid (5.7 mg). LCMS (M+H)=661.22. ¹H NMR (500 MHz, DMSO-d₆) δ 7.99 (d, J=6.7 Hz, 1H), 7.38-7.27 (m, 1H), 7.14 (br d, J=6.4 Hz, 1H), 7.02-6.90 (m, 1H), 5.85-5.68 (m, 1H), 4.98-4.83 (m, 1H), 4.80-4.69 (m, 1H), 4.03-3.84 (m, 4H), 2.92-2.85 (m, 3H), 2.84-2.73 (m, 2H), 2.47-2.39 (m, 4H), 2.15-2.04 (m, 4H), 1.91 (s, 3H), 1.81-1.71 (m, 1H), 1.47 (br t, J=12.7 Hz, 1H), 1.32-1.21 (m, 1H), 1.17 (br d, J=10.7 Hz, 1H), 1.14-1.07 (m, 9H), 1.04-0.90 (m, 1H), 0.87-0.77 (m, 3H), 0.58 (s, 1H), 0.49 (s, 2H).

### Example 39

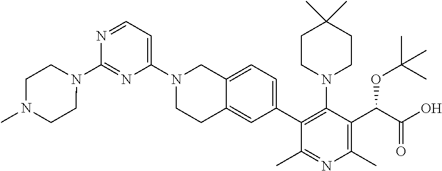 [SMILES: Cc1nc(C)c([C@H](OC(C)(C)C)C(=O)O)c(N2CCC(C)(C)CC2)c1-c1ccc2c(c1)CCN(c1ccnc(N3CCN(C)CC3)n1)C2]

### (2S)-2-(tert-Butoxy)-2-[4-(4,4-dimethylpiperidin-1-yl)-2,6-dimethyl-5-{2-[2-(4-methylpiperazin-1-yl)pyrimidin-4-yl]-1,2,3,4-tetrahydroisoquinolin-6-yl}pyridin-3-yl]acetic Acid

1-Methylpiperazine (7 μl, 0.06 mmol) was added to a stirring solution of (S)-isopropyl 2-(tert-butoxy)-2-(5-(2-(2-chloropyrimidin-4-yl)-1,2,3,4-tetrahydroisoquinolin-6-yl)-4-(4,4-dimethylpiperidin-1-yl)-2,6-dimethylpyridin-3-yl)acetate (25 mg, 0.039 mmol) and K₂CO₃ (16 mg, 0.12 mmol) in acetonitrile (1 ml) at rt. The reaction was stirred at 80° C. overnight. Then the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(tert-butoxy)-2-[4-(4,4-dimethylpiperidin-1-yl)-2,6-dimethyl-5-{2-[2-(4-methylpiperazin-1-yl)pyrimidin-4-yl]-1,2,3,4-tetrahydroisoquinolin-6-yl}pyridin-3-yl]acetic acid (23.8 mg). LCMS (M+H)=: 656.25. ¹H NMR (500 MHz, DMSO-d₆) δ 7.92 (d, J=6.2 Hz, 1H), 7.37-7.32 (m, 1H), 7.19-7.15 (m, 1H), 6.98 (s, 1H), 6.17 (d, J=5.9 Hz, 1H), 5.82 (br d, J=12.8 Hz, 1H), 4.67 (br d, J=17.2 Hz, 1H), 3.86-3.71 (m, 1H), 2.92 (br s, 2H), 2.81 (br s, 1H), 2.55 (s, 6H), 2.50-2.41 (m, 3H), 2.34 (br s, 4H), 2.21 (s, 3H), 2.12 (s, 2H), 2.07 (s, 1H), 1.91 (s, 3H), 1.25 (br s, 1H), 1.12 (d, J=2.9 Hz, 10H), 0.95 (br d, J=12.1 Hz, 1H), 0.87-0.78 (m, 3H), 0.58 (s, 1H), 0.48 (s, 2H).

### Example 40

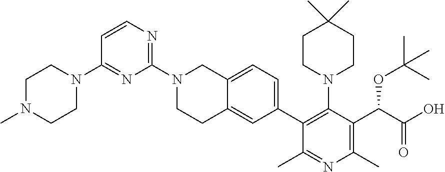 [SMILES: Cc1nc(C)c([C@H](OC(C)(C)C)C(=O)O)c(N2CCC(C)(C)CC2)c1-c1ccc2c(c1)CCN(c1nccc(N3CCN(C)CC3)n1)C2]

### (2S)-2-(tert-Butoxy)-2-[4-(4,4-dimethylpiperidin-1-yl)-2,6-dimethyl-5-{2-[4-(4-methylpiperazin-1-yl)pyrimidin-2-yl]-1,2,3,4-tetrahydroisoquinolin-6-yl}pyridin-3-yl]acetic Acid

2-Chloro-4-(4-methylpiperazin-1-yl)pyrimidine (20 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.144 mmol) in ACN (1 ml) at rt. Sodium iodide (14 mg, 0.096 mmol) was added and the reaction was stirred at 90° C. overnight.

The reaction was concentrated and then taken up in 1.5 mL of EtOH and treated with 0.1 mL of 5N NaOH and stirred at 100° C. for 5 hrs. The crude material was purified via preparative LC/MS to afford (2S)-2-(tert-butoxy)-2-[4-(4,4-dimethylpiperidin-1-yl)-2,6-dimethyl-5-{2-[4-(4-methylpiperazin-1-yl)pyrimidin-2-yl]-1,2,3,4-tetrahydroisoquinolin-6-yl}pyridin-3-yl]acetic acid (8.0 mg). LCMS (M+H)=656.27. ¹H NMR (500 MHz, DMSO-d₆) δ 7.92 (d, J=6.1 Hz, 1H), 7.36-7.28 (m, 1H), 7.17-7.08 (m, 1H), 6.99-6.89 (m, 1H), 6.12 (d, J=6.1 Hz, 1H), 5.78 (br d, J=13.1 Hz, 1H), 5.02-4.89 (m, 1H), 4.79 (t, J=17.5 Hz, 1H), 2.46-2.40 (m, 3H), 2.36 (br s, 4H), 2.21 (s, 4H), 2.12-2.05 (m, 4H), 1.91 (s, 8H), 1.82-1.73 (m, 1H), 1.53-1.39 (m, 1H), 1.33-1.20 (m, 1H), 1.19-1.14 (m, 1H), 1.12 (d, J=2.1 Hz, 9H), 1.14-1.07 (m, 1H), 1.04-0.91 (m, 1H), 0.87-0.76 (m, 3H), 0.59 (s, 1H), 0.50 (s, 2H).

### Example 41

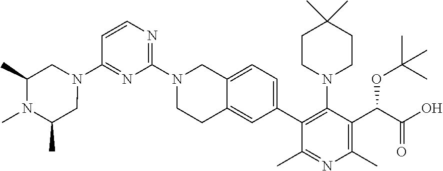 [SMILES: Cc1nc(C)c([C@H](OC(C)(C)C)C(=O)O)c(N2CCC(C)(C)CC2)c1-c1ccc2c(c1)CCN(c1nccc(N3C[C@@H](C)N(C)[C@@H](C)C3)n1)C2]

### (2S)-2-(tert-Butoxy)-2-[4-(4,4-dimethylpiperidin-1-yl)-2,6-dimethyl-5-(2-{4-[(3R,5S)-3,4,5-trimethylpiperazin-1-yl]pyrimidin-2-yl}-1,2,3,4-tetrahydroisoquinolin-6-yl)pyridin-3-yl]acetic Acid

(2S,6R)-1,2,6-Trimethylpiperazine (14 mg, 0.043 mmol) was added to a stirring solution of (S)-isopropyl 2-(tert-butoxy)-2-(5-(2-(4-chloropyrimidin-2-yl)-1,2,3,4-tetrahydroisoquinolin-6-yl)-4-(4,4-dimethylpiperidin-1-yl)-2,6-dimethylpyridin-3-yl)acetate (18 mg, 0.028 mmol) and K₂CO₃ (16 mg, 0.11 mmol) in acetonitrile (1 ml) at rt. The reaction was stirred at 80° C. overnight. Then, the sample was concentrated and taken up in 1.5 mL of EtOH and treated with 0.1 mL of 5 N NaOH. The mixture was then stirred at 80° C. overnight. The crude material was purified via preparative LC/MS to afford (2S)-2-(tert-butoxy)-2-[4-(4,4-dimethylpiperidin-1-yl)-2,6-dimethyl-5-(2-{4-[(3R,5S)-3,4,5-trimethylpiperazin-1-yl]pyrimidin-2-yl}-1,2,3,4-tetrahydroisoquinolin-6-yl)pyridin-3-yl]acetic acid (4.7 mg). LCMS (M+H)=684.27. ¹H NMR (500 MHz, DMSO-d₆) δ 7.91 (d, J=6.2 Hz, 1H), 7.35-7.30 (m, 1H), 7.16-7.12 (m, 1H), 6.97-6.93 (m, 1H), 6.15 (d, J=5.9 Hz, 1H), 5.74 (br d, J=12.5 Hz, 1H), 4.94 (t, J=16.7 Hz, 1H), 4.00-3.84 (m, 1H), 2.88 (br s, 2H), 2.61-2.53 (m, 7H), 2.43 (s, 3H), 2.17 (s, 3H), 2.12 (s, 2H), 2.07 (s, 3H), 1.91 (s, 5H), 1.48 (br s, 1H), 1.24 (br s, 1H), 1.17-1.04 (m, 14H), 0.96 (br s, 1H), 0.87-0.79 (m, 3H), 0.59 (s, 1H), 0.50 (s, 2H).

### Example 42

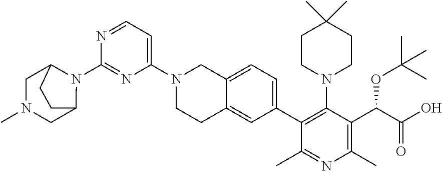 [SMILES: Cc1nc(C)c([C@H](OC(C)(C)C)C(=O)O)c(N2CCC(C)(C)CC2)c1-c1ccc2c(c1)CCN(c1ccnc(N3C4CCC3CN(C)C4)n1)C2]

### (2S)-2-(tert-Butoxy)-2-[4-(4,4-dimethylpiperidin-1-yl)-2,6-dimethyl-5-[2-(2-{3-methyl-3,8-diazabicyclo[3.2.1]octan-8-yl}pyrimidin-4-yl)-1,2,3,4-tetrahydroisoquinolin-6-yl]pyridin-3-yl]acetic Acid

(1R,5S)-8-(4-Chloropyrimidin-2-yl)-3-methyl-3,8-diazabicyclo[3.2.1]octane (23 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at rt. The reaction was allowed to stir at 80° C. for 2 days. Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(tert-butoxy)-2-[4-(4,4-dimethylpiperidin-1-yl)-2,6-dimethyl-5-[2-(2-{3-methyl-3,8-diazabicyclo[3.2.1]octan-8-yl}pyrimidin-4-yl)-1,2,3,4-tetrahydroisoquinolin-6-yl]pyridin-3-yl]acetic acid (19.0 mg). LCMS (M+H)=682.28. ¹H NMR (500 MHz, DMSO-d₆) δ 7.91 (d, J=5.9 Hz, 1H), 7.36-7.32 (m, 1H), 7.18-7.15 (m, 1H), 7.00-6.97 (m, 1H), 6.16 (d, J=6.2 Hz, 1H), 5.81 (br d, J=13.2 Hz, 1H), 4.62 (br s, 2H), 2.92 (br s, 2H), 2.60-2.53 (m, 8H), 2.44 (s, 3H), 2.19-2.07 (m, 8H), 1.91 (s, 3H), 1.86 (br d, J=6.6 Hz, 2H), 1.77 (br d, J=5.5 Hz, 2H), 1.47 (br s, 1H), 1.24 (br s, 1H), 1.18 (br s, 1H), 1.14-1.10 (m, 9H), 0.87-0.78 (m, 3H), 0.58 (s, 1H), 0.48 (s, 2H).

### Example 43

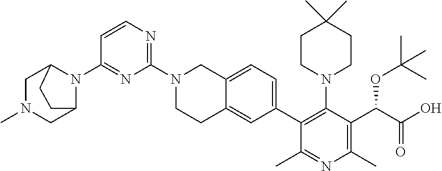 [SMILES: Cc1nc(C)c([C@H](OC(C)(C)C)C(=O)O)c(N2CCC(C)(C)CC2)c1-c1ccc2c(c1)CCN(c1nccc(N3C4CCC3CN(C)C4)n1)C2]

### (2S)-2-(tert-Butoxy)-2-[4-(4,4-dimethylpiperidin-1-yl)-2,6-dimethyl-5-[2-(4-{3-methyl-3,8-diazabicyclo[3.2.1]octan-8-yl}pyrimidin-2-yl)-1,2,3,4-tetrahydroisoquinolin-6-yl]pyridin-3-yl]acetic Acid

(1R,5S)-8-(2-Chloropyrimidin-4-yl)-3-methyl-3,8-diazabicyclo[3.2.1]octane (23 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) in ACN (1 ml) at rt. The reaction was allowed to stir at 80° C. for 2 days. Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (2S)-2-(tert-butoxy)-2-[4-(4,4-dimethylpiperidin-1-yl)-2,6-dimethyl-5-[2-(4-{3-methyl-3,8-diazabicyclo[3.2.1]octan-8-yl}pyrimidin-2-yl)-1,2,3,4-tetrahydroisoquinolin-6-yl]pyridin-3-yl]acetic acid (6.4 mg). LCMS (M+H)=682.23. ¹H NMR (500 MHz, DMSO-d₆) δ 7.90 (d, J=5.8 Hz, 1H), 7.35-7.29 (m, 1H), 7.16-7.12 (m, 1H), 6.98-6.94 (m, 1H), 6.04 (d, J=6.2 Hz, 1H), 5.82 (br d, J=14.3 Hz, 1H), 5.03-4.92 (m, 1H), 4.82-4.69 (m, 1H), 4.10-3.96 (m, 1H), 3.92-3.81 (m, 1H), 2.88 (br s, 2H), 2.61-2.54 (m, 7H), 2.44 (s, 3H), 2.18-2.06 (m, 8H), 1.97-1.84 (m, 4H), 1.81 (br s, 2H), 1.47 (br s, 1H), 1.25 (br s, 1H), 1.18-1.09 (m, 9H), 0.95 (br d, J=10.6 Hz, 1H), 0.87-0.78 (m, 3H), 0.59 (s, 1H), 0.49 (s, 2H).

### Example 44

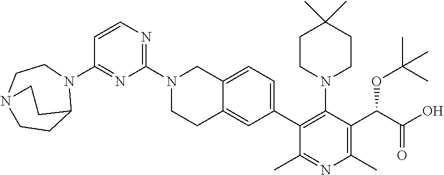 [SMILES: Cc1nc(C)c([C@H](OC(C)(C)C)C(=O)O)c(N2CCC(C)(C)CC2)c1-c1ccc2c(c1)CCN(c1nccc(N3CCN4CCC3CC4)n1)C2]

### (2S)-2-(tert-Butoxy)-2-{5-[2-(4-{1,4-diazabicyclo[3.2.2]nonan-4-yl}pyrimidin-2-yl)-1,2,3,4-tetrahydroisoquinolin-6-yl]-4-(4,4-dimethylpiperidin-1-yl)-2,6-dimethylpyridin-3-yl}acetic Acid

1,4-Diazabicyclo[3.2.2]nonane bis(4-methylbenzenesulfonate) (20 mg, 0.043 mmol) was added to a stirring solution of (S)-isopropyl 2-(tert-butoxy)-2-(5-(2-(4-chloropyrimidin-2-yl)-1,2,3,4-tetrahydroisoquinolin-6-yl)-4-(4,4-dimethylpiperidin-1-yl)-2,6-dimethylpyridin-3-yl)acetate (18 mg, 0.028 mmol) and K₂CO₃ (16 mg, 0.11 mmol) in acetonitrile (1 ml) at rt. The reaction was stirred at 80° C. overnight. Then, the sample was concentrated and taken up in 1.5 mL of EtOH and treated with 0.1 mL of 5 N NaOH. The mixture was then stirred at 80° C. overnight. The crude material was purified via preparative LC/MS to afford (2S)-2-(tert-butoxy)-2-{5-[2-(4-{1,4-diazabicyclo[3.2.2]nonan-4-yl}pyrimidin-2-yl)-1,2,3,4-tetrahydroisoquinolin-6-yl]-4-(4,4-dimethylpiperidin-1-yl)-2,6-dimethylpyridin-3-yl}acetic acid (2.4 mg). LCMS (M+H)=682.22. ¹H NMR (500 MHz, DMSO-d₆) δ 7.88 (d, J=6.2 Hz, 1H), 7.36-7.23 (m, 1H), 7.13 (d, J=7.3 Hz, 1H), 6.93 (br s, 1H), 6.08-5.89 (m, 1H), 5.77-5.61 (m, 1H), 5.06-4.86 (m, 1H), 4.84-4.71 (m, 1H), 3.92 (s, 2H), 2.96 (br s, 2H), 2.87 (br s, 6H), 2.55 (s, 6H), 2.43 (s, 3H), 2.11 (s, 2H), 2.07 (s, 2H), 1.91 (s, 6H), 1.77 (s, 2H), 1.15-1.07 (m, 9H), 0.87-0.78 (m, 3H), 0.59 (s, 1H), 0.50 (s, 2H).

### Example 45

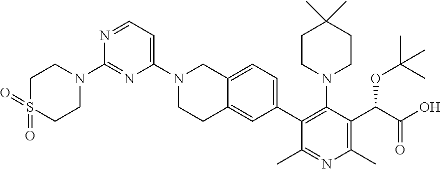 [SMILES: Cc1nc(C)c([C@H](OC(C)(C)C)C(=O)O)c(N2CCC(C)(C)CC2)c1-c1ccc2c(c1)CCN(c1ccnc(N3CCS(=O)(=O)CC3)n1)C2]

### (S)-2-(tert-Butoxy)-2-(4-(4,4-dimethylpiperidin-1-yl)-5-(2-(2-(1,1-dioxidothiomorpholino)pyrimidin-4-yl)-1,2,3,4-tetrahydroisoquinolin-6-yl)-2,6-dimethylpyridin-3-yl)acetic Acid

4-(4-Chloropyrimidin-2-yl)thiomorpholine 1,1-dioxide (24 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) and sodium iodide (14 mg, 0.096 mmol) in ACN (1 ml) at rt. The reaction was allowed to stir at 80° C. over the weekend (for 3 days). Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (S)-2-(tert-butoxy)-2-(4-(4,4-dimethylpiperidin-1-yl)-5-(2-(2-(1,1-dioxidothiomorpholino)pyrimidin-4-yl)-1,2,3,4-tetrahydroisoquinolin-6-yl)-2,6-dimethylpyridin-3-yl)acetic acid (16.3 mg). LCMS (M+H)=691.14. ¹H NMR (500 MHz, DMSO-d₆) δ 7.99 (d, J=5.9 Hz, 1H), 7.38-7.33 (m, 1H), 7.20-7.17 (m, 1H), 7.00 (s, 1H), 6.30 (br d, J=5.9 Hz, 1H), 5.81 (br d, J=12.5 Hz, 1H), 4.79 (br s, 1H), 4.20 (br s, 3H), 3.92 (s, 1H), 3.10 (br s, 3H), 2.94 (br s, 2H), 2.56 (s, 7H), 2.44 (s, 3H), 2.12 (s, 2H), 2.08 (s, 1H), 1.92 (s, 3H), 1.47 (br s, 1H), 1.25-1.10 (m, 9H), 1.06-0.91 (m, 1H), 0.87-0.78 (m, 3H), 0.58 (s, 1H), 0.48 (s, 2H).

### Example 46

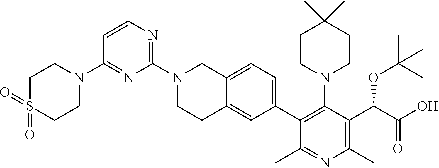 [SMILES: Cc1nc(C)c([C@H](OC(C)(C)C)C(=O)O)c(N2CCC(C)(C)CC2)c1-c1ccc2c(c1)CCN(c1nccc(N3CCS(=O)(=O)CC3)n1)C2]

### (S)-2-(tert-Butoxy)-2-(4-(4,4-dimethylpiperidin-1-yl)-5-(2-(4-(1,1-dioxidothiomorpholino)pyrimidin-2-yl)-1,2,3,4-tetrahydroisoquinolin-6-yl)-2,6-dimethylpyridin-3-yl)acetic Acid

4-(2-Chloropyrimidin-4-yl)thiomorpholine 1,1-dioxide (24 mg, 0.096 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25 mg, 0.048 mmol) and potassium carbonate (20 mg, 0.14 mmol) and sodium iodide (14 mg, 0.096 mmol) in ACN (1 ml) at rt. The reaction was allowed to stir at 80° C. over the weekend (for 3 days). Then, the sample was concentrated and taken up in 1.5 mL of EtOH and treated with 0.1 mL of 5 N NaOH. The mixture was then stirred at 100° C. for 5 hrs. The crude material was purified via preparative LC/MS to afford (S)-2-(tert-butoxy)-2-(4-(4,4-dimethylpiperidin-1-yl)-5-(2-(4-(1,1-dioxidothiomorpholino)pyrimidin-2-yl)-1,2,3,4-tetrahydroisoquinolin-6-yl)-2,6-dimethylpyridin-3-yl)acetic acid (14.0 mg). LCMS (M+H)=691.18. ¹H NMR (500 MHz, DMSO-d₆) δ 8.03 (d, J=5.9 Hz, 1H), 7.35-7.30 (m, 1H), 7.17-7.13 (m, 1H), 6.98-6.95 (m, 1H), 6.31 (d, J=5.7 Hz, 1H), 5.79 (br d, J=12.5 Hz, 1H), 4.95 (t, J=16.9 Hz, 1H), 4.13-4.02 (m, 3H), 3.99-3.93 (m, 1H), 3.18-3.10 (m, 3H), 2.94-2.84 (m, 2H), 2.55 (s, 7H), 2.49-2.38 (m, 2H), 2.11 (s, 2H), 2.07 (s, 1H), 1.91 (s, 3H), 1.48 (br s, 1H), 1.26 (br d, J=17.2 Hz, 1H), 1.12 (d, J=2.6 Hz, 9H), 1.05-0.90 (m, 1H), 0.88-0.78 (m, 3H), 0.59 (s, 1H), 0.49 (s, 2H).

### Example 47

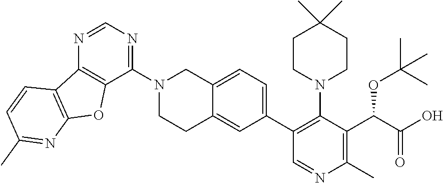 [SMILES: Cc1ccc2c(n1)oc1c(N3CCc4cc(-c5cnc(C)c([C@H](OC(C)(C)C)C(=O)O)c5N5CCC(C)(C)CC5)ccc4C3)ncnc12]

### (2S)-2-(tert-Butoxy)-2-[4-(4,4-dimethylpiperidin-1-yl)-2-methyl-5-(2-{11-methyl-8-oxa-3,5,10-triazatricyclo[7.4.0.0²,7]trideca-1(9),2,4,6,10,12-hexaen-6-yl}1,2,3,4tetrahydroisoquinolin-6-yl)pyridin-3-yl]acetic Acid

4-Chloro-7-methylpyrido[3′,2′:4,5]furo[3,2-d]pyrimidine (9 mg, 0.04 mmol) was added to a stirring solution of (S)-isopropyl 2-(tert-butoxy)-2-(4-(4,4-dimethylpiperidin-1-yl)-2-methyl-5-(1,2,3,4-tetrahydroisoquinolin-6-yl)pyridin-3-yl)acetate (20 mg, 0.039 mmol) and potassium carbonate (11 mg, 0.079 mmol) in ACN (0.8 ml) at rt. The reaction was allowed to stir at 80° C. overnight. Then, the sample was concentrated and taken up in 1.5 mL of EtOH and treated with 0.1 mL of 5 N NaOH. The mixture was then stirred at 100° C. for 5 hrs. Then the crude material was purified via preparative LC/MS to afford (2S)-2-(tert-butoxy)-2-[4-(4,4-dimethylpiperidin-1-yl)-2-methyl-5-(2-{11-methyl-8-oxa-3,5,10-triazatricyclo[7.4.0.0^{2,7}]trideca-1(9),2,4,6,10,12-hexaen-6-yl}1,2,3,4tetrahydroisoquinolin-6-yl)pyridin-3-yl]acetic acid (6.0 mg). LCMS (M+H)=649.29. ¹H NMR (500 MHz, DMSO-d₆) δ 8.58 (s, 1H), 8.45 (d, J=8.1 Hz, 1H), 8.05 (s, 1H), 7.48 (d, J=7.9 Hz, 1H), 7.44-7.41 (m, 1H), 7.20 (d, J=5.4 Hz, 2H), 7.19 (s, 2H), 5.85 (s, 1H), 5.24 (br d, J=7.0 Hz, 2H), 4.38-4.28 (m, 2H), 3.11 (br d, J=5.5 Hz, 1H), 2.68 (s, 3H), 1.30 (br s, 3H), 1.24 (s, 2H), 1.13 (s, 11H), 1.05 (s, 1H), 0.86 (br s, 1H), 0.77 (br s, 6H). Not all of the piperidine protons were well resolved.

### Example 48

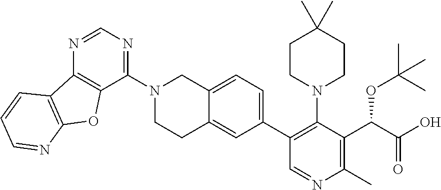 [SMILES: Cc1ncc(-c2ccc3c(c2)CCN(c2ncnc4c2oc2ncccc24)C3)c(N2CCC(C)(C)CC2)c1[C@H](OC(C)(C)C)C(=O)O]

### (2S)-2-(tert-Butoxy)-2-[4-(4,4-dimethylpiperidin-1-yl)-2-methyl-5-(2-{8-oxa-3,5,10-triazatricyclo[7.4.0.0²,7]trideca-1(9),2,4,6,10,12-hexaen-6-yl}-1,2,3,4-tetrahydroisoquinolin-6-yl)pyridin-3-yl]acetic Acid

4-Chloropyrido[3′,2′:4,5]furo[3,2-d]pyrimidine (20 mg, 0.098 mmol) was added to a stirring solution of (S)-isopropyl 2-(tert-butoxy)-2-(4-(4,4-dimethylpiperidin-1-yl)-2-methyl-5-(1,2,3,4-tetrahydroisoquinolin-6-yl)pyridin-3-yl)acetate (25 mg, 0.049 mmol) and potassium carbonate (20 mg, 0.15 mmol) in ACN (1 ml) at rt. The reaction was allowed to stir at 80° C. overnight. Then, the sample was concentrated and taken up in 1.5 mL of EtOH and treated with 0.1 mL of 5 N NaOH. The mixture was then stirred at 100° C. for 5 hrs. Then The crude material was purified via preparative LC/MS to afford (2S)-2-(tert-butoxy)-2-[4-(4,4-dimethylpiperidin-1-yl)-2-methyl-5-(2-{8-oxa-3,5,10-triazatricyclo[7.4.0.0^{2,7}]trideca-1(9),2,4,6,10,12-hexaen-6-yl}-1,2,3,4-tetrahydroisoquinolin-6-yl)pyridin-3-yl]acetic acid (13.4 mg). LCMS (M+H)=635.18. ¹H NMR (500 MHz, DMSO-d₆) δ 8.70 (d, J=4.8 Hz, 1H), 8.65-8.62 (m, 2H), 8.04 (s, 1H), 7.64 (dd, J=7.7, 4.8 Hz, 1H), 7.45 (d, J=8.1 Hz, 1H), 7.21 (br s, 2H), 5.75 (br s, 1H), 5.26 (br d, J=7.0 Hz, 2H), 4.36 (br s, 2H), 3.12 (br s, 2H), 2.55 (s, 2H), 2.49-2.47 (m, 3H), 1.92 (s, 1H), 1.29 (br d, J=9.2 Hz, 2H), 1.11 (s, 10H), 0.87 (br s, 3H), 0.70 (br s, 3H). Not all of the piperidine protons were well resolved.

### Example 49

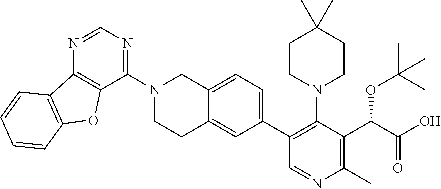 [SMILES: Cc1ncc(-c2ccc3c(c2)CCN(c2ncnc4c2oc2ccccc24)C3)c(N2CCC(C)(C)CC2)c1[C@H](OC(C)(C)C)C(=O)O]

### (2S)-2-(tert-Butoxy)-2-[4-(4,4-dimethylpiperidin-1-yl)-2-methyl-5-(2-{8-oxa-3,5-diazatricyclo[7.4.0.0^{2,7}]trideca-1(9),2(7),3,5,10,12-hexaen-6-yl}-1,2,3,4-tetrahydroisoquinolin-6-yl)pyridin-3-yl]acetic Acid

(S)-Isopropyl 2-(5-bromo-4-(4,4-dimethylpiperidin-1-yl)-2-methylpyridin-3-yl)-2-(tert-butoxy)acetate (25 mg, 0.055 mmol), 4-(6-(4,4,5,5-tetramethyl-1,3,2-dioxaborolan-2-yl)-3,4-dihydroisoquinolin-2(1H)-yl)benzofuro[3,2-d]pyrimidine (28 mg, 0.066 mmol), 2-dicyclohexylphosphino-2′,6′-dimethoxybiphenyl (5 mg, 11 μmol), potassium phosphate tribasic (87 mg, 0.41 mmol), Pd(OAc)₂ (1 mg, 5 μmol) were combined under N₂ (g). 1,4-Dioxane (1 ml) and water (0.2 ml) was added under N₂ (g). The reaction was stirred at 80° C. for 1 hr. The reaction was concentrated and subjected to hydrolysis (0.1 mL 5N NaOH in 1.5 mL EtOH) stirring for 4 hrs at 90° C. Then, the crude material was purified via preparative LC/MS to afford (2S)-2-(tert-butoxy)-2-[4-(4,4-dimethylpiperidin-1-yl)-2-methyl-5-(2-{8-oxa-3,5-diazatricyclo[7.4.0.0^{2,7}]trideca-1(9),2(7),3,5,10,12-hexaen-6-yl}-1,2,3,4-tetrahydroisoquinolin-6-yl)pyridin-3-yl]acetic acid (13.4 mg). LCMS (M+H)=634.16. ¹H NMR (500 MHz, DMSO-d₆) δ 8.59 (s, 1H), 8.13 (d, J=7.3 Hz, 1H), 8.06 (s, 1H), 7.89 (d, J=8.4 Hz, 1H), 7.73 (t, J=7.9 Hz, 1H), 7.52 (t, J=7.5 Hz, 1H), 7.44 (d, J=8.4 Hz, 1H), 7.21 (d, J=6.3 Hz, 2H), 7.20 (s, 1H), 5.82 (s, 1H), 5.25 (br d, J=16.5 Hz, 2H), 4.37 (br s, 2H), 3.10 (br d, J=7.7 Hz, 2H), 2.49-2.47 (m, 3H), 1.30 (br s, 2H), 1.23 (s, 3H), 1.12 (s, 10H), 0.85 (br d, J=6.6 Hz, 3H), 0.78 (br s, 5H).

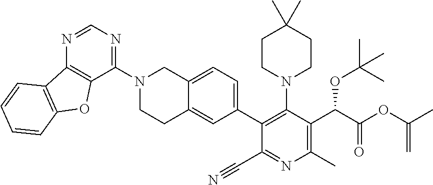 [SMILES: C=C(C)OC(=O)[C@@H](OC(C)(C)C)c1c(C)nc(C#N)c(-c2ccc3c(c2)CCN(c2ncnc4c2oc2ccccc24)C3)c1N1CCC(C)(C)CC1]

### (S)-Isopropyl 2-(5-(2-(benzofuro[3,2-d]pyrimidin-4-yl)-1,2,3,4-tetrahydroisoquinolin-6-yl)-6-cyano-4-(4,4-dimethylpiperidin-1-yl)-2-methylpyridin-3-yl)-2-(tert-butoxy)acetate

(S)-Isopropyl 2-(5-bromo-6-cyano-4-(4,4-dimethylpiperidin-1-yl)-2-methylpyridin-3-yl)-2-(tert-butoxy)acetate (30 mg, 0.062 mmol), 4-(6-(4,4,5,5-tetramethyl-1,3,2-dioxaborolan-2-yl)-3,4-dihydroisoquinolin-2(1H)-yl)benzofuro[3,2-d]pyrimidine (29 mg, 0.069 mmol), 2-dicyclohexylphosphino-2′,6′-dimethoxybiphenyl (5.1 mg, 0.012 mmol), Pd(OAc)₂ (1.4 mg, 6.2 μmol) and potassium phosphate tribasic (99 mg, 0.47 mmol) were combined under N₂. 1,4-Dioxane (1 ml) and water (0.2 ml) was added under N₂. The reaction was heated at 80° C. for 1 h. The reaction was then concentrated, adsorbed onto celite and was purified on silica gel (Biotage, EtOAc/hexanes gradient, 0-100% over 10 CVs) to afford (S)-isopropyl 2-(5-(2-(benzofuro[3,2-d]pyrimidin-4-yl)-1,2,3,4-tetrahydroisoquinolin-6-yl)-6-cyano-4-(4,4-dimethylpiperidin-1-yl)-2-methylpyridin-3-yl)-2-(tert-butoxy)acetate (40 mg, 0.057 mmol, 91% yield). LCMS (M+H)=701.25.

### Example 50

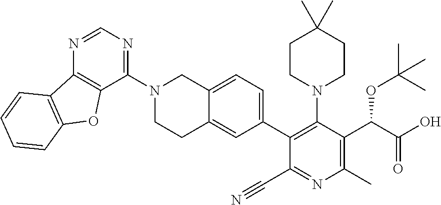 [SMILES: Cc1nc(C#N)c(-c2ccc3c(c2)CCN(c2ncnc4c2oc2ccccc24)C3)c(N2CCC(C)(C)CC2)c1[C@H](OC(C)(C)C)C(=O)O]

### (2S)-2-(tert-Butoxy)-2-[6-cyano-4-(4,4-dimethylpiperidin-1-yl)-2-methyl-5-(2-{8-oxa-3,5-diazatricyclo[7.4.0.0², 7trideca-1(9),2(7),3,5,10,12-hexaen-6-yl}-1,2,3,4-tetrahydroisoquinolin-6-yl)pyridin-3-yl]acetic Acid

Lithium hydroxide hydrate (3 mg, 0.06 mmol) dissolved in water (0.1 ml) was added to a stirring solution of (S)-isopropyl 2-(5-(2-(benzofuro[3,2-d]pyrimidin-4-yl)-1,2,3,4-tetrahydroisoquinolin-6-yl)-6-cyano-4-(4,4-dimethylpiperidin-1-yl)-2-methylpyridin-3-yl)-2-(tert-butoxy)acetate (40 mg, 0.057 mmol) in EtOH (1 ml) at rt. The reaction was stirred overnight at 70° C. Then, the crude material was purified via preparative LC/MS to afford title compound (6.4 mg). LCMS (M+H)=659.15. ¹H NMR (500 MHz, DMSO-d₆) δ 8.60 (s, 1H), 8.13 (d, J=7.7 Hz, 1H), 7.91-7.88 (m, 1H), 7.74 (t, J=7.9 Hz, 1H), 7.53 (t, J=7.3 Hz, 2H), 7.43 (br s, 1H), 7.19 (s, 1H), 5.72 (br s, 1H), 5.36 (br s, 1H), 4.36 (br s, 2H), 3.18 (s, 2H), 2.56-2.53 (m, 7H), 1.23 (br d, J=8.8 Hz, 3H), 1.12 (s, 10H), 0.81 (br s, 4H), 0.50 (br s, 3H).

### Example 51

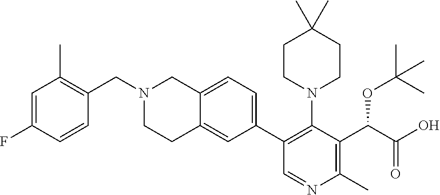 [SMILES: Cc1cc(F)ccc1CN1CCc2cc(-c3cnc(C)c([C@H](OC(C)(C)C)C(=O)O)c3N3CCC(C)(C)CC3)ccc2C1]

### (2S)-2-(tert-Butoxy)-2-[4-(4,4-dimethylpiperidin-1-yl)-5-{2-[(4-fluoro-2-methylphenyl)methyl]-1,2,3,4-tetrahydroisoquinolin-6-yl}-2-methylpyridin-3-yl]acetic Acid

(S)-Isopropyl 2-(5-bromo-4-(4,4-dimethylpiperidin-1-yl)-2-methylpyridin-3-yl)-2-(tert-butoxy)acetate (25 mg, 0.055 mmol), 2-(4-fluoro-2-methylbenzyl)-6-(4,4,5,5-tetramethyl-1,3,2-dioxaborolan-2-yl)-1,2,3,4-tetrahydroisoquinoline (31 mg, 0.082 mmol), 2-dicyclohexylphosphino-2′,6′-dimethoxybiphenyl (4.51 mg, 10.98 μmol), potassium phosphate tribasic (87 mg, 0.41 mmol), Pd(OAc)₂ (1 mg, 5 μmol) were combined under N₂ (g). 1,4-Dioxane (1 ml) and water (0.2 ml) was added under N₂ (g). The reaction was stirred at 80° C. for 1 hr. The reaction was concentrated and subjected to hydrolysis (0.1 mL 5N NaOH in 1.5 mL EtOH) stirring for 4 hrs at 90° C. Then, the crude material was purified via preparative LC/MS to afford (2S)-2-(tert-butoxy)-2-[4-(4,4-dimethylpiperidin-1-yl)-5-{2-[(4-fluoro-2-methylphenyl)methyl]-1,2,3,4-tetrahydroisoquinolin-6-yl}-2-methylpyridin-3-yl]acetic acid (5.5 mg). LCMS (M+H)=588.2. ¹H NMR (500 MHz, DMSO-d₆) δ 8.03 (s, 1H), 7.33 (t, J=7.3 Hz, 1H), 7.15-7.08 (m, 1H), 7.07-6.96 (m, 4H), 5.81 (s, 1H), 2.84 (br s, 2H), 2.74-2.68 (m, 2H), 2.55 (s, 6H), 2.49-2.45 (m, 3H), 2.36 (s, 3H), 1.30 (br d, J=10.3 Hz, 2H), 1.24 (s, 2H), 1.11 (s, 10H), 0.98-0.80 (m, 4H), 0.73 (br s, 3H).

### Example 52

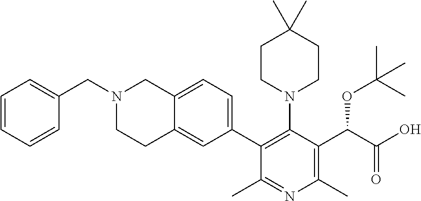 [SMILES: Cc1nc(C)c([C@H](OC(C)(C)C)C(=O)O)c(N2CCC(C)(C)CC2)c1-c1ccc2c(c1)CCN(Cc1ccccc1)C2]

### (2S)-2-[5-(2-Benzyl-1,2,3,4-tetrahydroisoquinolin-6-yl)-4-(4,4-dimethylpiperidin-1-yl)-2,6-dimethylpyridin-3-yl]-2-(tert-butoxy)acetic Acid

Benzaldehyde (20 μl, 0.17 mmol) was added to a stirring solution of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (30 mg, 0.058 mmol) in MeOH (1.2 ml) at rt. The reaction was stirred for 1 hr. Then, sodium cyanoborohydride (7.2 mg, 0.12 mmol) and zinc chloride (8 mg, 0.06 mmol) was added at once. The reaction was stirred for 1 hr. Then, 0.1 mL of 5N NaOH was added and the reaction was stirred overnight at 70° C. An additional 0.1 mL of 5N NaOH was added and the reaction was stirred over the weekend at 70° C. The crude material was purified via preparative LC/MS to afford (2S)-2-[5-(2-benzyl-1,2,3,4-tetrahydroisoquinolin-6-yl)-4-(4,4-dimethylpiperidin-1-yl)-2,6-dimethylpyridin-3-yl]-2-(tert-butoxy)acetic acid (29.2 mg). LCMS (M+H)=570.28. ¹H NMR (500 MHz, DMSO-d6) δ 7.40-7.34 (m, 4H), 7.32-7.27 (m, 1H), 7.14-7.09 (m, 1H), 7.07-7.03 (m, 1H), 6.90-6.86 (m, 1H), 5.85 (br d, J=10.6 Hz, 1H), 3.76-3.60 (m, 3H), 2.85 (br d, J=5.1 Hz, 3H), 2.72 (br d, J=17.6 Hz, 2H), 2.56 (s, 4H), 2.44 (s, 3H), 2.14-2.04 (m, 4H), 1.92 (s, 2H), 1.13 (s, 10H), 0.86 (s, 3H), 0.63 (br s, 3H).

### Example 53

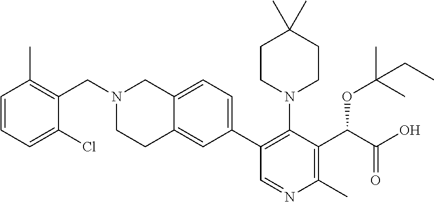 [SMILES: CCC(C)(C)O[C@H](C(=O)O)c1c(C)ncc(-c2ccc3c(c2)CCN(Cc2c(C)cccc2Cl)C3)c1N1CCC(C)(C)CC1]

### (2S)-2-(5-{2-[(2-Chloro-6-methylphenyl)methyl]-1,2,3,4-tetrahydroisoquinolin-6-yl}-4-(4,4-dimethylpiperidin-1-yl)-2-methylpyridin-3-yl)-2-[(2-methylbutan-2-yl)oxy]acetic Acid

(S)-Isopropyl 2-(5-bromo-4-(4,4-dimethylpiperidin-1-yl)-2-methylpyridin-3-yl)-2-(tert-pentyloxy)acetate (45 mg, 0.096 mmol), 2-(2-chloro-6-methylbenzyl)-6-(4,4,5,5-tetramethyl-1,3,2-dioxaborolan-2-yl)-1,2,3,4-tetrahydroisoquinoline (42 mg, 0.11 mmol), (2-dicyclohexylphosphino-2′,6′-dimethoxybiphenyl) [2-(2′-amino-1,1′-biphenyl)]palladium(II) methanesulfonate (7 mg, 10 μmol), potassium phosphate tribasic (61 mg, 0.29 mmol) were combined under N₂ (g). 1,4-Dioxane (1.6 ml) and water (0.32 ml) was added under N₂ (g). The reaction was stirred at 80° C. for 1 hr. The reaction was concentrated, adsorbed onto celite and was purified on silica gel (Biotage, EtOAc/hexanes gradient, 0-100% over 10 CVs). The major product was isolated and taken up in EtOH and 0.1 mL of 5 N NaOH was added and the reaction was stirred at 100° C. for 5 hrs. The crude material was purified via preparative LC/MS to afford (2S)-2-(5-{2-[(2-chloro-6-methylphenyl)methyl]-1,2,3,4-tetrahydroisoquinolin-6-yl}-4-(4,4-dimethylpiperidin-1-yl)-2-methylpyridin-3-yl)-2-[(2-methylbutan-2-yl)oxy]acetic acid (10.0 mg). LCMS (M+H)=618.3. ¹H NMR (500 MHz, DMSO-d₆) δ 8.01 (s, 1H), 7.30 (d, J=7.6 Hz, 1H), 7.24-7.17 (m, 2H), 7.12 (d, J=7.5 Hz, 1H), 7.06-7.01 (m, 2H), 5.80 (s, 1H), 3.84 (s, 2H), 3.70 (s, 2H), 2.81 (br dd, J=14.7, 4.8 Hz, 4H), 2.55-2.51 (m, 2H), 2.49-2.42 (m, 3H), 1.92 (s, 1H), 1.57-1.35 (m, 3H), 1.31 (br s, 2H), 1.26 (br s, 1H), 1.11 (s, 4H), 1.04 (s, 4H), 0.81 (br s, 6H), 0.71 (t, J=7.3 Hz, 5H). Not all of the piperidine protons were well resolved.

### Example 54

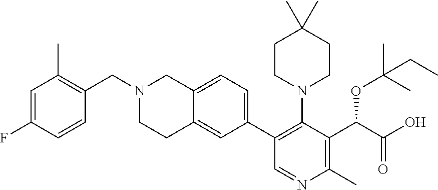 [SMILES: CCC(C)(C)O[C@H](C(=O)O)c1c(C)ncc(-c2ccc3c(c2)CCN(Cc2ccc(F)cc2C)C3)c1N1CCC(C)(C)CC1]

### (2S)-2-[4-(4,4-Dimethylpiperidin-1-yl)-5-{2-[(4-fluoro-2-methylphenyl)methyl]-1,2,3,4-tetrahydroisoquinolin-6-yl}-2-methylpyridin-3-yl]-2-[(2-methylbutan-2-yl)oxy]acetic Acid

(S)-Isopropyl 2-(5-bromo-4-(4,4-dimethylpiperidin-1-yl)-2-methylpyridin-3-yl)-2-(tert-pentyloxy)acetate (25 mg, 0.053 mmol), 2-(4-fluoro-2-methylbenzyl)-6-(4,4,5,5-tetramethyl-1,3,2-dioxaborolan-2-yl)-1,2,3,4-tetrahydroisoquinoline (22 mg, 0.059 mmol), (2-dicyclohexylphosphino-2′,6′-dimethoxybiphenyl) [2-(2′-amino-1,1′-biphenyl)]palladium(II) methanesulfonate (4 mg, 5 μmol), potassium phosphate tribasic (334 mg, 0.16 mmol) were combined under N₂. 1,4-Dioxane (1 ml) and water (0.2 ml) was added under N₂. The reaction was stirred at 80° C. for 1 hr. The reaction was concentrated, adsorbed onto celite and was purified on silica gel (Biotage, EtOAc/hexanes gradient, 0-100% over 10 CVs) to give the expected ester. This material was taken up in EtOH (1 mL) and 0.1 mL of 5 N NaOH was added and the reaction was stirred at 100° C. for 4 hours. The crude material was purified via preparative LC/MS to afford (2S)-2-[4-(4,4-dimethylpiperidin-1-yl)-5-{2-[(4-fluoro-2-methylphenyl)methyl]-1,2,3,4-tetrahydroisoquinolin-6-yl}-2-methylpyridin-3-yl]-2-[(2-methylbutan-2-yl)oxy]acetic acid (4.6 mg). LCMS (M+H)=602.26. ¹H NMR (500 MHz, DMSO-d₆) δ 8.00 (s, 1H), 7.43-7.29 (m, 1H), 7.26 (br s, 1H), 7.18-7.07 (m, 1H), 7.07-6.93 (m, 3H), 5.78 (br s, 1H), 3.62 (s, 2H), 2.85 (br s, 1H), 2.73 (br t, J=5.7 Hz, 1H), 2.49-2.35 (m, 3H), 1.91 (s, 7H), 1.77 (s, 1H), 1.56-1.33 (m, 3H), 1.31 (br s, 2H), 1.25 (br s, 1H), 1.10 (s, 3H), 1.08-0.95 (m, 4H), 0.93-0.77 (m, 6H), 0.77-0.58 (m, 5H).

### Example 55

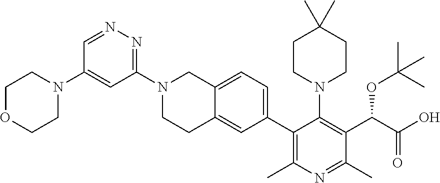 [SMILES: Cc1nc(C)c([C@H](OC(C)(C)C)C(=O)O)c(N2CCC(C)(C)CC2)c1-c1ccc2c(c1)CCN(c1cc(N3CCOCC3)cnn1)C2]

### (S)-2-(tert-Butoxy)-2-(4-(4,4-dimethylpiperidin-1-yl)-2,6-dimethyl-5-(2-(5-morpholinopyridazin-3-yl)-1,2,3,4-tetrahydroisoquinolin-6-yl)pyridin-3-yl)acetic Acid

To a reaction vial under nitrogen containing (S)-isopropyl 2-(tert-butoxy)-2-(4-(4,4-dimethylpiperidin-1-yl)-2,6-dimethyl-5-(4,4,5,5-tetramethyl-1,3,2-dioxaborolan-2-yl)pyridin-3-yl)acetate (25.2 mg, 0.049 mmol) was added 4-(6-(6-bromo-3,4-dihydroisoquinolin-2(1H)-yl)pyridazin-4-yl)morpholine (35 mg, 0.093 mmol) and THF (2.0 mL). The reaction was flushed with argon, treated with 0.5 M potassium phosphate tribasic (255 μL, 0.128 mmol) and 2nd Generation X-Phos precatalyst (6.5 mg, 8.26 μmol), securely capped and heated at 100° C. for 18 h. The solvent was removed under a stream of nitrogen and the residue was redissolved in ethanol (2.0 mL). The resulting solution was treated with 10 M sodium hydroxide (40 μL, 0.400 mmol), flushed briefly with nitrogen and heated at 100° C. for 18 h. The crude material was purified via preparative LC/MS to afford (S)-2-(tert-butoxy)-2-(4-(4,4-dimethylpiperidin-1-yl)-2,6-dimethyl-5-(2-(5-morpholinopyridazin-3-yl)-1,2,3,4-tetrahydroisoquinolin-6-yl)pyridin-3-yl)acetic acid, 3.5 mg (11%). LCMS (M+1)=643.2.

### Example 56

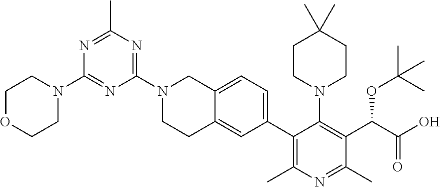 [SMILES: Cc1nc(N2CCOCC2)nc(N2CCc3cc(-c4c(C)nc(C)c([C@H](OC(C)(C)C)C(=O)O)c4N4CCC(C)(C)CC4)ccc3C2)n1]

### (S)-2-(tert-Butoxy)-2-(4-(4,4-dimethylpiperidin-1-yl)-2,6-dimethyl-5-(2-(4-methyl-6-morpholino-1,3,5-triazin-2-yl)-1,2,3,4-tetrahydroisoquinolin-6-yl)pyridin-3-yl)acetic Acid

To a reaction vial under nitrogen containing (S)-isopropyl 2-(tert-butoxy)-2-(4-(4,4-dimethylpiperidin-1-yl)-2,6-dimethyl-5-(4,4,5,5-tetramethyl-1,3,2-dioxaborolan-2-yl)pyridin-3-yl)acetate (20.2 mg, 0.039 mmol) was added 4-(4-(6-bromo-3,4-dihydroisoquinolin-2(1H)-yl)-6-methyl-1,3,5-triazin-2-yl)morpholine (29 mg, 0.074 mmol) and THF (2.0 mL). The reaction was flushed with argon for 5 min, treated with 0.5 M potassium phosphate tribasic (202 μL, 0.101 mmol), followed 2nd Generation X-Phos precatalyst (7 mg, 8.90 μmol), capped and heated at 100° C. for 18 h. The solvent was removed under a gentle stream of nitrogen and the residue was dissolved in ethanol (2.0 mL) and treated with sodium hydroxide (35 μL, 0.350 mmol). The reaction was flushed briefly with nitrogen, capped and heated to 100° C. for 18 h. The crude material was purified via preparative LC/MS to afford (S)-2-(tert-butoxy)-2-(4-(4,4-dimethylpiperidin-1-yl)-2,6-dimethyl-5-(2-(4-methyl-6-morpholino-1,3,5-triazin-2-yl)-1,2,3,4-tetrahydroisoquinolin-6-yl)pyridin-3-yl)acetic acid, 6.3 mg (25%). LCMS (M+1)=658.2.

### Example 57

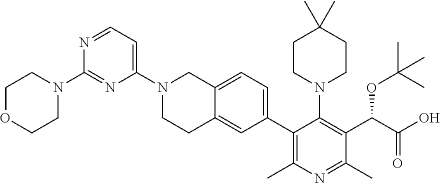 [SMILES: Cc1nc(C)c([C@H](OC(C)(C)C)C(=O)O)c(N2CCC(C)(C)CC2)c1-c1ccc2c(c1)CCN(c1ccnc(N3CCOCC3)n1)C2]

### (S)-2-(tert-Butoxy)-2-(4-(4,4-dimethylpiperidin-1-yl)-2,6-dimethyl-5-(2-(2-morpholinopyrimidin-4-yl)-1,2,3,4-tetrahydroisoquinolin-6-yl)pyridin-3-yl)acetic Acid

To a reaction vial under nitrogen was added (S)-isopropyl 2-(tert-butoxy)-2-(4-(4,4-dimethylpiperidin-1-yl)-2,6-dimethyl-5-(4,4,5,5-tetramethyl-1,3,2-dioxaborolan-2-yl)pyridin-3-yl)acetate (20.0 mg, 0.039 mmol), 4-(4-(6-bromo-3,4-dihydroisoquinolin-2(1H)-yl)pyrimidin-2-yl)morpholine (27 mg, 0.072 mmol) and THF (2.0 mL). The reaction flushed with argon, then treated with 0.5 M potassium phosphate tribasic (200 μL, 0.100 mmol), followed by 2nd Generation X-Phos precatalyst (6.7 mg, 8.52 μmol). The reaction was flushed again with argon, capped and heated at 100° C. for 18 h. The solvent was removed under gentle stream of nitrogen and the residue was dissolved in EtOH (2 mL) and treated with 10 M sodium hydroxide (35 μL, 0.350 mmol). The reaction was flushed briefly with nitrogen, capped and heated at 80° C. for 18 h. The reaction was treated with additional 10 M sodium hydroxide (18 μlit, 0.180 mmol) and heated to 100° C. for 3h. The crude material was purified via preparative LC/MS to afford (S)-2-(tert-butoxy)-2-(4-(4,4-dimethylpiperidin-1-yl)-2,6-dimethyl-5-(2-(2-morpholinopyrimidin-4-yl)-1,2,3,4-tetrahydroisoquinolin-6-yl)pyridin-3-yl)acetic acid, 6.1 mg (24%). LCMS (M+1)=643.2.

### Example 58

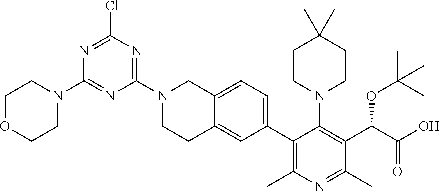 [SMILES: Cc1nc(C)c([C@H](OC(C)(C)C)C(=O)O)c(N2CCC(C)(C)CC2)c1-c1ccc2c(c1)CCN(c1nc(Cl)nc(N3CCOCC3)n1)C2]

### (S)-2-(tert-Butoxy)-2-(5-(2-(4-chloro-6-morpholino-1,3,5-triazin-2-yl)-1,2,3,4-tetrahydroisoquinolin-6-yl)-4-(4,4-dimethylpiperidin-1-yl)-2,6-dimethylpyridin-3-yl)acetic Acid

To a small pressure vial under nitrogen was added (S)-isopropyl 2-(tert-butoxy)-2-(4-(4,4-dimethylpiperidin-1-yl)-2,6-dimethyl-5-(4,4,5,5-tetramethyl-1,3,2-dioxaborolan-2-yl)pyridin-3-yl)acetate (25 mg, 0.048 mmol), 4-(4-(6-bromo-3,4-dihydroisoquinolin-2(1H)-yl)-6-chloro-1,3,5-triazin-2-yl)morpholine (27 mg, 0.066 mmol) and THF (1.2 mL). The reaction was flushed with argon, then treated with 0.5 M potassium phosphate tribasic (245 μL, 0.123 mmol), followed by 2nd Generation X-phos precatalyst (8 mg, 10.17 μmol). The reaction was again flushed with argon, capped and heated at 100° C. for 18 h. The solvent was removed under gentle stream of nitrogen and the residue was dissolved in EtOH (2 mL) and treated with 10 M sodium hydroxide (40 μL, 0.400 mmol). The reaction was flushed briefly with nitrogen, capped and heated at 100° C. for 18 h. The crude material was purified via preparative LC/MS to afford (S)-2-(tert-butoxy)-2-(5-(2-(4-chloro-6-morpholino-1,3,5-triazin-2-yl)-1,2,3,4-tetrahydroisoquinolin-6-yl)-4-(4,4-dimethylpiperidin-1-yl)-2,6-dimethylpyridin-3-yl)acetic acid, 2.2 mg (7%). LCMS (M+1)=660.2.

### Example 59

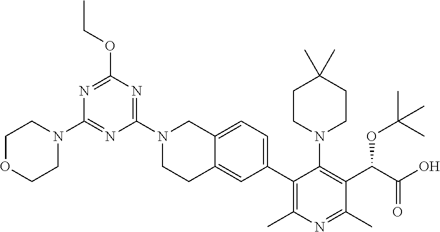 [SMILES: CCOc1nc(N2CCOCC2)nc(N2CCc3cc(-c4c(C)nc(C)c([C@H](OC(C)(C)C)C(=O)O)c4N4CCC(C)(C)CC4)ccc3C2)n1]

### (S)-2-(tert-Butoxy)-2-(4-(4,4-dimethylpiperidin-1-yl)-5-(2-(4-ethoxy-6-morpholino-1,3,5-triazin-2-yl)-1,2,3,4-tetrahydroisoquinolin-6-yl)-2,6-dimethylpyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (22.2 mg, 0.043 mmol), 2,4-dichloro-6-methoxy-1,3,5-triazine (15.5 mg, 0.086 mmol) and EtOH (2.0 mL). The reaction was flushed briefly with nitrogen, treated with N,N-diisopropylethylamine (25 μL, 0.143 mmol), capped and placed in a microwave heating unit at 80° C. for 1.5 h. The reaction was then treated with morpholine (40 μL, 0.459 mmol) and placed in a microwave heating unit at 100° C. for 1.5 h. The reaction was then treated with 10 M sodium hydroxide (35 μL, 0.350 mmol) heated at 100° C. for 4 h. Additional 10 M NaOH (18 μlit, 0.180 mmol) was added and the reaction was at 100° C. for 18 h. The crude material was purified via preparative LC/MS to afford (S)-2-(tert-butoxy)-2-(4-(4,4-dimethylpiperidin-1-yl)-5-(2-(4-ethoxy-6-morpholino-1,3,5-triazin-2-yl)-1,2,3,4-tetrahydroisoquinolin-6-yl)-2,6-dimethylpyridin-3-yl)acetic acid, 10.8 mg (37%). LCMS (M+1)=688.2.

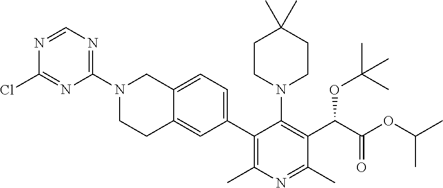 [SMILES: Cc1nc(C)c([C@H](OC(C)(C)C)C(=O)OC(C)C)c(N2CCC(C)(C)CC2)c1-c1ccc2c(c1)CCN(c1ncnc(Cl)n1)C2]

### Isopropyl (S)-2-(tert-butoxy)-2-(5-(2-(4-chloro-1,3,5-triazin-2-yl)-1,2,3,4-tetrahydroisoquinolin-6-yl)-4-(4,4-dimethylpiperidin-1-yl)-2,6-dimethylpyridin-3-yl)acetate

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (76 mg, 0.146 mmol), 2,4-dichloro-1,3,5-triazine (55 mg, 0.367 mmol) and EtOH (4 mL). The reaction was flushed briefly with nitrogen, treated with N,N-diisopropylethylamine (102 μL, 0.584 mmol), capped and placed in a microwave heating unit at 100° C. for 1 h to afford isopropyl (S)-2-(tert-butoxy)-2-(5-(2-(4-chloro-1,3,5-triazin-2-yl)-1,2,3,4-tetrahydroisoquinolin-6-yl)-4-(4,4-dimethylpiperidin-1-yl)-2,6-dimethylpyridin-3-yl)acetate, 93 mg (quant) that was used “as is” without purification in subsequent examples. LCMS (M+1)=635.4, 637.4.

### Example 60

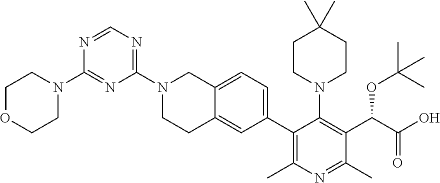 [SMILES: Cc1nc(C)c([C@H](OC(C)(C)C)C(=O)O)c(N2CCC(C)(C)CC2)c1-c1ccc2c(c1)CCN(c1ncnc(N3CCOCC3)n1)C2]

### (S)-2-(tert-Butoxy)-2-(4-(4,4-dimethylpiperidin-1-yl)-2,6-dimethyl-5-(2-(4-morpholino-1,3,5-triazin-2-yl)-1,2,3,4-tetrahydroisoquinolin-6-yl)pyridin-3-yl)acetic Acid

To a microwave vial was added a solution of (S)-isopropyl 2-(tert-butoxy)-2-(5-(2-(4-chloro-1,3,5-triazin-2-yl)-1,2,3,4-tetrahydroisoquinolin-6-yl)-4-(4,4-dimethylpiperidin-1-yl)-2,6-dimethylpyridin-3-yl)acetate (23.19 mg, 0.0365 mmol) in EtOH (1.0 mL), followed by morpholine (75 μL, 0.861 mmol). The vial was capped and heated in a microwave unit for 3.5 h at 135° C. The reaction was then treated with 10 M sodium hydroxide (75 μL, 0.750 mmol) and heated at 100° C. for 7 h. The crude material was purified via preparative LC/MS to afford. (S)-2-(tert-butoxy)-2-(4-(4,4-dimethylpiperidin-1-yl)-2,6-dimethyl-5-(2-(4-morpholino-1,3,5-triazin-2-yl)-1,2,3,4-tetrahydroisoquinolin-6-yl)pyridin-3-yl)acetic acid, 8.3 mg (35%). LCMS (M+1)=644.2.

### Example 61

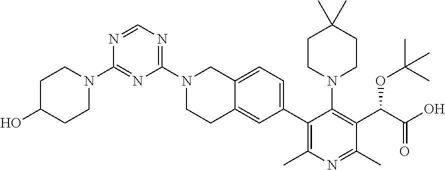 [SMILES: Cc1nc(C)c([C@H](OC(C)(C)C)C(=O)O)c(N2CCC(C)(C)CC2)c1-c1ccc2c(c1)CCN(c1ncnc(N3CCC(O)CC3)n1)C2]

### (S)-2-(tert-Butoxy)-2-(4-(4,4-dimethylpiperidin-1-yl)-5-(2-(4-(4-hydroxypiperidin-1-yl)-1,3,5-triazin-2-yl)-1,2,3,4-tetrahydroisoquinolin-6-yl)-2,6-dimethylpyridin-3-yl)acetic Acid

To a microwave vial was added a solution of (S)-isopropyl 2-(tert-butoxy)-2-(5-(2-(4-chloro-1,3,5-triazin-2-yl)-1,2,3,4-tetrahydroisoquinolin-6-yl)-4-(4,4-dimethylpiperidin-1-yl)-2,6-dimethylpyridin-3-yl)acetate (23.19 mg, 0.0365 mmol) in EtOH (1.0 mL), followed by 4-hydroxypiperidine (37 mg, 0.366 mmol) and N,N-diisopropylethylamine (35 μL, 0.200 mmol). The vial was capped and heated in a microwave unit for 3.5 h at 135° C. The reaction was then treated with 10 M sodium hydroxide (75 μL, 0.750 mmol) and heated at 100° C. for 7 h. The crude material was purified via preparative LC/MS to afford (S)-2-(tert-butoxy)-2-(4-(4,4-dimethylpiperidin-1-yl)-5-(2-(4-(4-hydroxypiperidin-1-yl)-1,3,5-triazin-2-yl)-1,2,3,4-tetrahydroisoquinolin-6-yl)-2,6-dimethylpyridin-3-yl)acetic acid, 10.5 mg (44%). LCMS (M+1)=658.3.

### Example 62

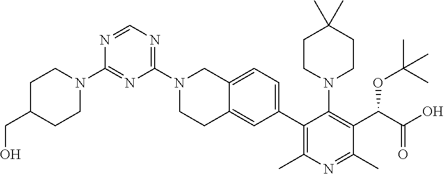 [SMILES: Cc1nc(C)c([C@H](OC(C)(C)C)C(=O)O)c(N2CCC(C)(C)CC2)c1-c1ccc2c(c1)CCN(c1ncnc(N3CCC(CO)CC3)n1)C2]

### (S)-2-(tert-Butoxy)-2-(4-(4,4-dimethylpiperidin-1-yl)-5-(2-(4-(4-(hydroxymethyl)piperidin-1-yl)-1,3,5-triazin-2-yl)-1,2,3,4-tetrahydroisoquinolin-6-yl)-2,6-dimethylpyridin-3-yl)acetic Acid

To a microwave vial was added a solution of (S)-isopropyl 2-(tert-butoxy)-2-(5-(2-(4-chloro-1,3,5-triazin-2-yl)-1,2,3,4-tetrahydroisoquinolin-6-yl)-4-(4,4-dimethylpiperidin-1-yl)-2,6-dimethylpyridin-3-yl)acetate (23.19 mg, 0.0365 mmol) in EtOH (1.0 mL), followed by 4-piperidinemethanol (42 mg, 0.365 mmol) and N,N-diisopropylethylamine (35 μL, 0.200 mmol). The vial was capped and heated in a microwave unit for 3.5 h at 135° C. The reaction was then treated with 10 M sodium hydroxide (75 μL, 0.750 mmol) and heated at 100° C. for 7 h. The crude material was purified via preparative LC/MS to afford (S)-2-(tert-butoxy)-2-(4-(4,4-dimethylpiperidin-1-yl)-5-(2-(4-(4-(hydroxymethyl)piperidin-1-yl)-1,3,5-triazin-2-yl)-1,2,3,4-tetrahydroisoquinolin-6-yl)-2,6-dimethylpyridin-3-yl)acetic acid, 7.8 mg (32%). LCMS (M+1)=670.2.

### Example 63

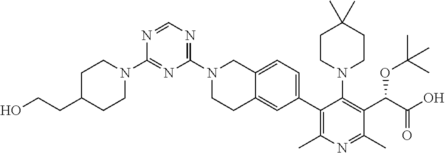 [SMILES: Cc1nc(C)c([C@H](OC(C)(C)C)C(=O)O)c(N2CCC(C)(C)CC2)c1-c1ccc2c(c1)CCN(c1ncnc(N3CCC(CCO)CC3)n1)C2]

### (S)-2-(tert-Butoxy)-2-(4-(4,4-dimethylpiperidin-1-yl)-5-(2-(4-(4-(2-hydroxyethyl)piperidin-1-yl)-1,3,5-triazin-2-yl)-1,2,3,4-tetrahydroisoquinolin-6-yl)-2,6-dimethylpyridin-3-yl)acetic Acid

To a microwave vial was added a solution of (S)-isopropyl 2-(tert-butoxy)-2-(5-(2-(4-chloro-1,3,5-triazin-2-yl)-1,2,3,4-tetrahydroisoquinolin-6-yl)-4-(4,4-dimethylpiperidin-1-yl)-2,6-dimethylpyridin-3-yl)acetate (23.19 mg, 0.0365 mmol) in EtOH (1.0 mL), followed by 4-piperidineethanol (47 mg, 0.364 mmol) and N,N-diisopropylethylamine (35 μL, 0.200 mmol). The vial was capped and heated in a microwave unit for 3.5 h at 135° C. The reaction was then treated with 10 M sodium hydroxide (75 μL, 0.750 mmol) and heated at 100° C. for 5 h. The crude material was purified via preparative LC/MS to afford (S)-2-(tert-butoxy)-2-(4-(4,4-dimethylpiperidin-1-yl)-5-(2-(4-(4-(2-hydroxyethyl)piperidin-1-yl)-1,3,5-triazin-2-yl)-1,2,3,4-tetrahydroisoquinolin-6-yl)-2,6-dimethylpyridin-3-yl)acetic acid, 13.6 mg (54%) LCMS (M+1)=686.2.

### Example 64

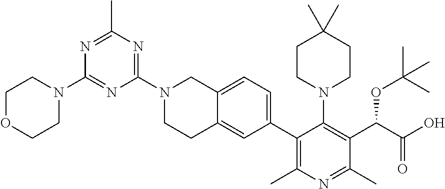 [SMILES: Cc1nc(N2CCOCC2)nc(N2CCc3cc(-c4c(C)nc(C)c([C@H](OC(C)(C)C)C(=O)O)c4N4CCC(C)(C)CC4)ccc3C2)n1]

### (S)-2-(tert-Butoxy)-2-(4-(4,4-dimethylpiperidin-1-yl)-2,6-dimethyl-5-(2-(2-methyl-6-morpholinopyrimidin-4-yl)-1,2,3,4-tetrahydroisoquinolin-6-yl)pyridin-3-yl)acetic Acid

To a microwave vial was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (15 mg, 0.029 mmol), 4,6-dichloro-2-methylpyrimidine (12 mg, 0.074 mmol), EtOH (1.5 mL) and N,N-diisopropylethylamine (22 μL, 0.126 mmol). The reaction was capped and heated in a microwave heating unit for 45 min at 100° C. The reaction was then treated morpholine (50 μL, 0.574 mmol) heated in a microwave heating unit for 4h at 150° C. The reaction was then treated with 10 M sodium hydroxide (45 μL, 0.450 mmol) and placed in a 100° C. sand bath for 5 h. The crude material was purified via preparative LC/MS to afford (S)-2-(tert-butoxy)-2-(4-(4,4-dimethylpiperidin-1-yl)-2,6-dimethyl-5-(2-(2-methyl-6-morpholinopyrimidin-4-yl)-1,2,3,4-tetrahydroisoquinolin-6-yl)pyridin-3-yl)acetic acid, 8.3 mg (44%). LCMS (M+1)=657.2.

Also isolated from this reaction was

### Example 65

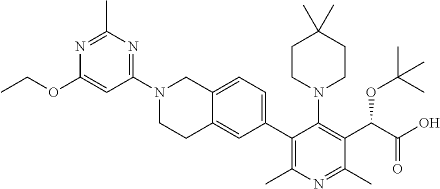 [SMILES: CCOc1cc(N2CCc3cc(-c4c(C)nc(C)c([C@H](OC(C)(C)C)C(=O)O)c4N4CCC(C)(C)CC4)ccc3C2)nc(C)n1]

### (S)-2-(tert-Butoxy)-2-(4-(4,4-dimethylpiperidin-1-yl)-5-(2-(6-ethoxy-2-methylpyrimidin-4-yl)-1,2,3,4-tetrahydroisoquinolin-6-yl)-2,6-dimethylpyridin-3-yl)acetic Acid

4.3 mg (24%). LCMS (M+1)=616.2.

### Example 66

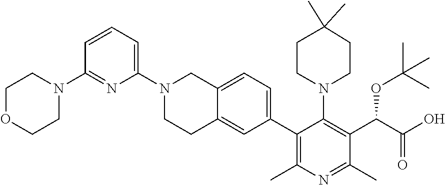 [SMILES: Cc1nc(C)c([C@H](OC(C)(C)C)C(=O)O)c(N2CCC(C)(C)CC2)c1-c1ccc2c(c1)CCN(c1cccc(N3CCOCC3)n1)C2]

### (S)-2-(tert-Butoxy)-2-(4-(4,4-dimethylpiperidin-1-yl)-2,6-dimethyl-5-(2-(6-morpholinopyridin-2-yl)-1,2,3,4-tetrahydroisoquinolin-6-yl)pyridin-3-yl)acetic Acid

To a reaction vial containing (S)-isopropyl 2-(tert-butoxy)-2-(4-(4,4-dimethylpiperidin-1-yl)-2,6-dimethyl-5-(4,4,5,5-tetramethyl-1,3,2-dioxaborolan-2-yl)pyridin-3-yl)acetate (29.3 mg, 0.057 mmol) was added 4-(6-(6-bromo-3,4-dihydroisoquinolin-2(1H)-yl)pyridin-2-yl)morpholine (40 mg, 0.107 mmol) and THF. The reaction was flushed with argon, then treated with 0.5 M potassium phosphate tribasic (290 μL, 0.145 mmol), followed 2^{nd} Generation X-Phos Precatalyst (8 mg, 10.17 μmol). The reaction was flushed with argon again, capped and heated at 100° C. for 18 h. The solvent was removed under gentle stream of nitrogen and the residue was dissolved in EtOH (2 mL) and treated with 10 M sodium hydroxide (50 μL, 0.500 mmol). The reaction was flushed briefly with nitrogen, capped and heated at 80° C. for 5.5 h. The reaction was treated with additional 10 M sodium hydroxide (25 μL, 0.250 mmol) and heated to 100° C. for 18 h. The crude material was purified via preparative LC/MS to afford (S)-2-(tert-butoxy)-2-(4-(4,4-dimethylpiperidin-1-yl)-2,6-dimethyl-5-(2-(6-morpholinopyridin-2-yl)-1,2,3,4-tetrahydroisoquinolin-6-yl)pyridin-3-yl)acetic acid, 6.9 mg (19%). LCMS (M+1)=642.3.

### Example 67

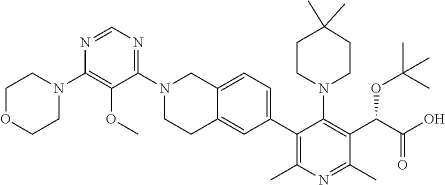 [SMILES: COc1c(N2CCOCC2)ncnc1N1CCc2cc(-c3c(C)nc(C)c([C@H](OC(C)(C)C)C(=O)O)c3N3CCC(C)(C)CC3)ccc2C1]

### (S)-2-(tert-Butoxy)-2-(4-(4,4-dimethylpiperidin-1-yl)-5-(2-(5-methoxy-6-morpholinopyrimidin-4-yl)-1,2,3,4-tetrahydroisoquinolin-6-yl)-2,6-dimethylpyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (21.8 mg, 0.042 mmol), 4,6-dichloro-5-methoxypyrimidine (15 mg, 0.084 mmol), and EtOH (1.5 mL). The reaction was flushed briefly with nitrogen, treated with N,N-diisopropylethylamine (25 μL, 0.143 mmol), capped and placed in a microwave heating unit at 80° C. for 90 min. The reaction was then treated with morpholine (65 μL, 0.746 mmol) and placed in a microwave heating unit at 120° C. for 5 h. Additional morpholine (40 μlit, 0.459 mmol) was added and the reaction was placed in a microwave heating unit at 150° C. for 5 h. The reaction was then treated with 10 M sodium hydroxide (45 μL, 0.450 mmol) and heated at 100° C. for 18 h. The crude material was purified via preparative LC/MS to afford (S)-2-(tert-butoxy)-2-(4-(4,4-dimethylpiperidin-1-yl)-5-(2-(5-methoxy-6-morpholinopyrimidin-4-yl)-1,2,3,4-tetrahydroisoquinolin-6-yl)-2,6-dimethylpyridin-3-yl)acetic acid, 10.2 mg (36%). LCMS (M+1)=673.2.

### Example 68

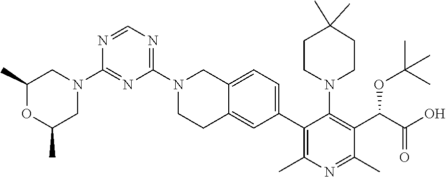 [SMILES: Cc1nc(C)c([C@H](OC(C)(C)C)C(=O)O)c(N2CCC(C)(C)CC2)c1-c1ccc2c(c1)CCN(c1ncnc(N3C[C@@H](C)O[C@@H](C)C3)n1)C2]

### (S)-2-(tert-Butoxy)-2-(5-(2-(4-((2R,6S)-2,6-dimethylmorpholino)-1,3,5-triazin-2-yl)-1,2,3,4-tetrahydroisoquinolin-6-yl)-4-(4,4-dimethylpiperidin-1-yl)-2,6-dimethylpyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (19 mg, 0.036 mmol), 2,4-dichloro-1,3,5-triazine (14 mg, 0.093 mmol) and EtOH (1.6 mL). The reaction was flushed briefly with nitrogen, treated with N,N-diisopropylethylamine (25 μL, 0.143 mmol), capped and placed in a microwave heating unit at 100° C. for 1 h. The reaction was then treated with cis-2,6-dimethylmorpholine (90 μL, 0.727 mmol) and placed in a microwave heating unit at 135° C. for 5 h. The reaction was then treated with 10 M sodium hydroxide (75 μL, 0.750 mmol) and heated at 100° C. for 7 h. The crude material was purified via preparative LC/MS to afford (S)-2-(tert-butoxy)-2-(5-(2-(4-((2R,6S)-2,6-dimethylmorpholino)-1,3,5-triazin-2-yl)-1,2,3,4-tetrahydroisoquinolin-6-yl)-4-(4,4-dimethylpiperidin-1-yl)-2,6-dimethylpyridin-3-yl)acetic acid, 6.8 mg (27%). LCMS (M+1)=672.3.

### Example 69

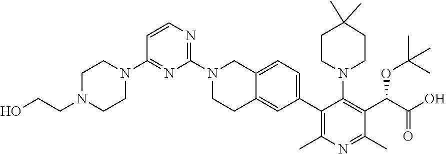 [SMILES: Cc1nc(C)c([C@H](OC(C)(C)C)C(=O)O)c(N2CCC(C)(C)CC2)c1-c1ccc2c(c1)CCN(c1nccc(N3CCN(CCO)CC3)n1)C2]

### (S)-2-(tert-Butoxy)-2-(4-(4,4-dimethylpiperidin-1-yl)-5-(2-(4-(4-(2-hydroxyethyl)piperazin-1-yl)pyrimidin-2-yl)-1,2,3,4-tetrahydroisoquinolin-6-yl)-2,6-dimethylpyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20 mg, 0.038 mmol), 2-(4-(2-chloropyrimidin-4-yl)piperazin-1-yl)ethanol (35 mg, 0.144 mmol), EtOH (1.5 mL) and N,N-diisopropylethylamine (70 μL, 0.401 mmol). The reaction was flushed briefly with nitrogen, capped and heated in a microwave reactor at 130-135° C. for 9 h. The reaction was treated with 10 M sodium hydroxide (80 μL, 0.800 mmol) and heated at 100° C. for 4 h. The crude material was purified via preparative LC/MS to afford (S)-2-(tert-butoxy)-2-(4-(4,4-dimethylpiperidin-1-yl)-5-(2-(4-(4-(2-hydroxyethyl)piperazin-1-yl)pyrimidin-2-yl)-1,2,3,4-tetrahydroisoquinolin-6-yl)-2,6-dimethylpyridin-3-yl)acetic acid, 16.5 mg (60%). LCMS (M+1)=686.2.

### Example 70

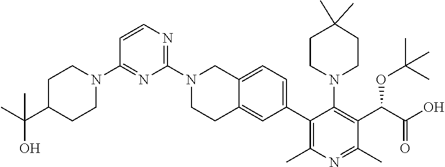 [SMILES: Cc1nc(C)c([C@H](OC(C)(C)C)C(=O)O)c(N2CCC(C)(C)CC2)c1-c1ccc2c(c1)CCN(c1nccc(N3CCC(C(C)(C)O)CC3)n1)C2]

### (S)-2-(tert-Butoxy)-2-(4-(4,4-dimethylpiperidin-1-yl)-5-(2-(4-(4-(2-hydroxypropan-2-yl)piperidin-1-yl)pyrimidin-2-yl)-1,2,3,4-tetrahydroisoquinolin-6-yl)-2,6-dimethylpyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20 mg, 0.038 mmol), 2-(1-(2-chloropyrimidin-4-yl)piperidin-4-yl)propan-2-ol (35 mg, 0.137 mmol), ethanol (1.5 mL) and N,N-diisopropylethylamine (70 μL, 0.401 mmol). The reaction was capped and placed in a microwave reactor for at 135° C. for 10 h. The reaction was treated with 10 M sodium hydroxide (80 μL, 0.800 mmol) and placed in 100° C. sand bath for 5 h. The crude material was purified via preparative LC/MS to afford (S)-2-(tert-butoxy)-2-(4-(4,4-dimethylpiperidin-1-yl)-5-(2-(4-(4-(2-hydroxypropan-2-yl)piperidin-1-yl)pyrimidin-2-yl)-1,2,3,4-tetrahydroisoquinolin-6-yl)-2,6-dimethylpyridin-3-yl)acetic acid, 12.1 mg (45%). LCMS (M+1)=699.2.

### Example 71

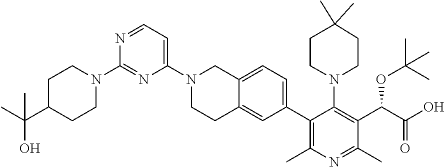 [SMILES: Cc1nc(C)c([C@H](OC(C)(C)C)C(=O)O)c(N2CCC(C)(C)CC2)c1-c1ccc2c(c1)CCN(c1ccnc(N3CCC(C(C)(C)O)CC3)n1)C2]

### (S)-2-(tert-Butoxy)-2-(4-(4,4-dimethylpiperidin-1-yl)-5-(2-(2-(4-(2-hydroxypropan-2-yl)piperidin-1-yl)pyrimidin-4-yl)-1,2,3,4-tetrahydroisoquinolin-6-yl)-2,6-dimethylpyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20 mg, 0.038 mmol), 2-(1-(4-chloropyrimidin-2-yl)piperidin-4-yl)propan-2-ol (35 mg, 0.137 mmol), ethanol (1.5 mL) and N,N-diisopropylethylamine (70 μL, 0.401 mmol). The reaction was capped and placed in a microwave reactor at 140° C. for 10h. The reaction was treated with 10 M sodium hydroxide (75 μL, 0.750 mmol) and placed in a 100° C. sand bath for 3.5 h. The crude material was purified via preparative LC/MS to afford (S)-2-(tert-butoxy)-2-(4-(4,4-dimethylpiperidin-1-yl)-5-(2-(2-(4-(2-hydroxypropan-2-yl)piperidin-1-yl)pyrimidin-4-yl)-1,2,3,4-tetrahydroisoquinolin-6-yl)-2,6-dimethylpyridin-3-yl)acetic acid, 6.6 mg (25%). LCMS (M+1)=699.2.

### Example 72

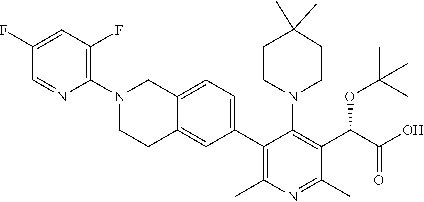 [SMILES: Cc1nc(C)c([C@H](OC(C)(C)C)C(=O)O)c(N2CCC(C)(C)CC2)c1-c1ccc2c(c1)CCN(c1ncc(F)cc1F)C2]

### (S)-2-(tert-Butoxy)-2-(5-(2-(3,5-difluoropyridin-2-yl)-1,2,3,4-tetrahydroisoquinolin-6-yl)-4-(4,4-dimethylpiperidin-1-yl)-2,6-dimethylpyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20 mg, 0.038 mmol), 2,3,5-trifluoropyridine (69.3 mg, 0.521 mmol), acetonitrile (1.5 mL) and N,N-diisopropylethylamine (35 μL, 0.200 mmol). The reaction was capped and placed in a microwave reactor at 165° C. for 10.5 h. The reaction was treated with EtOH (1.5 mL) and 10 M sodium hydroxide (60 μL, 0.600 mmol), capped and heated at 110° C. for 4 h. The crude material was purified via preparative LC/MS to afford (S)-2-(tert-butoxy)-2-(5-(2-(3,5-difluoropyridin-2-yl)-1,2,3,4-tetrahydroisoquinolin-6-yl)-4-(4,4-dimethylpiperidin-1-yl)-2,6-dimethylpyridin-3-yl)acetic acid, 6.9 mg (30%). LCMS (M+1)=593.1.

### Example 73

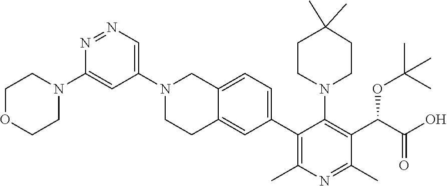 [SMILES: Cc1nc(C)c([C@H](OC(C)(C)C)C(=O)O)c(N2CCC(C)(C)CC2)c1-c1ccc2c(c1)CCN(c1cnnc(N3CCOCC3)c1)C2]

### (S)-2-(tert-Butoxy)-2-(4-(4,4-dimethylpiperidin-1-yl)-2,6-dimethyl-5-(2-(6-morpholinopyridazin-4-yl)-1,2,3,4-tetrahydroisoquinolin-6-yl)pyridin-3-yl)acetic Acid

A mixture of (S)-isopropyl 2-(tert-butoxy)-2-(4-(4,4-dimethylpiperidin-1-yl)-2,6-dimethyl-5-(4,4,5,5-tetramethyl-1,3,2-dioxaborolan-2-yl)pyridin-3-yl)acetate (29 mg, 0.056 mmol) and 4-(5-(6-bromo-3,4-dihydroisoquinolin-2(1H)-yl)pyridazin-3-yl)morpholine (43 mg, 0.115 mmol) in THF (2.5 mL) under argon was added 0.5 M potassium phosphate tribasic (0.3 mL, 0.150 mmol), followed by 2nd generation X-Phos precatalyst (6.5 mg, 8.26 μmol). The reaction was flushed with argon, sealed, stirred room temp for 5 min then heated at 100° C. for 18 h. The solvent was removed under a gentle stream of argon and the residue was dissolved in EtOH (2 mL). The resulting solution was treated with 10 M sodium hydroxide in water (45 μl, 0.450 mmol and heated at 100° C. for 18 h. The crude material was purified via preparative LC/MS to afford (S)-2-(tert-butoxy)-2-(4-(4,4-dimethylpiperidin-1-yl)-2,6-dimethyl-5-(2-(6-morpholinopyridazin-4-yl)-1,2,3,4-tetrahydroisoquinolin-6-yl)pyridin-3-yl)acetic acid, 8.6 mg (22%). LCMS (M+1)=643.2.

### Example 74

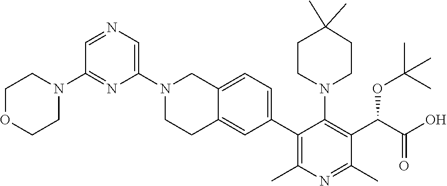 [SMILES: Cc1nc(C)c([C@H](OC(C)(C)C)C(=O)O)c(N2CCC(C)(C)CC2)c1-c1ccc2c(c1)CCN(c1cncc(N3CCOCC3)n1)C2]

### (S)-2-(tert-Butoxy)-2-(4-(4,4-dimethylpiperidin-1-yl)-2,6-dimethyl-5-(2-(6-morpholinopyrazin-2-yl)-1,2,3,4-tetrahydroisoquinolin-6-yl)pyridin-3-yl)acetic Acid

To a reaction vial under nitrogen containing (S)-isopropyl 2-(tert-butoxy)-2-(4-(4,4-dimethylpiperidin-1-yl)-2,6-dimethyl-5-(4,4,5,5-tetramethyl-1,3,2-dioxaborolan-2-yl)pyridin-3-yl)acetate (30 mg, 0.058 mmol) was added 4-(6-(6-bromo-3,4-dihydroisoquinolin-2(1H)-yl)pyrazin-2-yl)morpholine (41 mg, 0.109 mmol) and THF (2.0 mL). The reaction was flushed with argon, treated with 0.5 M potassium phosphate tribasic (0.3 mL, 0.150 mmol) and 2nd Generation X-Phos precatalyst (8.0 mg, 10.17 mol), securely capped, stirred at room temp for 5 min and then heated at 100° C. for 18 h. The solvent was removed under a stream of argon and the residue was redissolved in ethanol (2.0 mL). The resulting solution was treated with 10 M sodium hydroxide (50 μL, 0.500 mmol), flushed briefly with nitrogen and heated at 100° C. for 18 h. The crude material was purified via preparative LC/MS to afford (S)-2-(tert-butoxy)-2-(4-(4,4-dimethylpiperidin-1-yl)-2,6-dimethyl-5-(2-(6-morpholinopyrazin-2-yl)-1,2,3,4-tetrahydroisoquinolin-6-yl)pyridin-3-yl)acetic acid, 11.2 mg (30%). LCMS (M+1)=643.2.

### Example 75

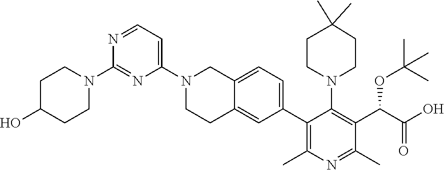 [SMILES: Cc1nc(C)c([C@H](OC(C)(C)C)C(=O)O)c(N2CCC(C)(C)CC2)c1-c1ccc2c(c1)CCN(c1ccnc(N3CCC(O)CC3)n1)C2]

### (S)-2-(tert-Butoxy)-2-(4-(4,4-dimethylpiperidin-1-yl)-5-(2-(2-(4-hydroxypiperidin-1-yl)pyrimidin-4-yl)-1,2,3,4-tetrahydroisoquinolin-6-yl)-2,6-dimethylpyridin-3-yl)acetic Acid

To a mixture of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20 mg, 0.038 mmol) and 2,4-dichloropyrimidine (12 mg, 0.081 mmol) in ethanol (2 mL) was added Hunig's base (35 μl, 0.200 mmol) under nitrogen in microwave vial. The reaction was capped, stirred room temp for 5 min then heated at 80° C. for 18 h. The reaction was then treated with solution of piperidin-4-ol (35 mg, 0.346 mmol) in 0.5 mL EtOH and heated at 100° C. sand bath shaker for 90 min, followed by heating in a microwave reactor at 122° C. for 4 h. The reaction was then treated 10 M sodium hydroxide (45 μl, 0.450 mmol) and heated at 100° C. for 18 h. The crude material was purified via preparative LC/MS to afford (S)-2-(tert-butoxy)-2-(4-(4,4-dimethylpiperidin-1-yl)-5-(2-(2-(4-hydroxypiperidin-1-yl)pyrimidin-4-yl)-1,2,3,4-tetrahydroisoquinolin-6-yl)-2,6-dimethylpyridin-3-yl)acetic acid, 19.1 mg (76%). LCMS (M+1)=657.3.

### Example 76

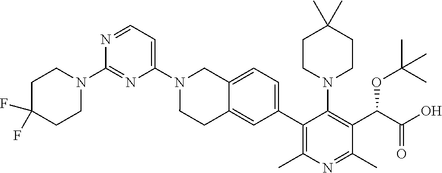 [SMILES: Cc1nc(C)c([C@H](OC(C)(C)C)C(=O)O)c(N2CCC(C)(C)CC2)c1-c1ccc2c(c1)CCN(c1ccnc(N3CCC(F)(F)CC3)n1)C2]

### (S)-2-(tert-Butoxy)-2-(5-(2-(2-(4,4-difluoropiperidin-1-yl)pyrimidin-4-yl)-1,2,3,4-tetrahydroisoquinolin-6-yl)-4-(4,4-dimethylpiperidin-1-yl)-2,6-dimethylpyridin-3-yl)acetic Acid

To a mixture of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (21 mg, 0.040 mmol) and 2,4-dichloropyrimidine (15.9 mg, 0.107 mmol) in ethanol (2 mL) was added Hunig's base (45 μl, 0.258 mmol) under nitrogen in a microwave vial. The reaction was capped, stirred rt 5 min then heated at 100° C. for 75 min. The reaction was then treated with 4,4-difluoropiperidine, HCl (60 mg, 0.381 mmol), additional Hunigs base (80 ulit) heated in a microwave reactor at 130° C. for 14 h. The reaction was then treated with 10 M sodium hydroxide (80 μl, 0.800 mmol) and heated at 105° C. for 5 h. The crude material was purified via preparative LC/MS to afford (S)-2-(tert-butoxy)-2-(5-(2-(2-(4,4-difluoropiperidin-1-yl)pyrimidin-4-yl)-1,2,3,4-tetrahydroisoquinolin-6-yl)-4-(4,4-dimethylpiperidin-1-yl)-2,6-dimethylpyridin-3-yl)acetic acid, 15.4 mg (57%). LCMS (M+1)=677.2.

### Example 77

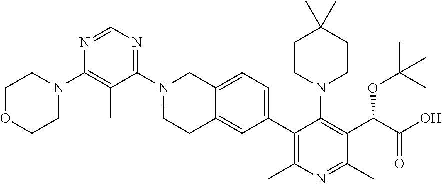 [SMILES: Cc1nc(C)c([C@H](OC(C)(C)C)C(=O)O)c(N2CCC(C)(C)CC2)c1-c1ccc2c(c1)CCN(c1ncnc(N3CCOCC3)c1C)C2]

### (S)-2-(tert-Butoxy)-2-(4-(4,4-dimethylpiperidin-1-yl)-2,6-dimethyl-5-(2-(5-methyl-6-morpholinopyrimidin-4-yl)-1,2,3,4-tetrahydroisoquinolin-6-yl)pyridin-3-yl)acetic Acid

To a mixture of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (21.8 mg, 0.042 mmol) and 4,6-dichloro-5-methylpyrimidine (25 mg, 0.153 mmol) in ethanol (1.5 mL) was added Hunig's base (55 μl, 0.315 mmol) under nitrogen in microwave vial. The reaction was capped, stirred at room temp for 5 min then heated in a microwave reactor at 100-110° C. for 75 min. The reaction was then treated morpholine (55 μl, 0.631 mmol) and heated at 100° C. for 18 h, followed by heating in a microwave reactor at 145° C. for 3 h. The reaction was then treated with 10 M sodium hydroxide (80 μl, 0.800 mmol) and heated at 100° C. for 1.75 h. The crude material was purified via preparative LC/MS to afford (S)-2-(tert-butoxy)-2-(4-(4,4-dimethylpiperidin-1-yl)-2,6-dimethyl-5-(2-(5-methyl-6-morpholinopyrimidin-4-yl)-1,2,3,4-tetrahydroisoquinolin-6-yl)pyridin-3-yl)acetic acid, 12.6 mg (44%). LCMS (M+1)=657.2.

Also isolated from this reaction was

### Example 78

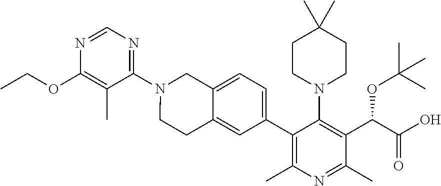 [SMILES: CCOc1ncnc(N2CCc3cc(-c4c(C)nc(C)c([C@H](OC(C)(C)C)C(=O)O)c4N4CCC(C)(C)CC4)ccc3C2)c1C]

### (S)-2-(tert-Butoxy)-2-(4-(4,4-dimethylpiperidin-1-yl)-5-(2-(6-ethoxy-5-methylpyrimidin-4-yl)-1,2,3,4-tetrahydroisoquinolin-6-yl)-2,6-dimethylpyridin-3-yl)acetic Acid 2.3 mg (9%)

LCMS (M+1)=616.2.

### Example 79

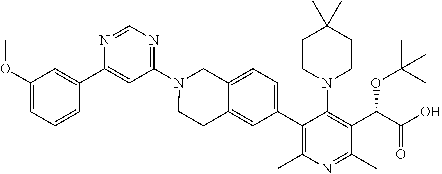 [SMILES: COc1cccc(-c2cc(N3CCc4cc(-c5c(C)nc(C)c([C@H](OC(C)(C)C)C(=O)O)c5N5CCC(C)(C)CC5)ccc4C3)ncn2)c1]

### (S)-2-(tert-Butoxy)-2-(4-(4,4-dimethylpiperidin-1-yl)-5-(2-(6-(3-methoxyphenyl)pyrimidin-4-yl)-1,2,3,4-tetrahydroisoquinolin-6-yl)-2,6-dimethylpyridin-3-yl)acetic Acid

To a mixture of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (21 mg, 0.040 mmol) and 4-chloro-6-(3-methoxyphenyl)pyrimidine (25.3 mg, 0.115 mmol) in ethanol (2 mL) was treated with Hunig's base (40 μl, 0.229 mmol) under nitrogen in a microwave vial. The reaction was capped, stirred at room temp 5 min then heated at 100° C. sand bath for 4 h. The reaction was treated with 10 M sodium hydroxide (60 μl, 0.600 mmol) and heated at 100° C. for 90 min. Additional 10 M sodium hydroxide (20 μl, 0.200 mmol) was added and the reaction was heated at 100° C. for 18 h. The crude material was purified via preparative LC/MS to afford (S)-2-(tert-butoxy)-2-(4-(4,4-dimethylpiperidin-1-yl)-5-(2-(6-(3-methoxyphenyl)pyrimidin-4-yl)-1,2,3,4-tetrahydroisoquinolin-6-yl)-2,6-dimethylpyridin-3-yl)acetic acid, 18.7 mg (64%). LCMS (M+1)=664.2.

### Example 80

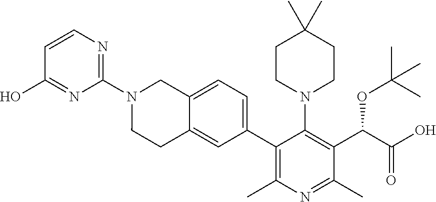 [SMILES: Cc1nc(C)c([C@H](OC(C)(C)C)C(=O)O)c(N2CCC(C)(C)CC2)c1-c1ccc2c(c1)CCN(c1nccc(O)n1)C2]

### (S)-2-(tert-Butoxy)-2-(4-(4,4-dimethylpiperidin-1-yl)-5-(2-(4-hydroxypyrimidin-2-yl)-1,2,3,4-tetrahydroisoquinolin-6-yl)-2,6-dimethylpyridin-3-yl)acetic Acid

To a mixture of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (22 mg, 0.042 mmol) and 4-chloro-2-(methylsulfonyl)pyrimidine (15.8 mg, 0.082 mmol) in ethanol (2 mL) was added Hunig's base (40 μl, 0.229 mmol) under nitrogen in a microwave vial. The resulting suspension was heated at 100° C. for 2 h, followed by heating in a microwave reactor at 140-155° C. for 5 h. The reaction was treated with 10 M sodium hydroxide (80 μl, 0.800 mmol) and heated at 100° C. for 7.5 h. The crude material was purified via preparative LC/MS to afford (S)-2-(tert-butoxy)-2-(4-(4,4-dimethylpiperidin-1-yl)-5-(2-(4-hydroxypyrimidin-2-yl)-1,2,3,4-tetrahydroisoquinolin-6-yl)-2,6-dimethylpyridin-3-yl)acetic acid, 1.9 mg (8%). LCMS (M+1)=574.2.

### Example 81

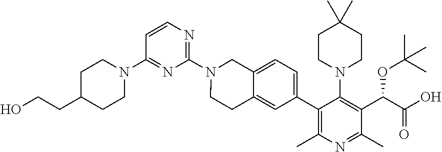 [SMILES: Cc1nc(C)c([C@H](OC(C)(C)C)C(=O)O)c(N2CCC(C)(C)CC2)c1-c1ccc2c(c1)CCN(c1nccc(N3CCC(CCO)CC3)n1)C2]

### (S)-2-(tert-Butoxy)-2-(4-(4,4-dimethylpiperidin-1-yl)-5-(2-(4-(4-(2-hydroxyethyl)piperidin-1-yl)pyrimidin-2-yl)-1,2,3,4-tetrahydroisoquinolin-6-yl)-2,6-dimethylpyridin-3-yl)acetic Acid

To a dry microwave vial under nitrogen was added 2,4-dichloropyrimidine (20 mg, 0.134 mmol) and 2-(piperidin-4-yl)ethanol (18 mg, 0.139 mmol), followed by a solution of Hunig's base (30 μl, 0.172 mmol) in ethanol (2 mL). The reaction was capped, stirred at room temp for 5 min then heated at 100° C. for 40 min. The reaction was then treated with (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (22 mg, 0.042 mmol), additional Hunig base (30 ulit) and heated at 100° C. for 18 h, followed by heating in a microwave reactor at 145° C. for 3 h. The reaction was then treated with 10 M sodium hydroxide (80 μl, 0.800 mmol) and heated at 100° C. for 18 h. The crude material was purified via preparative LC/MS to afford (S)-2-(tert-butoxy)-2-(4-(4,4-dimethylpiperidin-1-yl)-5-(2-(4-(4-(2-hydroxyethyl)piperidin-1-yl)pyrimidin-2-yl)-1,2,3,4-tetrahydroisoquinolin-6-yl)-2,6-dimethylpyridin-3-yl)acetic acid, 5.5 mg (19%). LCMS (M+1)=685.2.

### Example 82

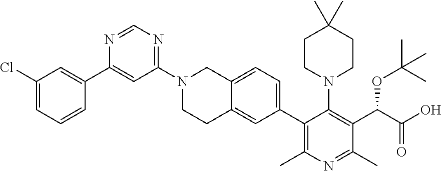 [SMILES: Cc1nc(C)c([C@H](OC(C)(C)C)C(=O)O)c(N2CCC(C)(C)CC2)c1-c1ccc2c(c1)CCN(c1cc(-c3cccc(Cl)c3)ncn1)C2]

### (S)-2-(tert-Butoxy)-2-(5-(2-(6-(3-chlorophenyl)pyrimidin-4-yl)-1,2,3,4-tetrahydroisoquinolin-6-yl)-4-(4,4-dimethylpiperidin-1-yl)-2,6-dimethylpyridin-3-yl)acetic Acid

To a dry pressure vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (21 mg, 0.040 mmol), 4-chloro-6-(3-chlorophenyl)pyrimidine (25.3 mg, 0.112 mmol), ethanol (2 mL) and Hunig's base (75 μl, 0.429 mmol). The reaction was capped, stirred at room temp for 5 min then heated at 100° C. for 18 h. The reaction was then treated with 10 M sodium hydroxide (60 μl, 0.600 mmol) and heated at 100° C. sand bath for 18 h. The crude material was purified via preparative LC/MS to afford (S)-2-(tert-butoxy)-2-(5-(2-(6-(3-chlorophenyl)pyrimidin-4-yl)-1,2,3,4-tetrahydroisoquinolin-6-yl)-4-(4,4-dimethylpiperidin-1-yl)-2,6-dimethylpyridin-3-yl)acetic acid, 3.5 mg (13%). LCMS (M+1)=688.1.

### Example 83

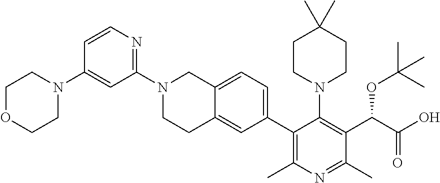 [SMILES: Cc1nc(C)c([C@H](OC(C)(C)C)C(=O)O)c(N2CCC(C)(C)CC2)c1-c1ccc2c(c1)CCN(c1cc(N3CCOCC3)ccn1)C2]

### (S)-2-(tert-Butoxy)-2-(4-(4,4-dimethylpiperidin-1-yl)-2,6-dimethyl-5-(2-(4-morpholinopyridin-2-yl)-1,2,3,4-tetrahydroisoquinolin-6-yl)pyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (22.6 mg, 0.043 mmol), 4-chloro-2-fluoropyridine (14 mg, 0.106 mmol), ethanol (2 mL) and Hunig's base (40 μl, 0.229 mmol). The reaction was capped and heated at 100° C. for 5 h. The reaction was treated with additional 4-chloro-2-fluoropyridine (15 mg, 0.114 mmol) and Hunig's base (60 μL, 0.343 mmol) and heated in a microwave reactor at 135° C. for 8 h The reaction was then treated with morpholine (70 l, 0.803 mmol) and heated in a microwave reactor at 150° C. for 19 h. The solvent was removed under a gentle stream of nitrogen and the residue was dissolved in NMP (0.8 mL), treated with additional morpholine (200 μL, 2.3 mmol) and heated in a microwave reactor at 175° C. for 24 h. The solvent was removed under a gentle stream of nitrogen and the residue was dissolved in ethanol (1.5 mL), treated with 10 M sodium hydroxide in water (100 lit, 1.000 mmol) and heated 100° C. for 18 h. The crude material was purified via preparative LC/MS to afford (S)-2-(tert-butoxy)-2-(4-(4,4-dimethylpiperidin-1-yl)-2,6-dimethyl-5-(2-(4-morpholinopyridin-2-yl)-1,2,3,4-tetrahydroisoquinolin-6-yl)pyridin-3-yl)acetic acid, 3.4 mg (10%). LCMS (M+1)=668.1.

### Example 84

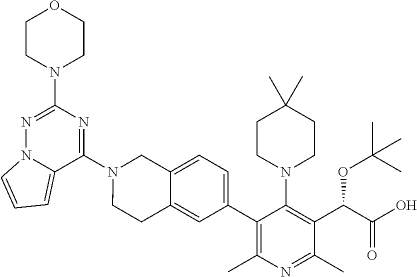 [SMILES: Cc1nc(C)c([C@H](OC(C)(C)C)C(=O)O)c(N2CCC(C)(C)CC2)c1-c1ccc2c(c1)CCN(c1nc(N3CCOCC3)nn3cccc13)C2]

### (S)-2-(tert-Butoxy)-2-(4-(4,4-dimethylpiperidin-1-yl)-2,6-dimethyl-5-(2-(2-morpholinopyrrolo[2,1-f][1,2,4]triazin-4-yl)-1,2,3,4-tetrahydroisoquinolin-6-yl)pyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (19.8 mg, 0.038 mmol), 2,4-dichloropyrrolo[2,1-f][1,2,4]triazine (13.5 mg, 0.072 mmol) and a solution of Hunig's base (22 μl, 0.126 mmol) in ethanol (2 mL). The reaction was capped, stirred at room temp for 5 min, then heated at 80° C. for 2 h. The reaction was then treated with morpholine (100 μl, 1.148 mmol) and heated at 100° C. for 18 h. The reaction was further heated in a microwave reactor at 135-155° C. for 19 h. The reaction was then treated 10 M sodium hydroxide in water (100 μl, 1.000 mmol) and heated at 100° C. for 6 h. The crude material was purified via preparative LC/MS to afford (S)-2-(tert-butoxy)-2-(4-(4,4-dimethylpiperidin-1-yl)-2,6-dimethyl-5-(2-(2-morpholinopyrrolo[2,1-f][1,2,4]triazin-4-yl)-1,2,3,4-tetrahydroisoquinolin-6-yl)pyridin-3-yl)acetic acid, 10.3 mg (40%). LCMS (M+1)=682.2.

### Example 85

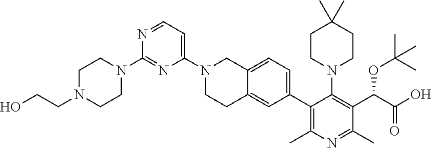 [SMILES: Cc1nc(C)c([C@H](OC(C)(C)C)C(=O)O)c(N2CCC(C)(C)CC2)c1-c1ccc2c(c1)CCN(c1ccnc(N3CCN(CCO)CC3)n1)C2]

### (S)-2-(tert-Butoxy)-2-(4-(4,4-dimethylpiperidin-1-yl)-5-(2-(2-(4-(2-hydroxyethyl)piperazin-1-yl)pyrimidin-4-yl)-1,2,3,4-tetrahydroisoquinolin-6-yl)-2,6-dimethylpyridin-3-yl)acetic Acid

To a dry microwave vial under nitrogen was added 2-(piperazin-1-yl)ethanol (22 mg, 0.169 mmol), 2,4-dichloropyrimidine (26 mg, 0.175 mmol), ethanol (2 mL), and Hunig's base (50 μl, 0.286 mmol). The reaction was capped and heated at 80-100° C. for 20 h. The reaction was then treated with (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20 mg, 0.038 mmol) and additional Hunigs base (50 ulit, 0.286 mmol) and heated in a microwave reactor at 155° C. for 15 h. The reaction was treated with additional 2-(piperazin-1-yl)ethanol and heated in a microwave reactor at 160° C. for 5 h. The reaction was then treated with 10 M sodium hydroxide in water (80 μl, 0.800 mmol) and heated at 100° C. for 7 h. The crude material was purified via preparative LC/MS to afford (S)-2-(tert-butoxy)-2-(4-(4,4-dimethylpiperidin-1-yl)-5-(2-(2-(4-(2-hydroxyethyl)piperazin-1-yl)pyrimidin-4-yl)-1,2,3,4-tetrahydroisoquinolin-6-yl)-2,6-dimethylpyridin-3-yl)acetic acid, 22.1 mg (82%). LCMS (M+1)=686.4.

### Example 86

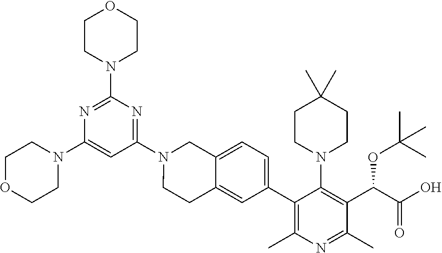 [SMILES: Cc1nc(C)c([C@H](OC(C)(C)C)C(=O)O)c(N2CCC(C)(C)CC2)c1-c1ccc2c(c1)CCN(c1cc(N3CCOCC3)nc(N3CCOCC3)n1)C2]

### (S)-2-(tert-Butoxy)-2-(4-(4,4-dimethylpiperidin-1-yl)-5-(2-(2,6-dimorpholinopyrimidin-4-yl)-1,2,3,4-tetrahydroisoquinolin-6-yl)-2,6-dimethylpyridin-3-yl)acetic Acid

To a dry reaction vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (35 mg, 0.067 mmol), 2,4,6-trichloropyrimidine (28.5 mg, 0.155 mmol), acetonitrile (2 mL) and Hunig's base (70 μl, 0.401 mmol). The reaction was capped and allowed to stir at room temp for 60h. The reaction was further heated at 80° C. for 35 min, then treated with morpholine (100 mlit, 1.148 mmol) and heated in a microwave reactor at 155-160° C. for 16 h. The solvent was removed under a gentle stream of nitrogen and the residue was dissolved in ethanol (2 mL), treated with 10 M sodium hydroxide in water (90 μl, 0.900 mmol) and heated at 105° C. for 18 h. The crude material was purified via preparative LC/MS to afford (S)-2-(tert-butoxy)-2-(4-(4,4-dimethylpiperidin-1-yl)-5-(2-(2,6-dimorpholinopyrimidin-4-yl)-1,2,3,4-tetrahydroisoquinolin-6-yl)-2,6-dimethylpyridin-3-yl)acetic acid, 7.6 mg (16%). LCMS (M+1)=728.3.

### Example 87

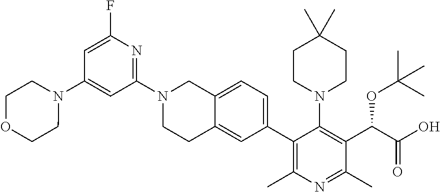 [SMILES: Cc1nc(C)c([C@H](OC(C)(C)C)C(=O)O)c(N2CCC(C)(C)CC2)c1-c1ccc2c(c1)CCN(c1cc(N3CCOCC3)cc(F)n1)C2]

### (S)-2-(tert-Butoxy)-2-(4-(4,4-dimethylpiperidin-1-yl)-5-(2-(6-fluoro-4-morpholinopyridin-2-yl)-1,2,3,4-tetrahydroisoquinolin-6-yl)-2,6-dimethylpyridin-3-yl)acetic Acid

A mixture of (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (25.6 mg, 0.049 mmol) and 4-(2,6-difluoropyridin-4-yl)morpholine (40.5 mg, 0.202 mmol) in a microwave vial was treated with ethanol (1.5 mL) and Hunig's base (80 μl, 0.458 mmol). The reaction was capped, stirred room temp for 30 min, heated at 105° C. for 18 h and then further heated in a microwave reactor at 170° C. for 13 h. The reaction was then treated with 10 M sodium hydroxide (70 μl, 0.700 mmol) and heated in a microwave reactor at 118° C. 3 h 15 min. The crude material was purified via preparative LC/MS to afford (S)-2-(tert-butoxy)-2-(4-(4,4-dimethylpiperidin-1-yl)-5-(2-(6-fluoro-4-morpholinopyridin-2-yl)-1,2,3,4-tetrahydroisoquinolin-6-yl)-2,6-dimethylpyridin-3-yl)acetic acid, 2.7 mg (8%). LCMS (M+1)=660.2.

### Example 88

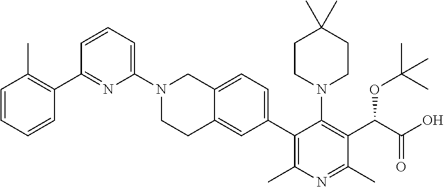 [SMILES: Cc1ccccc1-c1cccc(N2CCc3cc(-c4c(C)nc(C)c([C@H](OC(C)(C)C)C(=O)O)c4N4CCC(C)(C)CC4)ccc3C2)n1]

### (S)-2-(tert-Butoxy)-2-(4-(4,4-dimethylpiperidin-1-yl)-2,6-dimethyl-5-(2-(6-(o-tolyl)pyridin-2-yl)-1,2,3,4-tetrahydroisoquinolin-6-yl)pyridin-3-yl)acetic Acid

A dry microwave vial under nitrogen was charged with (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (28 mg, 0.054 mmol), 2-fluoro-6-(o-tolyl)pyridine (43 mg, 0.230 mmol), acetonitrile (0.9 mL) and Hunig's base (65 μl, 0.372 mmol). The reaction was capped and heated in microwave reactor at 155-170° C. for 26 h. The solvent was removed under a gentle stream of nitrogen and the residue was dissolved in ethanol (2 mL). The resulting solution was treated with 10 M sodium hydroxide in water (65 μl, 0.650 mmol) and the reaction was heated to 105° C. for 6 h. The crude material was purified via preparative LC/MS to afford (S)-2-(tert-butoxy)-2-(4-(4,4-dimethylpiperidin-1-yl)-2,6-dimethyl-5-(2-(6-(o-tolyl)pyridin-2-yl)-1,2,3,4-tetrahydroisoquinolin-6-yl)pyridin-3-yl)acetic acid, 5.4 mg (14%). LCMS (M+1)=657.1.

### Example 89

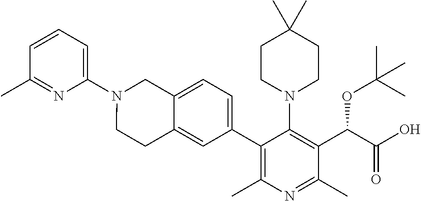 [SMILES: Cc1cccc(N2CCc3cc(-c4c(C)nc(C)c([C@H](OC(C)(C)C)C(=O)O)c4N4CCC(C)(C)CC4)ccc3C2)n1]

### (S)-2-(tert-Butoxy)-2-(4-(4,4-dimethylpiperidin-1-yl)-2,6-dimethyl-5-(2-(6-methylpyridin-2-yl)-1,2,3,4-tetrahydroisoquinolin-6-yl)pyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20 mg, 0.038 mmol), 2-fluoro-6-methylpyridine (60 mg, 0.540 mmol), and acetonitrile (1.2 mL). The reaction was flushed briefly with nitrogen, treated with Hunig's base (100 μl, 0.573 mmol), capped and heated in microwave reactor at 175° C. for 18 h. The solvent was removed under a gentle stream of nitrogen, and the residue was dissolved in ethanol (2 mL). The resulting solution was treated with 10 M sodium hydroxide (80 μl, 0.800 mmol) and heated at 105° C. for 18 h. The crude material was purified via preparative LC/MS to afford (S)-2-(tert-butoxy)-2-(4-(4,4-dimethylpiperidin-1-yl)-2,6-dimethyl-5-(2-(6-methylpyridin-2-yl)-1,2,3,4-tetrahydroisoquinolin-6-yl)pyridin-3-yl)acetic acid, 2.4 mg (10%). LCMS (M+1)=571.2.

### Example 90

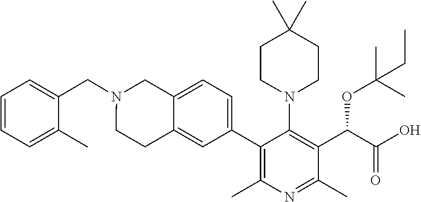 [SMILES: CCC(C)(C)O[C@H](C(=O)O)c1c(C)nc(C)c(-c2ccc3c(c2)CCN(Cc2ccccc2C)C3)c1N1CCC(C)(C)CC1]

### (S)-2-(4-(4,4-Dimethylpiperidin-1-yl)-2,6-dimethyl-5-(2-(2-methylbenzyl)-1,2,3,4-tetrahydroisoquinolin-6-yl)pyridin-3-yl)-2-(tert-pentyloxy)acetic Acid

To a dry reaction vial under nitrogen was added (S)-isopropyl 2-(5-bromo-4-(4,4-dimethylpiperidin-1-yl)-2,6-dimethylpyridin-3-yl)-2-(tert-pentyloxy)acetate (23.8 mg, 0.049 mmol), 2-(2-chloro-6-methylbenzyl)-6-(4,4,5,5-tetramethyl-1,3,2-dioxaborolan-2-yl)-1,2,3,4-tetrahydroisoquinoline (27 mg, 0.068 mmol) and THF (5 mL). The reaction was flushed with argon, then treated with 0.5 M potassium phosphate tribasic (450 μl, 0.225 mmol), followed by 2^{nd} generation X-phos precatalyst (10 mg, 0.013 mmol), capped and stirred at room temp for 18 h. The solvent was removed under a gentle stream of nitrogen and the residue was dissolved in EtOH (2 mL). The resulting solution was treated with 10 M sodium hydroxide in water (55 μl, 0.550 mmol), stirred at room temp for 45 min, and then heated at 105° C. for 18 h. The reaction was treated with additional 10 M sodium hydroxide in water (30 μl, 0.300 mmol) and heated at 105° C. for 18 h. The crude material was purified via preparative LC/MS to afford (S)-2-(4-(4,4-dimethylpiperidin-1-yl)-2,6-dimethyl-5-(2-(2-methylbenzyl)-1,2,3,4-tetrahydroisoquinolin-6-yl)pyridin-3-yl)-2-(tert-pentyloxy)acetic acid, 13 mg (44%). LCMS (M+1)=598.2.

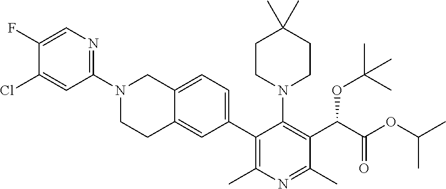 [SMILES: Cc1nc(C)c([C@H](OC(C)(C)C)C(=O)OC(C)C)c(N2CCC(C)(C)CC2)c1-c1ccc2c(c1)CCN(c1cc(Cl)c(F)cn1)C2]

### Isopropyl (S)-2-(tert-butoxy)-2-(5-(2-(4-chloro-5-fluoropyridin-2-yl)-1,2,3,4-tetrahydroisoquinolin-6-yl)-4-(4,4-dimethylpiperidin-1-yl)-2,6-dimethylpyridin-3-yl)acetate

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (26.4 mg, 0.051 mmol), 4-chloro-2,5-difluoropyridine (34 mg, 0.227 mmol), and acetonitrile (0.9 mL). The reaction was flushed briefly with nitrogen, treated with Hunig's base (60 μl, 0.344 mmol), capped and heated in a microwave reactor at 85° C. for 8 h. Added additional 4-chloro-2,5-difluoropyridine (20.4 mg, 0.136 mmol) and Hunig's base (70 μl, 0.401 mmol) and heated in a microwave reactor at 120° C. for 5 h, followed by 80° C. (sand bath) for 18 h. Treated the reaction with additional 4-chloro-2,5-difluoropyridine (72 mg, 0.482 mmol) and Hunig's base (70 μl, 0.401 mmol) and heated in a microwave reactor at 120° C. for 3 h, followed by 80° C. (sand bath) for 18 h. The crude reaction was purified via reverse phase Prep-HPLC to give isopropyl-(S)-2-(tert-butoxy)-2-(5-(2-(4-chloro-5-fluoropyridin-2-yl)-1,2,3,4-tetrahydroisoquinolin-6-yl)-4-(4,4-dimethylpiperidin-1-yl)-2,6-dimethylpyridin-3-yl)acetate, 10.3 mg (31%). LCMS (M+1)=651.4 and 653.4.

### Example 91

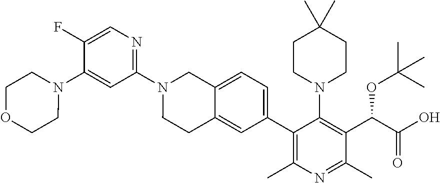 [SMILES: Cc1nc(C)c([C@H](OC(C)(C)C)C(=O)O)c(N2CCC(C)(C)CC2)c1-c1ccc2c(c1)CCN(c1cc(N3CCOCC3)c(F)cn1)C2]

### (S)-2-(tert-Butoxy)-2-(4-(4,4-dimethylpiperidin-1-yl)-5-(2-(5-fluoro-4-morpholinopyridin-2-yl)-1,2,3,4-tetrahydroisoquinolin-6-yl)-2,6-dimethylpyridin-3-yl)acetic Acid

To a dry microwave vial under nitrogen was added isopropyl-(S)-2-(tert-butoxy)-2-(5-(2-(4-chloro-5-fluoropyridin-2-yl)-1,2,3,4-tetrahydroisoquinolin-6-yl)-4-(4,4-dimethylpiperidin-1-yl)-2,6-dimethylpyridin-3-yl)acetate (8.6 mg, 0.013 mmol), morpholine (100 μl, 1.148 mmol) and EtOH (3 mL). The resulting solution was heated in a microwave reactor at 140° C. for 16 h. The solvent was removed under a gentle stream of nitrogen and the residue was dissolved in NMP (2.0 mL). The reaction was treated with additional morpholine (100 mlit, 1.148 mmol) and heated in a microwave reactor at 180° C. for 4 h. The reaction was treated with additional morpholine (100 mlit, 1.148 mmol) and heated in a microwave reactor at 200° C. for 16 h. The solvent was removed under a gentle stream of nitrogen and the residue was dissolved in ethanol (2.0 mL). The resulting solution was treated with 10 M sodium hydroxide (25 μl, 0.250 mmol) and heated at 105° C. for 18 h. The crude material was purified via preparative LC/MS to afford (S)-2-(tert-butoxy)-2-(4-(4,4-dimethylpiperidin-1-yl)-5-(2-(5-fluoro-4-morpholinopyridin-2-yl)-1,2,3,4-tetrahydroisoquinolin-6-yl)-2,6-dimethylpyridin-3-yl)acetic acid, 2.2 mg (25%). LCMS (M+1)=660.1.

### Example 92

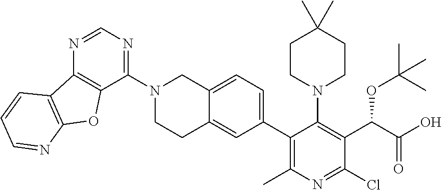 [SMILES: Cc1nc(Cl)c([C@H](OC(C)(C)C)C(=O)O)c(N2CCC(C)(C)CC2)c1-c1ccc2c(c1)CCN(c1ncnc3c1oc1ncccc13)C2]

### (S)-2-(tert-Butoxy)-2-(2-chloro-4-(4,4-dimethylpiperidin-1-yl)-5-(2-(pyrido[3′,2′:4,5]furo[3,2-d]pyrimidin-4-yl)-1,2,3,4-tetrahydroisoquinolin-6-yl)pyridin-3-yl)acetic Acid

To a dry reaction vial under nitrogen was added ethyl-(S)-2-(5-bromo-2-chloro-4-(4,4-dimethylpiperidin-1-yl)pyridin-3-yl)-2-(tert-butoxy)acetate (45.4 mg, 0.098 mmol), 4-(6-(4,4,5,5-tetramethyl-1,3,2-dioxaborolan-2-yl)-3,4-dihydroisoquinolin-2(1H)-yl)pyrido[3′,2′:4,5]furo[3,2-d]pyrimidine (50 mg, 0.117 mmol) and THF (4.5 mL). The reaction was then treated with 0.5 M potassium phosphate tribasic (615 μL, 0.308 mmol), 2^{nd} Generation X-Phos precatalyst (4.3 mg, 5.47 μmol), flushed with argon, capped and allowed to stir at room temp for 18 h. The reaction was treated with additional 0.5 M potassium phosphate tribasic (200 μlit, 0.100 mmol) and 2^{nd} generation X-phos precatalyst (2.7 mg, 0.035 mmol), flushed with argon and heated at 45-50° C. for 18 h. The solvent was removed under a gentle stream of air and the crude product was dissolved in dichloromethane (4 mL). The resulting solution was treated with QuadraSil AP, loading 1.5-2 mmol/gram (36 mg), stirred at room temperature for 10 min, filtered through a 45 μ frit and the solvent was removed under a gentle stream of air. The residue was dissolved in EtOH (2 mL), treated with 10 M sodium hydroxide (150 μL, 1.500 mmol) and heated at 105° C. for 3.5 h. The crude material was purified via preparative LC/MS to afford (S)-2-(tert-butoxy)-2-(2-chloro-4-(4,4-dimethylpiperidin-1-yl)-5-(2-(pyrido[3′,2′:4,5]furo[3,2-d]pyrimidin-4-yl)-1,2,3,4-tetrahydroisoquinolin-6-yl)pyridin-3-yl)acetic acid, 3.4 mg (5%). LCMS (M+1)=655.2.

### Example 93

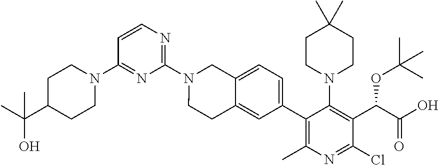 [SMILES: Cc1nc(Cl)c([C@H](OC(C)(C)C)C(=O)O)c(N2CCC(C)(C)CC2)c1-c1ccc2c(c1)CCN(c1nccc(N3CCC(C(C)(C)O)CC3)n1)C2]

### (S)-2-(tert-Butoxy)-2-(2-chloro-4-(4,4-dimethylpiperidin-1-yl)-5-(2-(4-(4-(2-hydroxypropan-2-yl)piperidin-1-yl)pyrimidin-2-yl)-1,2,3,4-tetrahydroisoquinolin-6-yl)pyridin-3-yl)acetic Acid

To a dry reaction vial under nitrogen was added (S)-ethyl 2-(5-bromo-2-chloro-4-(4,4-dimethylpiperidin-1-yl)pyridin-3-yl)-2-(tert-butoxy)acetate (30.4 mg, 0.066 mmol), 2-(1-(2-(6-(4,4,5,5-tetramethyl-1,3,2-dioxaborolan-2-yl)-3,4-dihydroisoquinolin-2(1H)-yl)pyrimidin-4-yl)piperidin-4-yl)propan-2-ol (38 mg, 0.079 mmol) and THF (4 mL). The reaction was flushed with argon, treated with 0.5 M potassium phosphate tribasic (490 μL The reaction was again flushed very well with argon, treated with 2^{nd} generation X-phos precatalyst (4 mg, 5.08 μmol), capped and stirred at room temp for 18 h. The solvent was removed under a gentle stream of air and the crude product was dissolved in dichloromethane (4 mL). The resulting solution was treated with QuadraSil AP, loading 1.5-2 mmol/gram (36 mg), stirred at room temperature for 10 min, filtered through a 45 μ frit and the solvent was removed under a gentle stream of air. The residue was dissolved in EtOH (4 mL), treated with 10 M sodium hydroxide (110 μL, 1.100 mmol) and heated at 105° C. for 3.5 h. The crude material was purified via preparative LC/MS to afford (S)-2-(tert-butoxy)-2-(2-chloro-4-(4,4-dimethylpiperidin-1-yl)-5-(2-(4-(4-(2-hydroxypropan-2-yl)piperidin-1-yl)pyrimidin-2-yl)-1,2,3,4-tetrahydroisoquinolin-6-yl)pyridin-3-yl)acetic acid, 22.3 mg (48%). LCMS (M+1)=705.3.

### Example 94

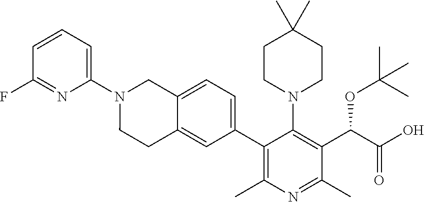 [SMILES: Cc1nc(C)c([C@H](OC(C)(C)C)C(=O)O)c(N2CCC(C)(C)CC2)c1-c1ccc2c(c1)CCN(c1cccc(F)n1)C2]

### (S)-2-(tert-Butoxy)-2-(4-(4,4-dimethylpiperidin-1-yl)-5-(2-(6-fluoropyridin-2-yl)-1,2,3,4-tetrahydroisoquinolin-6-yl)-2,6-dimethylpyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (30 mg, 0.058 mmol), acetonitrile (2.0 mL), 2,6-difluoropyridine (30 μL, 0.328 mmol) and N,N-diisopropylethylamine (100 μL, 0.573 mmol). The reaction was capped and heated in a microwave reactor at 120-140° C. for 11 h. The solvent was removed under a gentle stream of nitrogen and the residue was dissolved in ethanol (2.0 mL). The resulting solution was treated with 10 M sodium hydroxide (85 μL, 0.850 mmol) and heated at 105° C. for 18 h. The crude material was purified via preparative LC/MS to afford (S)-2-(tert-butoxy)-2-(4-(4,4-dimethylpiperidin-1-yl)-5-(2-(6-fluoropyridin-2-yl)-1,2,3,4-tetrahydroisoquinolin-6-yl)-2,6-dimethylpyridin-3-yl)acetic acid, 22.7 mg (69%). LCMS (M+1)=575.3.

### Example 95

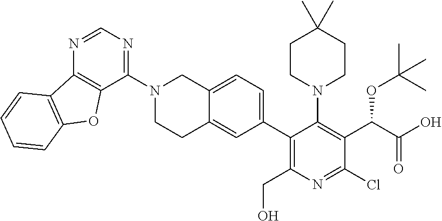 [SMILES: CC1(C)CCN(c2c(-c3ccc4c(c3)CCN(c3ncnc5c3oc3ccccc35)C4)c(CO)nc(Cl)c2[C@H](OC(C)(C)C)C(=O)O)CC1]

### (S)-2-(5-(2-(Benzofuro[3,2-d]pyrimidin-4-yl)-1,2,3,4-tetrahydroisoquinolin-6-yl)-2-chloro-4-(4,4-dimethylpiperidin-1-yl)-6-(hydroxymethyl)pyridin-3-yl)-2-(tert-butoxy)acetic acid

To a solution of (S)-ethyl 2-(5-bromo-2-chloro-4-(4,4-dimethylpiperidin-1-yl)-6-(hydroxymethyl)pyridin-3-yl)-2-(tert-butoxy)acetate (12 mg, 0.024 mmol) and 4-(6-(4,4,5,5-tetramethyl-1,3,2-dioxaborolan-2-yl)-3,4-dihydroisoquinolin-2(1H)-yl)benzofuro[3,2-d]pyrimidine (23 mg, 0.054 mmol) in THF (1.5 mL) was added 0.5 M potassium phosphate tribasic (250 μL, 0.125 mmol). The reaction was flushed with argon, treated with 2^{nd} generation X-phos precatalyst (4.0 mg, 5.08 μmol), capped and stirred at room temp for 18 h. The solvent was removed under a gentle stream of air and the crude product was dissolved in dichloromethane (4 mL). The resulting solution was treated with QuadraSil AP, loading 1.5-2 mmol/gram (36 mg), stirred at room temp for 10 min, filtered thru a 45μ frit and the solvent was removed under a gentle stream of air. The residue was dissolved in EtOH (3 mL), treated with 10 M sodium hydroxide (40 μL, 0.400 mmol) and heated at 105° C. for 130 min. The crude material was purified via preparative LC/MS to afford (S)-2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-(hydroxymethyl)pyridin-3-yl)-2-(tert-butoxy)acetic acid, 3.4 mg (20%). LCMS (M+1)=684.2.

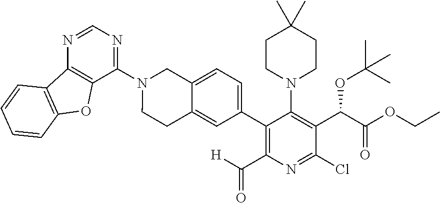 [SMILES: CCOC(=O)[C@@H](OC(C)(C)C)c1c(Cl)nc(C=O)c(-c2ccc3c(c2)CCN(c2ncnc4c2oc2ccccc24)C3)c1N1CCC(C)(C)CC1]

### Ethyl-(S)-2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-formylpyridin-3-yl)-2-(tert-butoxy)acetate

To a dry reaction vial under nitrogen was added (S)-ethyl 2-(5-bromo-2-chloro-4-(4,4-dimethylpiperidin-1-yl)-6-formylpyridin-3-yl)-2-(tert-butoxy)acetate (58.8 mg, 0.120 mmol), 4-(6-(4,4,5,5-tetramethyl-1,3,2-dioxaborolan-2-yl)-3,4-dihydroisoquinolin-2(1H)-yl)benzofuro[3,2-d]pyrimidine (74 mg, 0.173 mmol) and THF (5 mL). The resulting solution was flushed with argon, treated with 0.5 M potassium phosphate tribasic (850 μL, 0.425 mmol), followed by 2^{nd} generation X-phos precatalyst (10.5 mg, 0.013 mmol), capped and stirred at room temp for 18 h. The crude reaction was dissolved in EtOAc (45 mL), extracted with water (1×4 mL), brine (1×5 mL), dried over Na₂SO₄ and concentrated, The crude material was purified via silica gel chromatography chromatography (12 g SiO₂ column, dichloromethane:ethyl acetate 100:0->50:50) to afford ethyl (S)-2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-formylpyridin-3-yl)-2-(tert-butoxy)acetate, 74.6 mg (87%). LCMS (M+1)=710.4, 712.4.

### Example 96

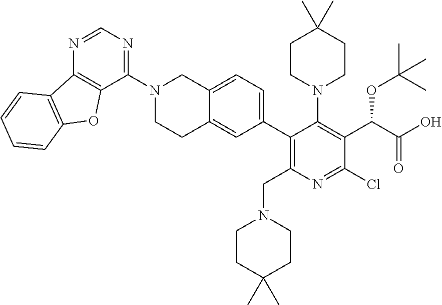 [SMILES: CC1(C)CCN(Cc2nc(Cl)c([C@H](OC(C)(C)C)C(=O)O)c(N3CCC(C)(C)CC3)c2-c2ccc3c(c2)CCN(c2ncnc4c2oc2ccccc24)C3)CC1]

### (S)-2-(5-(2-(Benzofuro[3,2-d]pyrimidin-4-yl)-1,2,3,4-tetrahydroisoquinolin-6-yl)-2-chloro-4-(4,4-dimethylpiperidin-1-yl)-6-((4,4-dimethylpiperidin-1-yl)methyl)pyridin-3-yl)-2-(tert-butoxy)acetic Acid

To a dry reaction vial was added (S)-ethyl 2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-formylpyridin-3-yl)-2-(tert-butoxy)acetate (74.6 mg, 0.105 mmol), 4,4-dimethylpiperidine (44.3 mg, 0.391 mmol), ClCH₂CH₂Cl (1.8 mL), EtOH (1.2 mL), acetic acid (25 μL, 0.437 mmol) and several pieces of activated 4A mol sieves. The reaction was flushed briefly with nitrogen, capped and allowed to stir at room temperature for 20 min. The reaction was then treated slowly (over 45 min) with sodium cyanoborohydride, 1.0M in THF (370 μL, 0.370 mmol). After the addition was complete, the reaction was stirred at room temp for 15 min, then the solvent was removed under a gentle stream of nitrogen. The residue was dissolved in EtOH (3.5 mL), treated with 10 M sodium hydroxide (150 μL, 150 mmol) and heated at 105° C. for 120 min. The crude material was purified via preparative LC/MS to afford (S)-2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-((4,4-dimethylpiperidin-1-yl)methyl)pyridin-3-yl)-2-(tert-butoxy)acetic acid, 12.3 mg (15%). LCMS (M+1)=779.3.

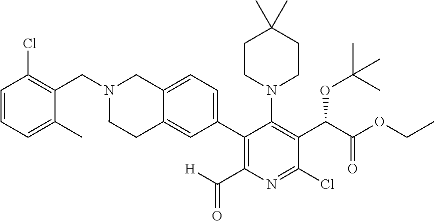 [SMILES: CCOC(=O)[C@@H](OC(C)(C)C)c1c(Cl)nc(C=O)c(-c2ccc3c(c2)CCN(Cc2c(C)cccc2Cl)C3)c1N1CCC(C)(C)CC1]

### Ethyl (S)-2-(tert-butoxy)-2-(2-chloro-5-(2-(2-chloro-6-methylbenzyl)-1,2,3,4-tetrahydroisoquinolin-6-yl)-4-(4,4-dimethylpiperidin-1-yl)-6-formylpyridin-3-yl)acetate

To a dry 40 mL reaction vial under nitrogen was added (S)-ethyl 2-(5-bromo-2-chloro-4-(4,4-dimethylpiperidin-1-yl)-6-formylpyridin-3-yl)-2-(tert-butoxy)acetate (109.2 mg, 0.223 mmol), 2-(2-chloro-6-methylbenzyl)-6-(4,4,5,5-tetramethyl-1,3,2-dioxaborolan-2-yl)-1,2,3,4-tetrahydroisoquinoline (145 mg, 0.365 mmol) and THF (9 mL). The reaction was flushed with argon, treated with 0.5 M potassium phosphate tribasic (1.65 mL, 0.825 mmol), followed by 2^{nd} generation X-Phos precatalyst (12.7 mg, 0.016 mmol), and stirred at room temp for 18 h. The reaction was diluted with ethyl acetate (100 mL), extracted with water (1×5 mL), brine (1×5 mL), dried over Na₂SO₄ and concentrated. The crude material was purified via silica gel chromatography (40 g SiO₂ column, hexane:ethyl acetate 100:0->0:100) to afford ethyl (S)-2-(tert-butoxy)-2-(2-chloro-5-(2-(2-chloro-6-methylbenzyl)-1,2,3,4-tetrahydroisoquinolin-6-yl)-4-(4,4-dimethylpiperidin-1-yl)-6-formylpyridin-3-yl)acetate, 40 mg (26%). LCMS (M+1)=680.3, 682.3.

### Example 97

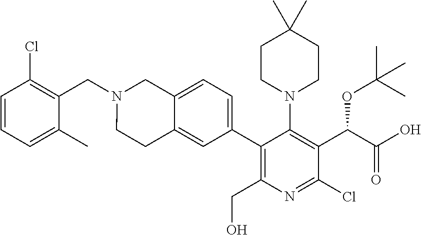 [SMILES: Cc1cccc(Cl)c1CN1CCc2cc(-c3c(CO)nc(Cl)c([C@H](OC(C)(C)C)C(=O)O)c3N3CCC(C)(C)CC3)ccc2C1]

### (S)-2-(tert-Butoxy)-2-(2-chloro-5-(2-(2-chloro-6-methylbenzyl)-1,2,3,4-tetrahydroisoquinolin-6-yl)-4-(4,4-dimethylpiperidin-1-yl)-6-(hydroxymethyl)pyridin-3-yl)acetic Acid

To a dry reaction vial under nitrogen was added (S)-ethyl 2-(tert-butoxy)-2-(2-chloro-5-(2-(2-chloro-6-methylbenzyl)-1,2,3,4-tetrahydroisoquinolin-6-yl)-4-(4,4-dimethylpiperidin-1-yl)-6-formylpyridin-3-yl)acetate (20 mg, 0.029 mmol) and EtOH (2 mL). The resulting solution was treated with sodium borohydride (4 mg, 0.106 mmol) and allowed to stir at room temp for 10 min. The reaction was then treated with 10 M sodium hydroxide (24 μL, 0.240 mmol), and heated at 105° C. for 2 h. The reaction was treated with additional 10 M sodium hydroxide (24 μL, 0.240 mmol) and heated 105° C. for 3 h.

The crude material was purified via preparative LC/MS to afford (S)-2-(tert-butoxy)-2-(2-chloro-5-(2-(2-chloro-6-methylbenzyl)-1,2,3,4-tetrahydroisoquinolin-6-yl)-4-(4,4-dimethylpiperidin-1-yl)-6-(hydroxymethyl)pyridin-3-yl)acetic acid, 8.9 mg (46%). LCMS (M+1)=654.2.

### Example 98

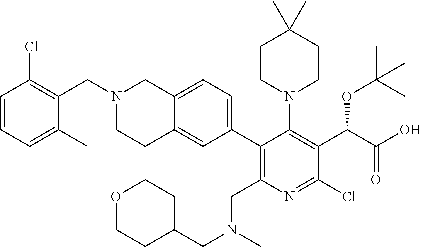 [SMILES: Cc1cccc(Cl)c1CN1CCc2cc(-c3c(CN(C)CC4CCOCC4)nc(Cl)c([C@H](OC(C)(C)C)C(=O)O)c3N3CCC(C)(C)CC3)ccc2C1]

### (S)-2-(tert-Butoxy)-2-(2-chloro-5-(2-(2-chloro-6-methylbenzyl)-1,2,3,4-tetrahydroisoquinolin-6-yl)-4-(4,4-dimethylpiperidin-1-yl)-6-((methyl((tetrahydro-2H-pyran-4-yl)methyl)amino)methyl)pyridin-3-yl)acetic Acid

To a dry vial under nitrogen containing (S)-ethyl 2-(tert-butoxy)-2-(2-chloro-5-(2-(2-chloro-6-methylbenzyl)-1,2,3,4-tetrahydroisoquinolin-6-yl)-4-(4,4-dimethylpiperidin-1-yl)-6-formylpyridin-3-yl)acetate (20 mg, 0.029 mmol) was added methyl-(tetrahydro-pyran-4-ylmethyl)-amine (12.5 mg, 0.097 mmol), ClCH₂CH₂Cl (1.25 mL), acetic acid (6.2 μL, 0.108 mmol) and several pieces of 4A mol sieves. The reaction was stirred at room temp for 10 min, treated with ethanol (0.625 mL) and stirred at room temp for 2.5 h. The reaction was then treated (slowly) with sodium cyanoborohydride, 1.0M in THF (118 μL, 0.118 mmol). After the addition was complete, the reaction was stirred at room temp for 10 min and the solvent removed under a gentle stream of nitrogen. The residue was dissolved in EtOH (2 mL), treated with 10 M sodium hydroxide (35 μL, 0.350 mmol) and heated at 105° C. for 3.5 h. Additional 10 M sodium hydroxide (35 μL, 0.350 mmol) was added and the reaction was heated at 105° C. for 3.5 h. The crude material was purified via preparative LC/MS to afford (S)-2-(tert-butoxy)-2-(2-chloro-5-(2-(2-chloro-6-methylbenzyl)-1,2,3,4-tetrahydroisoquinolin-6-yl)-4-(4,4-dimethylpiperidin-1-yl)-6-((methyl((tetrahydro-2H-pyran-4-yl)methyl)amino)methyl)pyridin-3-yl)acetic acid, 6.4 mg (28%). LCMS (M+1)=767.3.

### Example 99

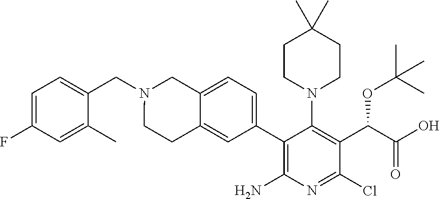 [SMILES: Cc1cc(F)ccc1CN1CCc2cc(-c3c(N)nc(Cl)c([C@H](OC(C)(C)C)C(=O)O)c3N3CCC(C)(C)CC3)ccc2C1]

### (S)-2-(6-Amino-2-chloro-4-(4,4-dimethylpiperidin-1-yl)-5-(2-(4-fluoro-2-methylbenzyl)-1,2,3,4-tetrahydroisoquinolin-6-yl)pyridin-3-yl)-2-(tert-butoxy)acetic Acid

To dry reaction vial under nitrogen was added (S)-ethyl 2-(6-amino-5-bromo-2-chloro-4-(4,4-dimethylpiperidin-1-yl)pyridin-3-yl)-2-(tert-butoxy)acetate (30.1 mg, 0.063 mmol), 2-(4-fluoro-2-methylbenzyl)-6-(4,4,5,5-tetramethyl-1,3,2-dioxaborolan-2-yl)-1,2,3,4-tetrahydroisoquinoline (35.5 mg, 0.093 mmol) and THF (2 mL). The reaction was flushed with argon, treated with 0.5 M potassium phosphate tribasic (500 μL, 0.250 mmol), followed by 2^{nd} generation X-Phos precatalyst (10 mg, 0.013 mmol), capped and stirred at room temp for 18 h. The reaction was diluted with ethyl acetate (75 mL), extracted with water (1×8 mL), brine (1×8 mL), dried over Na₂SO₄ and concentrated. The resulting crude intermediate (41 mg, 0.063 mmol) was dissolved in ethanol (3 mL), treated with 10 M sodium hydroxide (95 μL, 0.950 mmol) and heated at 105° C. for 4 h. The crude material was purified via preparative LC/MS to afford (S)-2-(6-amino-2-chloro-4-(4,4-dimethylpiperidin-1-yl)-5-(2-(4-fluoro-2-methylbenzyl)-1,2,3,4-tetrahydroisoquinolin-6-yl)pyridin-3-yl)-2-(tert-butoxy)acetic acid, 0.9 mg (2%). LCMS (M+1)=623.3.

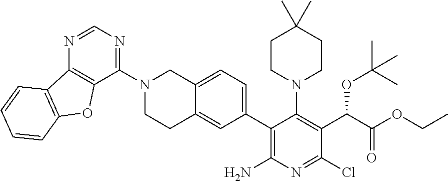 [SMILES: CCOC(=O)[C@@H](OC(C)(C)C)c1c(Cl)nc(N)c(-c2ccc3c(c2)CCN(c2ncnc4c2oc2ccccc24)C3)c1N1CCC(C)(C)CC1]

### Ethyl (S)-2-(6-amino-5-(2-(benzofuro[3,2-d]pyrimidin-4-yl)-1,2,3,4-tetrahydroisoquinolin-6-yl)-2-chloro-4-(4,4-dimethylpiperidin-1-yl)pyridin-3-yl)-2-(tert-butoxy)acetate

To a dry reaction vial under nitrogen was added (S)-ethyl 2-(6-amino-5-bromo-2-chloro-4-(4,4-dimethylpiperidin-1-yl)pyridin-3-yl)-2-(tert-butoxy)acetate (39.5 mg, 0.083 mmol), 4-(6-(4,4,5,5-tetramethyl-1,3,2-dioxaborolan-2-yl)-3,4-dihydroisoquinolin-2(1H)-yl)benzofuro[3,2-d]pyrimidine (60 mg, 0.140 mmol) and THF (4 mL). The reaction was flushed with argon, treated with 0.5 M potassium phosphate tribasic (625 μL, 0.313 mmol), followed by 2^{nd} generation X-phos precatalyst (10.6 mg, 0.013 mmol), capped and stirred at room temp for 18 h. The reaction was dissolved in EtOAc (75 mL), extracted with water (1×20 mL), brine (1×20 mL), dried over Na₂SO₄ and concentrated. The crude material was purified via silica gel chromatography (40 g SiO₂ column, dichloromethane:ethyl acetate 100:0->0:100) to afford ethyl (S)-2-(6-amino-5-(2-(benzofuro[3,2-d]pyrimidin-4-yl)-1,2,3,4-tetrahydroisoquinolin-6-yl)-2-chloro-4-(4,4-dimethylpiperidin-1-yl)pyridin-3-yl)-2-(tert-butoxy)acetate 45.5 mg (79%). LCMS (M+1)=699.2.

### Example 100

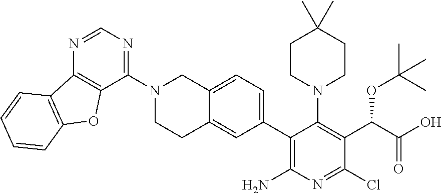 [SMILES: CC1(C)CCN(c2c(-c3ccc4c(c3)CCN(c3ncnc5c3oc3ccccc35)C4)c(N)nc(Cl)c2[C@H](OC(C)(C)C)C(=O)O)CC1]

### (S)-2-(6-Amino-5-(2-(benzofuro[3,2-d]pyrimidin-4-yl)-1,2,3,4-tetrahydroisoquinolin-6-yl)-2-chloro-4-(4,4-dimethylpiperidin-1-yl)pyridin-3-yl)-2-(tert-butoxy)acetic Acid

To a dry reaction vial under nitrogen was added ethyl (S)-2-(6-amino-5-(2-(benzofuro[3,2-d]pyrimidin-4-yl)-1,2,3,4-tetrahydroisoquinolin-6-yl)-2-chloro-4-(4,4-dimethylpiperidin-1-yl)pyridin-3-yl)-2-(tert-butoxy)acetate (45.5 mg, 0.065 mmol), ethanol (4 mL), and 10 M sodium hydroxide (70 μL, 0.700 mmol). The reaction was flushed briefly with nitrogen and heated at 105° C. for 3.5 h. The crude material was purified via preparative LC/MS to afford (S)-2-(6-amino-5-(2-(benzofuro[3,2-d]pyrimidin-4-yl)-1,2,3,4-tetrahydroisoquinolin-6-yl)-2-chloro-4-(4,4-dimethylpiperidin-1-yl)pyridin-3-yl)-2-(tert-butoxy)acetic acid, 11.7 mg (27%). LCMS (M+1)=699.2.

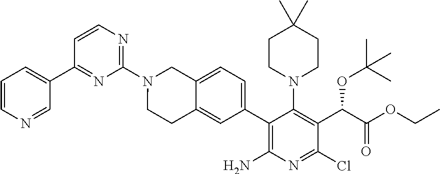 [SMILES: CCOC(=O)[C@@H](OC(C)(C)C)c1c(Cl)nc(N)c(-c2ccc3c(c2)CCN(c2nccc(-c4cccnc4)n2)C3)c1N1CCC(C)(C)CC1]

### Ethyl (S)-2-(6-amino-2-chloro-4-(4,4-dimethylpiperidin-1-yl)-5-(2-(4-(pyridin-3-yl)pyrimidin-2-yl)-1,2,3,4-tetrahydroisoquinolin-6-yl)pyridin-3-yl)-2-(tert-butoxy)acetate

To a dry reaction vial under nitrogen was added (S)-ethyl 2-(6-amino-5-bromo-2-chloro-4-(4,4-dimethylpiperidin-1-yl)pyridin-3-yl)-2-(tert-butoxy)acetate (33 mg, 0.069 mmol), 2-(4-(pyridin-3-yl)pyrimidin-2-yl)-6-(4,4,5,5-tetramethyl-1,3,2-dioxaborolan-2-yl)-1,2,3,4-tetrahydroisoquinoline (49.7 mg, 0.120 mmol) and THF (4 mL). The reaction was flushed with argon, treated with 0.5 M potassium phosphate tribasic (520 μL, 0.260 mmol), followed by 2^{nd} generation X-phos precatalyst (6.8 mg, 8.64 μmol), capped and stirred at room temp for 18 h. The reaction was diluted with EtOAc (75 mL), extracted with water (1×5 mL), brine (1×5 mL), dried over Na₂SO₄ and concentrated. The crude material was purified via silica gel chromatography (12 g SiO₂ column, dichloromethane:EtOAc 100:0->25:75) to afford ethyl (S)-2-(6-amino-2-chloro-4-(4,4-dimethylpiperidin-1-yl)-5-(2-(4-(pyridin-3-yl)pyrimidin-2-yl)-1,2,3,4-tetrahydroisoquinolin-6-yl)pyridin-3-yl)-2-(tert-butoxy)acetate, 39.4 mg (83%). LCMS (M+1)=684.4.

### Example 101

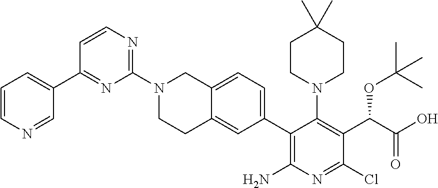 [SMILES: CC1(C)CCN(c2c(-c3ccc4c(c3)CCN(c3nccc(-c5cccnc5)n3)C4)c(N)nc(Cl)c2[C@H](OC(C)(C)C)C(=O)O)CC1]

### (S)-2-(6-Amino-2-chloro-4-(4,4-dimethylpiperidin-1-yl)-5-(2-(4-(pyridin-3-yl)pyrimidin-2-yl)-1,2,3,4-tetrahydroisoquinolin-6-yl)pyridin-3-yl)-2-(tert-butoxy)acetic Acid

To a dry reaction vial under nitrogen was added ethyl (S)-2-(6-amino-2-chloro-4-(4,4-dimethylpiperidin-1-yl)-5-(2-(4-(pyridin-3-yl)pyrimidin-2-yl)-1,2,3,4-tetrahydroisoquinolin-6-yl)pyridin-3-yl)-2-(tert-butoxy)acetate (39.4 mg, 0.058 mmol), ethanol (4.5 mL), and 10 M sodium hydroxide (75 μL, 0.750 mmol). The reaction was flushed briefly with nitrogen and heated at 105° C. for 4.5 h. The crude material was purified via preparative LC/MS to afford (S)-2-(6-amino-2-chloro-4-(4,4-dimethylpiperidin-1-yl)-5-(2-(4-(pyridin-3-yl)pyrimidin-2-yl)-1,2,3,4-tetrahydroisoquinolin-6-yl)pyridin-3-yl)-2-(tert-butoxy)acetic acid (25.7 mg, 66%). LCMS (M+1)=656.3.

### Example 102

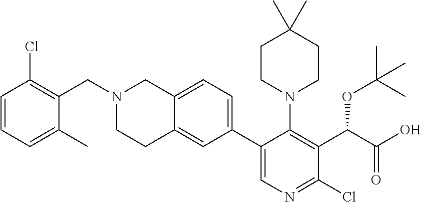 [SMILES: Cc1cccc(Cl)c1CN1CCc2cc(-c3cnc(Cl)c([C@H](OC(C)(C)C)C(=O)O)c3N3CCC(C)(C)CC3)ccc2C1]

### (S)-2-(tert-Butoxy)-2-(2-chloro-5-(2-(2-chloro-6-methylbenzyl)-1,2,3,4-tetrahydroisoquinolin-6-yl)-4-(4,4-dimethylpiperidin-1-yl)pyridin-3-yl)acetic Acid

To a dry reaction vial under nitrogen was added (S)-ethyl 2-(5-bromo-2-chloro-4-(4,4-dimethylpiperidin-1-yl)pyridin-3-yl)-2-(tert-butoxy)acetate (33 mg, 0.071 mmol), 2-(2-chloro-6-methylbenzyl)-6-(4,4,5,5-tetramethyl-1,3,2-dioxaborolan-2-yl)-1,2,3,4-tetrahydroisoquinoline (36.5 mg, 0.092 mmol) and THF (4 mL). The reaction was flushed with argon, treated 0.5 M potassium phosphate tribasic (450 μl, 0.225 mmol), followed by 2^{nd} generation X-phos precatalyst (5 mg, 6.35 μmol), capped and stirred at room temp for 18 h. The solvent was removed under a gentle stream of nitrogen and the crude product was dissolved in dichloromethane (4 mL). The resulting solution was treated with QuadraSil AP, loading 1.5-2 mmol/gram (39 mg), stirred at room temp for 10 min, filtered thru a 45C frit and the solvent was removed under a gentle stream of air. The residue was dissolved in EtOH (2 mL), treated with 10 M sodium hydroxide (110 μL, 1.100 mmol) and heated at 105° C. for 40 min. The crude material was purified via preparative LC/MS to afford (S)-2-(tert-butoxy)-2-(2-chloro-5-(2-(2-chloro-6-methylbenzyl)-1,2,3,4-tetrahydroisoquinolin-6-yl)-4-(4,4-dimethylpiperidin-1-yl)pyridin-3-yl)acetic acid, 24.8 mg (55%). LCMS (M+1)=624.1.

### Example 103

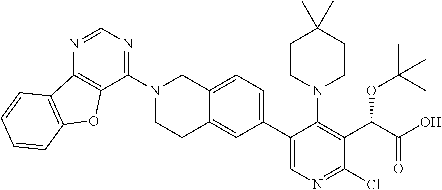 [SMILES: CC1(C)CCN(c2c(-c3ccc4c(c3)CCN(c3ncnc5c3oc3ccccc35)C4)cnc(Cl)c2[C@H](OC(C)(C)C)C(=O)O)CC1]

### (S)-2-(5-(2-(Benzofuro[3,2-d]pyrimidin-4-yl)-1,2,3,4-tetrahydroisoquinolin-6-yl)-2-chloro-4-(4,4-dimethylpiperidin-1-yl)pyridin-3-yl)-2-(tert-butoxy)acetic Acid

To a dry reaction vial under nitrogen was added (S)-ethyl 2-(5-bromo-2-chloro-4-(4,4-dimethylpiperidin-1-yl)pyridin-3-yl)-2-(tert-butoxy)acetate (29.8 mg, 0.065 mmol), 4-(6-(4,4,5,5-tetramethyl-1,3,2-dioxaborolan-2-yl)-3,4-dihydroisoquinolin-2(1H)-yl)benzofuro[3,2-d]pyrimidine (32 mg, 0.075 mmol) and THF (4 mL). The reaction was flushed with argon, treated with degassed 0.5 M potassium phosphate tribasic (450 μl, 0.225 mmol), followed by 2^{nd} generation X-phos precatalyst (5 mg, 6.35 μmol), capped and stirred at room temp for 18 h. The reaction was treated with additional 4-(6-(4,4,5,5-tetramethyl-1,3,2-dioxaborolan-2-yl)-3,4-dihydroisoquinolin-2(1H)-yl)benzofuro[3,2-d]pyrimidine (32 mg, 0.075 mmol), flushed with argon and stirred at room temp for 18 h. The solvent was removed under a gentle stream of nitrogen and the crude product was dissolved in dichloromethane (4 mL). The resulting solution was treated with QuadraSil AP, loading 1.5-2 mmol/gram (31 mg), stirred at room temp for 10 min, filtered thru a 45μ frit and the solvent was removed under a gentle stream of air. The residue was dissolved in EtOH (2 mL), treated with 10 M sodium hydroxide (100 μL, 1.000 mmol) and heated at 105° C. for 55 min. The crude material was purified via preparative LC/MS to afford (S)-2-(5-(2-(benzofuro[3,2-d]pyrimidin-4-yl)-1,2,3,4-tetrahydroisoquinolin-6-yl)-2-chloro-4-(4,4-dimethylpiperidin-1-yl)pyridin-3-yl)-2-(tert-butoxy)acetic acid, 10.9 mg (26%). LCMS (M+1)=654.0.

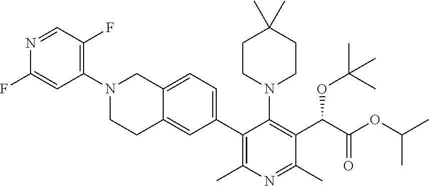 [SMILES: Cc1nc(C)c([C@H](OC(C)(C)C)C(=O)OC(C)C)c(N2CCC(C)(C)CC2)c1-c1ccc2c(c1)CCN(c1cc(F)ncc1F)C2]

### Isopropyl (S)-2-(tert-butoxy)-2-(5-(2-(2,5-difluoropyridin-4-yl)-1,2,3,4-tetrahydroisoquinolin-6-yl)-4-(4,4-dimethylpiperidin-1-yl)-2,6-dimethylpyridin-3-yl)acetate

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (46.8 mg, 0.090 mmol), 4-chloro-2,5-difluoropyridine (149.5 mg, 1.0 mmol), and acetonitrile (0.9 mL). The reaction was flushed briefly with nitrogen, treated with Hunig's base (230 μl, 1.37 mmol), capped and heated in a microwave reactor at 85-95° C. for 17h, followed by heating at 80° C. for 36h in a sand bath. The crude reaction was purified via reverse phase Prep-HPLC to afford isopropyl (S)-2-(tert-butoxy)-2-(5-(2-(2,5-difluoropyridin-4-yl)-1,2,3,4-tetrahydroisoquinolin-6-yl)-4-(4,4-dimethylpiperidin-1-yl)-2,6-dimethylpyridin-3-yl)acetate, 42 mg (quant). LCMS (M+1)=635.4.

### Example 104

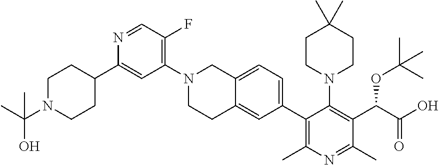 [SMILES: Cc1nc(C)c([C@H](OC(C)(C)C)C(=O)O)c(N2CCC(C)(C)CC2)c1-c1ccc2c(c1)CCN(c1cc(C3CCN(C(C)(C)O)CC3)ncc1F)C2]

### (S)-2-(tert-Butoxy)-2-(4-(4,4-dimethylpiperidin-1-yl)-5-(2-(5-fluoro-2-(4-(2-hydroxypropan-2-yl)piperidin-1-yl)pyridin-4-yl)-1,2,3,4-tetrahydroisoquinolin-6-yl)-2,6-dimethylpyridin-3-yl)acetic Acid

To a dry microwave vial under nitrogen was added (S)-isopropyl 2-(tert-butoxy)-2-(5-(2-(2,5-difluoropyridin-4-yl)-1,2,3,4-tetrahydroisoquinolin-6-yl)-4-(4,4-dimethylpiperidin-1-yl)-2,6-dimethylpyridin-3-yl)acetate (42 mg, 0.066 mmol), 2-(piperidin-4-yl)propan-2-ol (56 mg, 0.391 mmol), NMP (1.2 mL) and Hunig's base (50 μl, 0.286 mmol). The reaction was capped and heated in a microwave reactor at 190 −197° C. for 16 h. The solvent was removed under a gentle stream of nitrogen The residue was dissolved in EtOH (2 mL), treated with 10 M sodium hydroxide (85 μL, 0.850 mmol) and heated at 105° C. for 40 min. The crude material was purified via preparative LC/MS to afford (S)-2-(tert-butoxy)-2-(4-(4,4-dimethylpiperidin-1-yl)-5-(2-(5-fluoro-2-(4-(2-hydroxypropan-2-yl)piperidin-1-yl)pyridin-4-yl)-1,2,3,4-tetrahydroisoquinolin-6-yl)-2,6-dimethylpyridin-3-yl)acetic acid, 10.0 mg (21%). LCMS (M+1)=716.4.

### Example 105

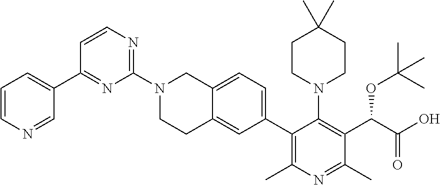 [SMILES: Cc1nc(C)c([C@H](OC(C)(C)C)C(=O)O)c(N2CCC(C)(C)CC2)c1-c1ccc2c(c1)CCN(c1nccc(-c3cccnc3)n1)C2]

### (S)-2-(tert-Butoxy)-2-(4-(4,4-dimethylpiperidin-1-yl)-2,6-dimethyl-5-(2-(4-(pyridin-3-yl)pyrimidin-2-yl)-1,2,3,4-tetrahydroisoquinolin-6-yl)pyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (48.7 mg, 0.093 mmol), 2-chloro-4-(pyridin-3-yl)pyrimidine (50 mg, 0.261 mmol) and ethanol (2 mL). The reaction was flushed with argon, treated with Hunig's base (100 μL, 0.573 mmol), capped and heated in a microwave reactor at 160° C. for 12 h. The reaction was treated with 10 M sodium hydroxide (95 μL, 0.950 mmol) and heated a 105° C. for 6 h. The crude material was purified via preparative LC/MS to afford (S)-2-(tert-butoxy)-2-(4-(4,4-dimethylpiperidin-1-yl)-2,6-dimethyl-5-(2-(4-(pyridin-3-yl)pyrimidin-2-yl)-1,2,3,4-tetrahydroisoquinolin-6-yl)pyridin-3-yl)acetic acid, 25.3 mg (43%). LCMS (M+1)=635.3.

### Example 106

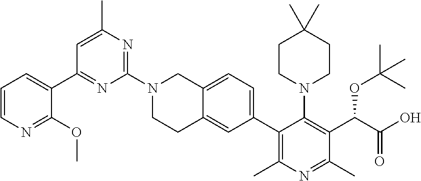 [SMILES: COc1ncccc1-c1cc(C)nc(N2CCc3cc(-c4c(C)nc(C)c([C@H](OC(C)(C)C)C(=O)O)c4N4CCC(C)(C)CC4)ccc3C2)n1]

### (S)-2-(tert-Butoxy)-2-(4-(4,4-dimethylpiperidin-1-yl)-5-(2-(4-(2-methoxypyridin-3-yl)-6-methylpyrimidin-2-yl)-1,2,3,4-tetrahydroisoquinolin-6-yl)-2,6-dimethylpyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20.4 mg, 0.039 mmol), 2-chloro-4-(2-methoxypyridin-3-yl)-6-methylpyrimidine (44.5 mg, 0.189 mmol) and ethanol (1.5 mL). The reaction was flushed with argon, treated with Hunig's base (60 μL, 0.344 mmol) and heated in a microwave reactor at 165° C. for 8 h. The reaction was treated with 10 M sodium hydroxide (60 μL, 0.600 mmol) and heated a 105° C. for 6.5 h. The crude material was purified via preparative LC/MS to afford (S)-2-(tert-butoxy)-2-(4-(4,4-dimethylpiperidin-1-yl)-5-(2-(4-(2-methoxypyridin-3-yl)-6-methylpyrimidin-2-yl)-1,2,3,4-tetrahydroisoquinolin-6-yl)-2,6-dimethylpyridin-3-yl)acetic acid, 18.5 mg (69%). LCMS (M+1)=679.3.

### Example 107

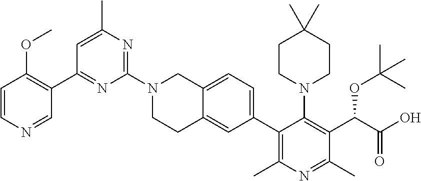 [SMILES: COc1ccncc1-c1cc(C)nc(N2CCc3cc(-c4c(C)nc(C)c([C@H](OC(C)(C)C)C(=O)O)c4N4CCC(C)(C)CC4)ccc3C2)n1]

### (S)-2-(tert-Butoxy)-2-(4-(4,4-dimethylpiperidin-1-yl)-5-(2-(4-(4-methoxypyridin-3-yl)-6-methylpyrimidin-2-yl)-1,2,3,4-tetrahydroisoquinolin-6-yl)-2,6-dimethylpyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (23.3 mg, 0.045 mmol), 2-chloro-4-(4-methoxypyridin-3-yl)-6-methylpyrimidine (49 mg, 0.208 mmol) and ethanol (1.5 mL). The reaction was flushed with argon, treated with Hunig's base (65 μL, 0.372 mmol), capped, heated at 90° C. (sand bath) for 1 h and then heated in a microwave reactor at 165° C. for 8 h. The reaction was treated with 10 M sodium hydroxide (60 μL, 0.600 mmol) and heated at 105° C. for 7 h. The crude material was purified via preparative LC/MS to afford (S)-2-(tert-butoxy)-2-(4-(4,4-dimethylpiperidin-1-yl)-5-(2-(4-(4-methoxypyridin-3-yl)-6-methylpyrimidin-2-yl)-1,2,3,4-tetrahydroisoquinolin-6-yl)-2,6-dimethylpyridin-3-yl)acetic acid, 7 mg (22%). LCMS (M+1)=679.4.

### Example 108

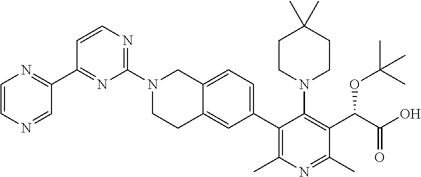 [SMILES: Cc1nc(C)c([C@H](OC(C)(C)C)C(=O)O)c(N2CCC(C)(C)CC2)c1-c1ccc2c(c1)CCN(c1nccc(-c3cnccn3)n1)C2]

### (S)-2-(tert-Butoxy)-2-(4-(4,4-dimethylpiperidin-1-yl)-2,6-dimethyl-5-(2-(4-(pyrazin-2-yl)pyrimidin-2-yl)-1,2,3,4-tetrahydroisoquinolin-6-yl)pyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (24.4 mg, 0.047 mmol), 2-chloro-4-(pyrazin-2-yl)pyrimidine (21.5 mg, 0.112 mmol), acetonitrile (1.5 mL) and Hunigs base. The reaction was capped and heated in a microwave reactor 170° C. for 17 h. The solvent was removed under a gentle stream of nitrogen and the residue was dissolved in ethanol (2.0 mL), treated with 10 M sodium hydroxide (70 μl, 0.700 mmol) and heated at 100-105° C. for 18 h. The crude material was purified via preparative LC/MS to afford (S)-2-(tert-butoxy)-2-(4-(4,4-dimethylpiperidin-1-yl)-2,6-dimethyl-5-(2-(4-(pyrazin-2-yl)pyrimidin-2-yl)-1,2,3,4-tetrahydroisoquinolin-6-yl)pyridin-3-yl)acetic acid, 24.1 mg (81%). LCMS (M+1)=636.3.

### Example 109

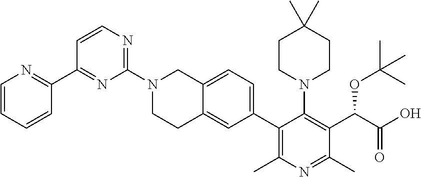 [SMILES: Cc1nc(C)c([C@H](OC(C)(C)C)C(=O)O)c(N2CCC(C)(C)CC2)c1-c1ccc2c(c1)CCN(c1nccc(-c3ccccn3)n1)C2]

### (S)-2-(tert-Butoxy)-2-(4-(4,4-dimethylpiperidin-1-yl)-2,6-dimethyl-5-(2-(4-(pyridin-2-yl)pyrimidin-2-yl)-1,2,3,4-tetrahydroisoquinolin-6-yl)pyridin-3-yl)acetic Acid

To a dry microwave vial under nitrogen was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (31.6 mg, 0.061 mmol), 2-chloro-4-(pyridin-2-yl)pyrimidine (34 mg, 0.177 mmol), acetonitrile (1.5 mL) and Hunigs base. The vial was capped and heated in a microwave reactor at 160° C. for 36 h. The solvent was removed under a gentle stream of nitrogen and the residue was dissolved in ethanol (1.5 mL), treated with 10 M sodium hydroxide (70 μl, 0.700 mmol) and heated at 105° C. for 18 h. The crude material was purified via preparative LC/MS to afford (S)-2-(tert-butoxy)-2-(4-(4,4-dimethylpiperidin-1-yl)-2,6-dimethyl-5-(2-(4-(pyridin-2-yl)pyrimidin-2-yl)-1,2,3,4-tetrahydroisoquinolin-6-yl)pyridin-3-yl)acetic acid, 18.6 mg (44%). LCMS (M+1)=635.3.

### Example 110

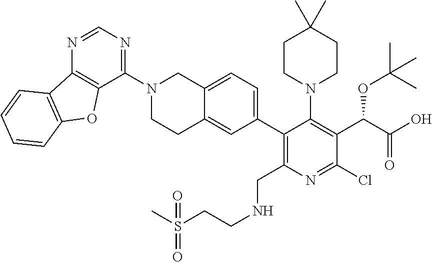 [SMILES: CC1(C)CCN(c2c(-c3ccc4c(c3)CCN(c3ncnc5c3oc3ccccc35)C4)c(CNCCS(C)(=O)=O)nc(Cl)c2[C@H](OC(C)(C)C)C(=O)O)CC1]

### (S)-2-(5-(2-(Benzofuro[3,2-d]pyrimidin-4-yl)-1,2,3,4-tetrahydroisoquinolin-6-yl)-2-chloro-4-(4,4-dimethylpiperidin-1-yl)-6-(((2-(methylsulfonyl)ethyl)amino)methyl)pyridin-3-yl)-2-(tert-butoxy)acetic Acid

To a dry vial under nitrogen was added (S)-ethyl 2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-formylpyridin-3-yl)-2-(tert-butoxy)acetate (73 mg, 0.103 mmol), 2-(methylsulfonyl)ethanamine, HCl (64.8 mg, 0.406 mmol), ClCH₂CH₂Cl (1.5 mL), Hunig's base (10 μL, 0.057 mmol) and 4 pieces of activated 4 A sieves. The reaction was flushed with argon treated, with ethanol (1.2 mL) and stirred at room temp for 25 min. The reaction was then treated (slowly) with sodium cyanoborohydride, 1.0 M in THF (103 μL, 0.103 mmol). After the addition was complete, the reaction was stirred at room temp for 45 min, then the solvent was removed under a gentle stream of nitrogen. The residue was dissolved in ethanol (3 mL), treated with 10 M sodium hydroxide (100 μl, 1.000 mmol), flushed with nitrogen, and heated at 105° C. for 40 min. The reaction was treated with additional 10 M sodium hydroxide (100 μl, 1.000 mmol) and heated at 105° C. for 1.5 h. The crude material was purified via preparative LC/MS to afford (S)-2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-(((2-(methylsulfonyl)ethyl)amino)methyl)pyridin-3-yl)-2-(tert-butoxy)acetic acid, 14.2 mg (18%). LCMS (M+1)=789.2.

### Example 111

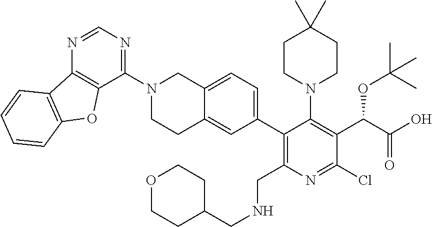 [SMILES: CC1(C)CCN(c2c(-c3ccc4c(c3)CCN(c3ncnc5c3oc3ccccc35)C4)c(CNCC3CCOCC3)nc(Cl)c2[C@H](OC(C)(C)C)C(=O)O)CC1]

### (S)-2-(5-(2-(Benzofuro[3,2-d]pyrimidin-4-yl)-1,2,3,4-tetrahydroisoquinolin-6-yl)-2-chloro-4-(4,4-dimethylpiperidin-1-yl)-6-((((tetrahydro-2H-pyran-4-yl)methyl)amino)methyl)pyridin-3-yl)-2-(tert-butoxy)acetic Acid

To a dry reaction vial under nitrogen was added ethyl-(S)-2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-formylpyridin-3-yl)-2-(tert-butoxy)acetate (73 mg, 0.103 mmol), (tetrahydro-2H-pyran-4-yl)methanamine (40 mg, 0.347 mmol), ClCH₂CH₂Cl (1.5 mL), acetic acid (21 μl, 0.367 mmol) and 4 pieces of activated 4 A sieves. The reaction was flushed with argon, treated with ethanol (0.5 mL) and stirred at room temp for 13 min. The reaction was then treated (slowly) with sodium cyanoborohydride 1 M in THF (400 μl, 0.400 mmol). After the addition was complete, the solvent was removed under a gentle stream of nitrogen. The residue was dissolved in ethanol (3 mL), treated with 10 M sodium hydroxide (100 μl, 1.000 mmol) and heated at 105° C. for 3.5 h. The crude material was purified via preparative LC/MS to afford (S)-2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-((((tetrahydro-2H-pyran-4-yl)methyl)amino)methyl)pyridin-3-yl)-2-(tert-butoxy)acetic acid, 21.3 mg (27%). LCMS (M+1)=781.3.

### Example 112

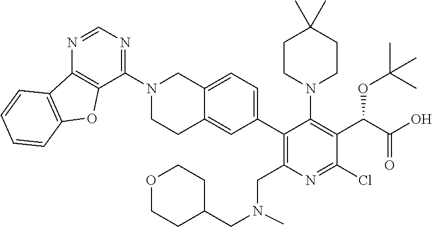 [SMILES: CN(Cc1nc(Cl)c([C@H](OC(C)(C)C)C(=O)O)c(N2CCC(C)(C)CC2)c1-c1ccc2c(c1)CCN(c1ncnc3c1oc1ccccc13)C2)CC1CCOCC1]

### (S)-2-(5-(2-(Benzofuro[3,2-d]pyrimidin-4-yl)-1,2,3,4-tetrahydroisoquinolin-6-yl)-2-chloro-4-(4,4-dimethylpiperidin-1-yl)-6-((methyl((tetrahydro-2H-pyran-4-yl)methyl)amino)methyl)pyridin-3-yl)-2-(tert-butoxy)acetic Acid

To a dry reaction vial under nitrogen was added (S)-ethyl 2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-formylpyridin-3-yl)-2-(tert-butoxy)acetate (73 mg, 0.103 mmol), N-methyl-1-(tetrahydro-2H-pyran-4-yl)methanamine (42 mg, 0.325 mmol), ClCH₂CH₂Cl (1.5 mL), acetic acid (20 μl, 0.349 mmol) and 4 pieces of activated 4 A sieves. The reaction was flushed with argon, treated with ethanol (0.5 mL) and stirred at room temp for 75 min. The reaction was then treated (slowly) with sodium cyanoborohydride 1 M in THF (350 μl, 0.350 mmol). After the addition was complete, the reaction was stirred at room temp for 75 min and then the solvent was removed under a gentle stream of nitrogen. The residue was dissolved in ethanol (3 mL), treated with 10 M sodium hydroxide (100 μl, 1.000 mmol), capped and heated at 105° C. for 1 h. Additional 10 M sodium hydroxide (100 μl, 1.000 mmol) was added and the reaction was heated at 105° C. for 45 min. The crude material was purified via preparative LC/MS to afford (S)-2-(5-(2-(benzofuro[3,2-d]pyrimidin-4-yl)-1,2,3,4-tetrahydroisoquinolin-6-yl)-2-chloro-4-(4,4-dimethylpiperidin-1-yl)-6-((methyl((tetrahydro-2H-pyran-4-yl)methyl)amino)methyl)pyridin-3-yl)-2-(tert-butoxy)acetic acid, 13.6 mg (18%). LCMS (M+1)=684.2.

### Example 113

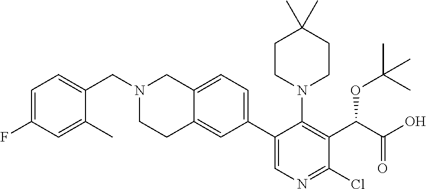 [SMILES: Cc1cc(F)ccc1CN1CCc2cc(-c3cnc(Cl)c([C@H](OC(C)(C)C)C(=O)O)c3N3CCC(C)(C)CC3)ccc2C1]

### (S)-2-(tert-Butoxy)-2-(2-chloro-4-(4,4-dimethylpiperidin-1-yl)-5-(2-(4-fluoro-2-methylbenzyl)-1,2,3,4-tetrahydroisoquinolin-6-yl)pyridin-3-yl)acetic Acid

To a dry reaction vial under argon was added (S)-ethyl 2-(5-bromo-2-chloro-4-(4,4-dimethylpiperidin-1-yl)pyridin-3-yl)-2-(tert-butoxy)acetate (71.5 mg, 0.155 mmol), 2-(4-fluoro-2-methylbenzyl)-6-(4,4,5,5-tetramethyl-1,3,2-dioxaborolan-2-yl)-1,2,3,4-tetrahydroisoquinoline (87 mg, 0.228 mmol) and THF (8 mL). The reaction was flushed with argon, treated with 0.5 M potassium phosphate tribasic (1 mL, 0.500 mmol), followed by 2^{nd} generation X-phos precatalyst (10 mg, 0.013 mmol), capped and stirred at room temp for 18 h. The reaction was diluted with ethyl acetate, extracted and the residue was dissolved in ethanol (5 mL), treated with 10 M sodium hydroxide (130 μL, 1.300 mmol) heated at 100° C. for 4 h. The crude material was purified via preparative LC/MS to afford (S)-2-(tert-butoxy)-2-(2-chloro-4-(4,4-dimethylpiperidin-1-yl)-5-(2-(4-fluoro-2-methylbenzyl)-1,2,3,4-tetrahydroisoquinolin-6-yl)pyridin-3-yl)acetic acid, 42.5 mg (45%). LCMS (M+1)=608.3.

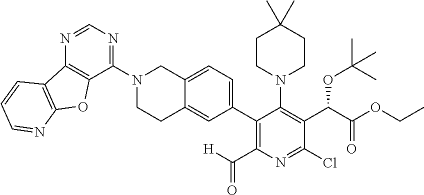 [SMILES: CCOC(=O)[C@@H](OC(C)(C)C)c1c(Cl)nc(C=O)c(-c2ccc3c(c2)CCN(c2ncnc4c2oc2ncccc24)C3)c1N1CCC(C)(C)CC1]

Ethyl (S)-2-(tert-butoxy)-2-(2-chloro-4-(4,4-dimethylpiperidin-1-yl)-6-formyl-5-(2-(pyrido[3′,2′:4,5]furo[3,2-d]pyrimidin-4-yl)-1,2,3,4-tetrahydroisoquinolin-6-yl)pyridin-3-yl)acetate

To a dry reaction vial under argon was added (S)-ethyl 2-(5-bromo-2-chloro-4-(4,4-dimethylpiperidin-1-yl)-6-formylpyridin-3-yl)-2-(tert-butoxy)acetate (117.5 mg, 0.240 mmol), 4-(6-(4,4,5,5-tetramethyl-1,3,2-dioxaborolan-2-yl)-3,4-dihydroisoquinolin-2(1H)-yl)pyrido[3′,2′:4,5]furo[3,2-d]pyrimidine (113 mg, 0.264 mmol), and THF (9 mL). The reaction was flushed with argon, treated with 0.5 M potassium phosphate tribasic (1.65 mL, 0.825 mmol), followed by 2^{nd} generation X-phos precatalyst (15 mg, 0.019 mmol), capped and stirred at room temp for 18 h. The crude reaction was diluted with ethyl acetate, extracted and purified via silica gel chromatography to afford ethyl (S)-2-(tert-butoxy)-2-(2-chloro-4-(4,4-dimethylpiperidin-1-yl)-6-formyl-5-(2-(pyrido[3′,2′:4,5]furo[3,2-d]pyrimidin-4-yl)-1,2,3,4-tetrahydroisoquinolin-6-yl)pyridin-3-yl)acetate, 134.9 mg (40%). LCMS (M+1)=711.4.

### Example 114

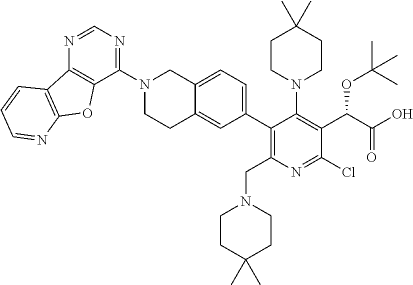 [SMILES: CC1(C)CCN(Cc2nc(Cl)c([C@H](OC(C)(C)C)C(=O)O)c(N3CCC(C)(C)CC3)c2-c2ccc3c(c2)CCN(c2ncnc4c2oc2ncccc24)C3)CC1]

### (S)-2-(tert-Butoxy)-2-(2-chloro-4-(4,4-dimethylpiperidin-1-yl)-6-((4,4-dimethylpiperidin-1-yl)methyl)-5-(2-(pyrido[3′,2′:4,5]furo[3,2-d]pyrimidin-4-yl)-1,2,3,4-tetrahydroisoquinolin-6-yl)pyridin-3-yl)acetic Acid

To a dry reaction vial under nitrogen was added (S)-ethyl 2-(tert-butoxy)-2-(2-chloro-4-(4,4-dimethylpiperidin-1-yl)-6-formyl-5-(2-(pyrido[3′,2′:4,5]furo[3,2-d]pyrimidin-4-yl)-1,2,3,4-tetrahydroisoquinolin-6-yl)pyridin-3-yl)acetate (67 mg, 0.094 mmol), 4,4-dimethylpiperidine (40 mg, 0.353 mmol), ClCH₂CH₂Cl (3 mL), acetic acid (21 μl, 0.367 mmol) and 4 pieces of activated 4 A sieves. The reaction was stirred at room temp for 5 min, treated with ethanol (0.5 mL) and stirred at room temp for 45 min. The reaction was then treated (slowly) with sodium cyanoborohydride 1 M in THF (370 μl, 0.370 mmol). After the addition was complete, the reaction was stirred at room temp for 5 min, then the solvent was removed under a gentle stream of nitrogen. The residue was redissolved in ethanol (4 mL), treated 10 M sodium hydroxide (110 μl, 1.100 mmol) and heated at 100° C. for 4.5 h. The reaction was treated with additional 10 M sodium hydroxide (30 μl, 0.300 mmol) and heated at 105° C. for 4.5h. The crude material was purified via preparative LC/MS to afford (S)-2-(tert-butoxy)-2-(2-chloro-4-(4,4-dimethylpiperidin-1-yl)-6-((4,4-dimethylpiperidin-1-yl)methyl)-5-(2-(pyrido[3′,2′:4,5]furo[3,2-d]pyrimidin-4-yl)-1,2,3,4-tetrahydroisoquinolin-6-yl)pyridin-3-yl)acetic acid, 11.4 mg (16%). LCMS (M+1)=780.3.

Also isolated from this reaction is

### Example 115

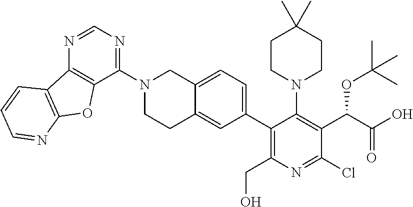 [SMILES: CC1(C)CCN(c2c(-c3ccc4c(c3)CCN(c3ncnc5c3oc3ncccc35)C4)c(CO)nc(Cl)c2[C@H](OC(C)(C)C)C(=O)O)CC1]

### (S)-2-(tert-Butoxy)-2-(2-chloro-4-(4,4-dimethylpiperidin-1-yl)-6-(hydroxymethyl)-5-(2-(pyrido[3′,2′:4,5]furo[3,2-d]pyrimidin-4-yl)-1,2,3,4-tetrahydroisoquinolin-6-yl)pyridin-3-yl)acetic Acid

6.2 mg (10%). LCMS (M+1)=685.3.

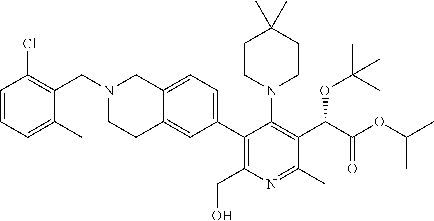 [SMILES: Cc1cccc(Cl)c1CN1CCc2cc(-c3c(CO)nc(C)c([C@H](OC(C)(C)C)C(=O)OC(C)C)c3N3CCC(C)(C)CC3)ccc2C1]

### (S)-Isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)-2-methylpyridin-3-yl)acetate

A 10 mL Schlenk flask equipped with a stir bar was placed under N₂ atm (vac/fill×3). To the flask was added THF (5 mL) and water (1.25) which had been degassed via N₂ bubbling for 5 minutes. To the flask was added Pd(OAc)₂ (5.6 mg); the solution was a light golden color. To the flask was added SPhos (20.6 mg); the solution was dark orange with a slight black hue. The flask was placed in a 60° C. oil bath with stirring for 10 minutes to afford a vibrant orange solution (no black coloring). Using a syringe, a portion of the solution (5.0 mL) was removed and discarded to leave 0.65 mL to 1.25 mL. The catalyst loading is 2-4% based on the amount of solvent remaining in the flask (not determined). To the flask was added in one portion a combined sampled of K₃PO₄ (262 mg, 1.236 mmol), 2-(2-chloro-6-methylbenzyl)-6-(4,4,5,5-tetramethyl-1,3,2-dioxaborolan-2-yl)-1,2,3,4-tetrahydroisoquinoline (164 mg, 0.412 mmol), (S)-isopropyl 2-(5-bromo-4-(4,4-dimethylpiperidin-1-yl)-6-(hydroxymethyl)-2-methylpyridin-3-yl)-2-(tert-butoxy)acetate (200 mg, 0.412 mmol). The solution was stirred at 60° C. Initially the reaction solution was dark orange. Within 5 minutes the solution turned a light red/orange color and the reaction stirred at 60° C. for 18 hrs. The reaction was then cooled to RT and diluted with EtOAc and water. The organic layer was washed with brine, collected, dried over MgSO₄, filtered and volatiles evaporated to afford the crude product. The crude product was purified on silica gel (40 g column, 5-60% EtOAc:Hex) to afford the product (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)-2-methylpyridin-3-yl)acetate (143 mg, 0.211 mmol, 51.3% yield) as a light orange oil. ¹H NMR (500 MHz, CDCl₃) δ 7.18-7.04 (m, 4H), 6.99-6.93 (m, 1H), 6.91-6.86 (m, 1H), 6.07-5.95 (m, 1H), 5.15-5.09 (m, 1H), 4.52-4.42 (m, 1H), 3.93-3.86 (m, 2H), 3.81-3.75 (m, 2H), 2.93-2.82 (m, 5H), 2.66 (s, 3H), 2.53 (s, 2H), 2.51 (s, 2H), 1.30-1.22 (m, 14H), 1.20 (d, J=2.7 Hz, 9H), 0.95-0.87 (m, 3H), 0.75-0.63 (m, 3H). LCMS (M+1)=676.4.

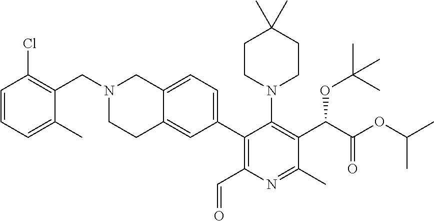 [SMILES: Cc1cccc(Cl)c1CN1CCc2cc(-c3c(C=O)nc(C)c([C@H](OC(C)(C)C)C(=O)OC(C)C)c3N3CCC(C)(C)CC3)ccc2C1]

### (S)-Isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-formyl-2-methylpyridin-3-yl)acetate

To a stirred solution of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)-2-methylpyridin-3-yl)acetate (143 mg, 0.211 mmol) in DCM (1922 μl) and acetonitrile (192 μl) was added Dess-Martin Periodinane (99 mg, 0.233 mmol) at once at rt. After 2 h, the reaction mixture was diluted with ether (50 mL), washed with 1M NaOH (2×15 ml), brine (25 mL), dried (MgSO₄), filtered and concentrated to give (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-formyl-2-methylpyridin-3-yl)acetate (155 mg, 0.230 mmol, 109% yield) as yellow solid. LCMS (M+1)=674.2.

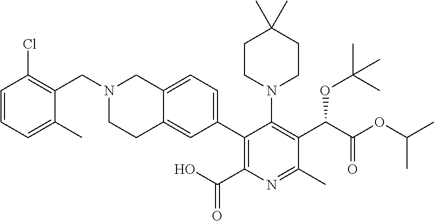 [SMILES: Cc1cccc(Cl)c1CN1CCc2cc(-c3c(C(=O)O)nc(C)c([C@H](OC(C)(C)C)C(=O)OC(C)C)c3N3CCC(C)(C)CC3)ccc2C1]

### (S)-5-(1-(tert-Butoxy)-2-isopropoxy-2-oxoethyl)-3-(2-(2-chloro-6-methylbenzyl)-1,2,3,4-tetrahydroisoquinolin-6-yl)-4-(4,4-dimethylpiperidin-1-yl)-6-methylpicolinic Acid

To a solution of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-formyl-2-methylpyridin-3-yl)acetate (0.155 g, 0.230 mmol) in DMSO (7 ml) was added potassium phosphate monobasic (0.094 g, 0.690 mmol) in water (1.0 mL) followed by sodium chlorite (0.062 g, 0.690 mmol) in water (0.5 mL) and the mixture was stirred for 1 hr. The reaction was diluted with EtOAc and water. The organic layer was washed with water (2×), brine, dried over MgSO₄, filtered and concentrated to afford the product (S)-5-(1-(tert-butoxy)-2-isopropoxy-2-oxoethyl)-3-(2-(2-chloro-6-methylbenzyl)-1,2,3,4-tetrahydroisoquinolin-6-yl)-4-(4,4-dimethylpiperidin-1-yl)-6-methylpicolinic acid (50 mg, 0.072 mmol, 31.5% yield) LCMS (M+1)=690.3.

### Example 116

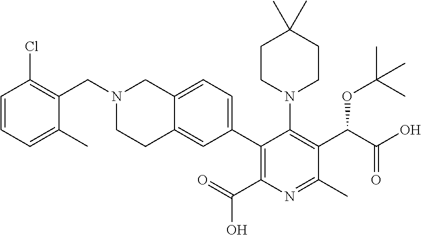 [SMILES: Cc1cccc(Cl)c1CN1CCc2cc(-c3c(C(=O)O)nc(C)c([C@H](OC(C)(C)C)C(=O)O)c3N3CCC(C)(C)CC3)ccc2C1]

### (S)-5-(tert-Butoxy(carboxy)methyl)-3-(2-(2-chloro-6-methylbenzyl)-1,2,3,4-tetrahydroisoquinolin-6-yl)-4-(4,4-dimethylpiperidin-1-yl)-6-methylpicolinic Acid

To a solution of (S)-5-(1-(tert-butoxy)-2-isopropoxy-2-oxoethyl)-3-(2-(2-chloro-6-methylbenzyl)-1,2,3,4-tetrahydroisoquinolin-6-yl)-4-(4,4-dimethylpiperidin-1-yl)-6-methylpicolinic acid (30 mg, 0.043 mmol) in EtOH (1 mL) and water (0.111 mL) was added lithium hydroxide monohydrate (18.24 mg, 0.435 mmol) and stirred at 75° C. for 18 hr. The reaction was cooled to RT and filtered through a 0.45 μ frit filter and purified via preparative LC/MS to afford the product (S)-5-(tert-butoxy(carboxy)methyl)-3-(2-(2-chloro-6-methylbenzyl)-1,2,3,4-tetrahydroisoquinolin-6-yl)-4-(4,4-dimethylpiperidin-1-yl)-6-methylpicolinic acid (12.6 mg, 0.019 mmol, 42.9% yield). The product appeared to be a mixture of atropisomers in a 63:37 ratio according to these reference peaks: ¹H NMR (500 MHz, DMSO-d₆) δ 7.95 (d, J=8.5 Hz, 0.6H), 7.89 (d, J=7.9 Hz, 1H), 7.71 (br d, J=1.2 Hz, 1H), 7.66 (s, 0.6H).

Full Spectrum ¹H NMR (500 MHz, DMSO-d₆) δ 7.97-7.84 (m, 1H), 7.73-7.64 (m, 1H), 7.38-7.22 (m, 4H), 7.21-7.02 (m, 3H), 5.65 (br d, J=9.8 Hz, 1H), 5.03-4.86 (m, 2H), 2.36-2.32 (m, 3H), 2.23-2.03 (m, 1H), 1.54-1.44 (m, 1H), 1.31-1.14 (m, 2H), 1.11 (s, 9H), 1.02-0.93 (m, 1H), 0.84 (br s, 3H), 0.63-0.54 (m, 3H). 9 protons (combination of piperidine and tetrahydroisoquinoline methylene protons) were not observed via ¹H NMR due to water in the experiment. LCMS (M+1)=648.1.

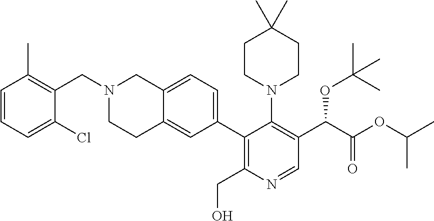 [SMILES: Cc1cccc(Cl)c1CN1CCc2cc(-c3c(CO)ncc([C@H](OC(C)(C)C)C(=O)OC(C)C)c3N3CCC(C)(C)CC3)ccc2C1]

### (S)-Isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)pyridin-3-yl)acetate

A mixture of (S)-isopropyl 2-(5-bromo-4-(4,4-dimethylpiperidin-1-yl)-6-(hydroxymethyl)pyridin-3-yl)-2-(tert-butoxy)acetate (50 mg, 0.106 mmol), 2-(2-chloro-6-methylbenzyl)-6-(4,4,5,5-tetramethyl-1,3,2-dioxaborolan-2-yl)-1,2,3,4-tetrahydroisoquinoline (63.3 mg, 0.159 mmol), 2-dicyclohexylphosphino-2′,6′-dimethoxybiphenyl (8.71 mg, 0.021 mmol), and potassium phosphate tribasic (169 mg, 0.795 mmol) in 1,4-dioxane (1768 μl) and water (354 μl) was bubbled with N₂ for 10 minutes. Pd(OAc)₂ (2.381 mg, 10.61 μmol) was added and the reaction was kept under positive pressure of N₂ for the duration of the reaction. The reaction was heated at 80° C. for 18 h. An extra equivalent of the boronate was added to the reaction and then heated for an additional 30 hrs. The reaction was cooled to RT and diluted with water and EtOAc. The organic layer was washed with brine, collected, dried over MgSO₄, filtered and volatiles evaporated to afford the crude product. The crude product was purified on silica gel (12 g column, 5-75% EtOAc:Hex) to afford the product (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)pyridin-3-yl)acetate (12 mg, 0.018 mmol, 17.08% yield) as a yellow oil. LCMS (M+1)=662.3.

### Example 117

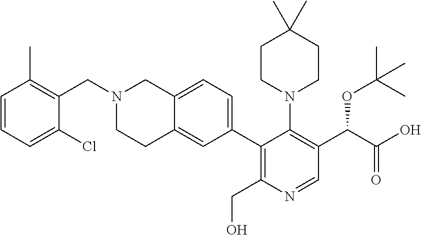 [SMILES: Cc1cccc(Cl)c1CN1CCc2cc(-c3c(CO)ncc([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)pyridin-3-yl)acetic Acid

To a solution of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)pyridin-3-yl)acetate (12 mg, 0.018 mmol) in EtOH (1 mL) and water (0.111 mL) was added lithium hydroxide monohydrate (7.60 mg, 0.181 mmol) and heated at 75° C. for 60 minutes. The reaction was cooled to RT, filtered through a nylon 0.45 μ frit filter and purified via preparative LC/MS to afford the product (S)-2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)pyridin-3-yl)acetic acid (5.2 mg, 8.30 μmol, 45.8% yield). The product appeared to be a mixture of atropisomers in a 55:45 ratio according to these reference peaks ¹H NMR (500 MHz, DMSO-d₆) δ 8.55 (s, 0.8H), 8.54 (s, 1H) Full spectrum: ¹H NMR (500 MHz, DMSO-d₆) δ 8.55 (d, J=8.2 Hz, 1H), 7.31 (d, J=7.3 Hz, 1H), 7.26-7.16 (m, 2H), 7.15-7.08 (m, 2H), 7.06-6.89 (m, 1H), 5.28 (d, J=13.1 Hz, 1H), 4.91-4.73 (m, 1H), 4.29-4.16 (m, 1H), 4.15-4.08 (m, 1H), 3.84-3.80 (m, 2H), 3.73-3.64 (m, 2H), 2.83-2.73 (m, 4H), 2.45 (d, J=3.4 Hz, 3H), 1.17 (d, J=15.0 Hz, 9H), 0.88-0.60 (m, 6H). 7 protons (combination of piperidine and tetrahydroisoquinoline methylene protons) were not observed via ¹H NMR due to water in the experiment. H NMR indicates atropisomers in a ratio of about 55:45. LCMS (M+1)=620.2.

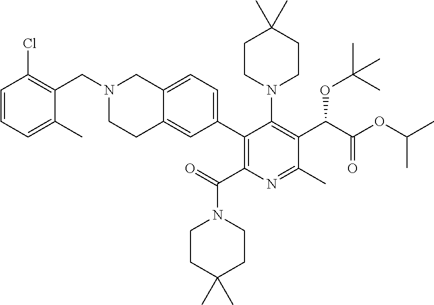 [SMILES: Cc1cccc(Cl)c1CN1CCc2cc(-c3c(C(=O)N4CCC(C)(C)CC4)nc(C)c([C@H](OC(C)(C)C)C(=O)OC(C)C)c3N3CCC(C)(C)CC3)ccc2C1]

### (S)-Isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(4,4-dimethylpiperidine-1-carbonyl)-2-methylpyridin-3-yl)acetate

A mixture of (S)-isopropyl 2-(5-bromo-4-(4,4-dimethylpiperidin-1-yl)-6-(4,4-dimethylpiperidine-1-carbonyl)-2-methylpyridin-3-yl)-2-(tert-butoxy)acetate (38 mg, 0.064 mmol), 2-(2-chloro-6-methylbenzyl)-6-(4,4,5,5-tetramethyl-1,3,2-dioxaborolan-2-yl)-1,2,3,4-tetrahydroisoquinoline (38.1 mg, 0.096 mmol), 2-dicyclohexylphosphino-2′,6′-dimethoxybiphenyl (5.25 mg, 0.013 mmol), Pd(OAc)₂ (1.435 mg, 6.39 μmol) and potassium phosphate tribasic (102 mg, 0.479 mmol) in 1,4-dioxane (1065 μl) and water (213 μl) under positive pressure of N₂. The reaction was heated at 80° C. for 18 h. The reaction was then diluted with EtOAc and water. The organic layer was washed with brine, collected, dried over MgSO₄, filtered and volatiles evaporated to afford the crude product. The crude product was purified via silica gel (12 g column, 5-75% EtOAc:Hex) to afford the semi pure product (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(4,4-dimethylpiperidine-1-carbonyl)-2-methylpyridin-3-yl)acetate (11 mg, 0.014 mmol, 21.91% yield) as a light brown oil. LCMS (M+1)=785.3.

### Example 118

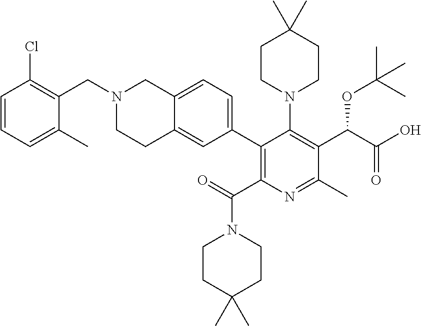 [SMILES: Cc1cccc(Cl)c1CN1CCc2cc(-c3c(C(=O)N4CCC(C)(C)CC4)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-6-(4,4-dimethylpiperidine-1-carbonyl)-2-methylpyridin-3-yl)acetic Acid

To a solution of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(4,4-dimethylpiperidine-1-carbonyl)-2-methylpyridin-3-yl)acetate (11 mg, 0.014 mmol) in EtOH (1 mL) and water (0.111 mL) was added lithium hydroxide monohydrate (5.88 mg, 0.140 mmol) and heated at 75° C. for 18 hrs. The reaction was cooled to RT, filtered through a nylon 0.45 μ frit filter purified via preparative LC/MS to afford the product (S)-2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(4,4-dimethylpiperidine-1-carbonyl)-2-methylpyridin-3-yl)acetic acid (3.1 mg, 4.13 μmol, 29.5% yield). The product appeared to be a mixture of atropisomers in a 56:44 ratio according to these reference peaks ¹H NMR (500 MHz, DMSO-d₆) δ 5.65 (br s, 1H), 5.62 (br s, 0.8H).

Full spectrum: ¹H NMR (500 MHz, DMSO-d₆) δ 7.29 (d, J=7.6 Hz, 1H), 7.23-7.15 (m, 2H), 7.13-7.01 (m, 2H), 6.89-6.84 (m, 1H), 5.64 (br d, J=13.4 Hz, 1H), 2.45-2.40 (m, 7H), 2.21-1.93 (m, 3H), 1.62-1.48 (m, 1H), 1.25 (br d, J=13.1 Hz, 2H), 1.10 (d, J=2.7 Hz, 9H), 0.90-0.78 (m, 7H), 0.74-0.59 (m, 7H). 15 protons (combination of piperidine and tetrahydroisoquinoline protons) were not resolved via ¹H NMR due to water peak. LCMS (M+1)=743.2.

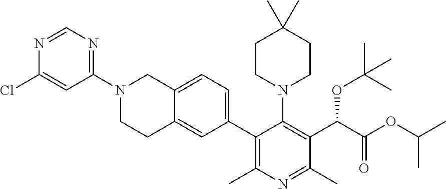 [SMILES: Cc1nc(C)c([C@H](OC(C)(C)C)C(=O)OC(C)C)c(N2CCC(C)(C)CC2)c1-c1ccc2c(c1)CCN(c1cc(Cl)ncn1)C2]

### Isopropyl (S)-2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate

In a pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (70 mg, 0.134 mmol) and 4,6-dichloropyrimidine (44.0 mg, 0.295 mmol) in ethanol (4 mL). Hunig's base (0.117 mL, 0.671 mmol) was added and the mixture was heated to 80° C. in an oil bath and allowed to stir for 16 hours overnight. The next day, LC/MS showed desired product as major. Cooled mixture to RT, and evaporated solvents to give 84 mg of crude mixture containing (S)-2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetic acid. The crude mixture was divided into portions to use for preparation of examples described below. LCMS (M+1)=634.45.

### Example 119

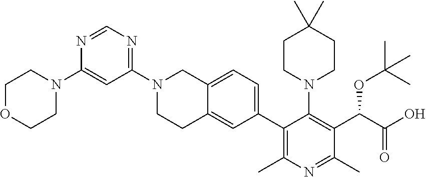 [SMILES: Cc1nc(C)c([C@H](OC(C)(C)C)C(=O)O)c(N2CCC(C)(C)CC2)c1-c1ccc2c(c1)CCN(c1cc(N3CCOCC3)ncn1)C2]

### (S)-2-(tert-Butoxy)-2-(4-(4,4-dimethylpiperidin-1-yl)-2,6-dimethyl-5-(2-(6-morpholinopyrimidin-4-yl)-1,2,3,4-tetrahydroisoquinolin-6-yl)pyridin-3-yl)acetic Acid

In a pressure vial equipped with a magnetic stirring bar was added isopropyl (S)-2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (28 mg, 0.044 mmol) and morpholine (23.08 mg, 0.265 mmol) in ethanol (2 mL). The mixture was heated to 100° C. in a preheated aluminum block and allowed to stir for 48 hours. LC/MS showed the desired ester intermediate. LCMS (M+1)=685. The reaction mixture was cooled to RT and 10M sodium hydroxide (0.022 mL, 0.221 mmol) was added. The vial was capped and heated to 80° C. for 3 hours. LC/MS showed that the hydrolysis was complete. The reaction mixture was cooled, then filtered and was purified via preparative LC/MS to give 5.5 mg (19%) of the title compound. LCMS (M+1)=664.3. ¹H NMR (500 MHz, DMSO-d₆) δ 8.14 (s, 1H), 7.37-7.27 (m, 1H), 7.17 (d, J=4.0 Hz, 1H), 7.02-6.93 (m, 1H), 6.02-5.92 (m, 1H), 5.73 (d, J=12.5 Hz, 1H), 4.86 (d, J=17.2 Hz, 1H), 4.69 (t, J=16.0 Hz, 1H), 3.85 (d, J=5.9 Hz, 1H), 3.66 (d, J=4.4 Hz, 3H), 3.54 (d, J=5.1 Hz, 2H), 2.90 (d, J=5.1 Hz, 2H), 2.79 (br. s., 1H), 2.55 (s, 6H), 2.43 (s, 3H), 2.11 (s, 3H), 1.91 (s, 3H), 1.48 (br. s., 1H), 1.27 (d, J=13.2 Hz, 1H), 1.16 (d, J=10.3 Hz, 1H), 1.14-1.06 (m, 8H), 1.04-0.90 (m, 1H), 0.86-0.77 (m, 3H), 0.59 (s, 1H), 0.50 (s, 2H)□

### Example 120

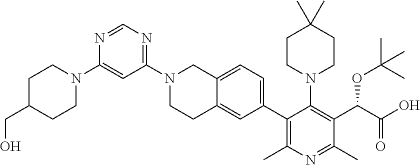 [SMILES: Cc1nc(C)c([C@H](OC(C)(C)C)C(=O)O)c(N2CCC(C)(C)CC2)c1-c1ccc2c(c1)CCN(c1cc(N3CCC(CO)CC3)ncn1)C2]

### (S)-2-(tert-Butoxy)-2-(4-(4,4-dimethylpiperidin-1-yl)-5-(2-(6-(4-(hydroxymethyl)piperidin-1-yl)pyrimidin-4-yl)-1,2,3,4-tetrahydroisoquinolin-6-yl)-2,6-dimethylpyridin-3-yl)acetic Acid

In a 20 mL pressure vial equipped with a magnetic stirring bar was added isopropyl (S)-2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (28 mg, 0.044 mmol) and piperidin-4-yl methanol (30.5 mg, 0.265 mmol) in ethanol (2 mL). The mixture was heated to 100° C. in a preheated aluminum block and allowed to stir for 48 hours. LC/MS showed the desired ester intermediate. After cooling to RT, 10M sodium hydroxide (0.022 mL, 0.221 mmol) was added, the vial was capped and heated to 80° C. for 3 hours. LC/MS showed that the hydrolysis was complete. The reaction mixture was cooled, then filtered and was purified via preparative LC/MS to give 7.2 mg (24%) of the title compound. LCMS (M+1)=671.3. ¹H NMR (500 MHz, DMSO-d₆) δ 8.09 (s, 1H), 7.39-7.29 (m, 1H), 7.16 (d, J=3.7 Hz, 1H), 7.06-6.90 (m, 1H), 6.01-5.88 (m, 1H), 5.75 (d, J=12.8 Hz, 1H), 4.92-4.76 (m, 1H), 4.67 (t, J=17.4 Hz, 1H), 4.42 (d, J=14.3 Hz, 2H), 3.91 (s, 1H), 3.82 (d, J=5.9 Hz, 1H), 3.26 (d, J=5.5 Hz, 1H), 2.91 (br. s., 2H), 2.78 (t, J=11.9 Hz, 3H), 2.55 (s, 6H), 2.44 (s, 3H), 2.11 (s, 2H), 2.07 (s, 1H), 1.91 (s, 4H), 1.70 (d, J=12.8 Hz, 2H), 1.65 (br. s., 1H), 1.48 (br. s., 1H), 1.31-1.20 (m, 1H), 1.16 (d, J=12.1 Hz, 1H), 1.12 (d, J=2.6 Hz, 8H), 1.07-0.97 (m, 1H), 0.94 (d, J=12.1 Hz, 1H), 0.87-0.78 (m, 2H), 0.59 (s, 1H), 0.49 (s, 1H).

### Example 121

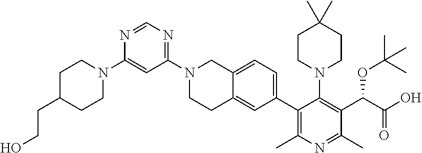 [SMILES: Cc1nc(C)c([C@H](OC(C)(C)C)C(=O)O)c(N2CCC(C)(C)CC2)c1-c1ccc2c(c1)CCN(c1cc(N3CCC(CCO)CC3)ncn1)C2]

### (S)-2-(tert-Butoxy)-2-(4-(4,4-dimethylpiperidin-1-yl)-5-(2-(6-(4-(2-hydroxyethyl)piperidin-1-yl)pyrimidin-4-yl)-1,2,3,4-tetrahydroisoquinolin-6-yl)-2,6-dimethylpyridin-3-yl)acetic Acid

In a 20 mL pressure vial equipped with a magnetic stirring bar was added isopropyl (S)-2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (28 mg, 0.044 mmol) and 2-(piperidin-4-yl)ethanol (34.2 mg, 0.265 mmol) in ethanol (2 mL). The mixture was heated to 100° C. in a preheated aluminum block and allowed to stir for 48 hours. LC/MS showed the desired ester intermediate. After cooling to RT, added 10M sodium hydroxide (0.022 mL, 0.221 mmol), capped vial and heated to 80° C. for 3 hours. LC/MS showed that the hydrolysis was complete. The reaction mixture was cooled, then filtered and was purified via preparative LC/MS to give 10.9 mg (35%) of the title compound. LCMS (M+1)=685.2. ¹H NMR (500 MHz, DMSO-d₆) δ 8.09 (s, 1H), 7.38-7.28 (m, 1H), 7.17 (d, J=3.3 Hz, 1H), 7.04-6.91 (m, 1H), 5.97-5.88 (m, 1H), 5.75 (d, J=12.5 Hz, 1H), 4.90-4.75 (m, 1H), 4.75-4.59 (m, 1H), 4.39 (d, J=13.2 Hz, 2H), 3.91 (s, 1H), 3.84-3.76 (m, 1H), 3.47 (t, J=6.6 Hz, 1H), 2.90 (br. s., 2H), 2.78 (t, J=12.3 Hz, 2H), 2.55 (s, 6H), 2.47-2.39 (m, 2H), 2.12 (s, 2H), 2.07 (s, 1H), 1.91 (s, 4H), 1.81-1.74 (m, 1H), 1.70 (d, J=12.1 Hz, 4H), 1.48 (br. s., 1H), 1.37 (d, J=6.6 Hz, 2H), 1.24 (br. s., 1H), 1.16 (d, J=13.2 Hz, 1H), 1.12 (d, J=2.6 Hz, 8H), 1.04 (br. s., 1H), 0.93 (d, J=11.4 Hz, 1H), 0.87-0.78 (m, 2H), 0.59 (s, 1H), 0.49 (s, 2H).

### Example 122

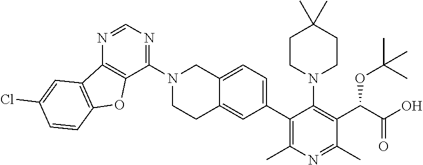 [SMILES: Cc1nc(C)c([C@H](OC(C)(C)C)C(=O)O)c(N2CCC(C)(C)CC2)c1-c1ccc2c(c1)CCN(c1ncnc3c1oc1ccc(Cl)cc13)C2]

### (S)-2-(tert-Butoxy)-2-(5-(2-(8-chlorobenzofuro[3,2-d]pyrimidin-4-yl)-1,2,3,4-tetrahydroisoquinolin-6-yl)-4-(4,4-dimethylpiperidin-1-yl)-2,6-dimethylpyridin-3-yl)acetic Acid

In a 20 mL pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (30 mg, 0.058 mmol), and 4,8-dichlorobenzofuro[3,2-d]pyrimidine (27.5 mg, 0.115 mmol) in ethanol (2 mL). Hunig's base (0.050 mL, 0.288 mmol) was added and the mixture was heated to 100° C. in a preheated aluminum block and allowed to stir for 48 hours. LC/MS showed the desired ester intermediate. After cooling to RT, 10 M sodium hydroxide (0.058 mL, 0.575 mmol) was added, the vial capped and heated to 80° C. for 3 hours. LC/MS showed that the hydrolysis was complete. The reaction mixture was cooled, then filtered and was purified via preparative LC/MS to give 16.1 mg (41%) of the title compound. LCMS (M+1)=682.1. ¹H NMR (500 MHz, DMSO-d₆) δ 8.60 (s, 1H), 8.15 (s, 1H), 8.01-7.89 (m, 1H), 7.76 (d, J=9.2 Hz, 1H), 7.52-7.37 (m, 1H), 7.28-7.18 (m, 1H), 7.03 (br. s., 1H), 5.84 (d, J=14.3 Hz, 1H), 5.33 (d, J=16.9 Hz, 1H), 5.28-5.05 (m, 1H), 4.45-4.27 (m, 2H), 3.16-3.01 (m, 2H), 2.82 (br. s., 1H), 2.55 (s, 1H), 2.45 (s, 3H), 2.13 (s, 2H), 2.09 (s, 1H), 1.92 (s, 1H), 1.45 (br. s., 1H), 1.24 (br. s., 1H), 1.17 (br. s., 1H), 1.13 (d, J=2.6 Hz, 10H), 0.99 (br. s., 1H), 0.91 (d, J=13.6 Hz, 1H), 0.85-0.75 (m, 3H), 0.54 (br. s., 1H), 0.41 (s, 2H).

### Example 123

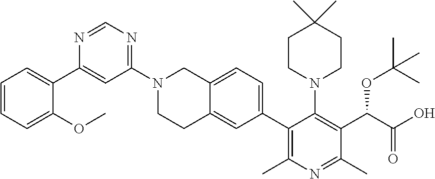 [SMILES: COc1ccccc1-c1cc(N2CCc3cc(-c4c(C)nc(C)c([C@H](OC(C)(C)C)C(=O)O)c4N4CCC(C)(C)CC4)ccc3C2)ncn1]

### (S)-2-(tert-Butoxy)-2-(4-(4,4-dimethylpiperidin-1-yl)-5-(2-(6-(2-methoxyphenyl)pyrimidin-4-yl)-1,2,3,4-tetrahydroisoquinolin-6-yl)-2,6-dimethylpyridin-3-yl)acetic Acid

In a 20 mL pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (29 mg, 0.056 mmol), and 4-chloro-6-(2-methoxyphenyl)pyrimidine (49.1 mg, 0.111 mmol) in ethanol (2 mL). Hunig's base (0.049 mL, 0.278 mmol) was added and the mixture was heated to 100° C. in a preheated aluminum block and allowed to stir for 16 hours. The reaction was followed by LC/MS. LC/MS showed the desired product. After cooling to RT, 10 M sodium hydroxide (0.056 mL, 0.556 mmol) was and the reaction was heated to 100° C. for 16 hours. LC/MS showed the desired acid as a minor product. The reaction mixture was cooled, then filtered and was purified via preparative LC/MS to give 2.9 mg (8%) of the title compound. LCMS (M+1)=664.2. ¹H NMR (500 MHz, DMSO-d₆) δ 8.73 (br. s., 1H), 7.74 (d, J=7.3 Hz, 1H), 7.53 (br. s., 1H), 7.47 (d, J=8.4 Hz, 1H), 7.34 (br. s., 2H), 7.31-7.19 (m, 5H), 7.19-7.09 (m, 5H), 7.09-7.00 (m, 4H), 5.62 (br. s., 1H), 5.03 (br. s., 1H), 4.90 (d, J=18.3 Hz, 1H), 4.04 (br. s., 1H), 3.95-3.80 (m, 4H), 3.03 (br. s., 2H), 2.67-2.58 (m, 3H), 2.55 (s, 9H), 2.26 (d, J=13.9 Hz, 3H), 1.31-1.17 (m, 5H), 1.14 (s, 11H), 1.07 (s, 1H), 0.76 (br. s., 3H), 0.71 (br. s., 4H).

### Example 124

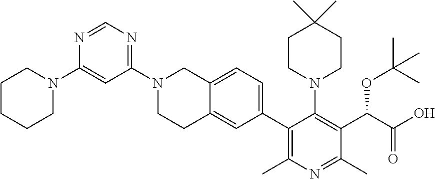 [SMILES: Cc1nc(C)c([C@H](OC(C)(C)C)C(=O)O)c(N2CCC(C)(C)CC2)c1-c1ccc2c(c1)CCN(c1cc(N3CCCCC3)ncn1)C2]

### (S)-2-(tert-Butoxy)-2-(4-(4,4-dimethylpiperidin-1-yl)-2,6-dimethyl-5-(2-(6-(piperidin-1-yl)pyrimidin-4-yl)-1,2,3,4-tetrahydroisoquinolin-6-yl)pyridin-3-yl)acetic Acid

In a 20 mL pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (29 mg, 0.056 mmol), and 4-chloro-6-(piperidin-1-yl)pyrimidine (21.97 mg, 0.111 mmol) in ethanol (2 mL). Hunig's base (0.049 mL, 0.278 mmol) was added and the mixture was heated to 100° C. in a preheated aluminum block and allowed to stir for 16 hours. LC/MS showed desired product and some remaining SM. Cooled reaction to RT, added 10 M sodium hydroxide (0.056 mL, 0.556 mmol) and heated to 100° C. for 16 hours. LC/MS showed the hydrolysis was complete. The reaction mixture was cooled, then filtered and was purified via preparative LC/MS to give 6.9 mg (19%) of the title compound. LCMS (M+1)=641.3. ¹H NMR (500 MHz, DMSO-d₆) δ 8.09 (s, 1H), 7.39-7.28 (m, 1H), 7.17 (br. s., 1H), 6.98 (s, 1H), 5.93 (d, J=4.4 Hz, 1H), 5.81 (d, J=12.1 Hz, 1H), 4.85 (d, J=16.9 Hz, 1H), 4.67 (t, J=16.7 Hz, 1H), 3.91 (s, 1H), 3.84-3.65 (m, 1H), 3.58 (br. s., 2H), 2.90 (br. s., 2H), 2.82 (br. s., 1H), 2.55 (s, 8H), 2.47-2.39 (m, 3H), 2.12 (s, 2H), 2.08 (s, 1H), 1.91 (s, 4H), 1.62 (d, J=4.0 Hz, 1H), 1.50 (br. s., 4H), 1.31-1.18 (m, 1H), 1.16-1.08 (m, 6H), 1.01 (d, J=12.8 Hz, 1H), 0.94 (d, J=10.6 Hz, 1H), 0.88-0.77 (m, 2H), 0.59 (s, 1H), 0.49 (s, 2H).

### Example 125

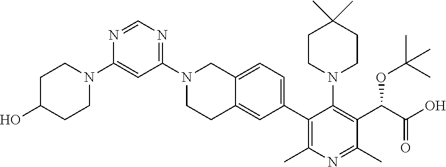 [SMILES: Cc1nc(C)c([C@H](OC(C)(C)C)C(=O)O)c(N2CCC(C)(C)CC2)c1-c1ccc2c(c1)CCN(c1cc(N3CCC(O)CC3)ncn1)C2]

### (S)-2-(tert-Butoxy)-2-(4-(4,4-dimethylpiperidin-1-yl)-5-(2-(6-(4-hydroxypiperidin-1-yl)pyrimidin-4-yl)-1,2,3,4-tetrahydroisoquinolin-6-yl)-2,6-dimethylpyridin-3-yl)acetic Acid

In a 20 mL pressure vial equipped with a magnetic stirring bar was added (S)-isopropyl 2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (30 mg, 0.047 mmol) and piperidin-4-ol (28.7 mg, 0.284 mmol) in ethanol (2 mL). The mixture was heated to 100° C. in a preheated aluminum block and allowed to stir for 16 hours. LC/MS showed desired product but also some remaining starting material. Continue to heat at 100° C. with stirring for 16 additional hours. LC/MS showed the desired ester intermediate and less starting material than the previous time point. After cooling to RT, 10M sodium hydroxide (0.022 mL, 0.221 mmol) was added, the vial capped and heated to 80° C. for 3 hours. LC/MS showed the hydrolysis was complete. The reaction mixture was cooled, then filtered and was purified via preparative LC/MS to give 10.4 mg (33%) of the title compound. LCMS (M+1)=657.3. ¹H NMR (500 MHz, DMSO-d₆) δ 8.10 (s, 1H), 7.39-7.27 (m, 1H), 7.16 (br. s., 1H), 7.04-6.91 (m, 1H), 6.01-5.93 (m, 1H), 5.80 (d, J=12.8 Hz, 1H), 4.85 (d, J=16.5 Hz, 1H), 4.67 (t, J=16.7 Hz, 1H), 4.07 (d, J=12.5 Hz, 2H), 3.82 (s, 1H), 3.72 (d, J=5.1 Hz, 1H), 3.19-3.07 (m, 2H), 2.91 (br. s., 2H), 2.55 (s, 6H), 2.44 (s, 3H), 2.12 (s, 2H), 2.08 (s, 1H), 1.91 (s, 3H), 1.76 (d, J=7.0 Hz, 2H), 1.47 (br. s., 1H), 1.39-1.21 (m, 2H), 1.17 (d, J=12.5 Hz, 1H), 1.12 (d, J=2.2 Hz, 10H), 0.96 (br. s., 1H), 0.87-0.78 (m, 2H), 0.59 (s, 1H), 0.49 (s, 2H).

### Example 126

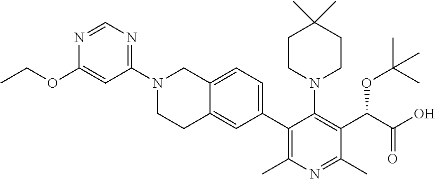 [SMILES: CCOc1cc(N2CCc3cc(-c4c(C)nc(C)c([C@H](OC(C)(C)C)C(=O)O)c4N4CCC(C)(C)CC4)ccc3C2)ncn1]

### (S)-2-(tert-Butoxy)-2-(4-(4,4-dimethylpiperidin-1-yl)-5-(2-(6-ethoxypyrimidin-4-yl)-1,2,3,4-tetrahydroisoquinolin-6-yl)-2,6-dimethylpyridin-3-yl)acetic Acid

In a 20 mL pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (29 mg, 0.056 mmol), and 4-chloro-6-(4-methyl-1H-imidazol-1-yl)pyrimidine (21.64 mg, 0.111 mmol) in ethanol (2 mL). Hunig's base (0.049 mL, 0.278 mmol) was added and the mixture was heated to 100° C. in a preheated aluminum block and allowed to stir for 16 hours. LC/MS showed desired coupled ester product as major. Cooled the reaction to RT, and 10 M sodium hydroxide (0.056 mL, 0.556 mmol) was added. The mixture was heated to 100° C. for 16 hours. LC/MS did not show the coupled hydrolysis product. The reaction mixture was cooled, then filtered and was purified via preparative LC/MS to give 14.2 mg (42%) of the title compound. LCMS (M+1)=602.2. ¹H NMR (500 MHz, DMSO-d₆) δ 8.28 (s, 1H), 7.40-7.31 (m, 1H), 7.24-7.14 (m, 1H), 7.00 (s, 1H), 6.12 (s, 1H), 5.85 (d, J=12.5 Hz, 1H), 4.87 (d, J=16.1 Hz, 1H), 4.68 (d, J=16.5 Hz, 1H), 4.30 (q, J=7.0 Hz, 2H), 3.85 (br. s., 1H), 2.93 (br. s., 2H), 2.82 (br. s., 1H), 2.55 (s, 3H), 2.44 (s, 3H), 2.12 (s, 2H), 2.08 (s, 1H), 1.92 (s, 1H), 1.47 (br. s., 1H), 1.30 (t, J=7.0 Hz, 4H), 1.25 (br. s., 1H), 1.17 (d, J=15.8 Hz, 1H), 1.16-1.09 (m, 8H), 1.01 (br. s., 1H), 0.94 (d, J=11.7 Hz, 1H), 0.88-0.76 (m, 3H), 0.57 (s, 1H), 0.46 (s, 2H). LCMS and NMR confirmed the title compound formed from the hydrolysis reaction in the presence of ethanol.

### Example 127

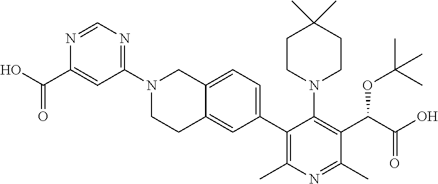 [SMILES: Cc1nc(C)c([C@H](OC(C)(C)C)C(=O)O)c(N2CCC(C)(C)CC2)c1-c1ccc2c(c1)CCN(c1cc(C(=O)O)ncn1)C2]

### (S)-6-(6-(5-(tert-Butoxy(carboxy)methyl)-4-(4,4-dimethylpiperidin-1-yl)-2,6-dimethylpyridin-3-yl)-3,4-dihydroisoquinolin-2(1H)-yl)pyrimidine-4-carboxylic Acid

In a 20 mL pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (29 mg, 0.056 mmol), and 2-(6-chloropyrimidin-4-yl)-5-methyl-1,3,4-oxadiazole (21.85 mg, 0.111 mmol) in ethanol (2 mL). Hunig's base (0.049 mL, 0.278 mmol) was added and the mixture was heated to 100° C. in a preheated aluminum block and allowed to stir for 16 hours. LC/MS showed desired coupling ester product as major. Cooled mixture to RT, and 10 M sodium hydroxide (0.056 mL, 0.556 mmol) was added. The reaction was heated to 100° C. for 16 hours. LC/MS did not show the coupled hydrolysis product. The reaction mixture was cooled, then filtered and was purified via preparative LC/MS to give 5.6 mg (15%) of the title compound. LCMS (M+1)=602.2. ¹H NMR (500 MHz, DMSO-d₆) δ 8.50 (br. s., 1H), 7.46-7.32 (m, 1H), 7.19 (br. s., 2H), 7.00 (s, 1H), 5.74 (d, J=11.4 Hz, 1H), 4.88 (br. s., 1H), 4.84-4.66 (m, 1H), 3.91 (s, 1H), 2.96 (br. s., 2H), 2.81 (br. s., 1H), 2.55 (s, 7H), 2.46-2.41 (m, 3H), 2.16-2.08 (m, 1H), 2.08-2.02 (m, 1H), 1.91 (s, 4H), 1.77 (s, 1H), 1.47 (br. s., 1H), 1.23 (d, J=9.9 Hz, 1H), 1.11 (s, 6H), 0.95 (d, J=11.7 Hz, 1H), 0.87-0.76 (m, 2H), 0.67-0.54 (m, 1H), 0.50 (s, 2H). LCMS and NMR confirmed the title compound formed from the hydrolysis reaction in the presence of ethanol.

### Example 128

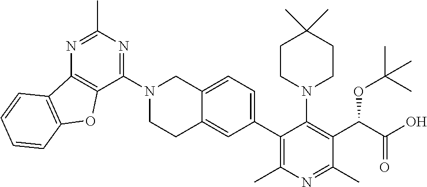 [SMILES: Cc1nc(N2CCc3cc(-c4c(C)nc(C)c([C@H](OC(C)(C)C)C(=O)O)c4N4CCC(C)(C)CC4)ccc3C2)c2oc3ccccc3c2n1]

### (S)-2-(tert-Butoxy)-2-(4-(4,4-dimethylpiperidin-1-yl)-2,6-dimethyl-5-(2-(2-methylbenzofuro[3,2-d]pyrimidin-4-yl)-1,2,3,4-tetrahydroisoquinolin-6-yl)pyridin-3-yl)acetic Acid

In a 20 mL pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20 mg, 0.038 mmol), and 4-chloro-2-methylbenzofuro[3,2-d]pyrimidine (10.48 mg, 0.048 mmol) in ethanol (2 mL). Hunig's base (0.033 mL, 0.192 mmol) was added and the mixture was heated to 100° C. in a preheated aluminum block and allowed to stir for 48 hours. LC/MS showed the desired ester intermediate. After cooling to RT, added 10M sodium hydroxide (0.038 mL, 0.383 mmol), capped vial and heated to 80° C. for 3 hours. LC/MS showed that the hydrolysis was complete. The reaction mixture was cooled, then filtered and was purified via preparative LC/MS to give 12.0 mg (47%) of the title compound. LCMS (M+1)=662.2. ¹H NMR (500 MHz, METHANOL-d4) δ 8.17 (d, J=7.7 Hz, 1H), 7.75 (d, J=8.4 Hz, 1H), 7.69 (t, J=7.9 Hz, 1H), 7.56-7.43 (m, 2H), 7.37-7.27 (m, 1H), 7.12-7.05 (m, 1H), 5.53 (br. s., 1H), 5.45-5.32 (m, 2H), 4.50 (br. s., 1H), 4.46 (br. s., 1H), 3.17 (br. s., 2H), 2.81 (d, J=15.0 Hz, 2H), 2.70 (s, 3H), 2.66 (s, 3H), 2.35 (s, 2H), 2.32 (s, 2H), 1.32 (br. s., 3H), 1.27 (s, 1H), 1.20 (d, J=3.7 Hz, 10H), 0.81 (s, 3H), 0.74 (br. s., 3H).

### Example 129

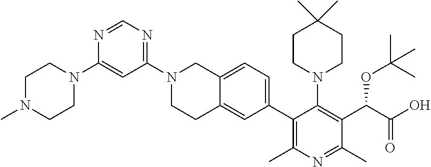 [SMILES: Cc1nc(C)c([C@H](OC(C)(C)C)C(=O)O)c(N2CCC(C)(C)CC2)c1-c1ccc2c(c1)CCN(c1cc(N3CCN(C)CC3)ncn1)C2]

### (S)-2-(tert-Butoxy)-2-(4-(4,4-dimethylpiperidin-1-yl)-2,6-dimethyl-5-(2-(6-(4-methylpiperazin-1-yl)pyrimidin-4-yl)-1,2,3,4-tetrahydroisoquinolin-6-yl)pyridin-3-yl)acetic Acid

In a 20 mL pressure vial equipped with a magnetic stirring bar was added (S)-isopropyl 2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (30 mg, 0.047 mmol) and 1-methylpiperazine (28.4 mg, 0.284 mmol) in ethanol (2 mL). The mixture was heated to 100° C. in a preheated aluminum block and allowed to stir for 16 hours. LC/MS showed desired product but also some remaining starting material. Continue to heat at 100° C. with stirring for an additional 16 hours. LC/MS now showed complete conversion to the desired ester intermediate. After cooling to RT, 10M sodium hydroxide (0.022 mL, 0.221 mmol) was added, the vial was capped and heated to 80° C. for 16 hours. LC/MS showed the hydrolysis was complete. The reaction mixture was cooled, then filtered and was purified via preparative LC/MS to give 9.6 mg (31%) of the title compound. LCMS (M+1)=656.3. ¹H NMR (500 MHz, DMSO-d6)) 8.11 (s, 1H), 7.38-7.28 (m, 1H), 7.16 (br. s., 1H), 7.05-6.94 (m, 1H), 6.03-5.93 (m, 1H), 5.82 (d, J=13.2 Hz, 1H), 4.86 (d, J=16.9 Hz, 1H), 4.68 (t, J=16.7 Hz, 1H), 3.91 (s, 1H), 3.82 (d, J=5.9 Hz, 1H), 3.56 (br. s., 1H), 2.91 (br. s., 1H), 2.81 (br. s., 1H), 2.55 (s, 6H), 2.47-2.40 (m, 1H), 2.36 (br. s., 4H), 2.21 (s, 4H), 2.12 (s, 3H), 2.08 (s, 2H), 1.91 (s, 1H), 1.47 (br. s., 1H), 1.32-1.21 (m, 1H), 1.17 (d, J=12.8 Hz, 1H), 1.12 (d, J=2.2 Hz, 9H), 1.01 (d, J=12.8 Hz, 1H), 0.94 (d, J=12.1 Hz, 1H), 0.88-0.77 (m, 3H), 0.58 (s, 1H), 0.49 (s, 1H).

### Example 130

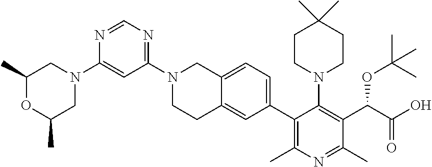 [SMILES: Cc1nc(C)c([C@H](OC(C)(C)C)C(=O)O)c(N2CCC(C)(C)CC2)c1-c1ccc2c(c1)CCN(c1cc(N3C[C@@H](C)O[C@@H](C)C3)ncn1)C2]

### (S)-2-(tert-Butoxy)-2-(5-(2-(6-((2S,6R)-2,6-dimethylmorpholino)pyrimidin-4-yl)-1,2,3,4-tetrahydroisoquinolin-6-yl)-4-(4,4-dimethylpiperidin-1-yl)-2,6-dimethylpyridin-3-yl)acetic Acid

In a 20 mL pressure vial equipped with a magnetic stirring bar was added (S)-isopropyl 2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (30 mg, 0.047 mmol) and cis-2,6-dimethylmorpholine (32.7 mg, 0.284 mmol) in ethanol (2 mL). The mixture was heated to 100° C. in a preheated aluminum block and allowed to stir for 48 hours. LC/MS showed desired product. After cooling to RT, 10M sodium hydroxide (0.022 mL, 0.221 mmol) was added, the vial capped and heated to 80° C. for 16 hours. LC/MS showed the hydrolysis was complete. The reaction mixture was cooled, then filtered and was purified via preparative LC/MS to give 1.6 mg (5%) of the title compound. LCMS (M+1)=671.2. ¹H NMR (500 MHz, DMSO-d₆) δ 8.12 (s, 1H), 7.38-7.29 (m, 1H), 7.17 (br. s., 1H), 6.98 (s, 1H), 5.98 (br. s., 1H), 5.77 (d, J=12.5 Hz, 1H), 4.87 (d, J=16.9 Hz, 1H), 4.70 (t, J=16.5 Hz, 1H), 4.28 (d, J=10.6 Hz, 2H), 4.02-3.85 (m, 1H), 3.83 (d, J=6.6 Hz, 1H), 3.56 (br. s., 1H), 2.91 (d, J=5.9 Hz, 2H), 2.80 (br. s., 1H), 2.55 (s, 3H), 2.46-2.35 (m, 4H), 2.15-2.04 (m, 4H), 1.48 (br. s., 1H), 1.26 (d, J=18.3 Hz, 1H), 1.16 (d, J=5.9 Hz, 8H), 1.13-1.05 (m, 10H), 1.01 (d, J=13.6 Hz, 1H), 0.94 (d, J=11.0 Hz, 1H), 0.89-0.77 (m, 2H), 0.59 (s, 1H), 0.50 (s, 2H).

### Example 131

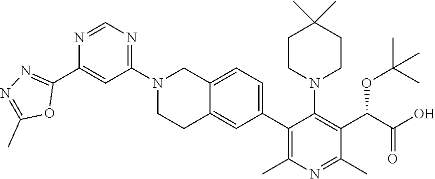 [SMILES: Cc1nnc(-c2cc(N3CCc4cc(-c5c(C)nc(C)c([C@H](OC(C)(C)C)C(=O)O)c5N5CCC(C)(C)CC5)ccc4C3)ncn2)o1]

### (S)-2-(tert-Butoxy)-2-(4-(4,4-dimethylpiperidin-1-yl)-2,6-dimethyl-5-(2-(6-(5-methyl-1,3,4-oxadiazol-2-yl)pyrimidin-4-yl)-1,2,3,4-tetrahydroisoquinolin-6-yl)pyridin-3-yl)acetic Acid

In a 20 mL pressure vial equipped with a magnetic stirring bar was added (S)-2-(tert-butoxy)-2-(4-(4,4-dimethylpiperidin-1-yl)-2,6-dimethyl-5-(1,2,3,4-tetrahydroisoquinolin-6-yl)pyridin-3-yl)acetic acid (15 mg, 0.031 mmol), and 2-(6-chloropyrimidin-4-yl)-5-methyl-1,3,4-oxadiazole (12.30 mg, 0.063 mmol) in ethanol (1.5 mL). Hunig's base (0.027 mL, 0.156 mmol) was added and the mixture was heated to 100° C. in a preheated aluminum block and allowed to stir for 16 hours. LC/MS showed desired product. The reaction mixture was cooled, then filtered and was purified via preparative LC/MS to give 12.1 mg (60%) of the title compound. LCMS (M+1)=640.2. ¹H NMR (500 MHz, DMSO-d₆) δ 8.71 (s, 1H), 7.52 (s, 1H), 7.43 (t, J=8.6 Hz, 1H), 7.22 (d, J=9.2 Hz, 1H), 7.10-6.99 (m, 1H), 5.81 (d, J=11.4 Hz, 1H), 4.87 (br. s., 1H), 3.91 (s, 1H), 3.01 (br. s., 2H), 2.82 (d, J=12.5 Hz, 1H), 2.64 (s, 3H), 2.55 (s, 3H), 2.44 (s, 3H), 2.12 (s, 3H), 2.08 (s, 1H), 1.92 (s, 2H), 1.48 (br. s., 1H), 1.26 (br. s., 1H), 1.18 (d, J=13.9 Hz, 1H), 1.15-1.08 (m, 10H), 1.05-0.89 (m, 1H), 0.88-0.77 (m, 2H), 0.58 (s, 1H), 0.50 (s, 2H).

### Example 132

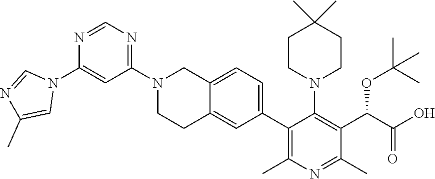 [SMILES: Cc1cn(-c2cc(N3CCc4cc(-c5c(C)nc(C)c([C@H](OC(C)(C)C)C(=O)O)c5N5CCC(C)(C)CC5)ccc4C3)ncn2)cn1]

### (S)-2-(tert-Butoxy)-2-(4-(4,4-dimethylpiperidin-1-yl)-2,6-dimethyl-5-(2-(6-(4-methyl-1H-imidazol-1-yl)pyrimidin-4-yl)-1,2,3,4-tetrahydroisoquinolin-6-yl)pyridin-3-yl)acetic Acid

In a 20 mL pressure vial equipped with a magnetic stirring bar was added (S)-2-(tert-butoxy)-2-(4-(4,4-dimethylpiperidin-1-yl)-2,6-dimethyl-5-(1,2,3,4-tetrahydroisoquinolin-6-yl)pyridin-3-yl)acetic acid (15 mg, 0.031 mmol), and 4-chloro-6-(4-methyl-1H-imidazol-1-yl)pyrimidine (12.17 mg, 0.063 mmol) in ethanol (1.5 mL). Hunig's base (0.027 mL, 0.156 mmol) was added and the mixture was heated to 100° C. in a preheated aluminum block and allowed to stir for 16 hours. LC/MS showed desired product. The reaction mixture was cooled, then filtered and was purified via preparative LC/MS to give 8.8 mg (44%) of the title compound. LCMS (M+1)=638.2. ¹H NMR (500 MHz, DMSO-d₆) δ 8.56-8.43 (m, 2H), 7.77 (br. s., 1H), 7.41-7.33 (m, 1H), 7.22 (s, 1H), 7.14-7.06 (m, 1H), 7.04 (s, 1H), 5.82 (d, J=13.6 Hz, 1H), 5.00 (br. s., 1H), 4.85 (br. s., 1H), 3.98 (br. s., 1H), 3.95-3.80 (m, 1H), 3.00 (d, J=7.3 Hz, 2H), 2.83 (br. s., 1H), 2.56 (s, 4H), 2.48-2.40 (m, 3H), 2.19 (s, 3H), 2.16-2.05 (m, 2H), 1.95-1.89 (m, 2H), 1.48 (br. s., 1H), 1.26 (br. s., 1H), 1.21-1.06 (m, 10H), 0.94 (d, J=11.0 Hz, 1H), 0.89-0.74 (m, 2H), 0.68-0.55 (m, 1H), 0.49 (s, 2H).

### Example 133

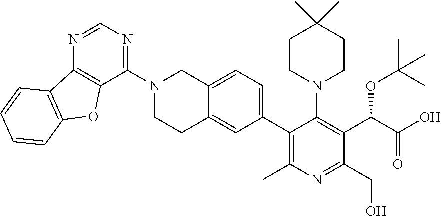 [SMILES: Cc1nc(CO)c([C@H](OC(C)(C)C)C(=O)O)c(N2CCC(C)(C)CC2)c1-c1ccc2c(c1)CCN(c1ncnc3c1oc1ccccc13)C2]

### (S)-2-(5-(2-(Benzofuro[3,2-d]pyrimidin-4-yl)-1,2,3,4-tetrahydroisoquinolin-6-yl)-4-(4,4-dimethylpiperidin-1-yl)-2-(hydroxymethyl)-6-methylpyridin-3-yl)-2-(tert-butoxy)acetic Acid

In a pressure vial equipped with a magnetic stirring bar was added (S)-isopropyl 2-(5-bromo-4-(4,4-dimethylpiperidin-1-yl)-2-(hydroxymethyl)-6-methylpyridin-3-yl)-2-(tert-butoxy)acetate (200 mg, 0.412 mmol), 4-(6-(4,4,5,5-tetramethyl-1,3,2-dioxaborolan-2-yl)-3,4-dihydroisoquinolin-2(1H)-yl)benzofuro[3,2-d]pyrimidine (264 mg, 0.618 mmol), palladium(II) acetate (11.56 mg, 0.051 mmol), S-Phos (52.8 mg, 0.103 mmol) and K₃PO₄ (656 mg, 3.09 mmol) in dioxane (6 mL) and water (1.200 mL). Argon was bubbled through the mixture for 5 minutes while sonicating. The flask was capped and heated to 80° C. within a preheated oil bath and allowed to continue for 2 hours. LC/MS showed a mixture of acid and ester products. The reaction mixture was cooled, then filtered and was purified via preparative LC/MS to give 15.0 mg (5%) of the title compound. LCMS (M+1)=664.3. ¹H NMR (500 MHz, DMSO-d₆) δ 8.59 (s, 1H), 8.13 (d, J=7.7 Hz, 1H), 7.89 (d, J=8.1 Hz, 1H), 7.74 (t, J=8.1 Hz, 1H), 7.53 (t, J=7.7 Hz, 1H), 7.49-7.42 (m, 1H), 7.29-7.20 (m, 1H), 7.10-6.98 (m, 1H), 5.76 (d, J=11.4 Hz, 1H), 5.40-5.25 (m, 1H), 5.25-5.18 (m, 1H), 4.67 (d, J=13.9 Hz, 1H), 4.56 (dd, J=13.9, 6.6 Hz, 1H), 4.37 (br. s., 1H), 3.16-3.02 (m, 2H), 2.79 (br. s., 1H), 2.55 (s, 6H), 2.24-2.07 (m, 4H), 1.47 (br. s., 1H), 1.35-1.18 (m, 1H), 1.11 (d, J=3.3 Hz, 10H), 0.99 (br. s., 1H), 0.89 (br. s., 1H), 0.84-0.73 (m, 1H), 0.67 (br. s., 1H), 0.53 (br. s., 1H), 0.40 (br. s., 1H).

### Example 134

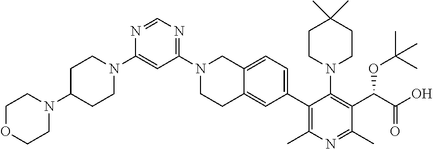 [SMILES: Cc1nc(C)c([C@H](OC(C)(C)C)C(=O)O)c(N2CCC(C)(C)CC2)c1-c1ccc2c(c1)CCN(c1cc(N3CCC(N4CCOCC4)CC3)ncn1)C2]

### (S)-2-(tert-Butoxy)-2-(4-(4,4-dimethylpiperidin-1-yl)-2,6-dimethyl-5-(2-(6-(4-morpholinopiperidin-1-yl)pyrimidin-4-yl)-1,2,3,4-tetrahydroisoquinolin-6-yl)pyridin-3-yl)acetic Acid

In a 40 mL pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (190 mg, 0.364 mmol) and 4,6-dichloropyrimidine (119 mg, 0.801 mmol) in ethanol (10 mL). Hunig's base (0.318 mL, 1.821 mmol) was added and the mixture was heated to 80° C. in an oil bath and allowed to stir for 16 hours. LC/MS showed desired product as major. Cooled to RT, and created a stock solution of 23 mg per 1 mL EtOH.

In a 20 mL pressure vial equipped with a magnetic stirring bar was added (S)-isopropyl 2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (23 mg, 0.036 mmol) and 4-(piperidin-4-yl)morpholine (24.70 mg, 0.145 mmol) in ethanol (2 mL). The mixture was heated to 100° C. in a preheated aluminum block and allowed to stir for 16 hours overnight. LC/MS showed remaining starting material, added another 4 equiv. of 4-(piperidin-4-yl)morpholine (24.70 mg, 0.145 mmol), capped and heated to 100° C. again overnight. LC/MS showed the desired ester intermediate and trace starting material. After cooling to RT, 10M sodium hydroxide (0.022 mL, 0.221 mmol) was added, the vial capped and heated to 80° C. for 3 hours. LC/MS showed that the hydrolysis was complete. The reaction mixture was cooled, then filtered and was purified via preparative LC/MS to give 9.3 mg (35%) of the title compound. LCMS (M+1)=726.2. ¹H NMR (500 MHz, DMSO-d₆) δ 8.11 (s, 1H), 7.38-7.28 (m, 1H), 7.17 (br. s., 1H), 7.00-6.93 (m, 1H), 6.00-5.95 (m, 1H), 5.77 (d, J=13.6 Hz, 1H), 4.92-4.75 (m, 1H), 4.75-4.59 (m, 1H), 4.41 (d, J=11.4 Hz, 2H), 3.85-3.77 (m, 1H), 3.56 (br. s., 2H), 2.91 (br. s., 2H), 2.81 (t, J=11.6 Hz, 3H), 2.55 (s, 4H), 2.44 (s, 3H), 2.12 (s, 2H), 2.08 (s, 1H), 1.96-1.88 (m, 4H), 1.88-1.73 (m, 3H), 1.48 (br. s., 1H), 1.30 (d, J=11.0 Hz, 3H), 1.25 (br. s., 1H), 1.17 (d, J=13.2 Hz, 1H), 1.15-1.06 (m, 10H), 1.06-0.91 (m, 1H), 0.87-0.78 (m, 4H), 0.59 (s, 1H), 0.49 (s, 2H).

### Example 135

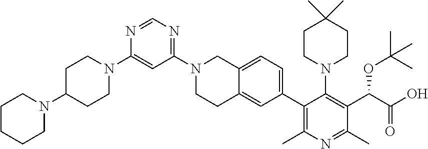 [SMILES: Cc1nc(C)c([C@H](OC(C)(C)C)C(=O)O)c(N2CCC(C)(C)CC2)c1-c1ccc2c(c1)CCN(c1cc(N3CCC(N4CCCCC4)CC3)ncn1)C2]

### (S)-2-(5-(2-(6-([1,4′-Bipiperidin]-1′-yl)pyrimidin-4-yl)-1,2,3,4-tetrahydroisoquinolin-6-yl)-4-(4,4-dimethylpiperidin-1-yl)-2,6-dimethylpyridin-3-yl)-2-(tert-butoxy)acetic Acid

In a 20 mL pressure vial equipped with a magnetic stirring bar was added (S)-isopropyl 2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (23 mg, 0.036 mmol) and 1,4′-bipiperidine (24.41 mg, 0.145 mmol) in ethanol (2 mL). The mixture was heated to 100° C. in a preheated aluminum block and allowed to stir for 16 hours overnight. LC/MS showed remaining starting material, added another 4 equiv. of 1,4′-bipiperidine (24.41 mg, 0.145 mmol), capped and heated to 100° C. again overnight. LC/MS showed the desired ester intermediate. After cooling to RT, 10M sodium hydroxide (0.022 mL, 0.221 mmol) was added, the vial capped and heated to 80° C. for 3 hours. LC/MS showed that the hydrolysis was complete. The reaction mixture was cooled, then filtered and was purified via preparative LC/MS to give 8.9 mg (34%) of the title compound. LCMS (M+1)=724.3. ¹H NMR (500 MHz, DMSO-d₆) δ 8.10 (s, 1H), 7.38-7.28 (m, 1H), 7.16 (d, J=3.3 Hz, 1H), 7.04-6.93 (m, 1H), 6.04-5.92 (m, 1H), 5.74 (d, J=12.1 Hz, 1H), 4.93-4.76 (m, 1H), 4.64 (d, J=16.9 Hz, 1H), 4.44 (d, J=10.3 Hz, 2H), 3.81 (d, J=6.6 Hz, 1H), 2.91 (br. s., 2H), 2.76 (t, J=11.7 Hz, 2H), 2.55 (s, 4H), 2.47 (br. s., 3H), 2.43 (s, 3H), 2.12 (s, 2H), 2.07 (s, 2H), 1.91 (s, 4H), 1.85-1.68 (m, 2H), 1.48 (br. s., 4H), 1.37 (br. s., 4H), 1.26 (d, J=15.8 Hz, 1H), 1.17 (br. s., 1H), 1.14-1.05 (m, 9H), 1.00 (d, J=12.1 Hz, 1H), 0.93 (d, J=12.8 Hz, 1H), 0.88-0.77 (m, 2H), 0.59 (s, 1H), 0.48 (s, 2H).

### Example 136

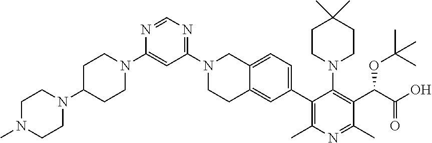 [SMILES: Cc1nc(C)c([C@H](OC(C)(C)C)C(=O)O)c(N2CCC(C)(C)CC2)c1-c1ccc2c(c1)CCN(c1cc(N3CCC(N4CCN(C)CC4)CC3)ncn1)C2]

### (S)-2-(tert-Butoxy)-2-(4-(4,4-dimethylpiperidin-1-yl)-2,6-dimethyl-5-(2-(6-(4-(4-methylpiperazin-1-yl)piperidin-1-yl)pyrimidin-4-yl)-1,2,3,4-tetrahydroisoquinolin-6-yl)pyridin-3-yl)acetic Acid

In a 20 mL pressure vial equipped with a magnetic stirring bar was added (S)-isopropyl 2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (23 mg, 0.036 mmol) and 1-methyl-4-(piperidin-4-yl)piperazine (26.6 mg, 0.145 mmol) in ethanol (2 mL). The mixture was heated to 100° C. in a preheated aluminum block and allowed to stir for 16 hours overnight. LC/MS showed remaining starting material, added another 4 equiv. of 1-methyl-4-(piperidin-4-yl)piperazine (26.6 mg, 0.145 mmol), capped vial and heated to 100° C. again overnight. LC/MS showed the desired ester intermediate. After cooling to RT, 10M sodium hydroxide (0.022 mL, 0.221 mmol) was added, the vial capped and heated to 80° C. for 3 hours. LC/MS showed that the hydrolysis was complete. The reaction mixture was cooled, then filtered and was purified via preparative LC/MS to give 14.2 mg (53%) of the title compound. LCMS (M+1)=739.3. ¹H NMR (500 MHz, DMSO-d₆) δ 8.10 (s, 1H), 7.38-7.27 (m, 1H), 7.16 (br. s., 1H), 7.01-6.92 (m, 1H), 6.03-5.90 (m, 1H), 5.73 (d, J=12.8 Hz, 1H), 4.94-4.76 (m, 1H), 4.64 (d, J=16.9 Hz, 1H), 4.42 (br. s., 2H), 3.90 (d, J=19.8 Hz, 1H), 3.82 (br. s., 1H), 2.91 (br. s., 2H), 2.80 (t, J=11.4 Hz, 2H), 2.55 (s, 4H), 2.46-2.40 (m, 4H), 2.31 (br. s., 1H), 2.20 (br. s., 1H), 2.13 (d, J=9.9 Hz, 6H), 2.08 (s, 2H), 1.91 (s, 6H), 1.85-1.71 (m, 2H), 1.48 (br. s., 1H), 1.31 (br. s., 2H), 1.25 (br. s., 1H), 1.17 (br. s., 1H), 1.14-1.05 (m, 9H), 1.05-0.89 (m, 1H), 0.89-0.76 (m, 2H), 0.59 (s, 1H), 0.48 (s, 2H).

### Example 137

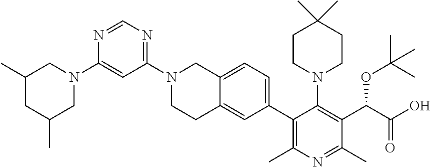 [SMILES: Cc1nc(C)c([C@H](OC(C)(C)C)C(=O)O)c(N2CCC(C)(C)CC2)c1-c1ccc2c(c1)CCN(c1cc(N3CC(C)CC(C)C3)ncn1)C2]

### (2S)-2-(tert-Butoxy)-2-(4-(4,4-dimethylpiperidin-1-yl)-5-(2-(6-(3,5-dimethylpiperidin-1-yl)pyrimidin-4-yl)-1,2,3,4-tetrahydroisoquinolin-6-yl)-2,6-dimethylpyridin-3-yl)acetic Acid

In a 20 mL pressure vial equipped with a magnetic stirring bar was added (S)-isopropyl 2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (23 mg, 0.036 mmol) and 3,5-dimethylpiperidine (30.8 mg, 0.272 mmol) in ethanol (2 mL). The mixture was heated to 100° C. in a preheated aluminum block and allowed to stir for 16 hours overnight. LC/MS showed the desired ester intermediate. After cooling to RT, 10M sodium hydroxide (0.022 mL, 0.221 mmol) was added, the vial capped and heated to 80° C. for 3 hours. LC/MS showed that the hydrolysis was complete. The reaction mixture was cooled, then filtered and was purified via preparative LC/MS to give 3.2 mg (13%) of the title compound. LCMS (M+1)=669.4. ¹H NMR (500 MHz, DMSO-d₆) δ 8.07 (s, 1H), 7.39-7.30 (m, 1H), 7.16 (br. s., 1H), 7.04-6.91 (m, 1H), 5.93 (d, J=2.9 Hz, 1H), 5.71 (d, J=12.5 Hz, 1H), 4.85 (d, J=13.2 Hz, 1H), 4.74-4.50 (m, 1H), 3.91 (s, 1H), 3.80 (br. s., 1H), 3.65 (d, J=13.9 Hz, 2H), 3.27 (dd, J=12.5, 6.6 Hz, 1H), 2.90 (br. s., 2H), 2.78 (br. s., 1H), 2.55 (s, 3H), 2.43 (s, 3H), 2.11 (s, 2H), 2.07 (s, 2H), 1.91 (s, 6H), 1.45 (t, J=5.3 Hz, 2H), 1.24 (br. s., 1H), 1.20-1.05 (m, 11H), 1.01 (br. s., 1H), 0.96-0.86 (m, 8H), 0.86-0.73 (m, 4H), 0.59 (s, 1H), 0.49 (s, 2H).

### Example 138

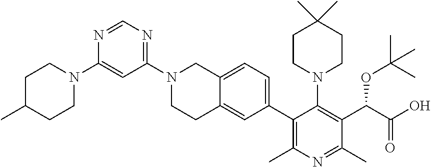 [SMILES: Cc1nc(C)c([C@H](OC(C)(C)C)C(=O)O)c(N2CCC(C)(C)CC2)c1-c1ccc2c(c1)CCN(c1cc(N3CCC(C)CC3)ncn1)C2]

### (S)-2-(tert-Butoxy)-2-(4-(4,4-dimethylpiperidin-1-yl)-2,6-dimethyl-5-(2-(6-(4-methylpiperidin-1-yl)pyrimidin-4-yl)-1,2,3,4-tetrahydroisoquinolin-6-yl)pyridin-3-yl)acetic Acid

In a 20 mL pressure vial equipped with a magnetic stirring bar was added (S)-isopropyl 2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (23 mg, 0.036 mmol) and 4-methylpiperidine (27.0 mg, 0.272 mmol) in ethanol (2 mL). The mixture was heated to 100° C. in a preheated aluminum block and allowed to stir for 16 hours overnight. LC/MS showed the desired ester intermediate. After cooling to RT, 10M sodium hydroxide (0.022 mL, 0.221 mmol) was added, the vial capped and heated to 80° C. for 3 hours. LC/MS showed that the hydrolysis was complete. The reaction mixture was cooled, then filtered and was purified via preparative LC/MS to give 7.0 mg (29%) of the title compound. LCMS (M+1)=655.3. ¹H NMR (500 MHz, DMSO-d₆) δ 8.10 (s, 1H), 7.40-7.29 (m, 1H), 7.17 (br. s., 1H), 7.05-6.93 (m, 1H), 6.00-5.91 (m, 1H), 5.82 (d, J=13.6 Hz, 1H), 4.92-4.78 (m, 1H), 4.75-4.60 (m, 1H), 4.39 (d, J=13.6 Hz, 2H), 4.03-3.85 (m, 1H), 3.82 (d, J=5.5 Hz, 1H), 2.90 (s, 3H), 2.86-2.76 (m, 2H), 2.56 (s, 2H), 2.44 (s, 3H), 2.12 (s, 3H), 2.08 (s, 1H), 1.92 (s, 1H), 1.66 (d, J=12.1 Hz, 2H), 1.48 (br. s., 1H), 1.27 (d, J=13.6 Hz, 1H), 1.18 (d, J=13.2 Hz, 1H), 1.15-1.09 (m, 10H), 1.09-0.98 (m, 3H), 0.98-0.87 (m, 4H), 0.87-0.76 (m, 3H), 0.59 (s, 1H), 0.49 (s, 2H).

### Example 139

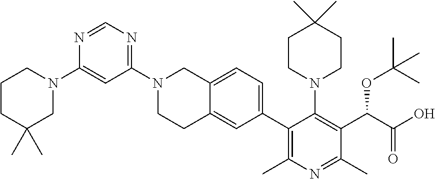 [SMILES: Cc1nc(C)c([C@H](OC(C)(C)C)C(=O)O)c(N2CCC(C)(C)CC2)c1-c1ccc2c(c1)CCN(c1cc(N3CCCC(C)(C)C3)ncn1)C2]

### (S)-2-(tert-Butoxy)-2-(4-(4,4-dimethylpiperidin-1-yl)-5-(2-(6-(3,3-dimethylpiperidin-1-yl)pyrimidin-4-yl)-1,2,3,4-tetrahydroisoquinolin-6-yl)-2,6-dimethylpyridin-3-yl)acetic Acid

In a 20 mL pressure vial equipped with a magnetic stirring bar was added 3,3-dimethylpiperidine, HCl (40.7 mg, 0.272 mmol) and Hunig's base (0.048 mL, 0.272 mmol) in ethanol (2 mL). (S)-isopropyl 2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (23 mg, 0.036 mmol) was then added. The mixture was heated to 100° C. in a preheated aluminum block and allowed to stir for 16 hours overnight. LC/MS showed the desired ester intermediate. After cooling to RT, 10M sodium hydroxide (0.022 mL, 0.221 mmol) was added, the vial capped and heated to 80° C. for 3 hours. LC/MS showed that the hydrolysis was complete. The reaction mixture was cooled, then filtered and was purified via preparative LC/MS to give 7.5 mg (21%) of the title compound as a mixture of two isomers. LCMS (M+1)=669.4. ¹H NMR (500 MHz, DMSO-d₆) δ 8.06 (s, 1H), 7.40-7.29 (m, 1H), 7.17 (br. s., 1H), 6.97 (s, 1H), 5.98-5.89 (m, 1H), 5.75 (d, J=13.2 Hz, 1H), 4.87 (d, J=16.5 Hz, 1H), 4.64 (d, J=17.2 Hz, 1H), 3.91 (s, 1H), 3.81 (s, 1H), 3.55 (br. s., 1H), 2.90 (s, 2H), 2.79 (br. s., 1H), 2.74 (s, 1H), 2.55 (s, 3H), 2.43 (s, 3H), 2.12 (s, 2H), 2.07 (s, 1H), 1.91 (s, 6H), 1.55 (br. s., 2H), 1.48 (br. s., 1H), 1.41 (br. s., 2H), 1.26 (d, J=18.3 Hz, 1H), 1.16 (d, J=9.5 Hz, 1H), 1.13-1.08 (m, 10H), 1.04-0.92 (m, 2H), 0.90 (s, 6H), 0.86-0.75 (m, 4H), 0.59 (s, 1H), 0.49 (s, 2H).

### Example 140

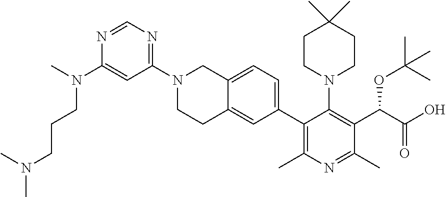 [SMILES: Cc1nc(C)c([C@H](OC(C)(C)C)C(=O)O)c(N2CCC(C)(C)CC2)c1-c1ccc2c(c1)CCN(c1cc(N(C)CCCN(C)C)ncn1)C2]

### (S)-2-(tert-Butoxy)-2-(5-(2-(6-((3-(dimethylamino)propyl)(methyl)amino)pyrimidin-4-yl)-1,2,3,4-tetrahydroisoquinolin-6-yl)-4-(4,4-dimethylpiperidin-1-yl)-2,6-dimethylpyridin-3-yl)acetic Acid

In a 20 mL pressure vial equipped with a magnetic stirring bar was added (S)-isopropyl 2-(tert-butoxy)-2-(5-(2-(6-chloropyrimidin-4-yl)-1,2,3,4-tetrahydroisoquinolin-6-yl)-4-(4,4-dimethylpiperidin-1-yl)-2,6-dimethylpyridin-3-yl)acetate (23 mg, 0.036 mmol) and N1,N1,N3-trimethylpropane-1,3-diamine (31.6 mg, 0.272 mmol) in ethanol (2 mL). The mixture was heated to 100° C. in a preheated aluminum block and allowed to stir for 16 hours overnight. LC/MS showed the desired ester intermediate. After cooling to RT, 10M sodium hydroxide (0.022 mL, 0.221 mmol) was added, the vial capped and heated to 80° C. for 3 hours. LC/MS showed that the hydrolysis was complete. The reaction mixture was cooled, then filtered and was purified via preparative LC/MS to give 4.3 mg (17%) of the title compound. LCMS (M+1)=672.4. ¹H NMR (500 MHz, DMSO-d₆) δ 8.09 (s, 1H), 7.96 (s, 1H), 7.43-7.28 (m, 1H), 7.23-7.11 (m, 1H), 6.97 (s, 1H), 5.81-5.68 (m, 2H), 4.92-4.76 (m, 1H), 4.64 (d, J=16.5 Hz, 1H), 3.91 (s, 1H), 3.84 (br. s., 1H), 3.02-2.95 (m, 3H), 2.95-2.86 (m, 4H), 2.79-2.66 (m, 2H), 2.55 (s, 2H), 2.47-2.38 (m, 2H), 2.30-2.22 (m, 2H), 2.21-2.15 (m, 4H), 2.15-2.01 (m, 4H), 1.91 (s, 8H), 1.81-1.71 (m, 1H), 1.71-1.59 (m, 2H), 1.46 (br. s., 1H), 1.36-1.21 (m, 1H), 1.11 (s, 10H), 0.99 (br. s., 1H), 0.92 (d, J=11.7 Hz, 1H), 0.86-0.76 (m, 2H), 0.55 (s, 1H), 0.46 (s, 2H).

### Example 141

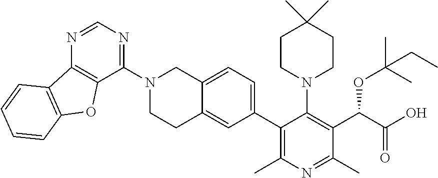 [SMILES: CCC(C)(C)O[C@H](C(=O)O)c1c(C)nc(C)c(-c2ccc3c(c2)CCN(c2ncnc4c2oc2ccccc24)C3)c1N1CCC(C)(C)CC1]

### (S)-2-(5-(2-(Benzofuro[3,2-d]pyrimidin-4-yl)-1,2,3,4-tetrahydroisoquinolin-6-yl)-4-(4,4-dimethylpiperidin-1-yl)-2,6-dimethylpyridin-3-yl)-2-(tert-pentyloxy)acetic Acid

A mixture of (S)-isopropyl 2-(5-bromo-4-(4,4-dimethylpiperidin-1-yl)-2,6-dimethylpyridin-3-yl)-2-(tert-pentyloxy)acetate (123 mg, 0.254 mmol) and 4-(6-(4,4,5,5-tetramethyl-1,3,2-dioxaborolan-2-yl)-3,4-dihydroisoquinolin-2(1H)-yl)benzofuro[3,2-d]pyrimidine (150 mg, 0.351 mmol) in distilled THF (13 mL) is flushed well with argon within a vial. Added degassed K₃PO₄ (0.5 M in water; 1.6 mL, 0.800 mmol) then 2G X-Phos precatalyst (20 mg, 0.025 mmol), sealed and stirred RT overnight. Stirred at RT for 2 days. LC/MS showed formation of the ester. The organic layer was decanted from the water layer, then removed the THF under nitrogen, take up residue in EtOAc, dry over MgSO₄, filter, and concentrate under vacuum. Purified by flash column chromatography to give 129 mg (72%) of a residue. Took up residue in ethanol (2 mL) and added 10M sodium hydroxide (0.183 mL, 1.833 mmol). The vial was capped and heated to 80° C. for 48 hours. LC/MS showed that the hydrolysis was complete. The reaction mixture was cooled, then filtered and was purified via preparative LC/MS to give 59.4 mg (47%) of the title compound. LCMS (M+1)=662.3. ¹H NMR (400 MHz, methanol-d4) δ 8.57 (s, 1H), 8.17 (d, J=7.8 Hz, 1H), 7.84-7.77 (m, 1H), 7.73 (t, J=7.8 Hz, 1H), 7.52 (t, J=7.8 Hz, 2H), 7.38-7.28 (m, 1H), 7.16-6.98 (m, 1H), 5.54 (s, 1H), 5.49-5.22 (m, 2H), 4.57-4.40 (m, 2H), 3.39-3.35 (m, 2H), 3.19 (br s, 2H), 2.83-2.63 (m, 5H), 2.32 (d, J=12.3 Hz, 3H), 1.69-1.56 (m, 1H), 1.49 (ddd, J=13.9, 7.4, 2.8 Hz, 1H), 1.42-1.27 (m, 3H), 1.23-1.15 (m, 4H), 1.13 (d, J=3.3 Hz, 4H), 0.84-0.64 (m, 9H).

### Example 142

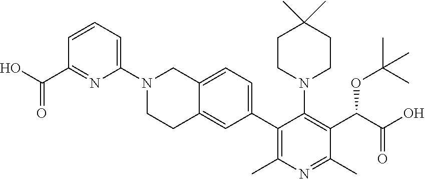 [SMILES: Cc1nc(C)c([C@H](OC(C)(C)C)C(=O)O)c(N2CCC(C)(C)CC2)c1-c1ccc2c(c1)CCN(c1cccc(C(=O)O)n1)C2]

### (S)-6-(6-(5-(tert-Butoxy(carboxy)methyl)-4-(4,4-dimethylpiperidin-1-yl)-2,6-dimethylpyridin-3-yl)-3,4-dihydroisoquinolin-2(1H)-yl)picolinic Acid

In a 20 mL pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20 mg, 0.038 mmol), and 6-fluoropicolinonitrile (46.8 mg, 0.383 mmol) in NMP (1 mL). Hunig's base (0.084 mL, 0.479 mmol) was added and the mixture was heated to 170° C. in a preheated aluminum block and allowed to stir for 16 hours overnight. LC/MS showed the desired ester intermediate. Removed NMP under a stream of nitrogen while heating at 100° C. After cooling to RT, added ethanol (1.5 mL) and 10M sodium hydroxide (0.038 mL, 0.383 mmol), capped vial and heated to 80° C. for 16 hours. LC/MS showed that the hydrolysis was complete but the nitrile had hydrolyzed.

The reaction mixture was cooled, then filtered and was purified via preparative LC/MS to give 12.0 mg (52%) of the title compound. LCMS (M+1)=601.1. ¹H NMR (500 MHz, DMSO-d₆) δ 7.68 (t, J=7.9 Hz, 1H), 7.41-7.32 (m, 1H), 7.27 (d, J=7.3 Hz, 1H), 7.20-7.14 (m, 1H), 7.05 (t, J=7.4 Hz, 1H), 6.99 (br s, 1H), 5.77 (br d, J=14.3 Hz, 1H), 4.91 (br d, J=16.5 Hz, 1H), 4.76-4.67 (m, 1H), 3.91 (s, 1H), 2.95 (br d, J=5.1 Hz, 2H), 2.55 (s, 3H), 2.49-2.41 (m, 3H), 2.18-2.04 (m, 2H), 1.91 (s, 6H), 1.48 (br s, 1H), 1.24 (br s, 1H), 1.12 (d, J=2.6 Hz, 10H), 0.88-0.77 (m, 3H), 0.59 (s, 1H), 0.49 (s, 2H).

### Example 143

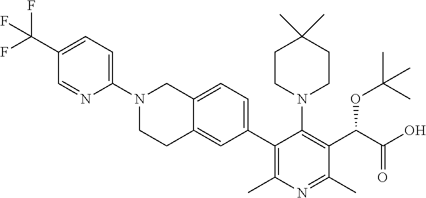 [SMILES: Cc1nc(C)c([C@H](OC(C)(C)C)C(=O)O)c(N2CCC(C)(C)CC2)c1-c1ccc2c(c1)CCN(c1ccc(C(F)(F)F)cn1)C2]

### (S)-2-(tert-butoxy)-2-(4-(4,4-dimethylpiperidin-1-yl)-2,6-dimethyl-5-(2-(5-(trifluoromethyl)pyridin-2-yl)-1,2,3,4-tetrahydroisoquinolin-6-yl)pyridin-3-yl)acetic Acid

In a 20 mL pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (20 mg, 0.038 mmol), and 2-fluoro-5-(trifluoromethyl)pyridine (63.3 mg, 0.383 mmol) in NMP (1 mL). Hunig's base (0.084 mL, 0.479 mmol) was added and the mixture was heated to 160° C. in a preheated aluminum block and allowed to stir for 16 hours overnight. LC/MS showed the desired MW product and no remaining starting material. Blew off NMP at 125° C. under a steady stream of nitrogen, took up residue in ethanol (1.5 mL) and added 10M NaOH (0.25 mL). Stirred at 90° C. overnight. LC/MS showed the desired product. The reaction mixture was cooled, then filtered and was purified via preparative LC/MS to give 10.5 mg (43%) of the title compound. LCMS (M+1)=625.1. ¹H NMR (500 MHz, DMSO-d₆) δ 8.47 (br s, 1H), 7.85 (br d, J=7.3 Hz, 1H), 7.46-7.35 (m, 1H), 7.22-7.17 (m, 1H), 7.04 (s, 1H), 7.01 (br s, 1H), 5.87-5.79 (m, 1H), 5.00-4.88 (m, 1H), 4.84-4.73 (m, 1H), 4.00-3.89 (m, 2H), 3.89-3.82 (m, 1H), 3.41 (br d, J=4.0 Hz, 1H), 3.02-2.92 (m, 2H), 2.84-2.77 (m, 1H), 2.49-2.41 (m, 3H), 2.26-2.17 (m, 1H), 2.13 (s, 2H), 2.08 (s, 1H), 1.92 (s, 1H), 1.51-1.42 (m, 1H), 1.30-1.08 (m, 11H), 1.04-0.89 (m, 1H), 0.88-0.75 (m, 3H), 0.56 (s, 1H), 0.43 (s, 2H).

### Example 144

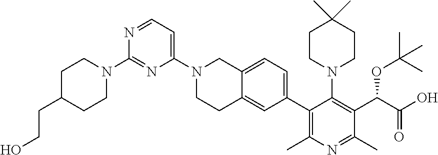 [SMILES: Cc1nc(C)c([C@H](OC(C)(C)C)C(=O)O)c(N2CCC(C)(C)CC2)c1-c1ccc2c(c1)CCN(c1ccnc(N3CCC(CCO)CC3)n1)C2]

### (S)-2-(tert-Butoxy)-2-(4-(4,4-dimethylpiperidin-1-yl)-5-(2-(2-(4-(2-hydroxyethyl)piperidin-1-yl)pyrimidin-4-yl)-1,2,3,4-tetrahydroisoquinolin-6-yl)-2,6-dimethylpyridin-3-yl)acetic Acid

In a 20 mL pressure vial equipped with a magnetic stirring bar was added (S)-isopropyl 2-(tert-butoxy)-2-(4-(4,4-dimethylpiperidin-1-yl)-2,6-dimethyl-5-(1,2,3,4-tetrahydroisoquinolin-6-yl)pyridin-3-yl)acetate (50 mg, 0.096 mmol), and 2-(1-(4-chloropyrimidin-2-yl)piperidin-4-yl)ethanol (46.3 mg, 0.192 mmol) in ethanol (2 mL). Hunig's base (0.084 mL, 0.479 mmol) was added and the mixture was heated to 100° C. in a preheated aluminum block and allowed to stir for 16 hours overnight. LC/MS showed the desired ester intermediate. After cooling to RT, 10M sodium hydroxide (0.096 mL, 0.958 mmol) was added, the vial capped and heated to 80° C. for 16 hours. LC/MS showed that the hydrolysis was complete. The reaction mixture was cooled, then filtered and was purified via preparative LC/MS to give 18.1 mg (27%) of the title compound. LCMS (M+1)=685.4. ¹H NMR (500 MHz, DMSO-d₆) δ 7.90 (d, J=5.9 Hz, 1H), 7.38-7.29 (m, 1H), 7.20-7.13 (m, 1H), 6.98 (s, 1H), 6.12 (br d, J=5.9 Hz, 1H), 5.81 (br d, J=12.5 Hz, 1H), 4.71-4.59 (m, 3H), 3.81 (br d, J=2.9 Hz, 2H), 3.47 (br t, J=6.6 Hz, 1H), 2.91 (br s, 2H), 2.86-2.68 (m, 3H), 2.55 (s, 2H), 2.46-2.35 (m, 3H), 2.16-2.04 (m, 3H), 1.92 (s, 1H), 1.81-1.59 (m, 4H), 1.47 (br s, 1H), 1.42-1.32 (m, 2H), 1.25 (br s, 1H), 1.21-1.08 (m, 10H), 1.08-0.98 (m, 4H), 0.94 (br d, J=12.1 Hz, 1H), 0.87-0.73 (m, 4H), 0.58 (s, 1H), 0.48 (s, 2H).

### Example 145

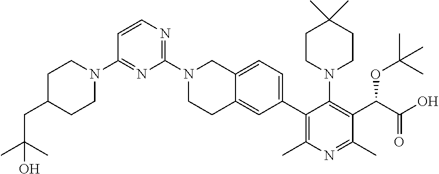 [SMILES: Cc1nc(C)c([C@H](OC(C)(C)C)C(=O)O)c(N2CCC(C)(C)CC2)c1-c1ccc2c(c1)CCN(c1nccc(N3CCC(CC(C)(C)O)CC3)n1)C2]

### (S)-2-(tert-Butoxy)-2-(4-(4,4-dimethylpiperidin-1-yl)-5-(2-(4-(4-(2-hydroxy-2-methylpropyl)piperidin-1-yl)pyrimidin-2-yl)-1,2,3,4-tetrahydroisoquinolin-6-yl)-2,6-dimethylpyridin-3-yl)acetic Acid

In a vial equipped with a magnetic stirring bar was added (S)-isopropyl 2-(tert-butoxy)-2-(4-(4,4-dimethylpiperidin-1-yl)-2,6-dimethyl-5-(4,4,5,5-tetramethyl-1,3,2-dioxaborolan-2-yl)pyridin-3-yl)acetate (56 mg, 0.108 mmol) and 1-(1-(2-(6-bromo-3,4-dihydroisoquinolin-2(1H)-yl)pyrimidin-4-yl)piperidin-4-yl)-2-methylpropan-2-ol (41.0 mg, 0.092 mmol). The solids were suspended in THF (3 mL). The mixture was treated with 0.5M K₃PO₄ (0.564 mL, 0.282 mmol) and X-Phos precatalyst G2 (14.93 mg, 0.019 mmol). Argon was streamed over the mixture for 5 minutes. The flask was capped/sealed and stirred at 100° C. within a sand bath for 16 hours. LC/MS showed the desired ester intermediate. After cooling to RT, removed solvent under nitrogen and took up residue in DCM and filtered through a 0.45 micron filter. The solvents were again removed under a stream of nitrogen to give a residue. The residue was dissolved in ethanol (2 mL) and 10M sodium hydroxide (0.108 mL, 1.084 mmol) was added. The vial was capped and heated to 80° C. for 16 hours. LC/MS showed that the hydrolysis was complete. The reaction mixture was cooled, then filtered and was purified via preparative LC/MS to give 23.9 mg (31%) of the title compound. LCMS (M+1)=713.4. ¹H NMR (500 MHz, DMSO-d₆) δ 7.88 (d, J=6.0 Hz, 1H), 7.35-7.26 (m, 1H), 7.15-7.07 (m, 1H), 7.01-6.90 (m, 1H), 6.06 (d, J=6.2 Hz, 1H), 5.85 (br d, J=11.7 Hz, 1H), 4.99-4.86 (m, 1H), 4.80 (t, J=16.5 Hz, 1H), 4.26 (br d, J=12.5 Hz, 2H), 4.09-3.99 (m, 1H), 3.99-3.83 (m, 2H), 2.93-2.78 (m, 4H), 2.55 (s, 2H), 2.48-2.40 (m, 3H), 2.15-2.04 (m, 3H), 1.92 (s, 3H), 1.87-1.69 (m, 3H), 1.56-1.38 (m, 1H), 1.38-1.29 (m, 2H), 1.28-1.19 (m, 1H), 1.19-1.05 (m, 17H), 0.98-0.90 (m, 1H), 0.90-0.73 (m, 3H), 0.66-0.42 (m, 3H).

### Example 146

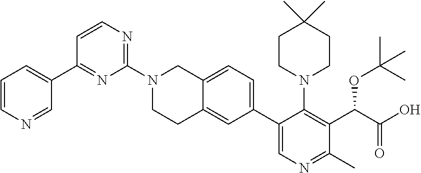 [SMILES: Cc1ncc(-c2ccc3c(c2)CCN(c2nccc(-c4cccnc4)n2)C3)c(N2CCC(C)(C)CC2)c1[C@H](OC(C)(C)C)C(=O)O]

### (S)-2-(tert-Butoxy)-2-(4-(4,4-dimethylpiperidin-1-yl)-2-methyl-5-(2-(4-(pyridin-3-yl)pyrimidin-2-yl)-1,2,3,4-tetrahydroisoquinolin-6-yl)pyridin-3-yl)acetic Acid

In a pressure vial equipped with a magnetic stirring bar was added (S)-isopropyl 2-(5-bromo-4-(4,4-dimethylpiperidin-1-yl)-2-methylpyridin-3-yl)-2-(tert-butoxy)acetate (58 mg, 0.127 mmol) and 2-(4-(pyridin-3-yl)pyrimidin-2-yl)-6-(4,4,5,5-tetramethyl-1,3,2-dioxaborolan-2-yl)-1,2,3,4-tetrahydroisoquinoline (79 mg, 0.191 mmol). The solids were suspended in distilled THF (4 mL). The mixture was treated with 0.5M K₃PO₄ (0.891 mL, 0.446 mmol) and X-Phos precatalyst G2 (8.52 mg, 10.82 μmol). Argon was streamed over and bubbled into the mixture for 5 minutes with sonication. The vial was capped and stirred at RT for 16 hours. LC/MS showed the desired ester intermediate. After cooling to RT, removed water layer, filtered reaction mixture then removed solvent under a stream of nitrogen. Took up residue in ethanol (2 mL). 10M sodium hydroxide (0.127 mL, 1.274 mmol) was added, then the vial was capped and heated to 70° C. for 16 hours overnight. LC/MS showed that the hydrolysis was complete. The reaction mixture was cooled, then filtered and was purified via preparative LC/MS to give 30.7 mg (38%) of the title compound. LCMS (M+1)=621.3. ¹H NMR (500 MHz, DMSO-d₆) δ 9.34 (s, 1H), 8.72 (d, J=4.8 Hz, 1H), 8.55 (d, J=4.8 Hz, 1H), 8.52 (d, J=8.2 Hz, 1H), 8.05 (s, 1H), 7.56 (dd, J=7.9, 5.0 Hz, 1H), 7.41 (br d, J=8.4 Hz, 1H), 7.31 (d, J=5.1 Hz, 1H), 7.23-7.10 (m, 2H), 5.83 (s, 1H), 5.10-4.99 (m, 2H), 4.14 (q, J=6.6 Hz, 2H), 3.08-2.92 (m, 2H), 2.55 (s, 1H), 1.44-1.24 (m, 8H), 1.23-1.10 (m, 12H), 0.92-0.69 (m, 6H).

### Example 147

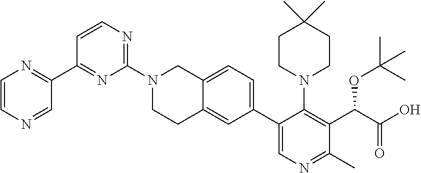 [SMILES: Cc1ncc(-c2ccc3c(c2)CCN(c2nccc(-c4cnccn4)n2)C3)c(N2CCC(C)(C)CC2)c1[C@H](OC(C)(C)C)C(=O)O]

### (S)-2-(tert-Butoxy)-2-(4-(4,4-dimethylpiperidin-1-yl)-2-methyl-5-(2-(4-(pyrazin-2-yl)pyrimidin-2-yl)-1,2,3,4-tetrahydroisoquinolin-6-yl)pyridin-3-yl)acetic Acid

In a pressure vial equipped with a magnetic stirring bar was added isopropyl (S)-2-(5-bromo-4-(4,4-dimethylpiperidin-1-yl)-2-methylpyridin-3-yl)-2-(tert-butoxy)acetate (50 mg, 0.110 mmol) and 2-(4-(pyrazin-2-yl)pyrimidin-2-yl)-6-(4,4,5,5-tetramethyl-1,3,2-dioxaborolan-2-yl)-1,2,3,4-tetrahydroisoquinoline (114 mg, 0.165 mmol). The solids were suspended in distilled THF (5 mL). The mixture was treated with 0.5M K₃PO₄ (0.768 mL, 0.384 mmol) and X-Phos precatalyst G2 (7.34 mg, 9.33 μmol). Argon was streamed over and bubbled into the mixture for 5 minutes with sonication. The vial was capped and stirred at RT for 48 hours. LC/MS showed the desired ester intermediate. Removed water layer and then removed solvent under vacuum. The residue was taken up in ethanol (2 mL). 10M sodium hydroxide (0.110 mL, 1.098 mmol) was added to the mixture and the vial capped and heated to 80° C. for 16 hours. LC/MS showed the hydrolysis product. The reaction mixture was purified via preparative LC/MS to give 13.2 mg (18%) of the title compound. LCMS (M+1)=620.3 (M+H)⁺. ¹H NMR (500 MHz, DMSO-d₆) δ 9.66 (s, 1H), 8.86-8.74 (m, 2H), 8.64 (d, J=4.8 Hz, 1H), 8.05 (s, 1H), 7.52 (d, J=5.1 Hz, 1H), 7.48-7.34 (m, 1H), 7.17 (s, 1H), 7.18 (d, J=5.5 Hz, 2H), 5.82 (s, 1H), 5.12-5.01 (m, 2H), 4.16 (q, J=6.1 Hz, 2H), 3.01 (br d, J=3.3 Hz, 3H), 1.91 (s, 1H), 1.37-1.21 (m, 4H), 1.13 (s, 12H), 0.90-0.66 (m, 8H).

### Example 148

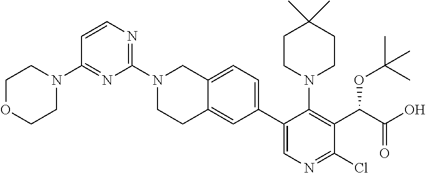 [SMILES: CC1(C)CCN(c2c(-c3ccc4c(c3)CCN(c3nccc(N5CCOCC5)n3)C4)cnc(Cl)c2[C@H](OC(C)(C)C)C(=O)O)CC1]

### (S)-2-(tert-Butoxy)-2-(2-chloro-4-(4,4-dimethylpiperidin-1-yl)-5-(2-(4-morpholinopyrimidin-2-yl)-1,2,3,4-tetrahydroisoquinolin-6-yl)pyridin-3-yl)acetic Acid

In a pressure vial equipped with a magnetic stirring bar was added (S)-ethyl 2-(5-bromo-2-chloro-4-(4,4-dimethylpiperidin-1-yl)pyridin-3-yl)-2-(tert-butoxy)acetate (60 mg, 0.130 mmol) and 4-(2-(6-(4,4,5,5-tetramethyl-1,3,2-dioxaborolan-2-yl)-3,4-dihydroisoquinolin-2(1H)-yl)pyrimidin-4-yl)morpholine (82 mg, 0.195 mmol). The solids were suspended in distilled THF (5 mL). The mixture was treated with 0.5M K₃PO₄ (0.909 mL, 0.455 mmol) and X-Phos precatalyst G2 (8.69 mg, 0.011 mmol). Argon was streamed over and bubbled into the mixture for 5 minutes with sonication. The vial was capped and stirred at RT for 16 hours. LC/MS showed the desired ester intermediate. Removed solvent under nitrogen. The residue was purified by flash column chromatography to give 120 mg of an off-white residue that was desired intermediate ester by LC/MS. The residue was transferred into a pressure vial using 2.5 mL of warm ethanol. 10M sodium hydroxide (0.130 mL, 1.299 mmol) was added and the vial capped and heated to 80° C. for 4 hours. LC/MS showed that the hydrolysis was complete. The reaction mixture was cooled, then filtered and was purified via preparative LC/MS to give 40.3 mg (47%) of the title compound. LCMS (M+1)=649.3. ¹H NMR (500 MHz, DMSO-d₆) δ 8.00 (s, 1H), 7.96 (d, J=6.2 Hz, 1H), 7.34 (d, J=8.4 Hz, 1H), 7.19-7.05 (m, 2H), 6.09 (d, J=5.9 Hz, 1H), 5.59 (s, 1H), 4.94-4.79 (m, 2H), 4.03-3.93 (m, 2H), 3.73-3.60 (m, 4H), 3.60-3.45 (m, 3H), 2.90 (br s, 2H), 2.59-2.53 (m, 3H), 1.92 (s, 2H), 1.39-1.21 (m, 4H), 1.17 (s, 10H), 0.78 (s, 6H).

### Example 149

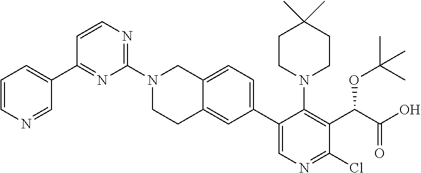 [SMILES: CC1(C)CCN(c2c(-c3ccc4c(c3)CCN(c3nccc(-c5cccnc5)n3)C4)cnc(Cl)c2[C@H](OC(C)(C)C)C(=O)O)CC1]

### (S)-2-(tert-Butoxy)-2-(2-chloro-4-(4,4-dimethylpiperidin-1-yl)-5-(2-(4-(pyridin-3-yl)pyrimidin-2-yl)-1,2,3,4-tetrahydroisoquinolin-6-yl)pyridin-3-yl)acetic Acid

In a pressure vial equipped with a magnetic stirring bar was added (S)-ethyl 2-(5-bromo-2-chloro-4-(4,4-dimethylpiperidin-1-yl)pyridin-3-yl)-2-(tert-butoxy)acetate (60 mg, 0.130 mmol) and 2-(4-(pyridin-3-yl)pyrimidin-2-yl)-6-(4,4,5,5-tetramethyl-1,3,2-dioxaborolan-2-yl)-1,2,3,4-tetrahydroisoquinoline (81 mg, 0.195 mmol). The solids were suspended in distilled THF (4 mL). The mixture was treated with 0.5M K₃PO₄ (0.909 mL, 0.455 mmol) and X-Phos precatalyst G2 (8.69 mg, 0.011 mmol). Argon was streamed over and bubbled into the mixture for 5 minutes with sonication. The vial was capped and stirred at RT for 16 hours. LC/MS showed the desired ester intermediate. After cooling to RT, removed water layer, filtered reaction mixture then removed solvent under a stream of nitrogen. Took up residue in ethanol (2 mL). 10M sodium hydroxide (0.130 mL, 1.299 mmol) was added, then the vial was capped and heated to 70° C. for 16 hours overnight. LC/MS showed that the hydrolysis was complete. The reaction mixture was cooled, then filtered and was purified via preparative LC/MS to give 14.4 mg (17%) of the title compound. LCMS (M+1)=641.3. ¹H NMR (500 MHz, DMSO-d₆) δ 9.34 (s, 1H), 8.72 (br d, J=4.4 Hz, 1H), 8.59-8.46 (m, 2H), 8.01 (s, 1H), 7.57 (dd, J=7.7, 4.8 Hz, 1H), 7.48-7.37 (m, 1H), 7.31 (d, J=5.1 Hz, 1H), 7.26-7.13 (m, 2H), 5.59 (s, 1H), 5.10-5.00 (m, 2H), 4.24-4.01 (m, 2H), 3.00 (br d, J=6.6 Hz, 2H), 2.55 (s, 1H), 1.92 (s, 4H), 1.88 (br s, 1H), 1.43-1.28 (m, 1H), 1.28-1.22 (m, 1H), 1.22-1.15 (m, 9H), 1.15-1.03 (m, 1H), 0.82-0.62 (m, 6H).

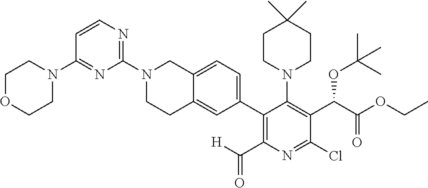 [SMILES: CCOC(=O)[C@@H](OC(C)(C)C)c1c(Cl)nc(C=O)c(-c2ccc3c(c2)CCN(c2nccc(N4CCOCC4)n2)C3)c1N1CCC(C)(C)CC1]

### (S)-ethyl 2-(tert-Butoxy)-2-(2-chloro-4-(4,4-dimethylpiperidin-1-yl)-6-formyl-5-(2-(4-morpholinopyrimidin-2-yl)-1,2,3,4-tetrahydroisoquinolin-6-yl)pyridin-3-yl)acetate

In a pressure vial equipped with a magnetic stirring bar was added (S)-ethyl 2-(5-bromo-2-chloro-4-(4,4-dimethylpiperidin-1-yl)-6-formylpyridin-3-yl)-2-(tert-butoxy)acetate (125 mg, 0.255 mmol) and 4-(2-(6-(4,4,5,5-tetramethyl-1,3,2-dioxaborolan-2-yl)-3,4-dihydroisoquinolin-2(1H)-yl)pyrimidin-4-yl)morpholine (162 mg, 0.383 mmol). The solids were suspended in distilled THF (10 mL). The mixture was treated with 0.5M K₃PO₄ (1.786 mL, 0.893 mmol) and X-Phos precatalyst G2 (17.07 mg, 0.022 mmol). Argon was streamed over and bubbled into the mixture for 5 minutes with sonication. The vial was capped and stirred at RT for 16 hours. LC/MS showed the desired aldehyde. Removed solvent under a stream of nitrogen. The residue was taken up in ethyl acetate and washed with brine, organic layer dried over MgSO₄, filtered and concentrated down. The residue was purified by flash column chromatography to give 40 mg (22%) of(S)-ethyl 2-(tert-butoxy)-2-(2-chloro-4-(4,4-dimethylpiperidin-1-yl)-6-formyl-5-(2-(4-morpholinopyrimidin-2-yl)-1,2,3,4-tetrahydroisoquinolin-6-yl)pyridin-3-yl)acetate (40 mg, 0.057 mmol) as a yellow residue. LCMS (M+1)=705.4.

### Example 150

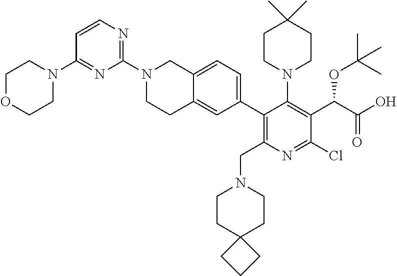 [SMILES: CC1(C)CCN(c2c(-c3ccc4c(c3)CCN(c3nccc(N5CCOCC5)n3)C4)c(CN3CCC4(CCC4)CC3)nc(Cl)c2[C@H](OC(C)(C)C)C(=O)O)CC1]

### (S)-2-(6-((7-Azaspiro[3.5]nonan-7-yl)methyl)-2-chloro-4-(4,4-dimethylpiperidin-1-yl)-5-(2-(4-morpholinopyrimidin-2-yl)-1,2,3,4-tetrahydroisoquinolin-6-yl)pyridin-3-yl)-2-(tert-butoxy)acetic Acid

A round bottom flask containing a magnetic stirring bar was added (S)-ethyl 2-(tert-butoxy)-2-(2-chloro-4-(4,4-dimethylpiperidin-1-yl)-6-formyl-5-(2-(4-morpholinopyrimidin-2-yl)-1,2,3,4-tetrahydroisoquinolin-6-yl)pyridin-3-yl)acetate (40 mg, 0.057 mmol), 7-azaspiro[3.5]nonane (23.08 mg, 0.184 mmol), DCE (2 mL), acetic acid (0.012 mL, 0.213 mmol) and several dry 4A molecular sieves. The mixture was stirred under nitrogen for 10 minutes before adding EtOH (1 mL). The reaction was stirred for 1 hour at RT. Sodium cyanoborohydride (0.227 mL, 0.227 mmol) (1M solution in THF) was then added dropwise over a 1 hour time period. After addition was complete, LC/MS showed desired amine product, and reduced methyl alcohol products. The solvents were removed under a stream of nitrogen and the reaction products were carried forward into the hydrolysis. In a pressure vial equipped with a magnetic stirring bar was added the previous reaction mixture. Took up the residue in ethanol (2.5 mL). 10 M sodium hydroxide (0.113 mL, 1.130 mmol) was added, then the vial was capped and heated to 80° C. for 16 hours overnight. LC/MS showed that the hydrolysis was complete. The reaction mixture was cooled, then filtered and was purified via preparative LC/MS to give 9.0 mg (20%) of the title compound. LCMS (M+1)=786.4. ¹H NMR (600 MHz, DMSO-d₆) δ 7.97-7.87 (m, 1H), 7.35-7.18 (m, 2H), 6.99-6.86 (m, 1H), 6.10 (br d, J=5.9 Hz, 1H), 5.52 (br s, 1H), 4.91-4.74 (m, 2H), 4.05-3.92 (m, 1H), 3.92-3.81 (m, 1H), 3.69 (br s, 3H), 3.67-3.48 (m, 4H), 3.15-3.06 (m, 1H), 3.06-2.93 (m, 1H), 2.91-2.71 (m, 3H), 2.23-2.12 (m, 1H), 2.09 (br s, 2H), 2.03 (br s, 1H), 1.90 (s, 3H), 1.81-1.68 (m, 3H), 1.67-1.52 (m, 4H), 1.46-1.28 (m, 4H), 1.28-1.19 (m, 1H), 1.13 (br s, 11H), 0.79 (br s, 4H), 0.57 (br s, 2H), 0.47 (br s, 2H). also isolated (S)-2-(tert-Butoxy)-2-(2-chloro-4-(4,4-dimethylpiperidin-1-yl)-6-(hydroxymethyl)-5-(2-(4-morpholinopyrimidin-2-yl)-1,2,3,4-tetrahydroisoquinolin-6-yl)pyridin-3-yl)acetic acid 8.4 mg (22%).

### Example 151

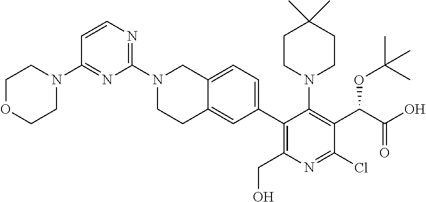 [SMILES: CC1(C)CCN(c2c(-c3ccc4c(c3)CCN(c3nccc(N5CCOCC5)n3)C4)c(CO)nc(Cl)c2[C@H](OC(C)(C)C)C(=O)O)CC1]

### (S)-2-(tert-Butoxy)-2-(2-chloro-4-(4,4-dimethylpiperidin-1-yl)-6-(hydroxymethyl)-5-(2-(4-morpholinopyrimidin-2-yl)-1,2,3,4-tetrahydroisoquinolin-6-yl)pyridin-3-yl)acetic Acid

LCMS (M+1)=679.3. ¹H NMR (600 MHz, DMSO-d₆) δ 7.97-7.87 (m, 1H), 7.35-7.18 (m, 2H), 6.99-6.86 (m, 1H), 6.10 (br d, J=5.9 Hz, 1H), 5.52 (br s, 1H), 4.91-4.74 (m, 2H), 4.05-3.92 (m, 1H), 3.92-3.81 (m, 1H), 3.69 (br s, 3H), 3.67-3.48 (m, 4H), 3.15-3.06 (m, 1H), 3.06-2.93 (m, 1H), 2.91-2.71 (m, 3H), 2.23-2.12 (m, 1H), 2.09 (br s, 2H), 2.03 (br s, 1H), 1.90 (s, 3H), 1.81-1.68 (m, 3H), 1.67-1.52 (m, 4H), 1.46-1.28 (m, 4H), 1.28-1.19 (m, 1H), 1.13 (br s, 11H), 0.79 (br s, 4H), 0.57 (br s, 2H), 0.47 (br s, 2H).

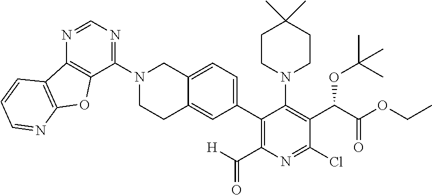 [SMILES: CCOC(=O)[C@@H](OC(C)(C)C)c1c(Cl)nc(C=O)c(-c2ccc3c(c2)CCN(c2ncnc4c2oc2ncccc24)C3)c1N1CCC(C)(C)CC1]

### (S)-Ethyl 2-(tert-butoxy)-2-(2-chloro-4-(4,4-dimethylpiperidin-1-yl)-6-formyl-5-(2-(pyrido[3′,2′:4,5]furo[3,2-d]pyrimidin-4-yl)-1,2,3,4-tetrahydroisoquinolin-6-yl)pyridin-3-yl)acetate

A mixture of (S)-ethyl 2-(5-bromo-2-chloro-4-(4,4-dimethylpiperidin-1-yl)-6-formylpyridin-3-yl)-2-(tert-butoxy)acetate (117.5 mg, 0.240 mmol) and 4-(6-(4,4,5,5-tetramethyl-1,3,2-dioxaborolan-2-yl)-3,4-dihydroisoquinolin-2(1H)-yl)pyrido[3′,2′:4,5]furo[3,2-d]pyrimidine in distilled THF (9 mL) is flushed well with argon, added degassed 0.5 M potassium phosphate tribasic (1.65 mL, 0.825 mmol) then 2nd generation xphos precatalyst (15 mg, 0.019 mmol) were added, the vial sealed and stirred at RT for 48 hours overnight. LC/MS showed desired product. Partitioned with water and EtOAc. Washed with brine, dried over Na₂SO₄ and concentrated down under vacuum. Purified crude material by flash column chromatography to give 134.9 mg of (S)-ethyl 2-(tert-butoxy)-2-(2-chloro-4-(4,4-dimethylpiperidin-1-yl)-6-formyl-5-(2-(pyrido[3′,2′:4,5]furo[3,2-d]pyrimidin-4-yl)-1,2,3,4-tetrahydroisoquinolin-6-yl)pyridin-3-yl)acetate. LCMS (M+1)=711.4.

### Example 152

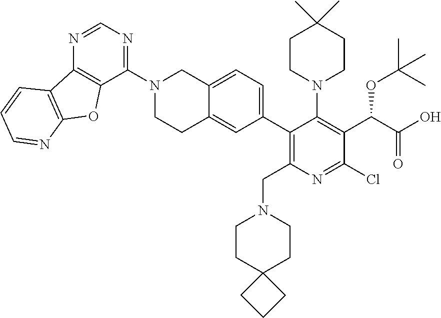 [SMILES: CC1(C)CCN(c2c(-c3ccc4c(c3)CCN(c3ncnc5c3oc3ncccc35)C4)c(CN3CCC4(CCC4)CC3)nc(Cl)c2[C@H](OC(C)(C)C)C(=O)O)CC1]

### (S)-2-(6-((7-Azaspiro[3.5]nonan-7-yl)methyl)-2-chloro-4-(4,4-dimethylpiperidin-1-yl)-5-(2-(pyrido[3′,2′:4,5]furo[3,2-d]pyrimidin-4-yl)-1,2,3,4-tetrahydroisoquinolin-6-yl)pyridin-3-yl)-2-(tert-butoxy)acetic Acid

A small pressure vial containing a magnetic stirring bar was added (S)-ethyl 2-(tert-butoxy)-2-(2-chloro-4-(4,4-dimethylpiperidin-1-yl)-6-formyl-5-(2-(pyrido[3′,2′:4,5]furo[3,2-d]pyrimidin-4-yl)-1,2,3,4-tetrahydroisoquinolin-6-yl)pyridin-3-yl)acetate (64 mg, 0.090 mmol), 7-azaspiro[3.5]nonane (36.6 mg, 0.292 mmol), DCE (3 mL), acetic acid (0.019 mL, 0.337 mmol) and several dry 4A molecular sieves. The mixture was stirred under nitrogen for 10 minutes before adding EtOH (1 mL). The reaction was stirred for 1 hour at RT. Sodium cyanoborohydride (0.360 mL, 0.360 mmol) (1M solution in THF) was then added dropwise over a 1 hour time period. After addition was complete, LC/MS showed desired amine product. The solvents were removed under a stream of nitrogen and the reaction mixture was carried into the hydrolysis. In a pressure vial equipped with a magnetic stirring bar was added the residue in ethanol (2.5 mL). 10M sodium hydroxide (0.180 mL, 1.804 mmol) was added, then the vial was capped and heated to 80° C. for 16 hours overnight. LC/MS showed that the hydrolysis was complete. The reaction mixture was cooled, then filtered and was purified via preparative LC/MS to give 1.4 mg (2%) of the title compound. LCMS (M+1)=792.3. ¹H NMR (600 MHz, DMSO-d₆) δ 8.71-8.64 (m, 1H), 8.64-8.54 (m, 2H), 7.62 (br dd, J=7.2, 5.0 Hz, 1H), 7.44-7.33 (m, 2H), 7.32 (br s, 1H), 6.99 (br s, 1H), 5.48 (br s, 1H), 5.33-5.16 (m, 2H), 4.36 (br s, 1H), 4.28 (br s, 1H), 3.78-3.64 (m, 2H), 3.15-3.03 (m, 2H), 3.00 (br s, 1H), 2.16 (br d, J=7.7 Hz, 1H), 2.08 (br s, 1H), 2.04 (br s, 1H), 2.00 (br s, 1H), 1.90 (s, 6H), 1.78-1.72 (m, 1H), 1.72-1.66 (m, 1H), 1.63 (br s, 1H), 1.59-1.39 (m, 4H), 1.39-1.26 (m, 4H), 1.22 (br s, 1H), 1.17-0.97 (m, 10H), 0.75 (br s, 2H), 0.61-0.46 (m, 1H), 0.39 (br s, 1H).

### Example 153

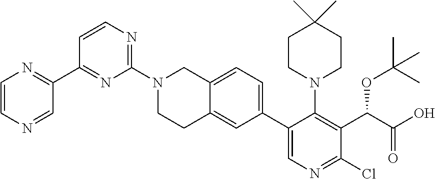 [SMILES: CC1(C)CCN(c2c(-c3ccc4c(c3)CCN(c3nccc(-c5cnccn5)n3)C4)cnc(Cl)c2[C@H](OC(C)(C)C)C(=O)O)CC1]

### (S)-2-(tert-Butoxy)-2-(2-chloro-4-(4,4-dimethylpiperidin-1-yl)-5-(2-(4-(pyrazin-2-yl)pyrimidin-2-yl)-1,2,3,4-tetrahydroisoquinolin-6-yl)pyridin-3-yl)acetic Acid

In a pressure vial equipped with a magnetic stirring bar was added ethyl (S)-2-(5-bromo-2-chloro-4-(4,4-dimethylpiperidin-1-yl)pyridin-3-yl)-2-(tert-butoxy)acetate (50 mg, 0.108 mmol) and 2-(4-(pyrazin-2-yl)pyrimidin-2-yl)-6-(4,4,5,5-tetramethyl-1,3,2-dioxaborolan-2-yl)-1,2,3,4-tetrahydroisoquinoline (112 mg, 0.162 mmol). The solids were suspended in distilled THF (5 mL). The mixture was treated with 0.5M K₃PO₄ (0.758 mL, 0.379 mmol) and X-Phos precatalyst G2 (7.24 mg, 9.20 μmol). Argon was streamed over and bubbled into the mixture for 5 minutes with sonication. The flask was capped/sealed and stirred at RT for 48 hours. LC/MS showed the desired ester intermediate. Removed water layer and then removed solvent under vacuum. The residue was taken up in ethanol (2 mL). 10M sodium hydroxide (0.108 mL, 1.083 mmol) was added to the mixture, and the vial capped and heated to 80° C. for 16 hours. LC/MS showed the hydrolysis product. The reaction mixture was purified via preparative LC/MS to give 10.0 mg (14%) of the title compound. LCMS (M+1)=642.2. ¹H NMR (500 MHz, DMSO-d₆) δ 9.70-9.58 (m, 1H), 8.79 (d, J=10.9 Hz, 2H), 8.64 (d, J=4.8 Hz, 1H), 8.00 (s, 1H), 7.52 (d, J=5.1 Hz, 1H), 7.45 (d, J=7.7 Hz, 1H), 7.21 (s, 1H), 7.20 (d, J=7.0 Hz, 2H), 5.57 (s, 1H), 5.12-5.01 (m, 2H), 4.15 (dq, J=12.1, 6.4 Hz, 2H), 3.06-2.90 (m, 2H), 1.35 (br s, 1H), 1.31-1.23 (m, 4H), 1.20 (br s, 1H), 1.20-1.06 (m, 11H), 0.91-0.69 (m, 6H).

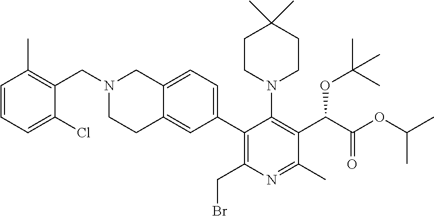 [SMILES: Cc1cccc(Cl)c1CN1CCc2cc(-c3c(CBr)nc(C)c([C@H](OC(C)(C)C)C(=O)OC(C)C)c3N3CCC(C)(C)CC3)ccc2C1]

### Isopropyl (S)-2-(6-(bromomethyl)-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetate

(S)-Isopropyl 2-(5-bromo-4-(4,4-dimethylpiperidin-1-yl)-6-(hydroxymethyl)-2-methylpyridin-3-yl)-2-(tert-butoxy)acetate (0.50 g, 1.0 mmol), 2-(2-chloro-6-methylbenzyl)-6-(4,4,5,5-tetramethyl-1,3,2-dioxaborolan-2-yl)-1,2,3,4-tetrahydroisoquinoline (0.41 g, 1.0 mmol), 2-dicyclohexylphosphino-2′,6′-dimethoxybiphenyl (0.085 g, 0.21 mmol), Pd(OAc)₂ (0.023 g, 0.10 mmol) and potassium phosphate tribasic (1.64 g, 7.72 mmol) were combined under N₂. 1,4-Dioxane (17.1 ml) and water (3.43 ml) were added under N₂. The reaction was heated at 80° C. with stirring for 2 h. The reaction mixture was concentrated, adsorbed onto Celite and was purified on silica gel (Biotage, EtOAc/hexanes gradient, 0-100% over 10 CVs) to afford (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)-2-methylpyridin-3-yl)acetate (0.420 g, 0.621 mmol, 60% yield).

To a solution of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)-2-methylpyridin-3-yl)acetate (400 mg, 0.591 mmol) in CH₂Cl₂ (5 mL) was added CBr₄ (255 mg, 0.769 mmol) followed by Ph₃P (202 mg, 0.769 mmol). The resulting mixture was stirred at room temperature for 2 h. Water (20 mL) was then added and the mixture was extracted with dichloromethane (2×20 mL), dried (Na₂SO₄), filtered and concentrated. The residue was then adsorbed onto Celite and then purified by Biotage (5-30% EtOAc/hexane) to afford (S)-isopropyl 2-(6-(bromomethyl)-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetate (135 mg, 0.183 mmol, 31% yield) as a mixture of diastereomers. ¹H NMR (500 MHz, DMSO-d6) δ 7.26-7.04 (m, 5H), 6.96-6.87 (m, 1H), 6.04 (br s, 1H), 5.17-5.01 (m, 1H), 4.41-4.27 (m, 1H), 4.21 (d, J=9.3 Hz, 1H), 3.91-3.69 (m, 3H), 3.23-3.11 (m, 1H), 2.93-2.76 (m, 4H), 2.63-2.57 (m, 3H), 2.53-2.45 (m, 3H), 2.21 (d, J=11.0 Hz, 1H), 1.89 (t, J=11.8 Hz, 1H), 1.59 (br s, 3H), 1.52 (br s, 1H), 1.28-1.20 (m, 7H), 1.18 (d, J=2.7 Hz, 9H), 0.89 (br s, 3H), 0.69-0.58 (m, 3H). LCMS (M+H)=738.1, 740.1.

### Example 154

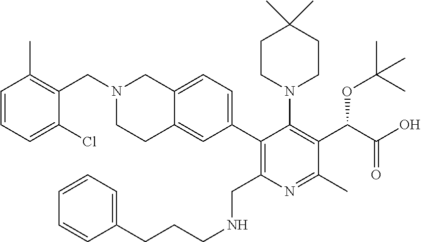 [SMILES: Cc1cccc(Cl)c1CN1CCc2cc(-c3c(CNCCCc4ccccc4)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((3-phenylpropyl)amino)methyl)pyridin-3-yl)acetic Acid

A solution of (S)-isopropyl 2-(6-(bromomethyl)-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetate (20 mg, 0.027 mmol), 3-phenylpropan-1-amine (37 mg, 0.27 mmol) and N-ethyl-N-isopropylpropan-2-amine (28 mg, 0.22 mmol) in EtOH (1 mL) was stirred at rt overnight. 5M NaOH (0.054 mL, 0.27 mmol) was added. The reaction mixture was stirred at 80° C. for 2 h, the mixture was then cooled and purified via preparative LC/MS to afford (S)-2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((3-phenylpropyl)amino)methyl)pyridin-3-yl)acetic acid (6.6 mg, 8.8 μmol, 33% yield). LCMS (M+H)=751.18.

### Example 155

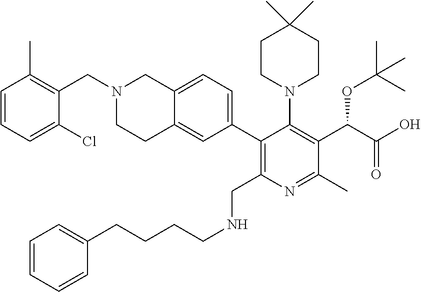 [SMILES: Cc1cccc(Cl)c1CN1CCc2cc(-c3c(CNCCCCc4ccccc4)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((4-phenylbutyl)amino)methyl)pyridin-3-yl)acetic Acid

A solution of (S)-isopropyl 2-(6-(bromomethyl)-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetate (20 mg, 0.027 mmol), 4-phenylbutan-1-amine (40 mg, 0.27 mmol) and N-ethyl-N-isopropylpropan-2-amine (28 mg, 0.22 mmol) in EtOH (1 mL) was stirred at rt overnight. 5M NaOH (0.054 mL, 0.27 mmol) was added. The reaction mixture was stirred at 80° C. for 2 h, and then the mixture was then cooled and purified via preparative LC/MS to give (S)-2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((4-phenylbutyl)amino)methyl)pyridin-3-yl)acetic acid (4.4 mg, 5.8 μmol, 21% yield). LCMS (M+H)=765.21.

### Example 156

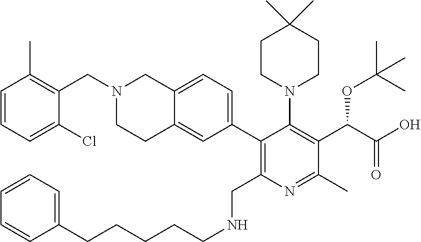 [SMILES: Cc1cccc(Cl)c1CN1CCc2cc(-c3c(CNCCCCCc4ccccc4)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((5-phenylpentyl)amino)methyl)pyridin-3-yl)acetic Acid

A solution of (S)-isopropyl 2-(6-(bromomethyl)-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetate (20 mg, 0.027 mmol), 5-phenylpentan-1-amine (44 mg, 0.27 mmol) and N-ethyl-N-isopropylpropan-2-amine (28 mg, 0.22 mmol) in EtOH (1 mL) was stirred at rt overnight. 5M NaOH (0.054 mL, 0.27 mmol) was added. The reaction mixture was stirred at 80° C. for 2 h, the mixture was then cooled and purified via preparative to give (S)-2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((5-phenylpentyl)amino)methyl)pyridin-3-yl)acetic acid (3.5 mg, 4.4 μmol, 17% yield). LCMS (M+H)=779.23.

### Example 157

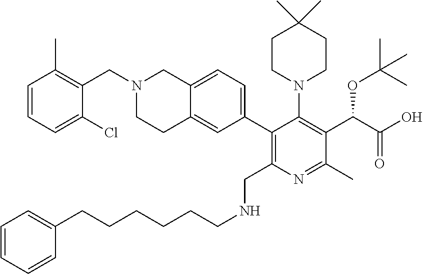 [SMILES: Cc1cccc(Cl)c1CN1CCc2cc(-c3c(CNCCCCCCc4ccccc4)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((6-phenylhexyl)amino)methyl)pyridin-3-yl)acetic Acid

A solution of (S)-isopropyl 2-(6-(bromomethyl)-5-(2-(2-chloro-6-methylbenzyl)-1,2,3,4-tetrahydroisoquinolin-6-yl)-4-(4,4-dimethylpiperidin-1-yl)-2-methylpyridin-3-yl)-2-(tert-butoxy)acetate (20 mg, 0.027 mmol), 6-phenylhexan-1-amine (9.6 mg, 0.054 mmol) and N-ethyl-N-isopropylpropan-2-amine (28 mg, 0.22 mmol) in EtOH (1 mL) was stirred at rt overnight. 5M NaOH (0.054 mL, 0.27 mmol) was added.

The reaction mixture was stirred at 80° C. for 2 h, and mixture was then cooled and purified via preparative LC/MS to give (S)-2-(tert-butoxy)-2-(5-(2-(2-chloro-6-methylbenzyl)-1,2,3,4-tetrahydroisoquinolin-6-yl)-4-(4,4-dimethylpiperidin-1-yl)-2-methyl-6-(((6-phenylhexyl)amino)methyl)pyridin-3-yl)acetic acid (6.7 mg, 8.4 μmol, 31% yield). LCMS (M+H)=793.26.

### Example 158

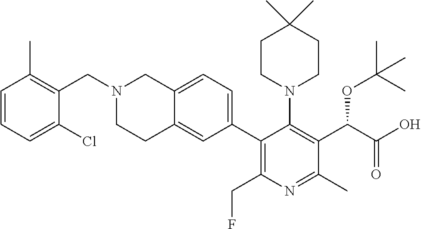 [SMILES: Cc1cccc(Cl)c1CN1CCc2cc(-c3c(CF)nc(C)c([C@H](OC(C)(C)C)C(=O)O)c3N3CCC(C)(C)CC3)ccc2C1]

### (S)-2-(tert-Butoxy)-2-(5-(2-(2-chloro-6-methylbenzyl)-1,2,3,4-tetrahydroisoquinolin-6-yl)-4-(4,4-dimethylpiperidin-1-yl)-6-(fluoromethyl)-2-methylpyridin-3-yl)acetic Acid

Bis(2-methoxyethyl)aminosulfur trifluoride (34 mg, 0.16 mmol) was added to a stirring solution of (S)-isopropyl 2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(hydroxymethyl)-2-methylpyridin-3-yl)acetate (35 mg, 0.052 mmol) in CH₂Cl₂ (1 mL) at rt. The mixture was allowed to stir at this temp overnight. Next, the mixture was concentrated to dryness and then taken up in EtOH (1 ml) and 5N NaOH (0.104 mL, 0.518 mmol) was added. The mixture was heated to 80° C. and stirred at this temp for 2 h. The mixture was filtered and purified via preparative LC/MS to give (S)-2-(tert-butoxy)-2-(5-(2-(2-chloro-6-methylbenzyl)-1,2,3,4-tetrahydroisoquinolin-6-yl)-4-(4,4-dimethylpiperidin-1-yl)-6-(fluoromethyl)-2-methylpyridin-3-yl)acetic acid (5.4 mg, 16% yield). LCMS (M+H)=636.25.

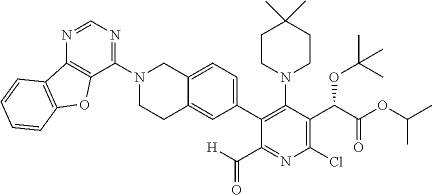 [SMILES: CC(C)OC(=O)[C@@H](OC(C)(C)C)c1c(Cl)nc(C=O)c(-c2ccc3c(c2)CCN(c2ncnc4c2oc2ccccc24)C3)c1N1CCC(C)(C)CC1]

### (S)-Isopropyl 2-(5-(2-(benzofuro[3,2-d]pyrimidin-4-yl)-1,2,3,4-tetrahydroisoquinolin-6-yl)-4-(4,4-dimethylpiperidin-1-yl)-6-formyl-2-methylpyridin-3-yl)-2-(tert-butoxy)acetate

(S)-Isopropyl 2-(5-bromo-4-(4,4-dimethylpiperidin-1-yl)-6-formyl-2-methylpyridin-3-yl)-2-(tert-butoxy)acetate (0.111 g, 0.230 mmol), 4-(6-(4,4,5,5-tetramethyl-1,3,2-dioxaborolan-2-yl)-3,4-dihydroisoquinolin-2(1H)-yl)benzofuro[3,2-d]pyrimidine (0.118 g, 0.276 mmol), 2-dicyclohexylphosphino-2′,6′-dimethoxybiphenyl (0.019 g, 0.046 mmol), Pd(OAc)₂ (5.2 mg, 0.023 mmol) and K₃PO₄ (0.366 g, 1.72 mmol) were combined under N₂. 1,4-Dioxane (3.8 ml) and water (0.77 ml) were added under N₂. The reaction was heated at 80° C. for 2 h. The reaction was concentrated, adsorbed onto Celite and was purified on silica gel (Biotage, EtOAc/hexanes gradient, 0-100% over 10 CVs) to give (S)-isopropyl 2-(5-(2-(benzofuro[3,2-d]pyrimidin-4-yl)-1,2,3,4-tetrahydroisoquinolin-6-yl)-4-(4,4-dimethylpiperidin-1-yl)-6-formyl-2-methylpyridin-3-yl)-2-(tert-butoxy)acetate (60 mg, 0.085 mmol, 37% yield) as a yellow residue. LCMS (M+H)=704.25.

### Example 159

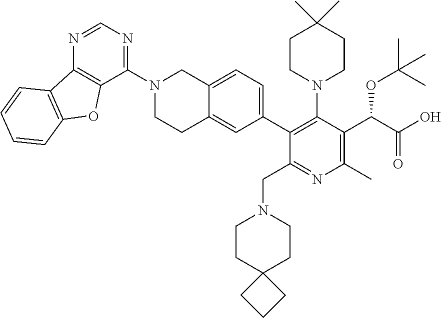 [SMILES: Cc1nc(CN2CCC3(CCC3)CC2)c(-c2ccc3c(c2)CCN(c2ncnc4c2oc2ccccc24)C3)c(N2CCC(C)(C)CC2)c1[C@H](OC(C)(C)C)C(=O)O]

### (S)-2-(6-(7-Azaspiro[3.5]nonan-7-ylmethyl)-5-(2-(benzofuro[3,2-d]pyrimidin-4-yl)-1,2,3,4-tetrahydroisoquinolin-6-yl)-4-(4,4-dimethylpiperidin-1-yl)-2-methylpyridin-3-yl)-2-(tert-butoxy)acetic Acid

7-Azaspiro[3.5]nonane (14.2 mg, 0.114 mmol) was added to a stirring solution of (S)-isopropyl 2-(5-(2-(benzofuro[3,2-d]pyrimidin-4-yl)-1,2,3,4-tetrahydroisoquinolin-6-yl)-4-(4,4-dimethylpiperidin-1-yl)-6-formyl-2-methylpyridin-3-yl)-2-(tert-butoxy)acetate (20 mg, 0.028 mmol) in MeOH (2 mL) at rt. The solution was allowed to stir at rt for 2 h. Next, sodium triacetoxyborohydride (12 mg, 0.057 mmol) was added. The mixture was stirred for an additional 1 h. LCMS shows conversion to the desired product. The mixture was adsorbed onto Celite and then purified on silica gel eluting with a 0-100% hexanes in EtOAc gradient to give the desired amine as a colorless residue. This colorless residue was taken up in EtOH (1 mL) and 5M NaOH (0.057 mL, 0.28 mmol) was added. The mixture was heated to 85° C. and stirred at this temp overnight. The mixture was filtered and purified via preparative LC/MS to give (S)-2-(6-(7-azaspiro[3.5]nonan-7-ylmethyl)-5-(2-(benzofuro[3,2-d]pyrimidin-4-yl)-1,2,3,4-tetrahydroisoquinolin-6-yl)-4-(4,4-dimethylpiperidin-1-yl)-2-methylpyridin-3-yl)-2-(tert-butoxy)acetic acid (1.0 mg, 5% yield). LCMS (M+H)=771.24.

### Example 160

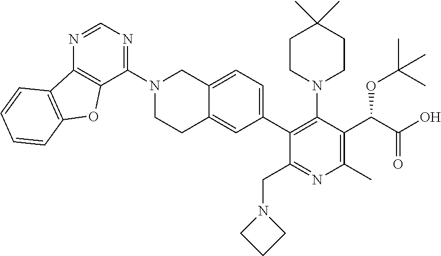 [SMILES: Cc1nc(CN2CCC2)c(-c2ccc3c(c2)CCN(c2ncnc4c2oc2ccccc24)C3)c(N2CCC(C)(C)CC2)c1[C@H](OC(C)(C)C)C(=O)O]

### (S)-2-(6-(Azetidin-1-ylmethyl)-5-(2-(benzofuro[3,2-d]pyrimidin-4-yl)-1,2,3,4-tetrahydroisoquinolin-6-yl)-4-(4,4-dimethylpiperidin-1-yl)-2-methylpyridin-3-yl)-2-(tert-butoxy)acetic Acid

Azetidine hydrochloride (23 mg, 0.24 mmol) was added to a stirring solution of (S)-isopropyl 2-(5-(2-(benzofuro[3,2-d]pyrimidin-4-yl)-1,2,3,4-tetrahydroisoquinolin-6-yl)-4-(4,4-dimethylpiperidin-1-yl)-6-formyl-2-methylpyridin-3-yl)-2-(tert-butoxy)acetate (17 mg, 0.024 mmol) in EtOH (2 mL) at rt. Next, AcOH (7.0 l, 0.12 mmol) was added, and the solution was allowed to stir at rt for 8 h. Next, sodium triacetoxyborohydride (20 mg, 0.097 mmol) was added. The mixture was stirred for an additional 1 h. LCMS shows conversion to the desired product. The mixture was adsorbed onto Celite and then purified on silica gel eluting with a 0-100% hexanes in EtOAc gradient to give the desired amine as a colorless residue. This residue was taken up in EtOH (1 mL) and 5M NaOH (0.048 mL, 0.24 mmol) was added. The mixture was heated to 85° C. and stirred at this temp overnight. The mixture was filtered and purified via preparative LC/MS to give (S)-2-(6-(azetidin-1-ylmethyl)-5-(2-(benzofuro[3,2-d]pyrimidin-4-yl)-1,2,3,4-tetrahydroisoquinolin-6-yl)-4-(4,4-dimethylpiperidin-1-yl)-2-methylpyridin-3-yl)-2-(tert-butoxy)acetic acid (2.2 mg, 12% yield). LCMS (M+H)=703.11.

### Example 161

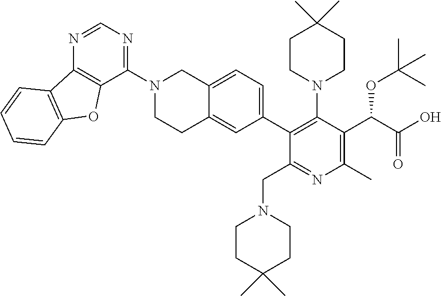 [SMILES: Cc1nc(CN2CCC(C)(C)CC2)c(-c2ccc3c(c2)CCN(c2ncnc4c2oc2ccccc24)C3)c(N2CCC(C)(C)CC2)c1[C@H](OC(C)(C)C)C(=O)O]

### (S)-2-(5-(2-(Benzofuro[3,2-d]pyrimidin-4-yl)-1,2,3,4-tetrahydroisoquinolin-6-yl)-4-(4,4-dimethylpiperidin-1-yl)-6-((4,4-dimethylpiperidin-1-yl)methyl)-2-methylpyridin-3-yl)-2-(tert-butoxy)acetic Acid

4,4-dimethylpiperidine (27 mg, 0.24 mmol) was added to a stirring solution of (S)-isopropyl 2-(5-(2-(benzofuro[3,2-d]pyrimidin-4-yl)-1,2,3,4-tetrahydroisoquinolin-6-yl)-4-(4,4-dimethylpiperidin-1-yl)-6-formyl-2-methylpyridin-3-yl)-2-(tert-butoxy)acetate (17 mg, 0.024 mmol) in EtOH (2 mL) at rt. Next, AcOH (7.0 μl, 0.12 mmol) was added, and the solution was allowed to stir at rt for 8 h. Next, sodium triacetoxyborohydride (10 mg, 0.048 mmol) was added. The mixture was stirred for an additional 1 h. LCMS shows conversion to the desired reductive amination product. The mixture was adsorbed onto Celite and then purified on silica gel eluting with a 0-100% hexanes in EtOAc gradient to give the desired amine as a colorless residue. This residue was taken up in EtOH (1 mL) and 5M NaOH (0.048 mL, 0.24 mmol) was added. The mixture was heated to 85° C. and stirred at this temp overnight. The mixture was then filtered and purified via preparative LC/MS to afford (S)-2-(5-(2-(benzofuro[3,2-d]pyrimidin-4-yl)-1,2,3,4-tetrahydroisoquinolin-6-yl)-4-(4,4-dimethylpiperidin-1-yl)-6-((4,4-dimethylpiperidin-1-yl)methyl)-2-methylpyridin-3-yl)-2-(tert-butoxy)acetic acid (7 mg, 9% yield). LCMS (M+H)=759.14.

### Example 162

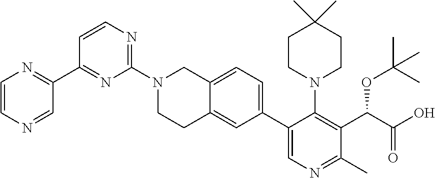 [SMILES: Cc1ncc(-c2ccc3c(c2)CCN(c2nccc(-c4cnccn4)n2)C3)c(N2CCC(C)(C)CC2)c1[C@H](OC(C)(C)C)C(=O)O]

### (S)-2-(tert-Butoxy)-2-(4-(4,4-dimethylpiperidin-1-yl)-2-methyl-5-(2-(4-(pyrazin-2-yl)pyrimidin-2-yl)-1,2,3,4-tetrahydroisoquinolin-6-yl)pyridin-3-yl)acetic Acid

In a pressure vial equipped with a magnetic stirring bar was added isopropyl (S)-2-(5-bromo-4-(4,4-dimethylpiperidin-1-yl)-2-methylpyridin-3-yl)-2-(tert-butoxy)acetate (50 mg, 0.110 mmol) and 2-(4-(pyrazin-2-yl)pyrimidin-2-yl)-6-(4,4,5,5-tetramethyl-1,3,2-dioxaborolan-2-yl)-1,2,3,4-tetrahydroisoquinoline (114 mg, 0.165 mmol). The solids were suspended in distilled THF (5 mL). The mixture was treated with 0.5 M K₃PO₄ (0.768 mL, 0.384 mmol) and X-Phos precatalyst G2 (7.34 mg, 9.33 μmol). Argon was streamed over and bubbled into the mixture for 5 minutes with sonication. The vial was capped and stirred at RT for 48 hours. LC/MS showed the desired ester intermediate. Removed water layer and then removed solvent under vacuum. The residue was taken up in ethanol (2 mL). 10 M sodium hydroxide (0.110 mL, 1.098 mmol) was added to the mixture and the vial capped and heated to 80° C. for 16 hours. LC/MS showed the hydrolysis product. The reaction mixture was purified via preparative LC/MS to give 13.2 mg (18%) of the title compound. LCMS (M+H)=620.3. ¹H NMR (500 MHz, DMSO-d₆) δ 9.66 (s, 1H), 8.86-8.74 (m, 2H), 8.64 (d, J=4.8 Hz, 1H), 8.05 (s, 1H), 7.52 (d, J=5.1 Hz, 1H), 7.48-7.34 (m, 1H), 7.17 (s, 1H), 7.18 (d, J=5.5 Hz, 2H), 5.82 (s, 1H), 5.12-5.01 (m, 2H), 4.16 (q, J=6.1 Hz, 2H), 3.01 (br d, J=3.3 Hz, 3H), 1.91 (s, 1H), 1.37-1.21 (m, 4H), 1.13 (s, 12H), 0.90-0.66 (m, 8H).

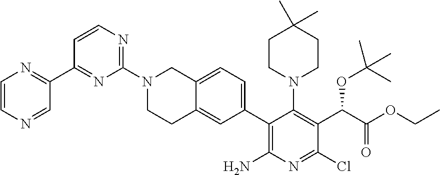 [SMILES: CCOC(=O)[C@@H](OC(C)(C)C)c1c(Cl)nc(N)c(-c2ccc3c(c2)CCN(c2nccc(-c4cnccn4)n2)C3)c1N1CCC(C)(C)CC1]

### Ethyl (S)-2-(6-amino-2-chloro-4-(4,4-dimethylpiperidin-1-yl)-5-(2-(4-(pyrazin-2-yl)pyrimidin-2-yl)-1,2,3,4-tetrahydroisoquinolin-6-yl)pyridin-3-yl)-2-(tert-butoxy)acetate

To a dry reaction vial under nitrogen was added (S)-ethyl 2-(6-amino-5-bromo-2-chloro-4-(4,4-dimethylpiperidin-1-yl)pyridin-3-yl)-2-(tert-butoxy)acetate (27 mg, 0.057 mmol), 2-(4-(pyrazin-2-yl)pyrimidin-2-yl)-6-(4,4,5,5-tetramethyl-1,3,2-dioxaborolan-2-yl)-1,2,3,4-tetrahydroisoquinoline (75 mg, 0.181 mmol) and THF (5 mL). The reaction is flushed with argon, treated with 0.5 M potassium phosphate tribasic (570 μL, 0.285 mmol), 2^{nd} generation X-phos precatalyst (5 mg, 6.35 μmol), capped, and stirred at room temp for 20 h. The reaction was diluted with ethyl acetate (75 mL), extracted with water (1×8 mL), brine (1×8 mL), dried over Na₂SO₄ and concentrated. The crude material was purified via silica gel chromatography (24 g SiO₂ column, CH₂Cl₂:EtOAc 100:0->0:100) to afford (S)-ethyl 2-(6-amino-2-chloro-4-(4,4-dimethylpiperidin-1-yl)-5-(2-(4-(pyrazin-2-yl)pyrimidin-2-yl)-1,2,3,4-tetrahydroisoquinolin-6-yl)pyridin-3-yl)-2-(tert-butoxy)acetate, 24.5 mg (63%). LCMS=685.4 (M+H).

### Example 163

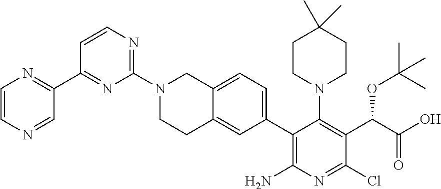 [SMILES: CC1(C)CCN(c2c(-c3ccc4c(c3)CCN(c3nccc(-c5cnccn5)n3)C4)c(N)nc(Cl)c2[C@H](OC(C)(C)C)C(=O)O)CC1]

### (S)-2-(6-Amino-2-chloro-4-(4,4-dimethylpiperidin-1-yl)-5-(2-(4-(pyrazin-2-yl)pyrimidin-2-yl)-1,2,3,4-tetrahydroisoquinolin-6-yl)pyridin-3-yl)-2-(tert-butoxy)acetic Acid

To a solution of ethyl (S)-2-(6-amino-2-chloro-4-(4,4-dimethylpiperidin-1-yl)-5-(2-(4-(pyrazin-2-yl)pyrimidin-2-yl)-1,2,3,4-tetrahydroisoquinolin-6-yl)pyridin-3-yl)-2-(tert-butoxy)acetate (24.5 mg, 0.036 mmol) in EtOH (4 mL) was added 10 M sodium hydroxide (50 μL, 0.500 mmol). The reaction is flushed briefly with nitrogen, capped and heated at 105° C. for 3.5 h. The reaction was treated with additional 10 M sodium hydroxide (25 mL, 0.250 mmol) and heated at 105° C. for 3.5 h. The crude material was purified via preparative LCMS to afford (S)-2-(6-amino-2-chloro-4-(4,4-dimethylpiperidin-1-yl)-5-(2-(4-(pyrazin-2-yl)pyrimidin-2-yl)-1,2,3,4-tetrahydroisoquinolin-6-yl)pyridin-3-yl)-2-(tert-butoxy)acetic acid, 8.8 mg (37%). LCMS=657.2 (M+H).

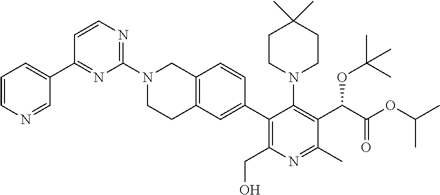 [SMILES: Cc1nc(CO)c(-c2ccc3c(c2)CCN(c2nccc(-c4cccnc4)n2)C3)c(N2CCC(C)(C)CC2)c1[C@H](OC(C)(C)C)C(=O)OC(C)C]

### Isopropyl (S)-2-(tert-butoxy)-2-(4-(4,4-dimethylpiperidin-1-yl)-6-(hydroxymethyl)-2-methyl-5-(2-(4-(pyridin-3-yl)pyrimidin-2-yl)-1,2,3,4-tetrahydroisoquinolin-6-yl)pyridin-3-yl)acetate

To a dry reaction vial under nitrogen was added (S)-isopropyl 2-(5-bromo-4-(4,4-dimethylpiperidin-1-yl)-6-(hydroxymethyl)-2-methylpyridin-3-yl)-2-(tert-butoxy)acetate (50 mg, 0.103 mmol), 2-(4-(pyridin-3-yl)pyrimidin-2-yl)-6-(4,4,5,5-tetramethyl-1,3,2-dioxaborolan-2-yl)-1,2,3,4-tetrahydroisoquinoline (60 mg, 0.145 mmol) and THF (5 mL). The reaction is flushed with argon, then treated with 0.5 M potassium phosphate tribasic water (824 μL, 0.412 mmol), followed by 2nd generation X-phos precatalyst (7.8 mg, 9.91 μmol). The reaction is flushed with argon, capped and stirred at room temp for 65 h. The reaction was diluted with EtOAc (85 mL) and extracted water (1×5 mL), brine (1×6 mL), dried over Na₂SO₄ and concentrated. The crude material was purified via silica gel chromatography (24 g SiO₂ column, CH₂Cl₂:EtOAc 100:0->0:100) to afford (S)-isopropyl 2-(tert-butoxy)-2-(4-(4,4-dimethylpiperidin-1-yl)-6-(hydroxymethyl)-2-methyl-5-(2-(4-(pyridin-3-yl)pyrimidin-2-yl)-1,2,3,4-tetrahydroisoquinolin-6-yl)pyridin-3-yl)acetate, 69.3 mg (97%). LCMS=693.4 (M+H).

### Example 164

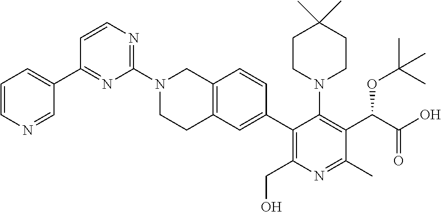 [SMILES: Cc1nc(CO)c(-c2ccc3c(c2)CCN(c2nccc(-c4cccnc4)n2)C3)c(N2CCC(C)(C)CC2)c1[C@H](OC(C)(C)C)C(=O)O]

### (S)-2-(tert-Butoxy)-2-(4-(4,4-dimethylpiperidin-1-yl)-6-(hydroxymethyl)-2-methyl-5-(2-(4-(pyridin-3-yl)pyrimidin-2-yl)-1,2,3,4-tetrahydroisoquinolin-6-yl)pyridin-3-yl)acetic Acid

To a solution of isopropyl (S)-2-(tert-butoxy)-2-(4-(4,4-dimethylpiperidin-1-yl)-6-(hydroxymethyl)-2-methyl-5-(2-(4-(pyridin-3-yl)pyrimidin-2-yl)-1,2,3,4-tetrahydroisoquinolin-6-yl)pyridin-3-yl)acetate (69.3 mg, 0.100 mmol) in ethanol (4 mL) was added 2 M lithium hydroxide (300 μL, 0.600 mmol). The reaction is flushed briefly with N₂, capped and heated at 105° C. for 30 min. The reaction was then treated with 10 M sodium hydroxide (150 μL, 1.500 mmol), flush with N₂, and heated at 105° C. sand bath for 2 h, followed by room temp for 18 h. The reaction was treated with additional 10 M NaOH (50 μlit, 0.050 mmol) and heated at 105° C. for 3.5 h. The crude material was purified via preparative LCMS to afford (S)-2-(tert-butoxy)-2-(4-(4,4-dimethylpiperidin-1-yl)-6-(hydroxymethyl)-2-methyl-5-(2-(4-(pyridin-3-yl)pyrimidin-2-yl)-1,2,3,4-tetrahydroisoquinolin-6-yl)pyridin-3-yl)acetic acid, 20.9 mg (32%). LC/MS=651.4 (M+H).

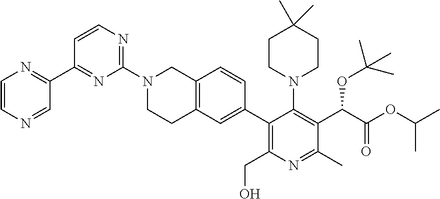 [SMILES: Cc1nc(CO)c(-c2ccc3c(c2)CCN(c2nccc(-c4cnccn4)n2)C3)c(N2CCC(C)(C)CC2)c1[C@H](OC(C)(C)C)C(=O)OC(C)C]

### (S)-Isopropyl 2-(tert-butoxy)-2-(4-(4,4-dimethylpiperidin-1-yl)-6-(hydroxymethyl)-2-methyl-5-(2-(4-(pyrazin-2-yl)pyrimidin-2-yl)-1,2,3,4-tetrahydroisoquinolin-6-yl)pyridin-3-yl)acetate

To a dry reaction flask under nitrogen was added isopropyl (S)-2-(5-bromo-4-(4,4-dimethylpiperidin-1-yl)-6-(hydroxymethyl)-2-methylpyridin-3-yl)-2-(tert-butoxy)acetate (200 mg, 0.412 mmol), 2-(4-(pyrazin-2-yl)pyrimidin-2-yl)-6-(4,4,5,5-tetramethyl-1,3,2-dioxaborolan-2-yl)-1,2,3,4-tetrahydroisoquinoline (290 mg, 0.698 mmol) and THF (50 mL). The reaction was flushed well with argon, treated with 0.5 M potassium phosphate tribasic (5 mL, 2.500 mmol), 2^{nd} generation X-phos precatalyst (40 mg, 0.051 mmol), capped and stirred at room temp for 18 h. Diluted reaction with ethyl acetate, extracted the organic layer with water and brine, dried over Na₂SO₄ and concentrated. The crude material was purified via silica gel chromatography (80 g SiO₂ column, dichloromethane:EtOAc 100:0->0:100) to afford isopropyl (S)-isopropyl 2-(tert-butoxy)-2-(4-(4,4-dimethylpiperidin-1-yl)-6-(hydroxymethyl)-2-methyl-5-(2-(4-(pyrazin-2-yl)pyrimidin-2-yl)-1,2,3,4-tetrahydroisoquinolin-6-yl)pyridin-3-yl)acetate, 48 mg (12%). LCMS=694.4 (M+H).

### Example 165

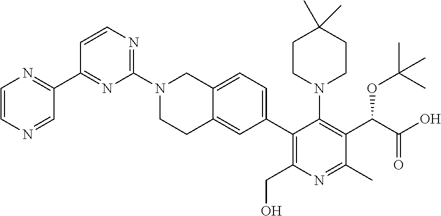 [SMILES: Cc1nc(CO)c(-c2ccc3c(c2)CCN(c2nccc(-c4cnccn4)n2)C3)c(N2CCC(C)(C)CC2)c1[C@H](OC(C)(C)C)C(=O)O]

### (S)-2-(tert-Butoxy)-2-(4-(4,4-dimethylpiperidin-1-yl)-6-(hydroxymethyl)-2-methyl-5-(2-(4-(pyrazin-2-yl)pyrimidin-2-yl)-1,2,3,4-tetrahydroisoquinolin-6-yl)pyridin-3-yl)acetic Acid

To a solution of isopropyl (S)-2-(tert-butoxy)-2-(4-(4,4-dimethylpiperidin-1-yl)-6-(hydroxymethyl)-2-methyl-5-(2-(4-(pyrazin-2-yl)pyrimidin-2-yl)-1,2,3,4-tetrahydroisoquinolin-6-yl)pyridin-3-yl)acetate (15 mg, 0.022 mmol) in EtOH (2 mL) was added 10 M sodium hydroxide (35 μl, 0.350 mmol) and the reaction was heated to 100° C. for 18 h. The reaction was treated with additional 10M sodium hydroxide (35 μl, 0.350 mmol) and heated to 100° C. for an additional 5 h. The crude material was purified via preparative LCMS to afford (S)-2-(tert-butoxy)-2-(4-(4,4-dimethylpiperidin-1-yl)-6-(hydroxymethyl)-2-methyl-5-(2-(4-(pyrazin-2-yl)pyrimidin-2-yl)-1,2,3,4-tetrahydroisoquinolin-6-yl)pyridin-3-yl)acetic acid, 3.5 mg (25%). LCMS=652.3 (M+H).

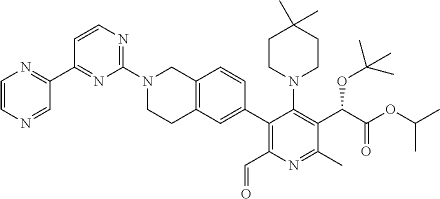 [SMILES: Cc1nc(C=O)c(-c2ccc3c(c2)CCN(c2nccc(-c4cnccn4)n2)C3)c(N2CCC(C)(C)CC2)c1[C@H](OC(C)(C)C)C(=O)OC(C)C]

### Isopropyl (S)-2-(tert-butoxy)-2-(4-(4,4-dimethylpiperidin-1-yl)-6-formyl-2-methyl-5-(2-(4-(pyrazin-2-yl)pyrimidin-2-yl)-1,2,3,4-tetrahydroisoquinolin-6-yl)pyridin-3-yl)acetate

To a magnetically stirred solution of isopropyl (S)-2-(tert-butoxy)-2-(4-(4,4-dimethylpiperidin-1-yl)-6-(hydroxymethyl)-2-methyl-5-(2-(4-(pyrazin-2-yl)pyrimidin-2-yl)-1,2,3,4-tetrahydroisoquinolin-6-yl)pyridin-3-yl)acetate (33 mg, 0.048 mmol) in CH₂Cl₂ (1.5 mL) and acetonitrile (0.1 mL) was added solid Dess-Martin periodinane (50 mg, 0.118 mmol) and the reaction was stirred at room temp 4 h. The reaction was diluted with Et₂O (100 mL) and extracted with 1N NaOH (1×50 mL), brine (1×40 mL), dried over Na₂SO₄ and concentrated to give isopropyl (S)-2-(tert-butoxy)-2-(4-(4,4-dimethylpiperidin-1-yl)-6-formyl-2-methyl-5-(2-(4-(pyrazin-2-yl)pyrimidin-2-yl)-1,2,3,4-tetrahydroisoquinolin-6-yl)pyridin-3-yl)acetate, 32 mg (97%) that was used in subsequent reactions without further purification. LCMS=723.6 (M+32).

### Example 166

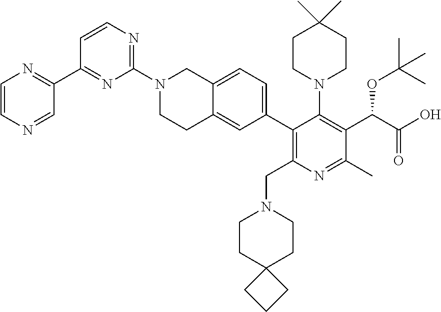 [SMILES: Cc1nc(CN2CCC3(CCC3)CC2)c(-c2ccc3c(c2)CCN(c2nccc(-c4cnccn4)n2)C3)c(N2CCC(C)(C)CC2)c1[C@H](OC(C)(C)C)C(=O)O]

### (S)-2-(6-(7-Azaspiro[3.5]nonan-7-ylmethyl)-4-(4,4-dimethylpiperidin-1-yl)-2-methyl-5-(2-(4-(pyrazin-2-yl)pyrimidin-2-yl)-1,2,3,4-tetrahydroisoquinolin-6-yl)pyridin-3-yl)-2-(tert-butoxy)acetic Acid

To a pressure vial containing isopropyl (S)-2-(tert-butoxy)-2-(4-(4,4-dimethylpiperidin-1-yl)-6-formyl-2-methyl-5-(2-(4-(pyrazin-2-yl)pyrimidin-2-yl)-1,2,3,4-tetrahydroisoquinolin-6-yl)pyridin-3-yl)acetate (32 mg, 0.046 mmol) and 7-azaspiro[3.5]nonane (35 mg, 0.280 mmol) was added ClCH₂CH₂Cl (2 mL), acetic acid (14 μl, 0.245 mmol), and 4 pieces of activated 4 A° molecular sieves. The reaction was stirred at room temp for 10 min, then treated with ethanol (1 mL) (previously dried over 4 A° molecular sieves). The reaction was stirred at room temp for 30 min, then treated (very slowly, dropwise, over 3.5 h) with 1 M sodium cyanoborohydride in THF (0.25 mL, 0.250 mmol). After the addition was complete, the solvent was removed under a gentle stream of N₂ and the residue was redissolved in EtOH (2.0 mL). The reaction was then treated with sodium hydroxide 10 M (100 μl, 1.000 mmol) and heated at 100° C. for 18 h. The crude material was purified via preparative LCMS to afford (S)-2-(6-((7-azaspiro[3.5]nonan-7-yl)methyl)-4-(4,4-dimethylpiperidin-1-yl)-2-methyl-5-(2-(4-(pyrazin-2-yl)pyrimidin-2-yl)-1,2,3,4-tetrahydroisoquinolin-6-yl)pyridin-3-yl)-2-(tert-butoxy)acetic acid, 7.1 mg (19%). LCMS=759.4 (M+H).

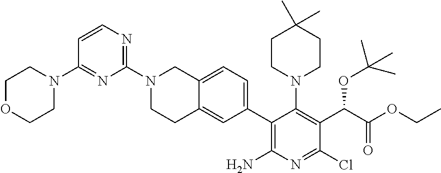 [SMILES: CCOC(=O)[C@@H](OC(C)(C)C)c1c(Cl)nc(N)c(-c2ccc3c(c2)CCN(c2nccc(N4CCOCC4)n2)C3)c1N1CCC(C)(C)CC1]

### Ethyl (S)-2-(6-amino-2-chloro-4-(4,4-dimethylpiperidin-1-yl)-5-(2-(4-morpholinopyrimidin-2-yl)-1,2,3,4-tetrahydroisoquinolin-6-yl)pyridin-3-yl)-2-(tert-butoxy)acetate

To a dry reaction vial under nitrogen was added ethyl (S)-2-(6-amino-5-bromo-2-chloro-4-(4,4-dimethylpiperidin-1-yl)pyridin-3-yl)-2-(tert-butoxy)acetate (34 mg, 0.071 mmol), 4-(2-(6-(4,4,5,5-tetramethyl-1,3,2-dioxaborolan-2-yl)-3,4-dihydroisoquinolin-2(1H)-yl)pyrimidin-4-yl)morpholine (90 mg, 0.213 mmol) and THF (5 mL). The reaction is flushed with argon, then treated with 0.5 M potassium phosphate tribasic (700 μL, 0.350 mmol), followed by 2^{nd} generation X-phos precatalyst (6 mg, 7.63 μmol). The reaction is again flushed with argon, capped, and stirred at room temp for 18 h. The reaction was diluted with ethyl acetate (75 mL), extracted with water (1×8 mL), brine (1×8 mL), dried over Na₂SO₄ and concentrated. The crude material was purified via silica gel chromatography (24 g SiO₂ column, CH₂Cl₂:EtOAc 100:0->0:100) to afford ethyl (S)-2-(6-amino-2-chloro-4-(4,4-dimethylpiperidin-1-yl)-5-(2-(4-morpholinopyrimidin-2-yl)-1,2,3,4-tetrahydroisoquinolin-6-yl)pyridin-3-yl)-2-(tert-butoxy)acetate, 44 mg (89%). LC/MS=692.4 (M+H).

### Example 167

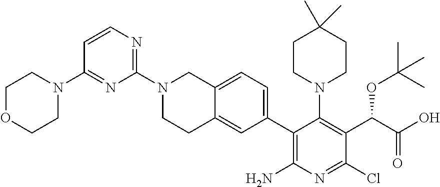 [SMILES: CC1(C)CCN(c2c(-c3ccc4c(c3)CCN(c3nccc(N5CCOCC5)n3)C4)c(N)nc(Cl)c2[C@H](OC(C)(C)C)C(=O)O)CC1]

### (S)-2-(6-Amino-2-chloro-4-(4,4-dimethylpiperidin-1-yl)-5-(2-(4-morpholinopyrimidin-2-yl)-1,2,3,4-tetrahydroisoquinolin-6-yl)pyridin-3-yl)-2-(tert-butoxy)acetic Acid

To a dry 20 mL pressure vial under N₂ was added ethyl (S)-2-(6-amino-2-chloro-4-(4,4-dimethylpiperidin-1-yl)-5-(2-(4-morpholinopyrimidin-2-yl)-1,2,3,4-tetrahydroisoquinolin-6-yl)pyridin-3-yl)-2-(tert-butoxy)acetate (44 mg, 0.064 mmol), ethanol (4.5 mL) and 10 M sodium hydroxide (95 μL, 0.950 mmol). The reaction was flushed briefly with _{N}2, capped and heated at 105° C. for 8 h. The crude material was purified via preparative LCMS to afford (S)-2-(tert-butoxy)-2-(4-(4,4-dimethylpiperidin-1-yl)-6-(hydroxymethyl)-2-methyl-5-(2-(4-(pyridin-3-yl)pyrimidin-2-yl)-1,2,3,4-tetrahydroisoquinolin-6-yl)pyridin-3-yl)acetic acid, 32.3 mg (75%). LCMS=664.3 (M+H).

The following compounds could be prepared by anyone skilled in the art by following the procedures described herewith for the examples listed herein or similar procedures from the literature.

### Example 168

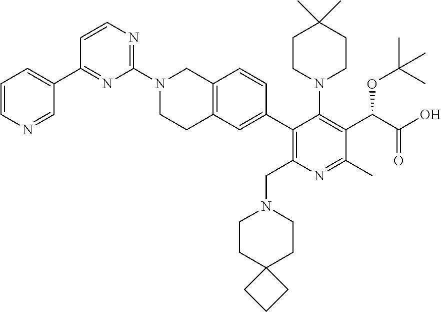 [SMILES: Cc1nc(CN2CCC3(CCC3)CC2)c(-c2ccc3c(c2)CCN(c2nccc(-c4cccnc4)n2)C3)c(N2CCC(C)(C)CC2)c1[C@H](OC(C)(C)C)C(=O)O]

### (S)-2-(6-((7-Azaspiro[3.5]nonan-7-yl)methyl)-4-(4,4-dimethylpiperidin-1-yl)-2-methyl-5-(2-(4-(pyridin-3-yl)pyrimidin-2-yl)-1,2,3,4-tetrahydroisoquinolin-6-yl)pyridin-3-yl)-2-(tert-butoxy)acetic Acid

### Example 169

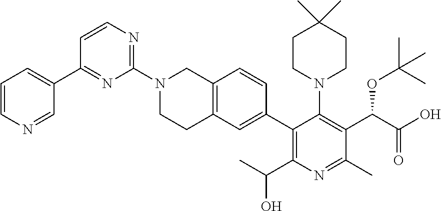 [SMILES: Cc1nc(C(C)O)c(-c2ccc3c(c2)CCN(c2nccc(-c4cccnc4)n2)C3)c(N2CCC(C)(C)CC2)c1[C@H](OC(C)(C)C)C(=O)O]

### (2S)-2-(tert-Butoxy)-2-(4-(4,4-dimethylpiperidin-1-yl)-6-(1-hydroxyethyl)-2-methyl-5-(2-(4-(pyridin-3-yl)pyrimidin-2-yl)-1,2,3,4-tetrahydroisoquinolin-6-yl)pyridin-3-yl)acetic Acid

### Example 170

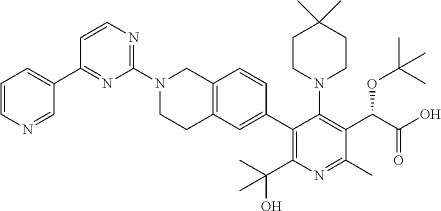 [SMILES: Cc1nc(C(C)(C)O)c(-c2ccc3c(c2)CCN(c2nccc(-c4cccnc4)n2)C3)c(N2CCC(C)(C)CC2)c1[C@H](OC(C)(C)C)C(=O)O]

### (S)-2-(tert-Butoxy)-2-(4-(4,4-dimethylpiperidin-1-yl)-6-(2-hydroxypropan-2-yl)-2-methyl-5-(2-(4-(pyridin-3-yl)pyrimidin-2-yl)-1,2,3,4-tetrahydroisoquinolin-6-yl)pyridin-3-yl)acetic Acid

### Example 171

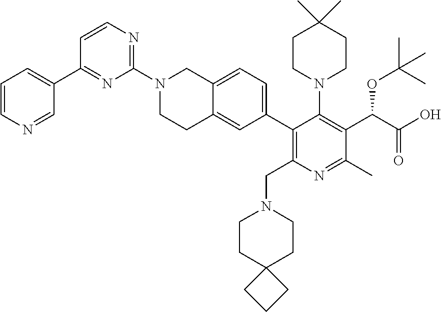 [SMILES: Cc1nc(CN2CCC3(CCC3)CC2)c(-c2ccc3c(c2)CCN(c2nccc(-c4cccnc4)n2)C3)c(N2CCC(C)(C)CC2)c1[C@H](OC(C)(C)C)C(=O)O]

### (S)-2-(6-(7-Azaspiro[3.5]nonan-7-ylmethyl)-4-(4,4-dimethylpiperidin-1-yl)-2-methyl-5-(2-(4-(pyridin-3-yl)pyrimidin-2-yl)-1,2,3,4-tetrahydroisoquinolin-6-yl)pyridin-3-yl)-2-(tert-butoxy)acetic Acid

### Example 172

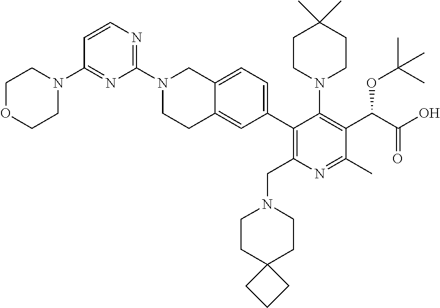 [SMILES: Cc1nc(CN2CCC3(CCC3)CC2)c(-c2ccc3c(c2)CCN(c2nccc(N4CCOCC4)n2)C3)c(N2CCC(C)(C)CC2)c1[C@H](OC(C)(C)C)C(=O)O]

### (S)-2-(6-(7-Azaspiro[3.5]nonan-7-ylmethyl)-4-(4,4-dimethylpiperidin-1-yl)-2-methyl-5-(2-(4-morpholinopyrimidin-2-yl)-1,2,3,4-tetrahydroisoquinolin-6-yl)pyridin-3-yl)-2-(tert-butoxy)acetic Acid

### Biological Methods

Inhibition of HIV Replication:

A recombinant NL-RLuc proviral clone was constructed in which a section of the nef gene form NL4-3 was replaced with the Renilla Luciferase gene. This virus is fully infectious and can undergo multiple cycles of replication in cell culture. In addition, the luciferous reporter provides a simple and easy method for quantitating the extent of virus growth and consequently, the antiviral activity of test compounds. The plasmid pNLRLuc contains the proviral NL-Rluc DNA cloned into pUC18 at the PvuII site. The NL-RLuc virus was prepared by transfection of 293T cells with the plasmid pNLRLuc. Transfections were performed using the LipofectAMINE PLUS kit form Invitrogen (Carlsbad, Calif.) according to the manufacturer and the virus generated was titered in MT-2 cells. For susceptibility analyses, the titrated virus was used to infect MT-2 cells in the presence of compound, and after 5 days of incubation, cells were processed and quantitated for virus growth by the amount of expressed luciferase. Assay media was RPMI 1640 supplemented with 10% heat inactivated fetal bovine serum (FBS), 100 units/ml penicillin G/100 units/ml streptomycin, 10 mM HEPES buffer pH 7.55 and 2 mM L-glutamine. The results form at least 2 experiments were used to calculate the EC₅₀ values. Luciferase was quantitated using the Dual Luciferase kit form Promega (Madison, Wis.). Susceptibility of viruses to compounds was determined by incubation in the presence of serial dilutions of the compound. The 50% effective concentration (EC₅₀) was calculated by using the exponential form of the median effect equation where (Fa)=1/[1+(ED₅₀/drug conc.)^{m}](Johnson V A, Byington R T. Infectivity Assay. In Techniques in HIV Research. ed. Aldovini A, Walker B D. 71-76. New York: Stockton Press. 1990). Results are shown in Table 1.

TABLE 1

| Example | EC₅₀ μM |
|---|---|
| 1 | 0.010 |
| 2 | 0.004 |
| 3 | 0.005 |
| 4 | 0.004 |
| 5 | 0.002 |
| 6 | 0.018 |
| 7 | 0.013 |
| 8 | 0.002 |
| 9 | 0.002 |
| 10 | 0.002 |
| 11 | 0.002 |
| 12 | 0.001 |
| 13 | 0.002 |
| 14 | nd |
| 15 | 0.002 |
| 16 | 0.002 |
| 17 | 0.001 |
| 18 | 0.001 |
| 19 | 0.001 |
| 20 | 0.002 |
| 21 | 0.002 |
| 22 | 0.003 |
| 23 | 0.003 |
| 24 | 0.008 |
| 25 | 0.005 |
| 26 | 0.0008 |
| 27 | 0.002 |
| 28 | 0.002 |
| 29 | 0.004 |
| 30 | 0.001 |
| 31 | 0.002 |
| 32 | 0.001 |
| 33 | 0.005 |
| 34 | 0.005 |
| 35 | 0.003 |
| 36 | 0.003 |
| 37 | 0.001 |
| 38 | 0.003 |
| 39 | 0.005 |
| 40 | 0.004 |
| 41 | 0.003 |
| 42 | 0.004 |
| 43 | 0.003 |
| 44 | 0.027 |
| 45 | 0.008 |
| 46 | 0.009 |
| 47 | 0.001 |
| 48 | 0.0006 |
| 49 | 0.001 |
| 50 | 0.004 |
| 51 | 0.004 |
| 52 | 0.003 |
| 53 | 0.006 |
| 54 | 0.012 |
| 55 | 0.021 |
| 56 | 0.001 |
| 57 | 0.0005 |
| 58 | 0.008 |
| 59 | 0.003 |
| 60 | 0.0006 |
| 61 | 0.002 |
| 62 | 0.003 |
| 63 | 0.004 |
| 64 | 0.008 |
| 65 | 0.019 |
| 66 | 0.013 |
| 67 | 0.002 |
| 68 | 0.001 |
| 69 | 0.018 |
| 70 | 0.005 |
| 71 | 0.004 |
| 72 | 0.008 |
| 73 | 0.069 |
| 74 | 0.001 |
| 75 | 0.003 |
| 76 | 0.003 |
| 77 | 0.0006 |
| 78 | 0.005 |
| 79 | 0.011 |
| 80 | 0.380 |
| 81 | 0.004 |
| 82 | 0.015 |
| 83 | 0.005 |
| 84 | 0.006 |
| 85 | 0.039 |
| 86 | 0.004 |
| 87 | 0.005 |
| 88 | 0.025 |
| 89 | 0.003 |
| 90 | 0.004 |
| 91 | 0.009 |
| 92 | 0.0006 |
| 93 | 0.008 |
| 94 | 0.004 |
| 95 | 0.001 |
| 96 | 0.0003 |
| 97 | 0.002 |
| 98 | 0.0007 |
| 99 | 0.004 |
| 100 | 0.004 |
| 101 | 0.002 |
| 102 | 0.008 |
| 103 | 0.002 |
| 104 | 0.010 |
| 105 | 0.001 |
| 106 | 0.008 |
| 107 | 0.001 |
| 108 | 0.002 |
| 109 | 0.001 |
| 110 | 0.110 |
| 111 | 0.0007 |
| 112 | 0.0003 |
| 113 | 0.003 |
| 114 | 0.0001 |
| 115 | 0.001 |
| 116 | 0.004 |
| 117 | 0.001 |
| 118 | 0.001 |
| 119 | 0.005 |
| 120 | 0.014 |
| 121 | 0.009 |
| 122 | 0.005 |
| 123 | 0.011 |
| 124 | 0.017 |
| 125 | 0.023 |
| 126 | 0.005 |
| 127 | 1.275 |
| 128 | 0.002 |
| 129 | 0.012 |
| 130 | 0.005 |
| 131 | 0.004 |
| 132 | 0.002 |
| 133 | 0.003 |
| 134 | 0.004 |
| 135 | 0.019 |
| 136 | 0.016 |
| 137 | 0.007 |
| 138 | 0.002 |
| 139 | 0.004 |
| 140 | 0.035 |
| 141 | 0.002 |
| 142 | 0.381 |
| 143 | 0.014 |
| 144 | 0.003 |
| 145 | 0.010 |
| 146 | 0.0005 |
| 147 | 0.002 |
| 148 | 0.0002 |
| 149 | 0.002 |
| 150 | 0.0003 |
| 151 | 0.002 |
| 152 | 0.0001 |
| 153 | 0.002 |
| 154 | 0.097 |
| 155 | 0.055 |
| 156 | 0.065 |
| 157 | 0.340 |
| 158 | 0.005 |
| 159 | 0.001 |
| 160 | 0.020 |
| 161 | 0.001 |
| 162 | 0.002 |
| 163 | 0.003 |
| 164 | 0.002 |
| 165 | 0.002 |
| 166 | 0.0006 |
| 167 | 0.004 |
| 168 | nd |
| 169 | nd |
| 170 | nd |
| 171 | nd |
| 172 | nd |
| 173 | nd |

nd: Not determined

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

1. A compound of Formula I 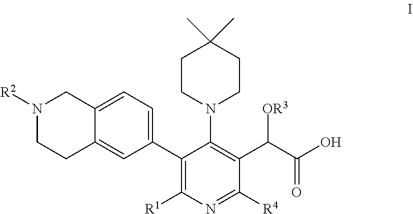
or a pharmaceutically acceptable salt thereof wherein:
R¹ is hydrogen, C₁₋₆alkyl, Ar¹, carboxy, cyano, hydroxy, C₁₋₆haloalkyl, —C₁₋₆alkyl-OH, —N(R⁵)(R⁶), —C(O)N(R⁷)(R⁸), or (R⁹)(R¹⁰)NC₁₋₆alkyl-;
R² is Ar³—C₁₋₆alkyl-, or Ar⁴;
R³ is C₁₋₆alkyl;
R⁴ is hydrogen, C₁₋₆alkyl, cyano, halo, C₁₋₆haloalkyl, or —C₁₋₆alkyl-OH;
R⁵ is hydrogen or C₁₋₆alkyl;
R⁶ is hydrogen, C₁₋₆alkyl, C₁₋₆alkyl-O—C₁₋₆alkyl, C₃₋₆cycloalkyl, C₃₋₆cycloalkyl-C₁₋₆alkyl-, 1-(C₁₋₆alkyl)piperidinyl-, (C₁₋₆alkyl)₂N—C₁₋₆alkyl-, (tetrahydropyranyl)C₁₋₆alkyl-, morpholinoC₁₋₆alkyl-, piperidinylC₁₋₆alkyl-, 1-(C₁₋₆alkyl)piperazinylC₁₋₆alkyl-, Ar²—C₁₋₆alkyl-, or 1-(C₁₋₆alkyl sulfonyl)piperidinyl-;
R⁷ is hydrogen or C₁₋₆alkyl;
R⁸ is hydrogen, C₁₋₆alkyl, C₃₋₆cycloalkyl, or C₁₋₆alkyl-C₃₋₆cycloalkyl-;
R⁹ is hydrogen or C₁₋₆alkyl;
R¹⁰ is hydrogen, C₁₋₆alkyl, Ar³—C₁₋₆alkyl-, or (tetrahydropyranyl) C₁₋₆alkyl-;
R¹¹ is azaspirononanyl, azetidinyl, 1,4-diazabicyclo[3.2.2]nonanyl, 3,8-diazabicyclo[3.2.1]octanyl, 3,7-dioxa-9-azabicyclo[3.3.1]nonanyl, 1,1-dioxidothiomorpholinyl, imidazolyl, morpholinyl, 3-oxa-9-azabicyclo[3.3.1]nonanyl, 8-oxa-3-azabicyclo[3.2.1]octanyl, oxadiazolyl, phenyl, piperazinyl, piperidinyl, pyrazinyl, pyridinyl, pyrrolidinyl, ((R⁵)(R⁹)NC₁₋₆alkyl)(R⁵)N—, (R⁵)(R⁹)N—, and is substituted with 0-3 substituents selected from C₁₋₆alkyl, —O—C₁₋₆alkyl, halo, C₁₋₆haloalky, —C₁₋₆alkyl-OH, morpholinyl, piperazinyl, or piperidinyl;
(R⁷)(R⁸)N taken together form an azetidinyl, pyrrolidinyl, piperidinyl, 1,1-dioxidothiomorpholinyl, or morpholinyl and is substituted with 0-3 C₁₋₆alkyl substituents;
(R⁹)(R¹⁰)N taken together form an azetidinyl, pyrrolidinyl, piperidinyl, or azaspirononanyl, and is substituted with 0-3 C₁₋₆alkyl substitutents;
Ar¹ is selected from imidazolyl, pyrazolyl, pyridinyl, pyrimidinyl, pyrrolyl and is substituted with 0-3 substitutents selected from amino, C₁₋₆alkyl, and C₃₋₆cycloalkyl;
Ar² is selected from imidazolyl, pyrazolyl, pyridinyl, pyrimidinyl, and pyrrolyl, substituted with 0-3 C₁₋₆alkyl and halo substitutents;
Ar³ is phenyl, and is substituted with 0-3 substituents selected from C₁₋₆alkyl, —O—C₁₋₆alkyl, cyano, halo, or C₁₋₆haloalkyl; and
Ar⁴ is selected from benzofuropyrimidinyl, pyrazinyl, pyridazinyl, pyridinyl, pyridofuropyrimidinyl, pyrimidinyl, pyrrolotriazinyl, triazinyl and is substituted with 0-3 substituents selected from R¹¹, C₁₋₆alkyl, —O—C₁₋₆alkyl, —CO₂H, cyano, halo, C₁₋₆haloalkyl, or hydroxy;
and wherein each reference to “haloalkyl includes all halogenated isomers from monohalo to perhalo.

2. A compound or salt according to claim 1 wherein R¹ is hydrogen, C₁₋₆alkyl, C₁₋₆haloalkyl, —C₁₋₆alkyl-OH, —N(R⁵)(R⁶), or (R⁹)(R¹⁰)NC₁₋₆alkyl-.

3. A compound or salt according to claim 2 wherein R¹ is hydrogen or (R⁹)(R¹⁰)NC₁₋₆alkyl-.

4. A compound or salt according to claim 1 wherein R² is Ar³—C₁₋₆alkyl- wherein Ar³ is as defined above; or Ar⁴ wherein Ar⁴ is selected from benzofuropyrimidinyl, pyrazinyl, pyridinyl, pyridofuropyrimidinyl, or pyrimidinyl, and is substituted with 0-3 substituents selected from R¹¹, C₁₋₆alkyl, —O—C₁₋₆alkyl, —CO₂H, cyano, halo, C₁₋₆haloalkyl, or hydroxy wherein R¹¹ is as defined above.

5. A compound or salt according to claim 4 wherein R² is Ar³—C₁₋₆alkyl- wherein Ar³ is as defined above; or Ar⁴ wherein Ar⁴ is selected from benzofuropyrimidinyl, pyridinyl, or pyridofuropyrimidinyl, and is substituted with 0-3 substituents selected from R¹¹, C₁₋₆alkyl, —O—C₁₋₆alkyl, —CO₂H, cyano, halo, C₁₋₆haloalkyl, or hydroxy.

6. A pharmaceutical composition comprising a compound or salt according to claim 1.

7. The composition of claim 6 further comprising at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier.

8. A method for treating HIV infection comprising administering a composition according to claim 6 to a patient in need thereof.

9. The method of claim 8 further comprising administering at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

10-12. (canceled)
